(19) United States
(12) Patent Application Publication
(10) Pub. No.: US 2023/0312481 A1
(43) Pub. Date: Oct. 5, 2023

(54) SUBSTITUTED (PHTHALAZIN-1-YLMETHYL)UREAS, SUBSTITUTED N-(PHTHALAZIN-1-YLMETHYL)AMIDES, AND ANALOGUES THEREOF

(71) Applicant: Arbutus Biopharma Corporation, Burnaby (CA)

(72) Inventors: Andrew G. Cole, Cranbury, NJ (US); Bruce D. Dorsey, Ambler, PA (US); Benjamin J. Dugan, Glen Mills, PA (US); Yi Fan, Doylestown, PA (US); Steven G. Kultgen, Warminster, PA (US); Eugen F. Mesaros, Wallingford, PA (US); Michael J. Sofia, Doylestown, PA (US)

(21) Appl. No.: 18/008,859

(22) PCT Filed: Jun. 7, 2021
(86) PCT No.: PCT/IB2021/000393

§ 371 Date: Dec. 7, 2022

Related U.S. Application Data
(60) Provisional application No. 63/036,096, filed on Jun. 8, 2020.

(51) Int. Cl. C07D 237/32; C07D 403/12; C07D 471/04; C07D 495/04; C07D 417/12; C07D 413/12; C07D 401/12; A61K 45/06

(52) U.S. Cl. CPC C07D 237/32; A61K 45/06; C07D 401/12; C07D 403/12; C07D 413/12; C07D 417/12; C07D 471/04; C07D 495/04; C07B 2200/07

(57) ABSTRACT

The present disclosure includes certain substituted (phthalazin-1-ylmethyl)ureas, N-(phthalazin-1-ylmethyl)amides, and analogues thereof, and compositions comprising the same, that can be used to treat or prevent hepatitis B virus (HBV) and/or hepatitis D virus (HDV) infections in a patient.

### CROSS-REFERENCE TO RELATED APPLICATIONS

This application claims priority under 35 U.S.C. § 119(e) to U.S. Provisional Pat. Application No. 63/036,096, filed Jun. 8, 2020, which is incorporated herein by reference in its entirety.

### BACKGROUND

Hepatitis B is one of the world’s most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit hepatitis B virus (HBV) DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe, and effective therapies for chronic hepatitis B.

HBV is a noncytopathic, liver tropic DNA virus belonging to Hepadnaviridae family. Pregenomic (pg) RNA is the template for reverse transcriptional replication of HBV DNA. The encapsidation of pg RNA, together with viral DNA polymerase, into a nucleocapsid is essential for the subsequent viral DNA synthesis. Inhibition of pg RNA encapsidation may block HBV replication and provide a new therapeutic approach to HBV treatment. A capsid inhibitor acts by inhibiting the expression and/or function of a capsid protein either directly or indirectly: for example, it may inhibit capsid assembly, induce formation of non-capsid polymers, promote excess capsid assembly or misdirected capsid assembly, affect capsid stabilization, and/or inhibit RNA encapsidation. A capsid inhibitor may also act by inhibiting capsid function in one or more downstream events within the replication process, such as, but not limited to, viral DNA synthesis, transport of relaxed circular DNA (rcDNA) into the nucleus, covalently closed circular DNA (cccDNA) formation, virus maturation, budding and/or release.

Clinically, inhibition of pg RNA encapsidation, or more generally inhibition of nucleocapsid assembly, may offer certain therapeutic advantages. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV variants resistant to the currently available DNA polymerase inhibitors. In yet another aspect, combination therapy of the pg RNA encapsidation inhibitors with DNA polymerase inhibitors may synergistically suppress HBV replication and prevent drug resistance emergence, thus offering a more effective treatment for chronic hepatitis B infection.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa given weekly for 12 to 18 months is the only licensed treatment for hepatitis D. Response to this therapy is limited, as only about one-quarter of patients is serum HDV RNA undetectable 6 months post therapy.

Clinically, inhibition of pg RNA encapsidation, or more generally inhibition of nucleocapsid assembly, may offer certain therapeutic advantages for treatment of hepatitis B and/or hepatitis D. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV and/or HDV variants resistant to the currently available DNA polymerase inhibitors. In yet another aspect, combination therapy of the pg RNA encapsidation inhibitors with DNA polymerase inhibitors may synergistically suppress HBV and/or HDV replication and prevent drug resistance emergence, thus offering a more effective treatment for chronic hepatitis B and/or hepatis D infection.

There is thus a need in the art for the identification of novel compounds that can be used to treat and/or prevent HBV and/or HDV infection in a subject. In certain embodiments, the novel compounds inhibit HBV and/or HDV nucleocapsid assembly. In other embodiments, the novel compounds can be used in patients that are HBV and/or HBV-HDV infected, patients who are at risk of becoming HBV and/or HBV-HDV infected, and/or patients that are infected with drug-resistant HBV and/or HDV. The present disclosure addresses this need.

### BRIEF SUMMARY

The disclosure provides certain compounds of formula (Ia) or (Ib), or a salt, solvate, geometric isomer, stereoisomer, tautomer and any mixtures thereof, wherein the substituents in (Ia) and (Ib) are defined elsewhere herein:

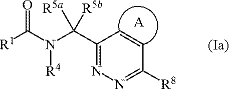

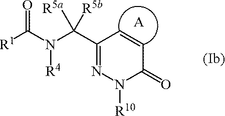

The disclosure further provides pharmaceutical compositions comprising at least one compound of the disclosure. In certain embodiments, the pharmaceutical compositions further comprise at least one pharmaceutically acceptable carrier. In other embodiments, the pharmaceutical compositions further comprise at least one additional agent that treats or prevents hepatitis virus infection. In yet other embodiments, the hepatitis virus is hepatitis B virus (HBV). In yet other embodiments, the hepatitis virus is hepatitis D virus (HDV).

The disclosure further provides a method of treating, ameliorating, and/or preventing hepatitis virus infection in a subject. In certain embodiments, the method comprises administering to the subject a therapeutically effective amount of a compound of the disclosure, or a salt, solvate, prodrug, stereoisomer, tautomer, or any mixtures thereof. In certain embodiments, the method comprises administering to the subject a therapeutically effective amount of the pharmaceutical composition of the present disclosure. In other embodiments, the subject is infected with HBV. In yet other embodiments, the subject is infected with HDV. In yet other embodiments, the subject is infected with HBV and HDV. In yet other embodiments, the subject is further administered at least one additional agent useful for treating, ameliorating, and/or preventing the hepatitis virus infection. In yet other embodiments, the subject is in need of the treatment or prevention.

The disclosure further provides a method of inhibiting expression and/or function of a viral capsid protein directly or indirectly. In certain embodiments, the method comprises administering to the subject a therapeutically effective amount of a compound of the disclosure, or a salt, solvate, prodrug, stereoisomer, tautomer, or any mixtures thereof. In certain embodiments, the method comprises administering to the subject a therapeutically effective amount of the pharmaceutical composition of the present disclosure. In other embodiments, the subject is infected with HBV. In yet other embodiments, the subject is infected with HDV. In yet other embodiments, the subject is infected with HBV and HDV. In yet other embodiments, the subject is further administered at least one additional agent useful for treating, ameliorating, and/or preventing the hepatitis virus infection.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawing(s). It should be understood, however, that the disclosure is not limited to the precise arrangements and instrumentalities of the embodiments shown in the drawings.

FIG. 1 provides the ORTEP representation of (R)-3-(3-chloro-4-fluorophenyl)-1-(1-(6,7-difluoro-4-oxo-3,4-dihydrophthalazin-1-yl)ethyl)-1-methylurea with 50% probability thermal ellipsoids displayed.

### DETAILED DESCRIPTION

The disclosure relates, in certain aspects, to the discovery of certain substituted (phthalazin-1-ylmethyl)ureas, N-(phthalazin-1-ylmethyl)amides, and analogues thereof, which are useful to treat and/or prevent hepatitis B virus (HBV) and/or hepatitis D virus (HDV) infection and related conditions in a subject. In certain embodiments, the compounds of the disclosure are viral capsid inhibitors.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂—phenyl or —CH₂—phenyl (or benzyl). Specific examples are aryl—CH₂— and aryl—CH(CH₃)—. The term “substituted aryl-(C₁-C₆)alkyl” refers to an aryl-(C₁-C₆)alkyl functional group in which the aryl group is substituted. A specific example is substituted aryl(CH₂)-. Similarly, the term “heteroaryl-(C₁-C₆)alkyl” refers to a functional group wherein a one-to-three carbon alkylene chain is attached to a heteroaryl group, e.g., —CH₂CH₂—pyridyl. A specific example is heteroaryl—(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl—(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject’s health continues to deteriorate.

As used herein, a “disorder” in a subject is a state of health in which the subject is able to maintain homeostasis, but in which the subject’s state of health is less favorable than it would be in the absence of the disorder. Left untreated, a disorder does not necessarily cause a further decrease in the subject’s state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer’s solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington’s Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), -SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: ACN, acetonitrile; AcOH, acetic acid; cccDNA, covalently closed circular DNA; DAD, diode array detector; DCE, 1,2-dichloroethane; DCM, dichloromethane; DIEA or DIPEA, diisopropylethylamine; DMF, N,N-dimethylformamide; DMSO, dimethylsulfoxide; EDCI, N-(3-dimethylaminopropyl)-N′ethylcarbodiimide; EtOAc, ethyl acetate; EtOH, ethanol; HATU, hexafluorophosphate azabenzotriazole tetramethyl uronium; HOBt, 1-hydroxybenzotriazole, HBsAg, HBV surface antigen; HBV, hepatitis B virus; HDV, hepatitis D virus; HPLC, high pressure liquid chromatography; IPA, isopropanol (2-propanol); LCMS, liquid chromatography mass spectrometry; LG, leaving group; MeOH, methanol; MeCN, acetonitrile; NARTI or NRTI, reverse-transcriptase inhibitor; NMR, Nuclear Magnetic Resonance; NtARTI or NtRTI, nucleotide analog reverse-transcriptase inhibitor; pg RNA, pregenomic RNA; rcDNA, relaxed circular DNA; RT, retention time; sAg, surface antigen; SEM, 2-(trimethylsilyl)ethoxymethyl; SEM-Cl, 2-(trimethylsilyl)ethoxymethyl chloride; SFC, supercritical fluid chromatography; STAB, sodium triacetoxyborohydride; TFA, trifluoroacetic acid; THF, tetrahydrofuran; TLC, thin layer chromatography; TMSOTf, trimethylsilyl trifluoromethylsulfonate.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds

The disclosure includes a compound of formula (Ia) or (Ib), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

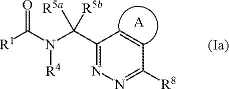

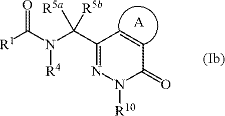

wherein in (Ia) or (Ib):
ring A

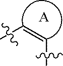

is selected from the group consisting of:

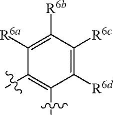

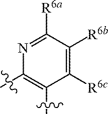

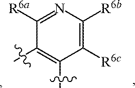

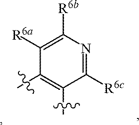

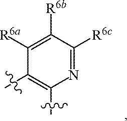

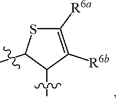

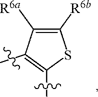

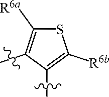

(wherein there is no bridgehead double bond in the bicyclic structure including ring A),

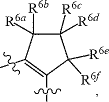

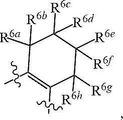

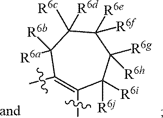

or ring A is absent and

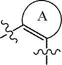

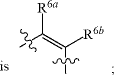

R¹ is selected from the group consisting of —NR²R³,

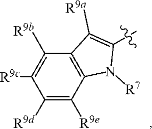

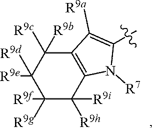

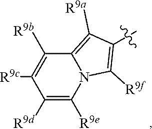

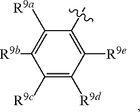

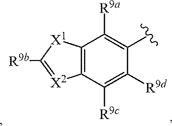

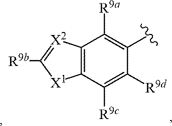

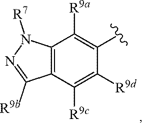

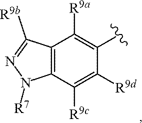

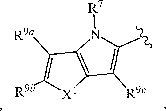

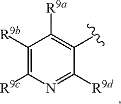

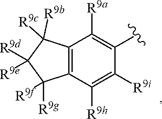

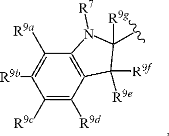

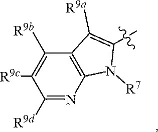

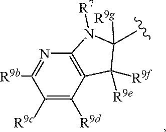

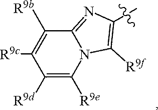

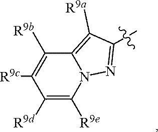

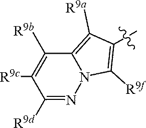

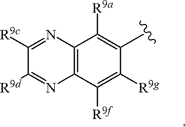

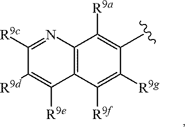

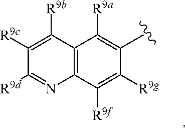

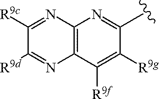

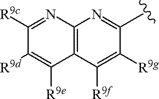

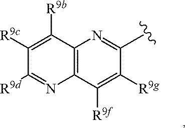

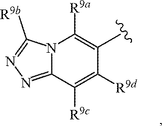

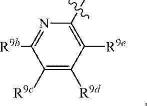

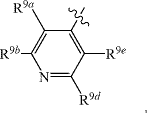

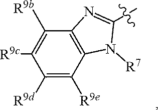

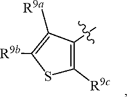

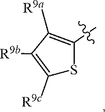

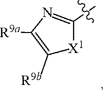

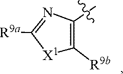

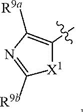

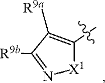

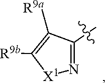

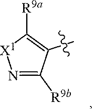

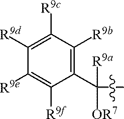

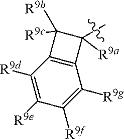

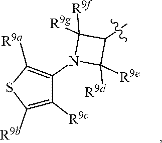

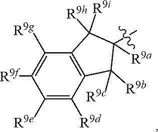

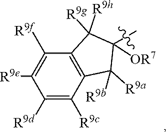

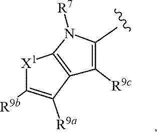

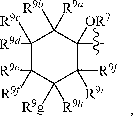

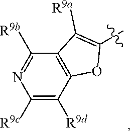

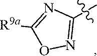

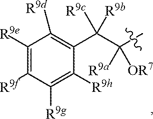

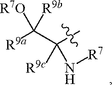

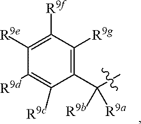

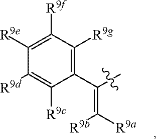

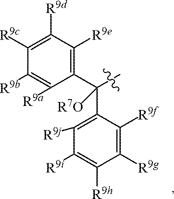

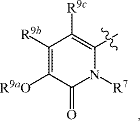

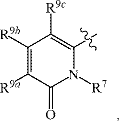

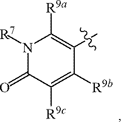

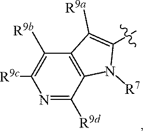

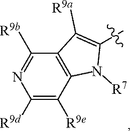

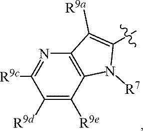

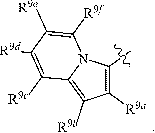

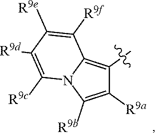

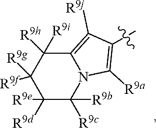

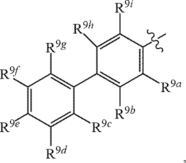

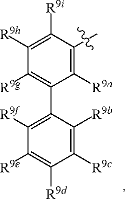

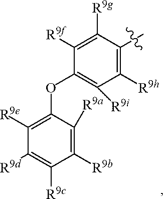

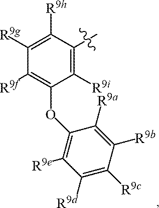

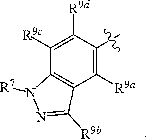

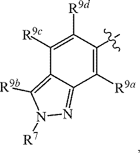

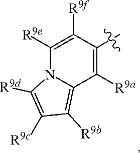

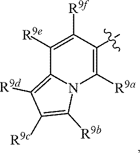

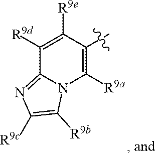

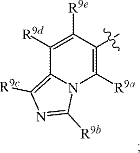

X¹ is selected from the group consisting of O, S, and N(R⁷);
X² is selected from the group consisting of N and CR^{9e};
R² is selected from the group consisting of optionally substituted C₃-C₈ cycloalkyl, optionally substituted phenyl, optionally substituted benzyl, optionally substituted heteroaryl, and -(CH₂)(optionally substituted heteroaryl);
R³ is selected from the group consisting of H and optionally substituted C₁-C₆ alkyl;
R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁷, —OC(═O)R⁷, —SR⁷, —S(═O)R⁷, —S(═O)₂R⁷, —S(═O)₂NR⁷R⁷, —S(═O)₂NHC(═O)NHR⁷, —N(R⁷)S(═O)₂R⁷, —N(R⁷)C(═O)R⁷, —C(═O)NR⁷R⁷, and —NR⁷R⁷;
R^{5a} is selected from the group consisting of H and optionally substituted C₁-C₆ alkyl;
R^{5b} is selected from the group consisting of H and optionally substituted C₁-C₆ alkyl;
each occurrence of R^{6a}, R^{6b}, R^{6c}, R^{6d}, R^{6e}, R^{6f}, R^{6g}, R^{6h}, R⁶ⁱ, and R^{6j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), -N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁷ is independently selected from the group consisting of H, optionally substituted C₁-C₆ alkyl, optionally substituted C₃-C₈ cycloalkyl, optionally substituted phenyl, and optionally substituted hetereoaryl;
R⁸ is selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, R^{9d}, R^{9e}, R^{9f}, R^{9g}, R^{9h}, R⁹ⁱ, and R^{9j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), -N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
R¹⁰ is selected from the group consisting of H, C₁-C₆ alkyl, optionally substituted C₃-C₈ cycloalkyl, optionally substituted C₁-C₆ alkoxy, and optionally substituted C₃-C₈ cycloalkoxy.

In certain embodiments, the compound of formula (Ia) is a compound of formula (Ia-1a):

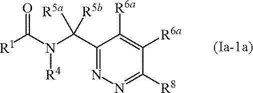

In certain embodiments, the compound of formula (Ia) is a compound of formula (Ia-1b):

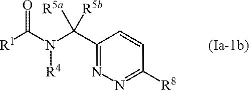

In certain embodiments, the compound of formula (Ia) is a compound of formula (Ia-2):

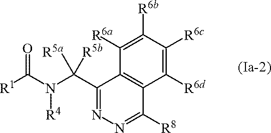

In certain embodiments, the compound of formula (Ia) is a compound of formula (Ia-3):

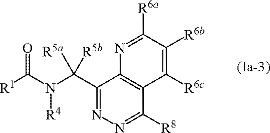

In certain embodiments, the compound of formula (Ia) is a compound of formula (Ia-4):

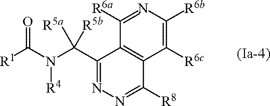

In certain embodiments, the compound of formula (Ia) is a compound of formula (Ia-5):

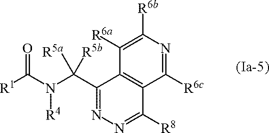

. In certain embodiments, the compound of formula (Ia) is a compound of formula (Ia-6):

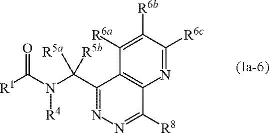

. In certain embodiments, the compound of formula (Ia) is a compound of formula (Ia-7):

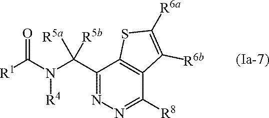

In certain embodiments, the compound of formula (Ia) is a compound of formula (Ia-8):

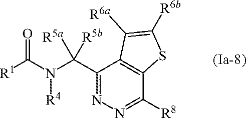

In certain embodiments, the compound of formula (Ia) is a compound of formula (Ia-9):

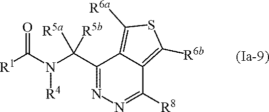

.

In certain embodiments, the compound of formula (Ia) is a compound of formula (Ia-10):

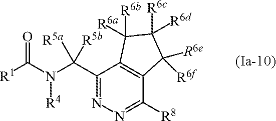

In certain embodiments, the compound of formula (Ia) is a compound of formula (Ia-11):

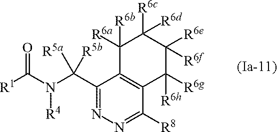

In certain embodiments, the compound of formula (Ia) is a compound of formula (Ia-12):

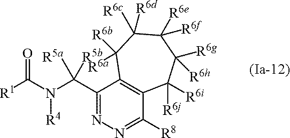

In certain embodiments, the compound of formula (Ia) is a compound of formula (Ia-13):

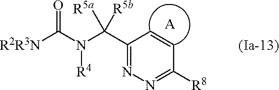

In certain embodiments, the compound of formula (Ia) is a compound of formula (Ia-14):

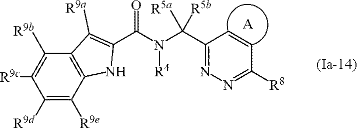

In certain embodiments, the compound of formula (Ia) is a compound of formula (Ia-15):

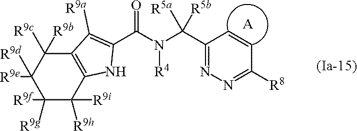

. In certain embodiments, the compound of formula (Ia) is a compound of formula (Ia-16):

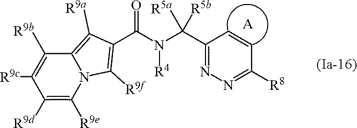

. In certain embodiments, the compound of formula (Ia) is a compound of formula (Ia-17):

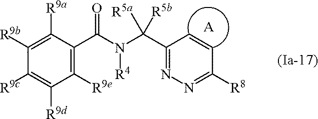

.

In certain embodiments, the compound of formula (Ia) is a compound of formula (Ia-18):

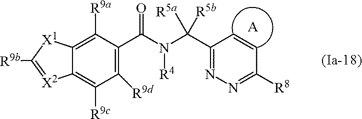

In certain embodiments, the compound of formula (Ia) is a compound of formula (Ia-19):

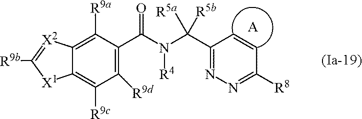

. In certain embodiments, the compound of formula (Ia) is a compound of formula (Ia-20):

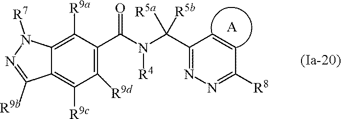

. In certain embodiments, the compound of formula (Ia) is a compound of formula (Ia-21):

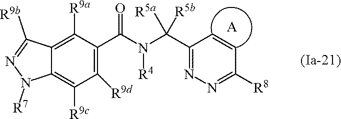

In certain embodiments, the compound of formula (Ia) is a compound of formula (Ia-22):

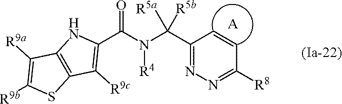

In certain embodiments, the compound of formula (Ia) is a compound of formula (Ia-23):

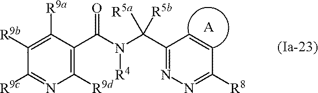

. In certain embodiments, the compound of formula (Ia) is a compound of formula (Ia-24):

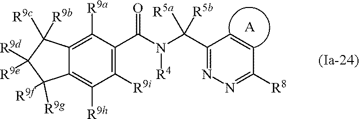

In certain embodiments, the compound of formula (Ia) is a compound of formula (Ia-25):

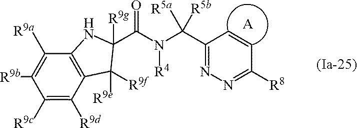

In certain embodiments, the compound of formula (Ia) is a compound of formula (Ia-26):

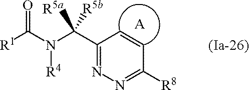

. In certain embodiments, the compound of formula (Ia) is a compound of formula (Ia-27):

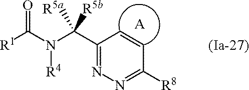

In certain embodiments, the compound of formula (Ia) is a compound of formula (Ia-28):

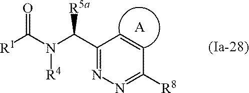

. In certain embodiments, the compound of formula (Ia) is a compound of formula (Ia-29):

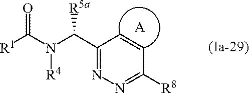

In certain embodiments, the compound of formula (Ib) is a compound of formula (Ib-1a):

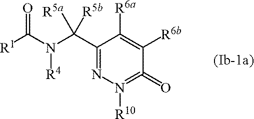

In certain embodiments, the compound of formula (Ib) is a compound of formula (Ib-1b):

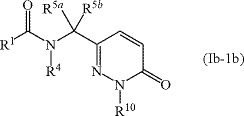

In certain embodiments, the compound of formula (Ib) is a compound of formula (Ib-2):

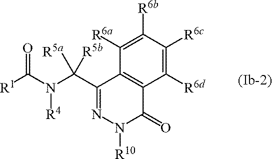

In certain embodiments, the compound of formula (Ib) is a compound of formula (Ib-3):

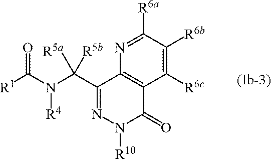

In certain embodiments, the compound of formula (Ib) is a compound of formula (Ib-4):

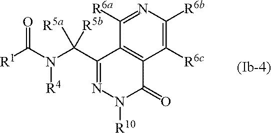

In certain embodiments, the compound of formula (Ib) is a compound of formula (Ib-5):

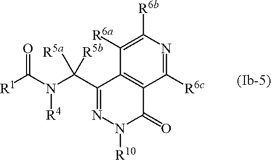

In certain embodiments, the compound of formula (Ib) is a compound of formula (Ib-6):

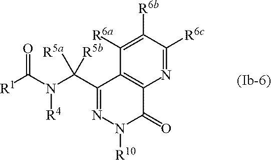

In certain embodiments, the compound of formula (Ib) is a compound of formula (Ib-7):

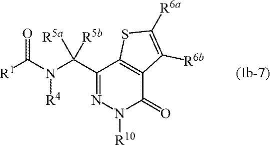

In certain embodiments, the compound of formula (Ib) is a compound of formula (Ib-8):

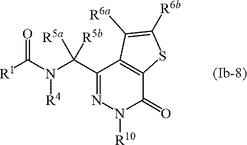

In certain embodiments, the compound of formula (Ib) is a compound of formula (Ib-9):

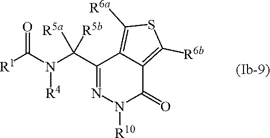

In certain embodiments, the compound of formula (Ib) is a compound of formula (Ib-10):

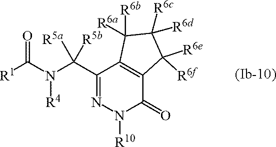

In certain embodiments, the compound of formula (Ib) is a compound of formula (Ib-11):

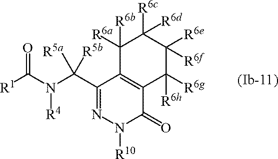

In certain embodiments, the compound of formula (Ib) is a compound of formula (Ib-12):

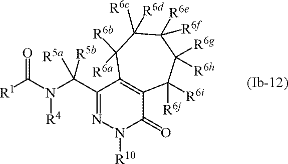

In certain embodiments, the compound of formula (Ib) is a compound of formula (Ib-13):

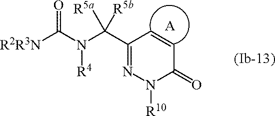

In certain embodiments, the compound of formula (Ib) is a compound of formula (Ib-14):

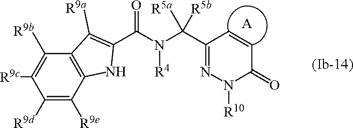

In certain embodiments, the compound of formula (Ib) is a compound of formula (Ib-15):

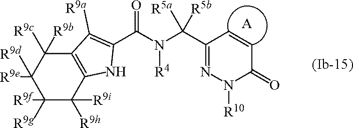

In certain embodiments, the compound of formula (Ib) is a compound of formula (Ib-16):

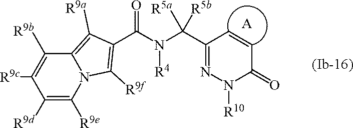

In certain embodiments, the compound of formula (Ib) is a compound of formula (Ib-17):

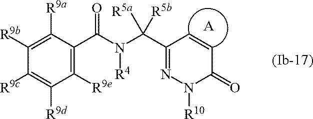

In certain embodiments, the compound of formula (Ib) is a compound of formula (Ib-18):

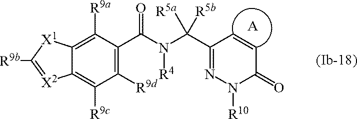

In certain embodiments, the compound of formula (Ib) is a compound of formula (Ib-19):

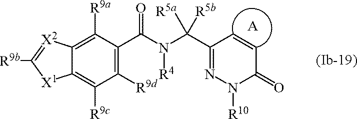

In certain embodiments, the compound of formula (Ib) is a compound of formula (Ib-20):

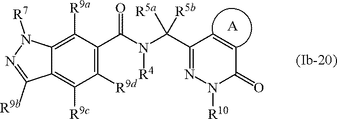

In certain embodiments, the compound of formula (Ib) is a compound of formula (Ib-21):

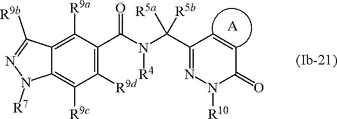

In certain embodiments, the compound of formula (Ib) is a compound of formula (Ib-22):

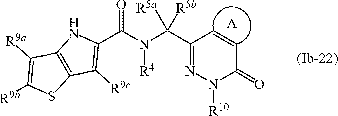

. In certain embodiments, the compound of formula (Ib) is a compound of formula (Ib-23):

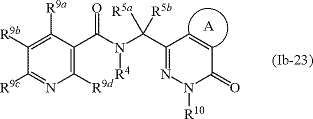

In certain embodiments, the compound of formula (Ib) is a compound of formula (Ib-24):

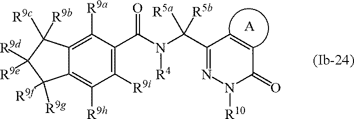

In certain embodiments, the compound of formula (Ib) is a compound of formula (Ib-25):

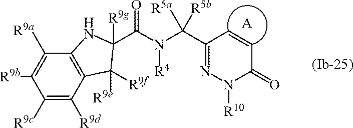

In certain embodiments, the compound of formula (Ib) is a compound of formula (Ib-26):

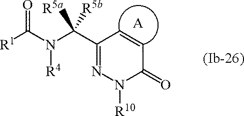

. In certain embodiments, the compound of formula (Ib) is a compound of formula (Ib-27):

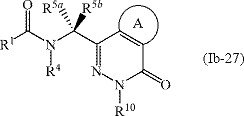

In certain embodiments, the compound of formula (Ib) is a compound of formula (Ib-28):

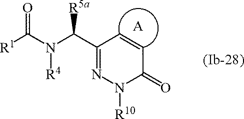

. In certain embodiments, the compound of formula (Ib) is a compound of formula (Ib-29):

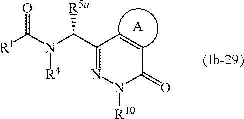

.

In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), -

C(═O)OR^{b}, —OC(═O)R^{b}, —SR^{b}, —S(═O)R^{b}, —S(═O)₂R^{b}, —N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O-.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, —C(═O)N(R^{b})(R^{b}), —C(═O)OR^{b}, —OC(═O)R^{b}, —SR^{b}, —S(═O)R^{b}, —S(═O)₂R^{b}, and —N(R^{b})S(═O)₂R^{b}, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form -O(CH₂)₁₋₃O-.

In certain embodiments, the alkyl, alkenyl, alkynyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, oxadiazolyl (including 1,2,3-, 1,2,4-, 1,2,5-, and 1,3,4-oxadiazole), and triazolyl (such as 1,2,3-triazolyl and 1,2,4-triazolyl).

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxazolidinyl, azetidinyl, and the corresponding oxo analogues (where a methylene ring group is replaced with a carbonyl) thereof.

In certain embodiments, ring A is absent and

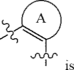

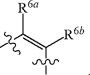

In certain embodiments, ring A is absent and

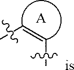

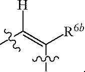

In certain embodiments, ring A is absent and

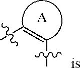

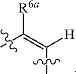

In certain embodiments, ring A is absent and

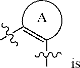

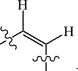

In certain embodiments, ring A is

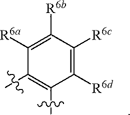

In certain embodiments, ring A is

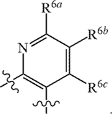

In certain embodiments, ring A is

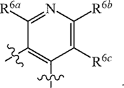

In certain embodiments, ring A is

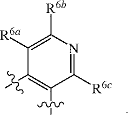

In certain embodiments, ring A is

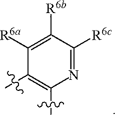

In certain embodiments, ring A is

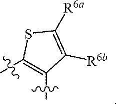

In certain embodiments, ring A is

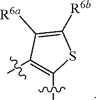

In certain embodiments, ring A is

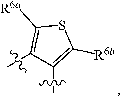

wherein there is no bridgehead double bond the bicyclic structure including ring A. In certain embodiments, ring A is

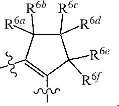

In certain embodiments, ring A is

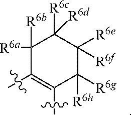

In certain embodiments, ring A is

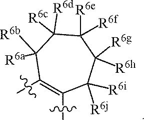

In certain embodiments, ring A is

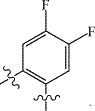

In certain embodiments, ring A is

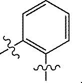

In certain embodiments, ring A is

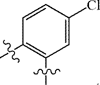

In certain embodiments, ring A is

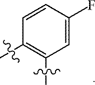

In certain embodiments, ring A is

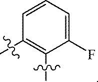

In certain embodiments, R¹ is

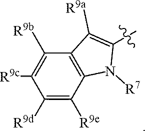

In certain embodiments, R¹ is

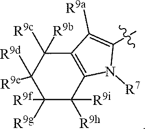

In certain embodiments, R¹ is

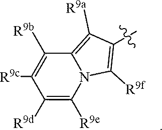

In certain embodiments, R¹ is

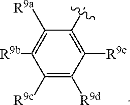

In certain embodiments, R¹ is

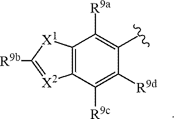

In certain embodiments, R¹ is

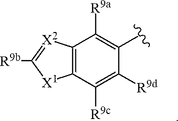

In certain embodiments, R¹ is

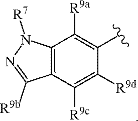

In certain embodiments, R¹ is

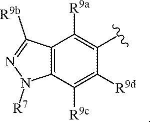

In certain embodiments, R¹ is

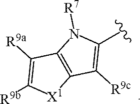

In certain embodiments, R¹ is

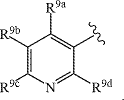

In certain embodiments, R¹ is

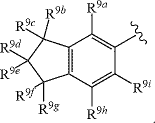

In certain embodiments, R¹ is

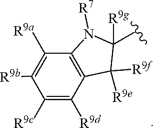

In certain embodiments, R¹ is

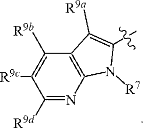

In certain embodiments, R¹ is

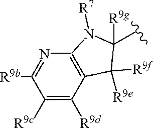

In certain embodiments, R¹ is

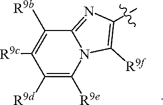

In certain embodiments, R¹ is

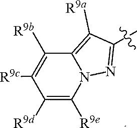

In certain embodiments, R¹ is

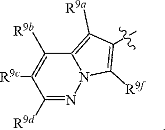

In certain embodiments, R¹ is

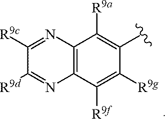

In certain embodiments, R¹ is

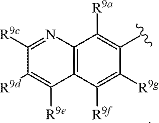

In certain embodiments, R¹ is

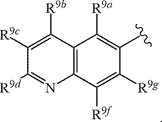

In certain embodiments, R¹ is

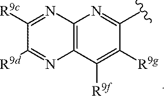

In certain embodiments, R¹ is

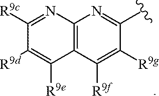

In certain embodiments, R¹ is

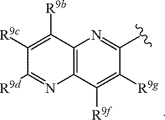

In certain embodiments, R¹ is

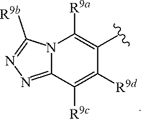

In certain embodiments, R¹ is

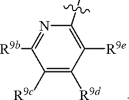

In certain embodiments, R¹ is

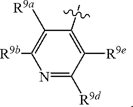

In certain embodiments, R¹ is

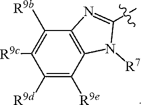

In certain embodiments, R¹ is

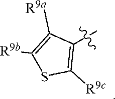

In certain embodiments, R¹ is

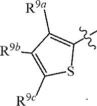

In certain embodiments, R¹ is

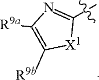

In certain embodiments, R¹ is

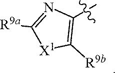

In certain embodiments, R¹ is

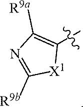

In certain embodiments, R¹ is

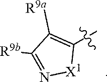

In certain embodiments, R¹ is

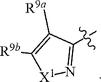

In certain embodiments, R¹ is

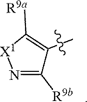

In certain embodiments, R¹ is

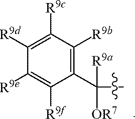

In certain embodiments, R¹ is

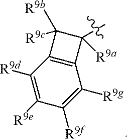

In certain embodiments, R¹ is

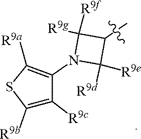

In certain embodiments, R¹ is

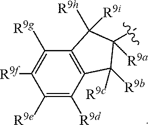

In certain embodiments, R¹ is

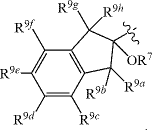

In certain embodiments, R¹ is

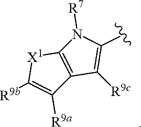

In certain embodiments, R¹ is

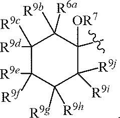

In certain embodiments, R¹ is

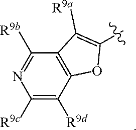

In certain embodiments, R¹ is

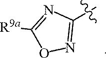

In certain embodiments, R¹ is

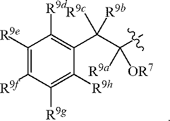

In certain embodiments, R¹ is

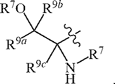

In certain embodiments, R¹ is

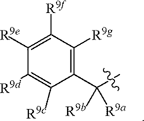

In certain embodiments, R¹ is

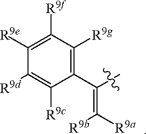

In certain embodiments, R¹ is

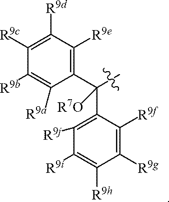

In certain embodiments, R¹ is

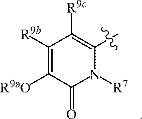

In certain embodiments, R¹ is

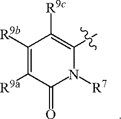

In certain embodiments, R¹ is

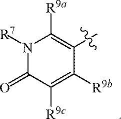

In certain embodiments, R¹ is

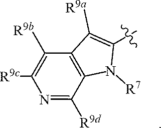

In certain embodiments, R¹ is

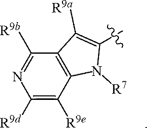

In certain embodiments, R¹ is

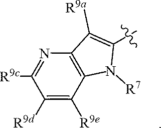

In certain embodiments, R¹ is

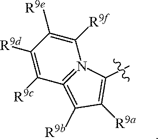

In certain embodiments, R¹ is

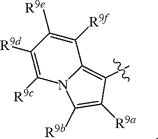

In certain embodiments, R¹ is

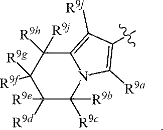

In certain embodiments, R¹ is

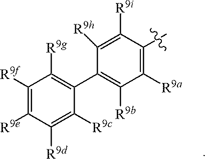

In certain embodiments, R¹ is

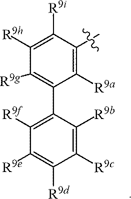

In certain embodiments, R¹ is

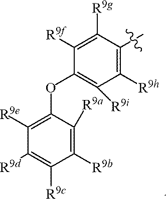

In certain embodiments, R¹ is

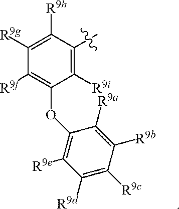

In certain embodiments, R¹ is

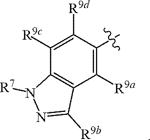

In certain embodiments, R¹ is

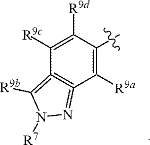

In certain embodiments, R¹ is

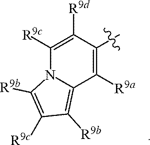

In certain embodiments, R¹ is

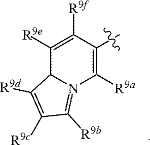

In certain embodiments, R¹ is

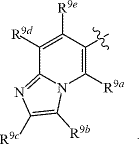

In certain embodiments, R¹ is

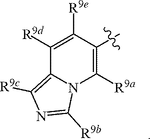

In certain embodiments, R¹ is phenyl optionally substituted with at least one selected from the group consisting of C₁-C₆ alkyl (such as, for example, methyl, ethyl, and isopropyl), halo (such as, for example, F, Cl, Br, and I), C₁-C₃ haloalkyl (such as, for example, monofluoromethyl, difluoromethyl, and trifluoromethyl), and —CN.

In certain embodiments, R¹ is selected from the group consisting of: phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl. In other embodiments, R¹ is 3,4-difluorophenyl.

In yet other embodiments, R¹ is 3-fluoro-4-methylphenyl. In yet other embodiments, R¹ is 4-fluoro-3-methylphenyl. In yet other embodiments, R¹ is 3-cyano-4-fluorophenyl. In yet other embodiments, R¹ is 4-difluoromethyl-3-fluorophenyl. In yet other embodiments, R¹ is 3-fluoro-4-trifluoromethylphenyl.

In certain embodiments, R¹ is

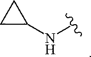

In certain embodiments, R¹ is

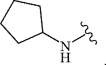

In certain embodiments, R¹ is

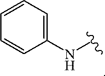

In certain embodiments, R¹ is

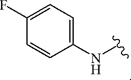

In certain embodiments, R¹ is

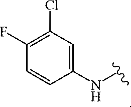

In certain embodiments, R¹ is

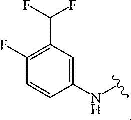

In certain embodiments, R¹ is

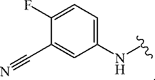

In certain embodiments, R¹ is

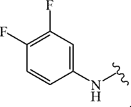

In certain embodiments, R¹ is

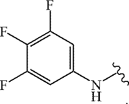

In certain embodiments, R¹ is

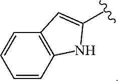

In certain embodiments, R¹ is

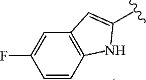

In certain embodiments, R¹ is

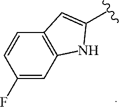

In certain embodiments, R¹ is

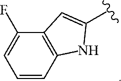

In certain embodiments, R¹ is

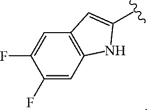

In certain embodiments, R¹ is

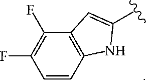

In certain embodiments, R¹ is

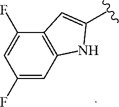

In certain embodiments, R¹ is

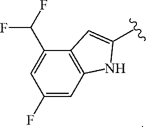

In certain embodiments, R¹ is

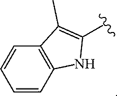

In certain embodiments, R¹ is

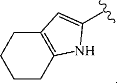

In certain embodiments, R¹ is

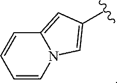

In certain embodiments, R¹ is

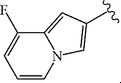

In certain embodiments, R¹ is

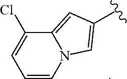

In certain embodiments, R¹ is

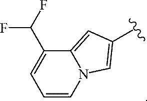

In certain embodiments, R¹ is

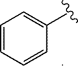

In certain embodiments, R¹ is

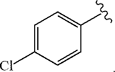

In certain embodiments, R¹ is

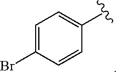

In certain embodiments, R¹ is

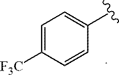

In certain embodiments, R¹ is

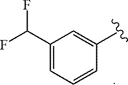

In certain embodiments, R¹ is

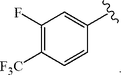

In certain embodiments, R¹ is

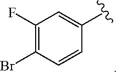

In certain embodiments, R¹ is

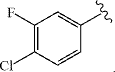

In certain embodiments, R¹ is

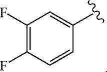

In certain embodiments, R¹ is

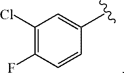

In certain embodiments, R¹ is

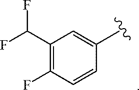

In certain embodiments, R¹ is

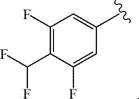

In certain embodiments, R¹ is

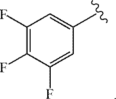

In certain embodiments, R¹ is

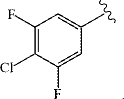

In certain embodiments, R¹ is

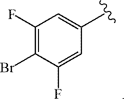

In certain embodiments, R¹ is

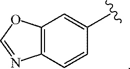

In certain embodiments, R¹ is

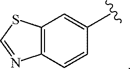

In certain embodiments, R¹ is

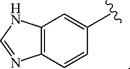

In certain embodiments, R¹ is

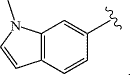

In certain embodiments, R¹ is

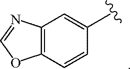

In certain embodiments, R¹ is

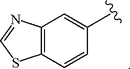

In certain embodiments, R¹ is

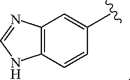

In certain embodiments, R¹ is

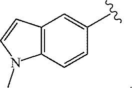

In certain embodiments, R¹ is

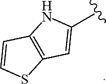

In certain embodiments, R¹ is

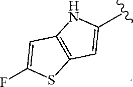

In certain embodiments, R¹ is

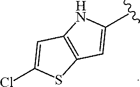

In certain embodiments, R¹ is

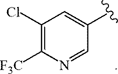

In certain embodiments, R¹ is

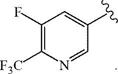

In certain embodiments, R¹ is

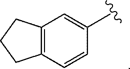

In certain embodiments, R¹ is

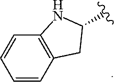

In certain embodiments, R¹ is

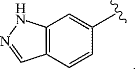

In certain embodiments, R¹ is

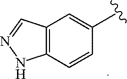

In certain embodiments, R¹ is

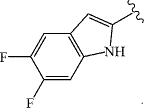

In certain embodiments, R¹ is

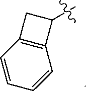

In certain embodiments, R¹ is

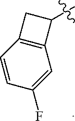

In certain embodiments, R¹ is

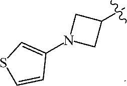

In certain embodiments, R¹ is

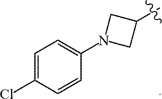

In certain embodiments, R¹ is

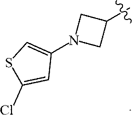

In certain embodiments, R¹ is

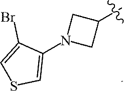

In certain embodiments, R¹ is

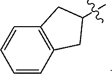

In certain embodiments, R¹ is

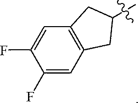

In certain embodiments, R¹ is

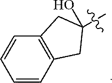

In certain embodiments, R¹ is

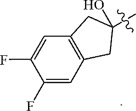

In certain embodiments, R¹ is

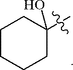

In certain embodiments, R¹ is

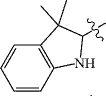

In certain embodiments, R¹ is

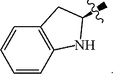

In certain embodiments, R¹ is

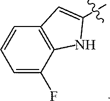

In certain embodiments, R¹ is

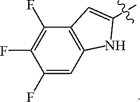

In certain embodiments, R¹ is

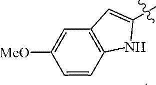

In certain embodiments, R¹ is

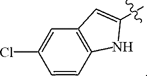

In certain embodiments, R¹ is

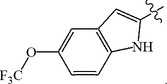

In certain embodiments, R¹ is

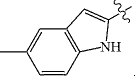

In certain embodiments, R¹ is

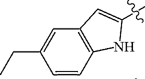

In certain embodiments, R¹ is

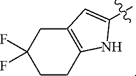

In certain embodiments, R¹ is

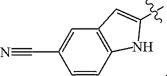

In certain embodiments, R¹ is

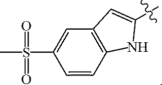

In certain embodiments, R¹ is

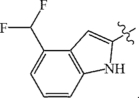

In certain embodiments, R¹ is

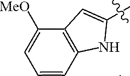

In certain embodiments, R¹ is

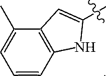

In certain embodiments, R¹ is

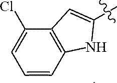

In certain embodiments, R¹ is

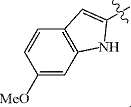

In certain embodiments, R¹ is

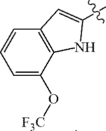

In certain embodiments, R¹ is

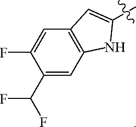

In certain embodiments, R¹ is

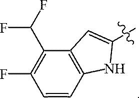

In certain embodiments, R¹ is

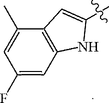

In certain embodiments, R¹ is

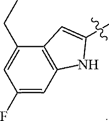

In certain embodiments, R¹ is

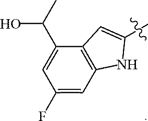

In certain embodiments, R¹ is

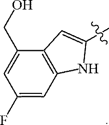

In certain embodiments, R¹ is

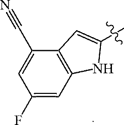

In certain embodiments, R¹ is

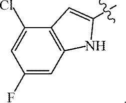

In certain embodiments, R¹ is

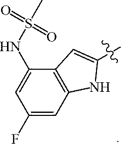

In certain embodiments, R¹ is

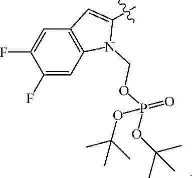

In certain embodiments, R¹ is

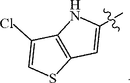

In certain embodiments, R¹ is

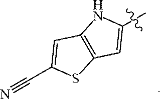

In certain embodiments, R¹ is

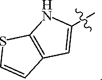

In certain embodiments, R¹ is

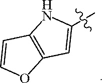

In certain embodiments, R¹ is

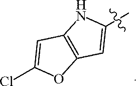

In certain embodiments, R¹ is

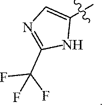

In certain embodiments, R¹ is

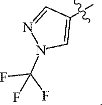

In certain embodiments, R¹ is

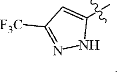

In certain embodiments, R¹ is

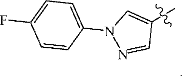

In certain embodiments, R¹ is

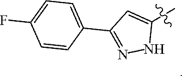

In certain embodiments, R¹ is

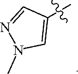

In certain embodiments, R¹ is

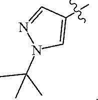

In certain embodiments, R¹ is

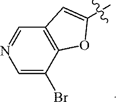

In certain embodiments, R¹ is

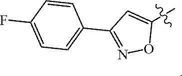

In certain embodiments, R¹ is

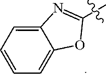

In certain embodiments, R¹ is

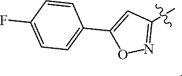

In certain embodiments, R¹ is

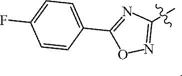

In certain embodiments, R¹ is

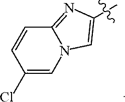

In certain embodiments, R¹ is

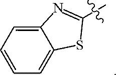

In certain embodiments, R¹ is

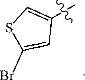

In certain embodiments, R¹ is

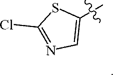

In certain embodiments, R¹ is

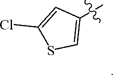

In certain embodiments, R¹ is

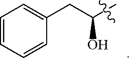

In certain embodiments, R¹ is

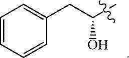

In certain embodiments, R¹ is

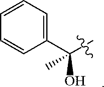

In certain embodiments, R¹ is

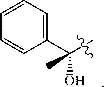

In certain embodiments, R¹ is

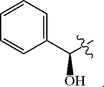

In certain embodiments, R¹ is

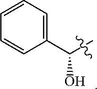

In certain embodiments, R¹ is

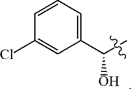

In certain embodiments, R¹ is

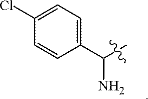

\

In certain embodiments, R¹ is

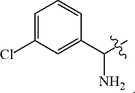

In certain embodiments, R¹ is

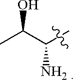

In certain embodiments, R¹ is

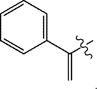

In certain embodiments, R¹ is

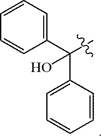

In certain embodiments, R¹ is

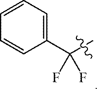

In certain embodiments, R¹ is

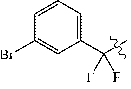

In certain embodiments, R¹ is

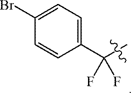

In certain embodiments, R¹ is

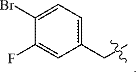

In certain embodiments, R¹ is

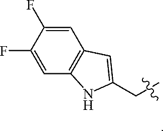

In certain embodiments, R¹ is

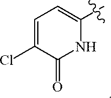

In certain embodiments, R¹ is

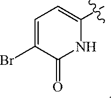

In certain embodiments, R¹ is

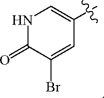

In certain embodiments, R¹ is

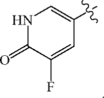

In certain embodiments, R¹ is

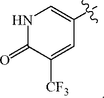

In certain embodiments, R¹ is

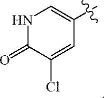

In certain embodiments, R¹ is

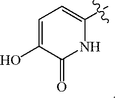

In certain embodiments, R¹ is

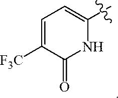

In certain embodiments, R¹ is

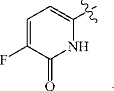

In certain embodiments, R¹ is

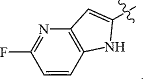

In certain embodiments, R¹ is

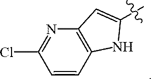

In certain embodiments, R¹ is

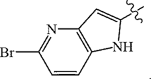

In certain embodiments, R¹ is

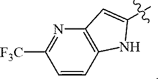

In certain embodiments, R¹ is

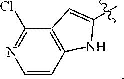

In certain embodiments, R¹ is

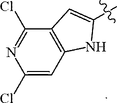

In certain embodiments, R¹ is

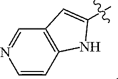

In certain embodiments, R¹ is

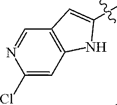

In certain embodiments, R¹ is

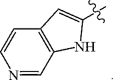

In certain embodiments, R¹ is

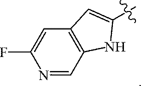

In certain embodiments, R¹ is

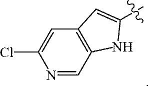

In certain embodiments, R¹ is

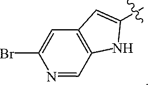

In certain embodiments, R¹ is

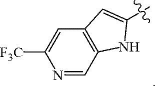

In certain embodiments, R¹ is

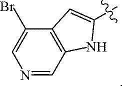

In certain embodiments, R¹ is

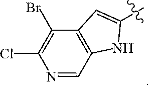

In certain embodiments, R¹ is

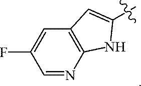

In certain embodiments, R¹ is

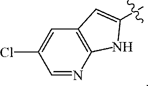

In certain embodiments, R¹ is

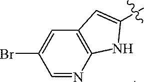

In certain embodiments, R¹ is

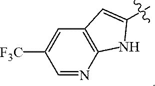

In certain embodiments, R¹ is

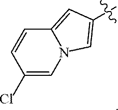

In certain embodiments, R¹ is

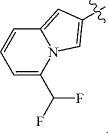

In certain embodiments, R¹ is

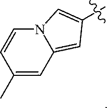

In certain embodiments, R¹ is

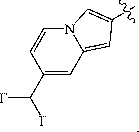

In certain embodiments, R¹ is

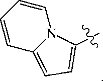

In certain embodiments, R¹ is

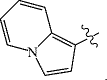

In certain embodiments, R¹ is

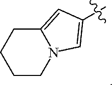

In certain embodiments, R¹ is

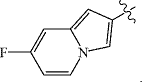

In certain embodiments, R¹ is

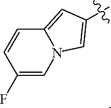

In certain embodiments, R¹ is

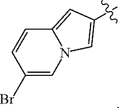

In certain embodiments, R¹ is

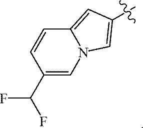

In certain embodiments, R¹ is

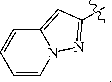

In certain embodiments, R¹ is

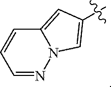

In certain embodiments, R¹ is

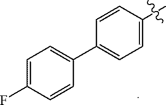

In certain embodiments, R¹ is

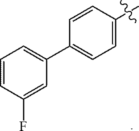

In certain embodiments, R¹ is

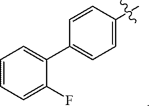

In certain embodiments, R¹ is

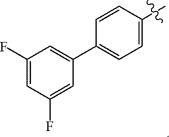

In certain embodiments, R¹ is

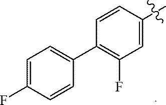

In certain embodiments, R¹ is

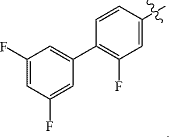

In certain embodiments, R¹ is

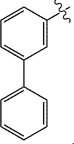

In certain embodiments, R¹ is

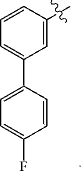

In certain embodiments, R¹ is

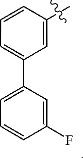

In certain embodiments, R¹ is

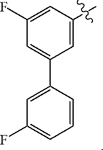

In certain embodiments, R¹ is

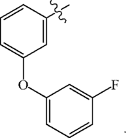

In certain embodiments, R¹ is

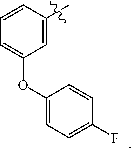

In certain embodiments, R¹ is

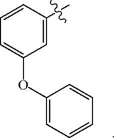

In certain embodiments, R¹ is

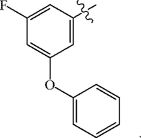

In certain embodiments, R¹ is

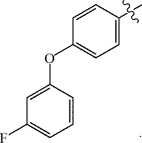

In certain embodiments, R¹ is

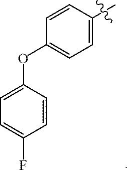

In certain embodiments, R¹ is

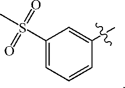

In certain embodiments, R¹ is

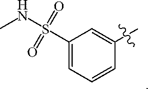

In certain embodiments, R¹ is

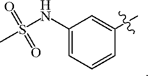

In certain embodiments, R¹ is

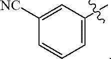

In certain embodiments, R¹ is

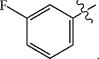

In certain embodiments, R¹ is

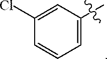

In certain embodiments, R¹ is

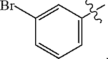

In certain embodiments, R¹ is

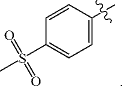

In certain embodiments, R¹ is

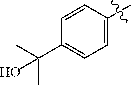

In certain embodiments, R¹ is

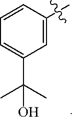

In certain embodiments, R¹ is

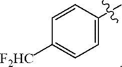

In certain embodiments, R¹ is

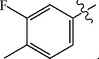

In certain embodiments, R¹ is

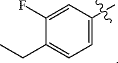

In certain embodiments, R¹ is

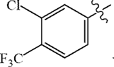

In certain embodiments, R¹ is

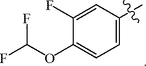

In certain embodiments, R¹ is

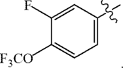

In certain embodiments, R¹ is

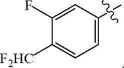

In certain embodiments, R¹ is

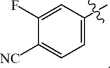

In certain embodiments, R¹ is

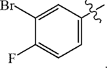

In certain embodiments, R¹ is

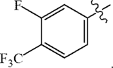

In certain embodiments, R¹ is

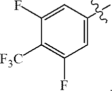

In certain embodiments, R¹ is

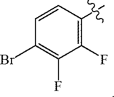

In certain embodiments, R¹ is

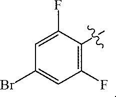

In certain embodiments, R¹ is

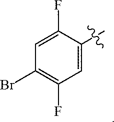

In certain embodiments, R¹ is

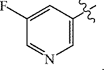

In certain embodiments, R¹ is

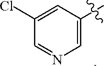

In certain embodiments, R¹ is

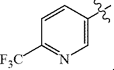

In certain embodiments, R¹ is

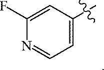

In certain embodiments, R¹ is

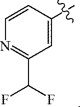

In certain embodiments, R¹ is

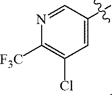

In certain embodiments, R¹ is

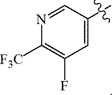

In certain embodiments, R¹ is

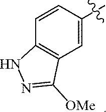

In certain embodiments, R¹ is

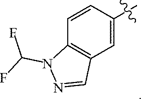

In certain embodiments, R¹ is

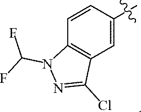

In certain embodiments, R¹ is

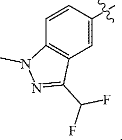

In certain embodiments, R¹ is

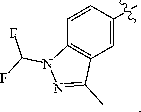

In certain embodiments, R¹ is

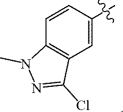

In certain embodiments, R¹ is

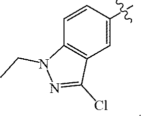

In certain embodiments, R¹ is

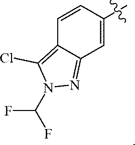

In certain embodiments, R¹ is

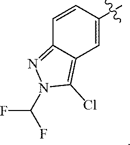

In certain embodiments, R¹ is

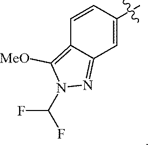

In certain embodiments, R¹ is

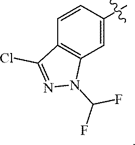

In certain embodiments, R¹ is

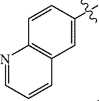

In certain embodiments, R¹ is

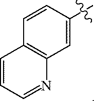

In certain embodiments, R¹ is

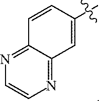

In certain embodiments, R¹ is

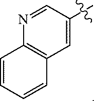

In certain embodiments, R¹ is

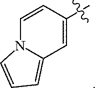

In certain embodiments, R¹ is

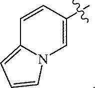

In certain embodiments, R¹ is

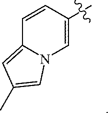

In certain embodiments, R¹ is

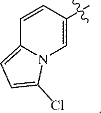

In certain embodiments, R¹ is

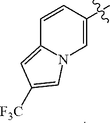

In certain embodiments, R¹ is

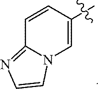

In certain embodiments, R¹ is

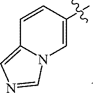

In certain embodiments, R¹ is

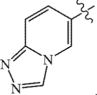

In certain embodiments, R² is optionally substituted C₃-C₈ cycloalkyl, such as but not limited to optionally substituted cyclopropyl, optionally substituted cycbutyl, optionally substituted cyclopentyl, optionally substituted cyclohexyl, optionally substituted cycloheptyl, and optionally substituted cyclooctyl.

In certain embodiments, R² is selected from the group consisting of optionally substituted phenyl, optionally substituted benzyl, and -(CH₂)(optionally substituted heteroaryl), wherein the phenyl, benzyl, or heteroaryl is optionally substituted with at least one selected from the group consisting of C₁-C₆ alkyl (such as, for example, methyl, ethyl, and isopropyl), halo (such as, for example, F, Cl, Br, and I), C₁-C₃ haloalkyl (such as, for example, monofluoromethyl, difluoromethyl, and trifluoromethyl), and —CN.

In certain embodiments, R² is selected from the group consisting of: cyclopropyl, cyclopentyl, phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl.

In other embodiments, R² is 3,4-difluorophenyl. In yet other embodiments, R² is 3-chloro-4-fluorophenyl. In yet other embodiments, R² is cyclopropyl. In yet other embodiments, R² is cyclopentyl. In yet other embodiments, R² is phenyl. In yet other embodiments, R² is 4-chloro-3-fluorophenyl. In yet other embodiments, R² is 4-chlorophenyl. In yet other embodiments, R² is 4-bromophenyl. In yet other embodiments, R² is 4-trifluoromethylphenyl. In yet other embodiments, R² is 3-fluoro-4-methylphenyl. In yet other embodiments, R² is 4-fluoro-3-methylphenyl. In yet other embodiments, R² is 3-cyano-4-fluorophenyl. In yet other embodiments, R² is 3-difluoromethylphenyl. In yet other embodiments, R² is 3-difluoromethyl-4-fluorophenyl. In yet other embodiments, R² is 4-difluoromethyl-3-fluorophenyl. In yet other embodiments, R² is 3-fluoro-4-trifluoromethylphenyl. In yet other embodiments, R² is 3-fluoro-4-bromophenyl. In yet other embodiments, R² is 3,4,5-trifluorophenyl.

In certain embodiments, each occurrence of R³ is independently selected from the group consisting of H and methyl. In other embodiments, R³ is H. In yet other embodiments, R³ is methyl.

In certain embodiments, R⁴ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁—C₆ alkyl), —CH₂S(═O)₂NHC(═O)NH(optionally substituted phenyl), optionally substituted benzyl, and optionally substituted phenyl.

In certain embodiments, R⁴ is methyl. In certain embodiments, R⁴ is sec-butyl. In certain embodiments, R⁴ is —CH₂CH₂CH₂OH. In certain embodiments, R⁴ is

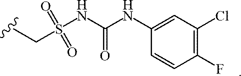

In certain embodiments, R^{5a} is selected from the group consisting of H and methyl. In other embodiments, R^{5a} is H. In other embodiments, R^{5a} is methyl. In certain embodiments, R^{5b} is selected from the group consisting of H and methyl. In other embodiments, R^{5b} is H. In other embodiments, R^{5b} is methyl.

In certain embodiments, R^{6a} is H. In certain embodiments, R^{6a} is Cl. In certain embodiments, R^{6a} is F. In certain embodiments, R^{6b} is H. In certain embodiments, R^{6b} is Cl. In certain embodiments, R^{6b} is F. In certain embodiments, R^{6c} is H. In certain embodiments, R^{6c} is Cl. In certain embodiments, R^{6c} is F. In certain embodiments, R^{6d} is H. In certain embodiments, R^{6d} is Cl. In certain embodiments, R^{6d} is F.

In certain embodiments, R⁷ is 3-chloro-4-fluorophenyl. In certain embodiments, R⁷ is H.

In certain embodiments, R^{9a} is F. In certain embodiments, R^{9a} is methyl. In certain embodiments, R^{9a} is CF₃. In certain embodiments, R^{9a} is Br. In certain embodiments, R^{9a} is Cl. In certain embodiments, R^{9a} is CH₂F. In certain embodiments, R^{9a} is CN. In certain embodiments, R^{9b} is F. In certain embodiments, R^{9b} is methyl. In certain embodiments, R^{9b} is CF₃. In certain embodiments, R^{9b} is Br. In certain embodiments, R^{9b} is Cl. In certain embodiments, R^{9b} is CH₂F. In certain embodiments, R^{9b} is CN. In certain embodiments, R^{9c} is F. In certain embodiments, R^{9c} is methyl. In certain embodiments, R^{9c} is CF₃. In certain embodiments, R^{9c} is Br. In certain embodiments, R^{9c} is Cl. In certain embodiments, R^{9c} is CH₂F. In certain embodiments, R^{9c} is CN. In certain embodiments, R^{9d} is F. In certain embodiments, R^{9d} is methyl. In certain embodiments, R^{9d} is CF₃. In certain embodiments, R^{9d} is Br. In certain embodiments, R^{9d} is Cl. In certain embodiments, R^{9d} is CH₂F. In certain embodiments, R^{9d} is CN. In certain embodiments, R^{9e} is F. In certain embodiments, R^{9e} is methyl. In certain embodiments, R^{9e} is CF₃. In certain embodiments, R^{9e} is Br. In certain embodiments, R^{9e} is Cl. In certain embodiments, R^{9e} is CH₂F. In certain embodiments, R^{9e} is CN. In certain embodiments, R^{9f} is F. In certain embodiments, R^{9f} is methyl. In certain embodiments, R^{9f} is CF₃. In certain embodiments, R^{9f} is Br. In certain embodiments, R^{9f} is Cl. In certain embodiments, R^{9f} is CH₂F. In certain embodiments, R^{9f} is CN. In certain embodiments, R^{9g} is F. In certain embodiments, R^{9g} is methyl. In certain embodiments, R^{9g} is CF₃. In certain embodiments, R^{9g} is Br. In certain embodiments, R^{9g} is Cl. In certain embodiments, R^{9g} is CH₂F. In certain embodiments, R^{9g} is CN. In certain embodiments, R^{9h} is F. In certain embodiments, R^{9h} is methyl. In certain embodiments, R^{9h} is CF₃. In certain embodiments, R^{9h} is Br. In certain embodiments, R^{9h} is Cl. In certain embodiments, R^{9h} is CH₂F. In certain embodiments, R^{9h} is CN. In certain embodiments, R⁹ⁱ is F. In certain embodiments, R⁹ⁱ is methyl. In certain embodiments, R⁹ⁱ is CF₃. In certain embodiments, R⁹ⁱ is Br. In certain embodiments, R⁹ⁱ is Cl. In certain embodiments, R⁹ⁱ is CH₂F. In certain embodiments, R⁹ⁱ is CN. In certain embodiments, R^{9j} is F. In certain embodiments, R^{9j} is methyl. In certain embodiments, R^{9j} is CF₃. In certain embodiments, R^{9j} is Br. In certain embodiments, R^{9j} is Cl. In certain embodiments, R^{9j} is CH₂F. In certain embodiments, R^{9j} is CN.

In certain embodiments, R¹⁰ is H.

In certain embodiments, the compound of the disclosure is any compound disclosed herein, or a salt, solvate, prodrug, isotopically labelled, stereoisomer, any mixture of stereoisomers, tautomer, and/or any mixture of tautomers thereof.

In certain embodiments, the compound is at least one selected from Table 4, or a salt, solvate, prodrug, isotopically labelled, stereoisomer, any mixture of stereoisomers, tautomer, and/or any mixture of tautomers thereof.

In certain embodiments, the compound is at least one of:
3-(3-chloro-4-fluorophenyl)-1-methyl-1-(1-(4-oxo-3,4-dihydrophthalazin-1-yl)ethyl)urea;
3-(3-chloro-4-fluorophenyl)-1-isobutyl-1-(1-(4-oxo-3,4-dihydrophthalazin-1-yl)ethyl)urea;
3-(3-chloro-4-fluorophenyl)-1-isobutyl-1-(1-(3-methyl-4-oxo-3,4-dihydrophthalazin-1-yl)ethyl)urea;
3-(4-fluorophenyl)-1-isobutyl-1-(1-(3-methyl-4-oxo-3,4-dihydrophthalazin-1-yl)ethyl)urea;
3-(3-chloro-4-fluorophenyl)-1-(1-(6-chloro-4-oxo-3,4-dihydrophthalazin-1-yl)ethyl)-1-isobutylurea;
1-(1-(6-chloro-4-oxo-3,4-dihydrophthalazin-1-yl)ethyl)-3-(4-fluorophenyl)-1-isobutylurea;
3-(3-chloro-4-fluorophenyl)-1-(1-(6-chloro-4-oxo-3,4-dihydrophthalazin-1-yl)ethyl)-1-methylurea;
3-(3-chloro-4-fluorophenyl)-1-(1-(6-chloro-4-oxo-3,4-dihydrophthalazin-1-yl)ethyl)-1-(3-hydroxypropyl)urea;
1-(1-(6-chloro-4-oxo-3,4-dihydrophthalazin-1-yl)ethyl)-3-(4-fluorophenyl)-1-(3-hydroxypropyl)urea;
3-(3-chloro-4-fluorophenyl)-1-(1-(6-fluoro-4-oxo-3,4-dihydrophthalazin-1-yl)ethyl)-1-isobutylurea;
1-(1-(6-fluoro-4-oxo-3,4-dihydrophthalazin-1-yl)ethyl)-3-(4-fluorophenyl)-1-isobutylurea;
3-(3-chloro-4-fluorophenyl)-1-(1-(6-fluoro-4-oxo-3,4-dihydrophthalazin-1-yl)ethyl)-1-methylurea;
1-(1-(6-fluoro-4-oxo-3,4-dihydrophthalazin-1-yl)ethyl)-3-(4-fluorophenyl)-1-methylurea;
3-(4-fluorophenyl)-1-isobutyl-1-(1-(4-oxo-3,4-dihydrophthalazin-1-yl)ethyl)urea;
3-(3-chloro-4-fluorophenyl)-1-(1-(6,7-difluoro-3-methyl-4-oxo-3,4-dihydrophthalazin-1-yl)ethyl)-1-methylurea;
3-(3-chloro-4-fluorophenyl)-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-3-(4-fluorophenyl)-1-isobutylurea;
3 -(3 -chloro-4-fluorophenyl)-1 -(1 -(6,7-difluoro-3-methyl-4-oxo-3,4-dihydrophthalazin-1-yl)ethyl)-1-(3-hydroxypropyl)urea;
3-(4-fluorophenyl)- 1 -(1 -(6,7-difluoro-3 -methyl-4-oxo-3,4-dihydrophthalazin-1-yl)ethyl)-1-(3 -hydroxypropyl)urea;
3 -(3 -chloro-4-fluorophenyl)-1 -(1 -(6,7-difluoro-4-oxo-3,4-dihydrophthalazin- 1 -yl)ethyl)-1 -isobutylurea;
1-(1-(6,7-difluoro-4-oxo-3,4-dihydrophthalazin-1-yl)ethyl)-1-isobutyl-3-phenylurea;
3 -(3 -chloro-4-fluorophenyl)-1 -(1 -(6,7-difluoro-4-oxo-3,4-dihydrophthalazin- 1 -yl)ethyl)-1 -methylurea;
2-(3 -(3 -chloro-4-fluorophenyl)-1-(1 -(6,7-difluoro-3 -methyl-4-oxo-3,4-dihydrophthalazin-1-yl)ethyl)ureido)-N-((3-chloro-4-fluorophenyl)carbamoyl)ethane-1-sulfonamide;
3-cyclopropyl-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-1-isobutyl-3-phenylurea;
3 -cyclopentyl-1 -(1 -(6,7-difluoro-3-methyl-4-oxo-3,4-dihydrophthalazin-1 -yl)ethyl)- 1 -isobutylurea;
1-(1-(6,7-difluoro-3-methyl-4-oxo-3,4-dihydrophthalazin-1-yl)ethyl)-3-(4-fluorophenyl)-1-methylurea;
1-(1-(6,7-difluoro-4-oxo-3,4-dihydrophthalazin-1-yl)ethyl)-3-(3,4-difluorophenyl)-1-methylurea;
1-(1-(6,7-difluoro-4-oxo-3,4-dihydrophthalazin-1-yl)ethyl)-1-methyl-3-(3,4,5-trifluorophenyl)urea;
1-(1-(6,7-difluoro-4-oxo-3,4-dihydrophthalazin-1-yl)ethyl)-3 -(4-fluorophenyl)-1-methylurea;
3 -(3 -chloro-4-fluorophenyl)-1-(1 -(5 -fluoro-4-oxo-3,4-dihydrophthalazin-1-yl)ethyl)-1-methylurea;
3 -(3,4-difluorophenyl)-1 -(1 -(5 -fluoro-4-oxo-3,4-dihydrophthalazin-1 -yl)ethyl)-1 -methylurea;
N-(1-(6,7-difluoro-4-oxo-3,4-dihydrophthalazin-1-yl)ethyl)-N-methyl-1H-indole-2-carboxamide;
N-(1-(6,7-difluoro-4-oxo-3,4-dihydrophthalazin-1-yl)ethyl)-N-ethyl-5-fluoro-1H-indole-2-carboxamide;
N-(1-(6,7-difluoro-4-oxo-3,4-dihydrophthalazin-1-yl)ethyl)-N-ethyl-1H-indole-2-carboxamide;
N-(1-(6,7-difluoro-4-oxo-3,4-dihydrophthalazin-1-yl)ethyl)-N-ethyl-4,5-difluoro-1H-indole-2-carboxamide;
N-(1-(6,7-difluoro-4-oxo-3,4-dihydrophthalazin-1-yl)ethyl)-5,6-difluoro-N-methyl-1H-indole-2-carboxamide;
N-(1-(6,7-difluoro-4-oxo-3,4-dihydrophthalazin-1-yl)ethyl)-5-fluoro-N-methyl-1H-indole-2-carboxamide;
N-(1-(6,7-difluoro-4-oxo-3,4-dihydrophthalazin-1-yl)ethyl)-6-fluoro-N-methyl-1H-indole-2-carboxamide;
N-(1-(6,7-difluoro-4-oxo-3,4-dihydrophthalazin-1-yl)ethyl)-4-fluoro-N-methyl-1H-indole-2-carboxamide;
N-(1-(6,7-difluoro-4-oxo-3,4-dihydrophthalazin-1-yl)ethyl)-4,5-difluoro-N-methyl-1H-indole-2-carboxamide;
N-(1-(6,7-difluoro-4-oxo-3,4-dihydrophthalazin-1-yl)ethyl)-N,3-dimethyl-1H-indole-2-carboxamide;
N-(1-(6,7-difluoro-4-oxo-3,4-dihydrophthalazin-1-yl)ethyl)-N-isobutyl-1H-indole-2-carboxamide;
N-(1-(6,7-difluoro-4-oxo-3,4-dihydrophthalazin-1-yl)ethyl)-N-methyl-4,5,6,7-tetrahydro-1H-indole-2-carboxamide;
N-(1-(6,7-difluoro-4-oxo-3,4-dihydrophthalazin-1-yl)ethyl)-N-methylindolizine-2-carboxamide;
N-(1-(6,7-difluoro-4-oxo-3,4-dihydrophthalazin-1-yl)ethyl)-4,6-difluoro-N-methyl-1H-indole-2-carboxamide;
N-(1-(6,7-difluoro-4-oxo-3,4-dihydrophthalazin-1-yl)ethyl)-3-fluoro-N-methyl-4-(trifluoromethyl)benzamide;
4-bromo-N-(1-(6,7-difluoro-4-oxo-3,4-dihydrophthalazin-1-yl)ethyl)-3-fluoro-N-methylbenzamide;
4-chloro-N-(1-(6,7-difluoro-4-oxo-3,4-dihydrophthalazin-1-yl)ethyl)-3-fluoro-N-methylbenzamide;
4-bromo-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-N-methyl-4-(trifluoromethyl)benzamide;
N-(1-(6,7-difluoro-4-oxo-3,4-dihydrophthalazin-1-yl)ethyl)-3,4,5-trifluoro-N-methylbenzamide;
3-chloro-N-(1-(6,7-difluoro-4-oxo-3,4-dihydrophthalazin-1-yl)ethyl)-4-fluoro-N-methylbenzamide;
4-chloro-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-3,4-difluoro-N-methylbenzamide;
N-(1-(6,7-difluoro-4-oxo-3,4-dihydrophthalazin-1-yl)ethyl)-3-(difluoromethyl)-N-methylbenzamide;
N-(1-(6,7-difluoro-4-oxo-3,4-dihydrophthalazin-1-yl)ethyl)-3-(difluoromethyl)-4-fluoro-N-methylbenzamide;
1-(1-(6,7-difluoro-4-oxo-3,4-dihydrophthalazin-1-yl)ethyl)-3 -(3 -(difluoromethyl)-4-fluorophenyl)-1-methylurea;
N-(1-(6,7-difluoro-4-oxo-3,4-dihydrophthalazin-1-yl)ethyl)-N-methylbenzamide;
8-chloro-N-(1-(6,7-difluoro-4-oxo-3,4-dihydrophthalazin-1-yl)ethyl)-N-methylindolizine-2-carboxamide;
3-(3-cyano-4-fluorophenyl)-1-(1-(6,7-difluoro-4-oxo-3,4-dihydrophthalazin-1-yl)ethyl)-1-methylurea;
N-(1-(6,7-difluoro-4-oxo-3,4-dihydrophthalazin-1-yl)ethyl)-8-fluoro-N-methylindolizine-2-carboxamide;
N-(1-(6,7-difluoro-4-oxo-3,4-dihydrophthalazin-1-yl)ethyl)-N-methylindoline-2-carboxamide;
2-chloro-N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-2,3-dihydro-1H-indene-5-carboxamide;
N-(1-(6,7-difluoro-4-oxo-3,4-dihydrophthalazin-1-yl)ethyl)-N-methylbenzo[d]thiazole-5-carboxamide;
N-(1-(6,7-difluoro-4-oxo-3,4-dihydrophthalazin-1-yl)ethyl)-N-methylbenzo[d]thiazole-6-carboxamide;
N-(1-(6,7-difluoro-4-oxo-3,4-dihydrophthalazin-1-yl)ethyl)-N-methylbenzo[d]oxazole-5-carboxamide;
N-(1-(6,7-difluoro-4-oxo-3,4-dihydrophthalazin-1-yl)ethyl)-N-methylbenzo[d]oxazole-6-carboxamide;
5-chloro-N-(1-(6,7-difluoro-4-oxo-3,4-dihydrophthalazin-1-yl)ethyl)-N-methyl-6-(trifluoromethyl)nicotinamide;
4-chloro-N-(1-(6,7-difluoro-4-oxo-3,4-dihydrophthalazin-1-yl)ethyl)-3,5-difluoro-N-methylbenzamide;
N-(1-(6,7-difluoro-4-oxo-3,4-dihydrophthalazin-1-yl)ethyl)-4-(difluoromethyl)-6-fluoro-N-methyl-1H-indole-2-carboxamide;
N-(1-(6,7-difluoro-4-oxo-3,4-dihydrophthalazin-1-yl)ethyl)-8-(difluoromethyl)-N-methylindolizine-2-carboxamide;
4-bromo-N-(1-(6,7-difluoro-4-oxo-3,4-dihydrophthalazin-1-yl)ethyl)-3,5-difluoro-N-methylbenzamide;
N-(1-(6,7-difluoro-4-oxo-3,4-dihydrophthalazin-1-yl)ethyl)-2-fluoro-N-methyl-4H-thieno[3,2-b]pyrrole-5-carboxamide;
N-(1-(6,7-difluoro-4-oxo-3,4-dihydrophthalazin-1-yl)ethyl)-N,1-dimethyl-1H-indole-6-carboxamide;
N-(1-(6,7-difluoro-4-oxo-3,4-dihydrophthalazin-1-yl)ethyl)-N,1-dimethyl-1H-indole-5-carboxamide;
N-(1-(6,7-difluoro-4-oxo-3,4-dihydrophthalazin-1-yl)ethyl)-4-(difluoromethyl)-3,5-difluoro-N-methylbenzamide;
N-(1-(6,7-difluoro-4-oxo-3,4-dihydrophthalazin-1-yl)ethyl)-N-methyl-1H-indazole-5-carboxamide;
N-(1-(6,7-difluoro-4-oxo-3,4-dihydrophthalazin-1-yl)ethyl)-N-methyl-1H-indazole-6-carboxamide;
N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled, stereoisomer, any mixture of stereoisomers, tautomer, and/or any mixture of tautomers thereof.

In certain embodiments, the compound is at least one of the following:
(R)-3-(3-chloro-4-fluorophenyl)-1-methyl-1-(1-(4-oxo-3,4-dihydrophthalazin-1-yl)ethyl)urea;
(S)-3-(3-chloro-4-fluorophenyl)-1-methyl-1-(1-(4-oxo-3,4-dihydrophthalazin-1-yl)ethyl)urea;
(R)-3-(3-chloro-4-fluorophenyl)-1-isobutyl-1-(1-(4-oxo-3,4-dihydrophthalazin-1-yl)ethyl)urea;
(S)-3-(3-chloro-4-fluorophenyl)-1-isobutyl-1-(1-(4-oxo-3,4-dihydrophthalazin-1-yl)ethyl)urea;
(R)-3(3-chloro-4-fluorophenyl)-1-isobutyl-1-(1-(3-methyl-4-oxo-3,4-dihydrophthalazin-1-yl)ethyl)urea;
(S)-3-(3-chloro-4-fluorophenyl)-1-isobutyl-1-(1-(3-methyl-4-oxo-3,4-dihydrophthalazin-1-yl)ethyl)urea;
(R)-3-(4-fluorophenyl)-1-isobutyl-1-(1-(3-methyl-4-oxo-3,4-dihydrophthalazin-1-yl)ethyl)urea;
(S)-3-(4-fluorophenyl)-1-isobutyl-1-(1-(3-methyl-4-oxo-3,4-dihydrophthalazin-1-yl)ethyl)urea;
(R)-3-(3-chloro-4-fluorophenyl)-1-(1-(6-chloro-4-oxo-3,4-dihydrophthalazin-1-yl)ethyl)-1-isobutylurea;
(S)-3-(3-chloro-4-fluorophenyl)-1-(1-(6-chloro-4-oxo-3,4-dihydrophthalazin-1-yl)ethyl)-1-isobutylurea;
(R)-1-(1-(6-chloro-4-oxo-3,4-dihydrophthalazin-1-yl)ethyl)-3-(4-fluorophenyl)-1-isobutylurea;
(S)-1-(1-(6-chloro-4-oxo-3,4-dihydrophthalazin-1-yl)ethyl)-3-(4-fluorophenyl)-1-isobutylurea;
(R)-3-(3-chloro-4-fluorophenyl)-1-(1-(6-chloro-4-oxo-3,4-dihydrophthalazin-1-yl)ethyl)-1-methylurea;
(S)-3-(3-chloro-4-fluorophenyl)-1-(1-(6-chloro-4-oxo-3,4-dihydrophthalazin-1-yl)ethyl)-1-methylurea;
(R)-3-(3-chloro-4-fluorophenyl)-1-(1-(6-chloro-4-oxo-3,4-dihydrophthalazin-1-yl)ethyl)-1-(3-hydroxypropyl)urea;
(S)-3-(3-chloro-4-fluorophenyl)-1-(1-(6-chloro-4-oxo-3,4-dihydrophthalazin-1-yl)ethyl)-1-(3-hydroxypropyl)urea;
(R)-1-(1-(6-chloro-4-oxo-3,4-dihydrophthalazin-1-yl)ethyl)-3-(4-fluorophenyl)-1-(3-hydroxypropyl)urea;
(S)-1-(1-(6-chloro-4-oxo-3,4-dihydrophthalazin-1-yl)ethyl)-3-(4-fluorophenyl)-1-(3-hydroxypropyl)urea;
(R)-3-(3-chloro-4-fluorophenyl)-1-(1-(6-fluoro-4-oxo-3,4-dihydrophthalazin-1-yl)ethyl)-1-isobutylurea;
(S)-3-(3-chloro-4-fluorophenyl)-1-(1-(6-fluoro-4-oxo-3,4-dihydrophthalazin-1-yl)ethyl)-1-isobutylurea;
(R)-1-(1-(6-fluoro-4-oxo-3,4-dihydrophthalazin-1-yl)ethyl)-3-(4-fluorophenyl)-1-isobutylurea;
(S)-1-(1-(6-fluoro-4-oxo-3,4-dihydrophthalazin-1-yl)ethyl)-3-(4-fluorophenyl)-1-isobutylurea;
(R)-3-(3-chloro-4-fluorophenyl)-1-(1-(6-fluoro-4-oxo-3,4-dihydrophthalazin-1-yl)ethyl)-1-methylurea;
(S)-3-(3-chloro-4-fluorophenyl)-1-(1-(6-fluoro-4-oxo-3,4-dihydrophthalazin-1-yl)ethyl)-1-methylurea;
(R)-1-(1-(6-fluoro-4-oxo-3,4-dihydrophthalazin-1-yl)ethyl)-3-(4-fluorophenyl)-1-methylurea;
(S)-1-(1-(6-fluoro-4-oxo-3,4-dihydrophthalazin-1-yl)ethyl)-3-(4-fluorophenyl)-1-methylurea;
(R)-3-(4-fluorophenyl)-1-isobutyl-1-(1-(4-oxo-3,4-dihydrophthalazin-1-yl)ethyl)urea;
(S)-3-(4-fluorophenyl)-1-isobutyl-1-(1-(4-oxo-3,4-dihydrophthalazin-1-yl)ethyl)urea;
(R)-3-(3-chloro-4-fluorophenyl)-1-(1-(6,7-difluoro-3-methyl-4-oxo-3,4-dihydrophthalazin-1-yl)ethyl)-1-methylurea;
(S)-3-(3-chloro-4-fluorophenyl)-1-(1-(6,7-difluoro-3-methyl-4-oxo-3,4-dihydrophthalazin-1-yl)ethyl)-1-methylurea;
(R)-3-(3-chloro-4-fluorophenyl)-1-(1-(6,7-difluoro-3-methyl-4-oxo-3,4-dihydrophthalazin-1-yl)ethyl)-1-isobutylurea;
(S)-3-(3-chloro-4-fluorophenyl)-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthalazin-1-yl)ethyl)-3-(4-fluorophenyl)-1-isobutylurea;
(S)-1-(1-(6,7-difluoro-3-methyl-4-oxo-3,4-dihydrophthalazin-1-yl)ethyl)-3-(4-fluorophenyl)-1-isobutylurea;
(R)-3-(3-chloro-4-fluorophenyl)-1-(1-(6,7-difluoro-3-methyl-4-oxo-3,4-dihydrophthalazin-1-yl)ethyl)-1-(3-hydroxypropyl)urea;
(S)-3-(3-chloro-4-fluorophenyl)-1-(1-(6,7-difluoro-3-methyl-4-oxo-3,4-dihydrophthalazin-1-yl)ethyl)-1-(3-hydroxypropyl)urea;
(R)-3-(4-fluorophenyl)-1-(1-(6,7-difluoro-3-methyl-4-oxo-3,4-dihydrophthalazin-1-yl)ethyl)-1-(3-hydroxypropyl)urea;
(S)-3-(4-fluorophenyl)-1-(1-(6,7-difluoro-3-methyl-4-oxo-3,4-dihydrophthalazin-1-yl)ethyl)-1-(3-hydroxypropyl)urea;
(R)-3-(3-chloro-4-fluorophenyl)-1-(1-(6,7-difluoro-4-oxo-3,4-dihydrophthalazin-1-yl)ethyl)-1-isobutylure a;
(S)-3-(3-chloro-4-fluorophenyl)-1-(1-(6,7-difluoro-4-oxo-3,4-dihydrophthalazin-1-yl)ethyl)-1-isobutylurea;
(R)-1-(1-(6,7-difluoro-4-oxo-3,4-dihydrophthalazin-1-yl)ethyl)-1-isobutyl-3-phenylurea;
(S)-1-(1-(6,7-difluoro-4-oxo-3,4-dihydrophthalazin-1-yl)ethyl)-1-isobutyl-3-phenylurea;
(R)-3-(3-chloro-4-fluorophenyl)-1-(1-(6,7-difluoro-4-oxo-3,4-dihydrophthalazin-1-yl)ethyl)-1-methylurea;
(S)-3-(3-chloro-4-fluorophenyl)-1-(1-(6,7-difluoro-4-oxo-3,4-dihydrophthalazin-1-yl)ethyl)-1-methylurea;
(R)-2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide;
(S)-2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide;
(R)-3-cyclopropyl-1-(1-(6,7-difluoro-3-methyl-4-oxo-3,4-dihydrophthalazin-1-yl)ethyl)-1-isobutylurea;
(S)-3-cyclopropyl-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthal azin-1-yl)ethyl)-1-isobutyl-3-phenylurea;
(S)-1-(1-(6,7-difluoro-3-methyl-4-oxo-3,4-dihydrophthalazin-1-yl)ethyl)-1-isobutyl-3-phenylurea;
(R)-3-cyclopentyl-1-(1-(6,7-difluoro-3-methyl-4-oxo-3,4-dihydrophthalazin-1-yl)ethyl)-1-isobutylurea;
(S)-3-cyclopentyl-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthalazin-1-yl)ethyl)-3-(4-fluorophenyl)-1-methylurea;
(S)-1-(1-(6,7-difluoro-3-methyl-4-oxo-3,4-dihydrophthalazin-1-yl)ethyl)-3-(4-fluorophenyl)-1-methylurea;
(R)-1-(1-(6,7-difluoro-4-oxo-3,4-dihydrophthalazin-1-yl)ethyl)-3-(3,4-difluorophenyl)-1-methylurea;
(S)-1-(1-(6,7-difluoro-4-oxo-3,4-dihydrophthalazin-1-yl)ethyl)-3-(3,4-difluorophenyl)-1-methylurea;
(R)-1-(1-(6,7-difluoro-4-oxo-3,4-dihydrophthalazin-1-yl)ethyl)-1-methyl-3-(3,4,5-trifluorophenyl)urea;
(S)-1-(1-(6,7-difluoro-4-oxo-3,4-dihydrophthalazin-1-yl)ethyl)-1-methyl-3-(3,4,5-trifluorophenyl)urea;
(R)-1-(1-(6,7-difluoro-4-oxo-3,4-dihydrophthalazin-1-yl)ethyl)-3-(4-fluorophenyl)-1-methylurea;
(S)-1-(1-(6,7-difluoro-4-oxo-3,4-dihydrophthalazin-1-yl)ethyl)-3-(4-fluorophenyl)-1-methylurea;
(R)-3-(3-chloro-4-fluorophenyl)-1-(1-(5-fluoro-4-oxo-3,4-dihydrophthalazin-1-yl)ethyl)-1-methylurea;
(S)-3-(3-chloro-4-fluorophenyl)-1-(1-(5-fluoro-4-oxo-3,4-dihydrophthalazin-1-yl)ethyl)-1-methylurea;
(R)-3-(3,4-difluorophenyl)-1-(1-(5-fluoro-4-oxo-3,4-dihydrophthalazin-1-yl)ethyl)-1-methylurea;
(S)-3-(3,4-difluorophenyl)-1-(1-(5-fluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-N-methyl-1H-indole-2-carboxamide;
(S)-N-(1-(6,7-difluoro-4-oxo-3,4-dihydrophthalazin-1-yl)ethyl)-N-methyl-1H-indole-2-carboxamide;
(R)-N-(1-(6,7-difluoro-4-oxo-3,4-dihydrophthalazin-1-yl)ethyl)-N-ethyl-5-fluoro-1H-indole-2-carboxamide;
(S)-N-(1-(6,7-difluoro-4-oxo-3,4-dihydrophthalazin-1-yl)ethyl)-N-ethyl-5-fluoro-1H-indole-2-carboxamide;
(R)-N-(1-(6,7-difluoro-4-oxo-3,4-dihydrophthalazin-1-yl)ethyl)-N-ethyl-1H-indole-2-carboxamide;
(S)-N-(1-(6,7-difluoro-4-oxo-3,4-dihydrophthalazin-1-yl)ethyl)-N-ethyl-1H-indole-2-carboxamide;
(R)-N-(1-(6,7-difluoro-4-oxo-3,4-dihydrophthalazin-1-yl)ethyl)-N-ethyl-4,5-difluoro-1H-indole-2-carboxamide;
(S)-N-(1-(6,7-difluoro-4-oxo-3,4-dihydrophthalazin-1-yl)ethyl)-N-ethyl-4,5-difluoro-1H-indole-2-carboxamide;
(R)-N-(1-(6,7-difluoro-4-oxo-3,4-dihydrophthalazin-1-yl)ethyl)-5,6-difluoro-N-methyl-1H-indole-2-carboxamide;
(S)-N-(1-(6,7-difluoro-4-oxo-3,4-dihydrophthalazin-1-yl)ethyl)-5,6-difluoro-N-methyl-1H-indole-2-carboxamide;
(R)-N-(1-(6,7-difluoro-4-oxo-3,4-dihydrophthalazin-1-yl)ethyl)-5-fluoro-N-methyl-1H-indole-2-carboxamide;
(S)-N-(1-(6,7-difluoro-4-oxo-3,4-dihydrophthalazin-1-yl)ethyl)-5-fluoro-N-methyl-1H-indole-2-carboxamide;
(R)-N-(1-(6,7-difluoro-4-oxo-3,4-dihydrophthalazin-1-yl)ethyl)-6-fluoro-N-methyl-1H-indole-2-carboxamide;
(S)-N-(1-(6,7-difluoro-4-oxo-3,4-dihydrophthalazin-1-yl)ethyl)-6-fluoro-N-methyl-1H-indole-2-carboxamide;
(R)-N-(1-(6,7-difluoro-4-oxo-3,4-dihydrophthalazin-1-yl)ethyl)-4-fluoro-N-methyl-1H-indole-2-carboxamide;
(S)-N-(1-(6,7-difluoro-4-oxo-3,4-dihydrophthalazin-1-yl)ethyl)-4-fluoro-N-methyl-1H-indole-2-carboxamide;
(R)-N-(1-(6,7-difluoro-4-oxo-3,4-dihydrophthalazin-1-yl)ethyl)-4,5-difluoro-N-methyl-1H-indole-2-carboxamide;
(S)-N-(1-(6,7-difluoro-4-oxo-3,4-dihydrophthalazin-1-yl)ethyl)-4,5-difluoro-N-methyl-1H-indole-2-carboxamide;
(R)-N-(1-(6,7-difluoro-4-oxo-3,4-dihydrophthalazin-1-yl)ethyl)-N,3-dimethyl-1H-indole-2-carboxamide;
(S)-N-(1-(6,7-difluoro-4-oxo-3,4-dihydrophthalazin-1-yl)ethyl)-N,3-dimethyl-1H-indole-2-carboxamide;
(R)-N-(1-(6,7-difluoro-4-oxo-3,4-dihydrophthalazin-1-yl)ethyl)-N-isobutyl-1H-indole-2-carboxamide;
(S)-N-(1-(6,7-difluoro-4-oxo-3,4-dihydrophthalazin-1-yl)ethyl)-N-isobutyl-1H-indole-2-carboxamide;
(R)-N-(1-(6,7-difluoro-4-oxo-3,4-dihydrophthalazin-1-yl)ethyl)-N-methyl-4,5,6,7-tetrahydro-1H-indole-2-carboxamide;
(S)-N-(1-(6,7-difluoro-4-oxo-3,4-dihydrophthalazin-1-yl)ethyl)-N-methyl-4, 5,6,7-tetrahydro-1H-indole-2-carboxamide;
(R)-N-(1-(6,7-difluoro-4-oxo-3,4-dihydrophthalazin-1-yl)ethyl)-N-methylindolizine-2-carboxamide;
(S)-N-(1-(6,7-difluoro-4-oxo-3,4-dihydrophthalazin-1-yl)ethyl)-N-methylindolizine-2-carboxamide;
(R)-N-(1-(6,7-difluoro-4-oxo-3,4-dihydrophthalazin-1-yl)ethyl)-4,6-difluoro-N-methyl-1H-indole-2-carboxamide;
(S)-N-(1-(6,7-difluoro-4-oxo-3,4-dihydrophthalazin-1-yl)ethyl)-4,6-difluoro-N-methyl-1H-indole-2-carboxamide;
(R)-N-(1-(6,7-difluoro-4-oxo-3,4-dihydrophthalazin-1-yl)ethyl)-3-fluoro-N-methyl-4-(trifluoromethyl)benzamide;
(S)-N-(1-(6,7-difluoro-4-oxo-3,4-dihydrophthalazin-1-yl)ethyl)-3-fluoro-N-methyl-4-(trifluoromethyl)benzamide;
(R)-4-bromo-N-(1-(6,7-difluoro-4-oxo-3,4-dihydrophthalazin-1-yl)ethyl)-3-fluoro-N-methylbenzamide;
(S)-4-bromo-N-(1-(6,7-difluoro-4-oxo-3,4-dihydrophthalazin-1-yl)ethyl)-3-fluoro-N-methylbenzamide;
(R)-4-chloro-N-(1-(6,7-difluoro-4-oxo-3,4-dihydrophthalazin-1-yl)ethyl)-3-fluoro-N-methylbenzamide;
(S)-4-chloro-N-(1-(6,7-difluoro-4-oxo-3,4-dihydrophthalazin-1-yl)ethyl)-3-fluoro-N-methylbenzamide;
(R)-4-bromo-N-(1-(6,7-difluoro-4-oxo-3,4-dihydrophthalazin-1-yl)ethyl)-N-methylbenzamide;
(S)-4-bromo-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-N-methyl-4-(trifluoromethyl)benzamide;
(S)-N-(1-(6,7-difluoro-4-oxo-3,4-dihydrophthalazin-1-yl)ethyl)-N-methyl-4-(trifluoromethyl)benzamide;
(R)-N-(1-(6,7-difluoro-4-oxo-3,4-dihydrophthalazin-1-yl)ethyl)-3,4,5-trifluoro-N-methylbenzamide;
(S)-N-(1-(6,7-difluoro-4-oxo-3,4-dihydrophthalazin-1-yl)ethyl)-3,4,5-trifluoro-N-methylbenzamide;
(R)-3-chloro-N-(1-(6,7-difluoro-4-oxo-3,4-dihydrophthalazin-1-yl)ethyl)-4-fluoro-N-methylbenzamide;
(S)-3-chloro-N-(1-(6,7-difluoro-4-oxo-3,4-dihydrophthalazin-1-yl)ethyl)-4-fluoro-N-methylbenzamide;
(R)-4-chloro-N-(1-(6,7-difluoro-4-oxo-3,4-dihydrophthalazin-1-yl)ethyl)-N-methylbenzamide;
(S)-4-chloro-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-3,4-difluoro-N-methylbenzamide;
(S)-N-(1-(6,7-difluoro-4-oxo-3,4-dihydrophthalazin-1-yl)ethyl)-3,4-difluoro-N-methylbenzamide;
(R)-N-(1-(6,7-difluoro-4-oxo-3,4-dihydrophthalazin-1-yl)ethyl)-3-(difluoromethyl)-N-methylbenzamide;
(S)-N-(1-(6,7-difluoro-4-oxo-3,4-dihydrophthalazin-1-yl)ethyl)-3-(difluoromethyl)-N-methylbenzamide;
(R)-N-(1-(6,7-difluoro-4-oxo-3,4-dihydrophthalazin-1-yl)ethyl)-3-(difluoromethyl)-4-fluoro-N-methylbenzamide;
(S)-N-(1-(6,7-difluoro-4-oxo-3,4-dihydrophthalazin-1-yl)ethyl)-3-(difluoromethyl)-4-fluoro-N-methylbenzamide;
(R)-1-(1-(6,7-difluoro-4-oxo-3,4-dihydrophthalazin-1-yl)ethyl)-3-(3-(difluoromethyl)-4-fluorophenyl)-1-methylurea;
(S)-1-(1-(6,7-difluoro-4-oxo-3,4-dihydrophthalazin-1-yl)ethyl)-3-(3-(difluoromethyl)-4-fluorophenyl)-1-methylurea;
(R)-N-(1-(6,7-difluoro-4-oxo-3,4-dihydrophthalazin-1-yl)ethyl)-N-methylbenzamide;
(S)-N-(1-(6,7-difluoro-4-oxo-3,4-dihydrophthalazin-1-yl)ethyl)-N-methylbenzamide;
(R)-8-chloro-N-(1-(6,7-difluoro-4-oxo-3,4-dihydrophthalazin-1-yl)ethyl)-N-methylindolizine-2-carboxamide;
(S)-8-chloro-N-(1-(6,7-difluoro-4-oxo-3,4-dihydrophthalazin-1-yl)ethyl)-N-methylindolizine-2-carboxamide;
(R)-3-(3-cyano-4-fluorophenyl)-1-(1-(6,7-difluoro-4-oxo-3,4-dihydrophthalazin-1-yl)ethyl)-1-methylurea;
(S)-3-(3-cyano-4-fluorophenyl)-1-(1-(6,7-difluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-8-fluoro-N-methylindolizine-2-carboxamide;
(S)-N-(1-(6,7-difluoro-4-oxo-3,4-dihydrophthalazin-1-yl)ethyl)-8-fluoro-N-methylindolizine-2-carboxamide;
(2S)-N-(1-(6,7-difluoro-4-oxo-3,4-dihydrophthalazin-1-yl)ethyl)-N-methylindoline-2-carboxamide;
(S)-N-((S)-1-(6,7-difluoro-4-oxo-3,4-dihydrophthalazin-1-yl)ethyl)-N-methylindoline-2-carboxamide;
(S)-N-((R)-1-(6,7-difluoro-4-oxo-3,4-dihydrophthalazin-1-yl)ethyl)-N-methylindoline-2-carboxamide;
(2R)-N-(1-(6,7-difluoro-4-oxo-3,4-dihydrophthalazin-1-yl)ethyl)-N-methylindoline-2-carboxamide;
(R)-N-((R)-1-(6,7-difluoro-4-oxo-3,4-dihydrophthalazin-1-yl)ethyl)-N-methylindoline-2-carboxamide;
(R)-N-((S)-1-(6,7-difluoro-4-oxo-3,4-dihydrophthalazin-1-yl)ethyl)-N-methylindoline-2-carboxamide;
(R)-2-chloro-N-(1-(6,7-difluoro-4-oxo-3,4-dihydrophthalazin-1-yl)ethyl)-N-methyl-4H-thieno[3,2-b]pyrrole-5-carboxamide;
(S)-2-chloro-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-4H-thieno[3,2-b]pyrrole-5-carboxamide;
(S)-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-2,3-dihydro-1H-indene-5-carboxamide;
(S)-N-(1-(6,7-difluoro-4-oxo-3,4-dihydrophthalazin-1-yl)ethyl)-N-methyl-2,3-dihydro-1H-indene-5-carboxamide;
(R)-N-(1-(6,7-difluoro-4-oxo-3,4-dihydrophthalazin-1-yl)ethyl)-N-methylbenzo[d]thiazole-5-carboxamide;
(S)-N-(1-(6,7-difluoro-4-oxo-3,4-dihydrophthalazin-1-yl)ethyl)-N-methylbenzo[d]thiazole-5-carboxamide;
(R)-N-(1-(6,7-difluoro-4-oxo-3,4-dihydrophthalazin-1-yl)ethyl)-N-methylbenzo[d]thiazole-6-carboxamide;
(S)-N-(1-(6,7-difluoro-4-oxo-3,4-dihydrophthalazin-1-yl)ethyl)-N-methylbenzo[d]thiazole-6-carboxamide;
(R)-N-(1-(6,7-difluoro-4-oxo-3,4-dihydrophthalazin-1-yl)ethyl)-N-methylbenzo[d]oxazole-5-carboxamide;
(S)-N-(1-(6,7-difluoro-4-oxo-3,4-dihydrophthalazin-1-yl)ethyl)-N-methylbenzo[d]oxazole-5-carboxamide;
(R)-N-(1-(6,7-difluoro-4-oxo-3,4-dihydrophthalazin-1-yl)ethyl)-N-methylbenzo[d]oxazole-6-carboxamide;
(S)-N-(1-(6,7-difluoro-4-oxo-3,4-dihydrophthalazin-1-yl)ethyl)-N-methylbenzo[d]oxazole-6-carboxamide;
(R)-5-chloro-N-(1-(6,7-difluoro-4-oxo-3,4-dihydrophthalazin-1-yl)ethyl)-N-methyl-6-(trifluoromethyl)nicotinamide;
(S)-5-chloro-N-(1-(6,7-difluoro-4-oxo-3,4-dihydrophthalazin-1-yl)ethyl)-N-methyl-6-(trifluoromethyl)nicotinamide;
(R)-4-chloro-N-(1-(6,7-difluoro-4-oxo-3,4-dihydrophthalazin-1-yl)ethyl)-3,5-difluoro-N-methylbenzamide;
(S)-4-chloro-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-4-(difluoromethyl)-6-fluoro-N-methyl-1H-indole-2-carboxamide;
(S)-N-(1-(6,7-difluoro-4-oxo-3,4-dihydrophthalazin-1-yl)ethyl)-4-(difluoromethyl)-6-fluoro-N-methyl-1H-indole-2-carboxamide;
(R)-N-(1-(6,7-difluoro-4-oxo-3,4-dihydrophthalazin-1-yl)ethyl)-8-(difluoromethyl)-N-methylindolizine-2-carboxamide;
(S)-N-(1-(6,7-difluoro-4-oxo-3,4-dihydrophthalazin-1-yl)ethyl)-8-(difluoromethyl)-N-methylindolizine-2-carboxamide;
(R)-4-bromo-N-(1-(6,7-difluoro-4-oxo-3,4-dihydrophthalazin-1-yl)ethyl)-3,5-difluoro-N-methylbenzamide;
(S)-4-bromo-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(S)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(R)-N-(1-(6,7-difluoro-4-oxo-3,4-dihydrophthalazin-1-yl)ethyl)-N,1-dimethyl-1H-indole-6-carboxamide;
(S)-N-(1-(6,7-difluoro-4-oxo-3,4-dihydrophthalazin-1-yl)ethyl)-N,1-dimethyl-1H-indole-6-carboxamide;
(R)-N-(1-(6,7-difluoro-4-oxo-3,4-dihydrophthalazin-1-yl)ethyl)-N,1-dimethyl-1H-indole-5-carboxamide;
(S)-N-(1-(6,7-difluoro-4-oxo-3,4-dihydrophthalazin-1-yl)ethyl)-N,1-dimethyl-1H-indole-5-carboxamide;
(R)-N-(1-(6,7-difluoro-4-oxo-3,4-dihydrophthalazin-1-yl)ethyl)-4-(difluoromethyl)-3,5-difluoro-N-methylbenzamide;
(S)-N-(1-(6,7-difluoro-4-oxo-3,4-dihydrophthalazin-1-yl)ethyl)-4-(difluoromethyl)-3, 5-difluoro-N-methylbenzamide;
(R)-N-(1-(6,7-difluoro-4-oxo-3,4-dihydrophthalazin-1-yl)ethyl)-N-methyl-1H-indazole-5-carboxamide;
(S)-N-(1-(6,7-difluoro-4-oxo-3,4-dihydrophthalazin-1-yl)ethyl)-N-methyl-1H-indazole-5-carboxamide;
(R)-N-(1-(6,7-difluoro-4-oxo-3,4-dihydrophthalazin-1-yl)ethyl)-N-methyl-1H-indazole-6-carboxamide;
(S)-N-(1-(6,7-difluoro-4-oxo-3,4-dihydrophthalazin-1-yl)ethyl)-N-methyl-1H-indazole-6-carboxamide;
(R)-N-(1-(6,7-difluoro-4-oxo-3,4-dihydrophthalazin-1-yl)ethyl)-5-fluoro-N-methyl-6-(trifluoromethyl)nicotinamide;
(S)-N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled, stereoisomer, any mixture of stereoisomers, tautomer, and/or any mixture of tautomers thereof.

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸ F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the disclosure are useful within the methods of the disclosure in combination with one or more additional agents useful for treating HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); (g) GalNAc-siRNA conjugates targeted against an HBV gene transcript; and (h) therapeutic vaccine.

### (A) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,SR)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, U.S. Pat. Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Pat. Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Pat. Application Publications No. US 2011/0245484 A1 and US 2008/0286230 A1, all of which incorporated herein in their entireties by reference.

### (B) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int′l Patent Application Publication No. WO 2013144129), AT-61 (see Int′l Patent Application Publication No. WO 1998033501; and King, et al., 1998, Antimicrob. Agents Chemother. 42(12):3179-3186), DVR-01 and DVR-23 (see Int′l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Pat. Application Publication Nos. US 2015/0225355, US 2015/0132258, US 2016/0083383, US 2016/0052921, US 2019/0225593, and Int′l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156, WO 2018052967, WO 2018172852, WO 2020023710, WO 2020123674 and are incorporated herein in their entirety by reference.

### (C) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int′l Patent Application Publication No. WO 2013130703, and are incorporated herein in their entirety by reference.

In addition, reported cccDNA formation inhibitors include, but are not limited to, those generally and specifically described in U.S. Pat. Application Publication No. US 2015/0038515 A1, and are incorporated herein in their entirety by reference.

### (D) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Pat. Application Publication Nos. US 2015/0087659 and US 2013/0303552, all of which are incorporated herein in their entireties by reference.

In addition, reported RNA destabilizers include, but are not limited to, those generally and specifically described in Int′l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, WO 2019177937, WO 2019222238, WO 2020150366, WO 2021025976 and are incorporated herein in their entirety by reference.

### (E) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

### (F) Immunostimulators

### Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

### (G) GaINAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) and PCT/US2018/0226918 (PCT Application Publication number WO/2018/191278, published on Oct. 18, 2018), all of which are hereby incorporated by reference in their entireties.

### (H) Therapeutic Vaccines

In certain embodiments, administration of a therapeutic vaccine is useful in the practice of the present disclosure for the treatment of a viral disease in a subject. In certain embodiments, the viral disease is a hepatitis virus. In certain embodiments, the hepatitis virus is at least one selected from the group consisting of hepatitis B virus (HBV) and hepatitis D virus (HDV). In certain embodiments, the subject is a human.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent’s freezing temperature to the solvent’s boiling temperature. A given reaction can be carried out in one solvent or a mixture of more than one solvent. Depending on the particular reaction step, suitable solvents for a particular reaction step can be selected.

A compound of formula (Ia) or (Ib) can be prepared from commercially available or previously documented starting materials, for example, according to the synthetic methods outlined herein.

A compound of formula (Ib) can be prepared from commercially available or previously documented starting materials, for example, according to the synthetic methods outlined in Scheme 1. (Un)substituted ortho-formylbenzoic acids (II) can be commercially acquired. Condensation with hydrazine as described in J. Org. Chem., 2016, 81:1520-11526, provides III. Bromination of III with, in a non-limiting example, benzyl trimethylammonium tribromide as described in J. Org. Chem., 2016, 81:1520-11526 provides IV. Ketone V can be synthesized from bromophthalazinone IV via, for example, palladium catalyzed coupling with a vinyl stannane followed by hydrolysis of the resulting enol ether. Reductive alkylation utilizing V subsequently provides VI, which can be functionalized to afford Ib. When V is an aldehyde or a ketone, reductive alkylation can be achieved by reacting that compound with a primary amine to form an imine, which is then reacted with a reducing agent, such as but not limited to sodium borohydride, or a carbon-based nucleophile, such as but not limited to a Grignard reagent or an alkyl/aryl lithium.

Alternatively, commercially available phthalazin-1(2H)-one-4-carboxilic acids (VII) may be converted to Weinreb amide VIII. Reaction with a carbon-based nucleophile, such as but not limited to a Grignard reagent or an alkyl/aryl lithium or a reducing agent, such as but not limited to lithium aluminum hydride, provides V (Scheme 2).

Alternatively, commercially available or previously documented phthalic anhydrides (IX) can be reacted with hydrazine or N-substituted hydrazines to provide 2,3-dihydrophthalazine-1,4-diones (X). Reaction of X with trifluoromethanesulfonic anhydride or N-phenyl-bis(trifluoromethanesulfonimide) provides 4-oxo-3,4-dihydrophthalazin-1-yl trifluoromethanesulfonates (XI). In the case of unsymmetrically substituted phthalic anhydrides (IX), two possible regioisomers may be formed and separated. V can be synthesized from 4-oxo-3,4-dihydrophthalazin-1-yl trifluoromethanesulfonates XI via, for example, palladium catalyzed coupling with a vinyl stannane followed by hydrolysis of the resulting enol ether (Scheme 3).

Alternatively, reductive alkylation of V can be achieved by reacting that compound with a primary sulfinamide to form a sulfinimine, which is subsequently reacted with a reducing agent, such as but not limited to L-Selectride, or a carbon-based nucleophile, such as but not limited to a Grignard reagent or an alkyl/aryl lithium. In certain embodiments, the primary sulfinamide can be racemic, scalemic, or enantiopure, and can be used to influence the stereochemical outcome of the sulfinimine reduction. The resulting secondary sulfinamide can be further functionalized with an electrophile, such as but not limited to an alkyl halide, in the presence of base, such as but not limited to sodium hydride, and the sulfinamido group can be deprotected to provide VI (Scheme 4).

In certain embodiments, V can be N-functionalized with an electrophile, such as but not limited to 2-(trimethylsilyl)ethoxymethyl chloride, in the presence of base, such as but not limited to N,N-diisopropylethylamine. Under certain conditions, sulfinamido deprotection can be concomitant with N-dealkylation to provide VI (Scheme 5).

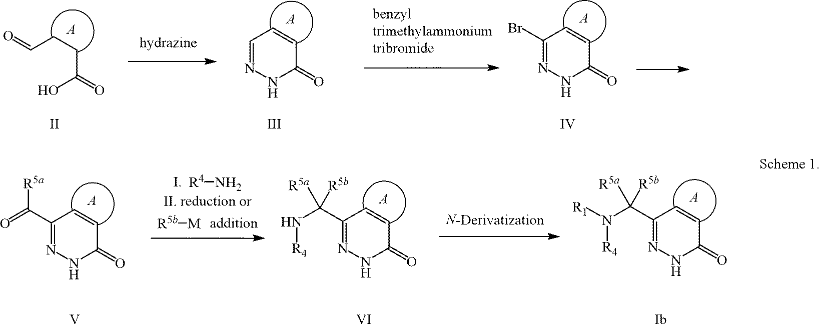

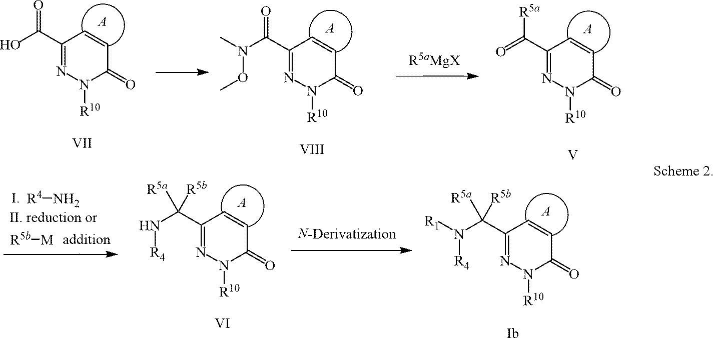

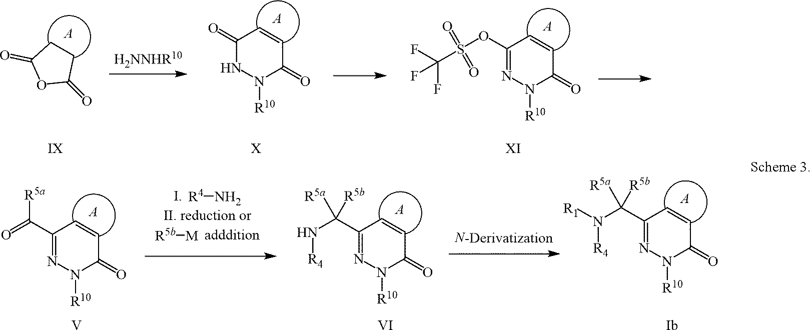

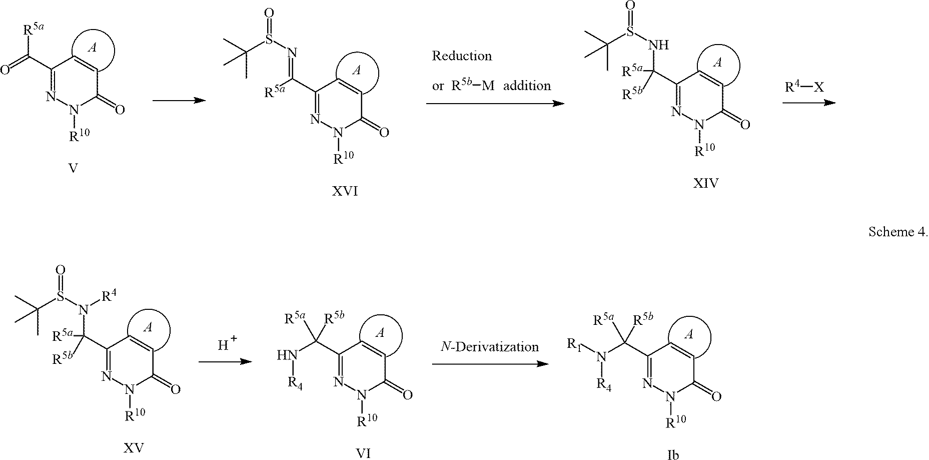

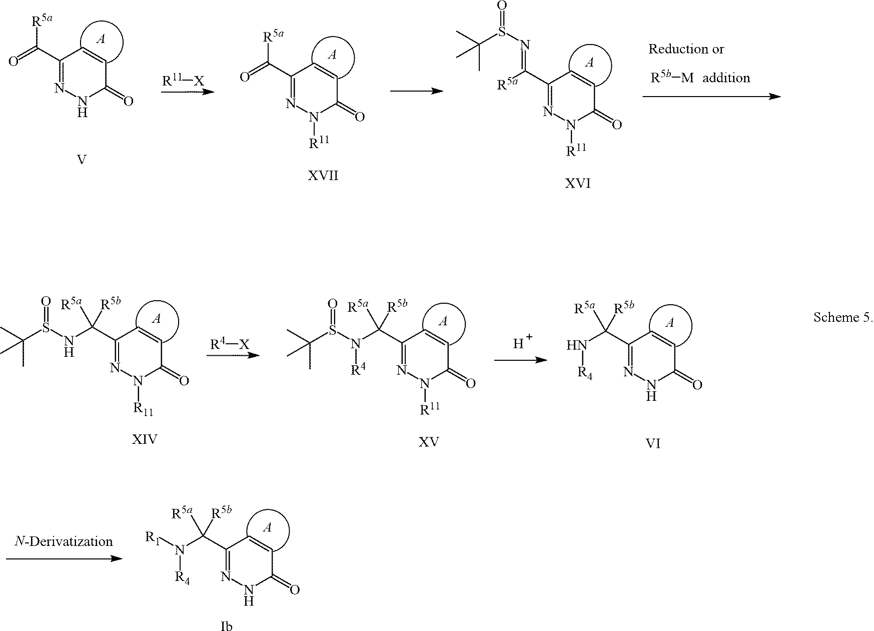

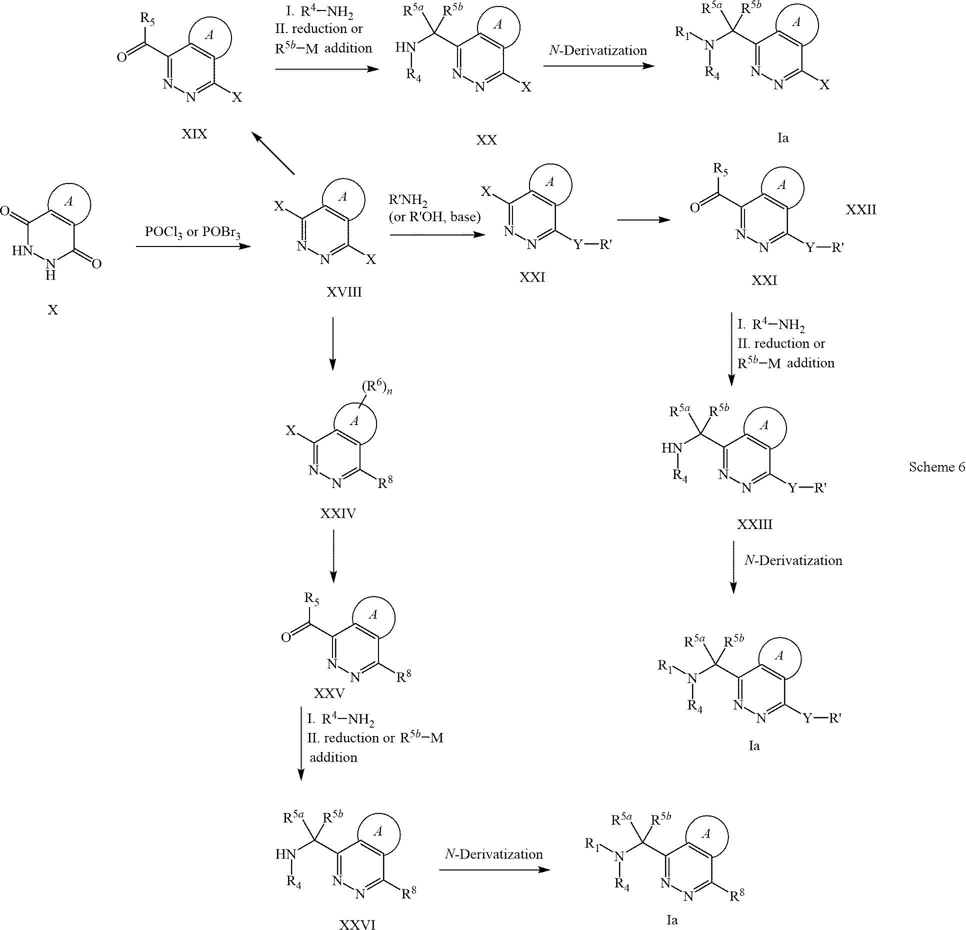

A compound of formula (Ia) can be prepared from commercially available or previously documented starting materials, for example, according to the synthetic methods outlined in Scheme 6. Dihalogenation of X with, in a non-limiting example, phosphorus oxychloride as described in US 2014/0303168, provides 1,4-dihalophthalazines (XVIII). Ketone XIX can be synthesized from 1,4-dihalophthalazines (XVIII) via palladium catalyzed coupling with a vinyl stannane followed by hydrolysis of the resulting enol ether. Reductive alkylation utilizing XIX subsequently provide XX which can be functionalized to afford Ia.

In certain embodiments, mono-halogen displacement of XVIII with, for example, an amine (Y=N), in a non-limiting example, as described in Bioorg. Med. Chem., 2009, 17:731-740, provides XXI. Ketone XXII can be synthesized from 1-halophthalazines (XXI) via palladium catalyzed coupling with a vinyl stannane followed by hydrolysis of the resulting enol ether. Reductive alkylation utilizing XXII subsequently provides XXIII which can be functionalized to afford Ia. In certain embodiments, palladium catalyzed coupling of XVIII with an aryl boronic acid or ester, in non-limiting examples, as described in US 2014/0303168, provides XXIV. Ketone XXV can be synthesized from XXIV via palladium catalyzed coupling with a vinyl stannane followed by hydrolysis of the resulting enol ether. Reductive alkylation utilizing XXV subsequently provide XXVI which can be functionalized to afford Ia.

The protocols incorporated elsewhere herein exemplify synthesis of representative compounds of the present disclosure. Analogous compounds can be synthesized in a similar fashion to those exemplified using the appropriately substituted intermediates and reagents. The disclosures of PCT Application No. PCT/US2019/065756 filed Dec. 11, 2019, U.S. Provisional Applications No. 62/896,237 filed Sep. 5, 2019, and U.S. Provisional Application No.62/778,471 filed Dec. 12, 2018, are incorporated herein by reference in their entireties.

### Methods

The disclosure provides a method of treating, ameliorating, or preventing hepatitis virus infection in a subject. In certain embodiments, the infection comprises hepatitis B virus (HBV) infection. In other embodiments, the method comprises administering to the subject in need thereof a therapeutically effective amount of at least one compound of the disclosure. In yet other embodiments, the at least one compound of the disclosure is the only antiviral agent administered to the subject. In yet other embodiments, the at least one compound is administered to the subject in a pharmaceutically acceptable composition. In yet other embodiments, the subject is further administered at least one additional agent useful for treating the hepatitis infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

The disclosure further provides a method of inhibiting expression and/or function of a viral capsid protein either directly or indirectly in a subject. In certain embodiments, the method comprises administering to the subject in need thereof a therapeutically effective amount of at least one compound of the disclosure. In other embodiments, the at least one compound is administered to the subject in a pharmaceutically acceptable composition. In yet other embodiments, the at least one compound of the disclosure is the only antiviral agent administered to the subject. In yet other embodiments, the subject is further administered at least one additional agent useful for treating HBV infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington’s Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 µg to about 7,500 mg, about 20 µg to about 7,000 mg, about 40 µg to about 6,500 mg, about 80 µg to about 6,000 mg, about 100 µg to about 5,500 mg, about 200 µg to about 5,000 mg, about 400 µg to about 4,000 mg, about 800 µg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 µg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound’s ability to perform its intended function, e.g., treating, ameliorating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); nonaqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer’s solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Pat. Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials & Methods

The following procedures can be utilized in evaluating and selecting compounds that inhibit hepatitis B virus infection.

### HepDE19 Assay With bDNA Quantitation of HBV rcDNA

HepDE19 cell culture system is a HepG2 (human hepatocarcinoma) derived cell line that supports HBV DNA replication and cccDNA formation in a tetracycline (Tet)-regulated manner and produces HBV rcDNA and a detectable reporter molecule dependent on the production and maintenance of cccDNA (Guo, et al.,2007, J. Virol. 81:12472-12484).

HepDE19 (50,000 cells/well) were plated in 96-well collagen-coated tissue-culture treated microtiter plates in DMEM/F12 medium supplemented with 10% fetal bovine serum, 1% penicillin-streptomycin and 1 µg/mL tetracycline and incubated in a humidified incubator at 37° C. and 5% CO₂ overnight. Next day, the cells were switched to fresh medium without tetracycline and incubated for 4 hours at 37° C. and 5% CO₂. The cells were treated with fresh Tet-free medium with compounds at concentrations starting at 25 µM and a serial, ½ log, 8-point, titration series in duplicate. The final DMSO concentration in the assay was 0.5%. The plates were incubated for 7 days in a humidified incubator at 37° C. and 5% CO₂. Following a 7 day-incubation, the level of rcDNA present in the inhibitor-treated wells was measured using a Quantigene 2.0 bDNA assay kit (Affymetrix, Santa Clara, CA) with HBV specific custom probe set and manufacturers instructions. Concurrently, the effect of compounds on cell viability was assessed using replicate plates, plated at a density of 5,000 cells/well and incubated for 4 days, to determine the ATP content as a measure of cell viability using the cell-titer glo reagent (CTG; Promega Corporation, Madison, WI) as per manufacturer’s instructions. The plates were read using a Victor luminescence plate reader (PerkinElmer Model 1420 Multilabel counter) and the relative luminescence units (RLU) data generated from each well was calculated as % inhibition of the untreated control wells and analyzed using XL-Fit module in Microsoft Excel to determine EC₅₀ and EC₉₀ (bDNA) and CC₅₀ (CTG) values using a 4-parameter curve fitting algorithm.

### LCMS Methods

LCMS Method A: Waters Acquity UPLC system employing a Waters Acquity UPLC BEH C18, 1.7 µm, 50 × 2.1 mm column with an aqueous acetonitrile based solvent gradient of 2-98% CH₃CN/H₂O (0.05% TFA) over 9.5 mins. Flow rate = 0.8 mL/min.

LCMS Method B: Waters Acquity UPLC system employing a Waters Acquity UPLC BEH C18, 1.7 µm, 50 × 2.1 mm column with an aqueous acetonitrile based solvent gradient of 2-98% CH₃CN/H₂O (0.05% TFA) over 1.0 mins. Flow rate = 0.8 mL/min.

LCMS Method C: Shimadzu UFLC system employing an ACE UltraCore Super PhenylHexyl, 2.5 µm, 50 × 2.1 mm column with an aqueous acetonitrile based solvent gradient of 5-100% CH₃CN/H₂O (0.05% Formic acid) over 5.0 mins. Flow rate = 1.0 mL/min.

LCMS Method D: Waters Acquity UPLC system employing a Waters Acquity UPLC BEH C18, 1.7 µm, 50 × 2.1 mm column with an aqueous acetonitrile based solvent gradient of 2-98% CH₃CN/H₂O (0.05% TFA) over 5.0 mins. Flow rate = 0.8 mL/min

LCMS Method E: Waters Acquity UPLC system employing a Waters Acquity UPLC C18, 1.7 µm, 50 × 2.1 mm column with an aqueous acetonitrile based solvent gradient of 5-95% CH₃CN/H₂O (0.05% Formic acid) over 3.7 mins. Flow rate = 0.6 mL/min

LCMS Method F: Waters Acquity UPLC system employing an XBridge BEH C18 2.5 µm, 50 × 2.1 mm column with an aqueous acetonitrile based solvent gradient of 3-95% CH₃CN/10 mM aqueous ammonium bicarbonate over 3.7 mins. Flow rate = 0.5 mL/min

As described herein, “Enantiomer I” or “Diastereomer I” refers to the first enantiomer or diastereomer eluded from the chiral column under the specific chiral analytical conditions detailed for examples provided elsewhere herein; and “Enantiomer II” or “Diastereomer II” refers to the second enantiomer or diastereomer eluded from the chiral column under the specific chiral analytical conditions detailed for examples provided elsewhere herein. Such nomenclature does not imply or impart any particular relative and/or absolute configuration for these compounds.

### EXAMPLE 1: Compounds

### 4-acetyl-2H-phthalazin-1-one (Va)

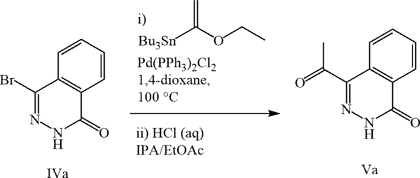

Step i: A suspension of 4-bromo-2H-phthalazin-1-one (IVa, 230 mg, 1.02 mmol) in anhydrous 1,4-dioxane (4.5 mL) was prepared in a pressure vessel. The mixture was degassed with nitrogen for 5 minutes. Tributyl(1-ethoxyvinyl)stannane (0.49 mL, 1.33 mmol) and dichlorobis(triphenylphosphine)palladium(II) (57 mg, 0.08 mmol) were added. The vessel was sealed and heated to 100° C. in a heating block for 3 h. Upon cooling, the mixture was diluted with EtOAc (15 mL) and MeCN (2 mL) and filtered through a pad of CELITE®. The CELITE® cake was washed with an additional portion of EtOAc (15 mL). The combined filtrate was evaporated under reduced pressure. The product was isolated by flash-chromatography (silica gel, EtOAc/hexanes 5 - 50% gradient) to provide 4-(1-ethoxyvinyl)-2H-phthalazin-1-one (153 mg, 69% yield). ¹H NMR (400 MHz, Chloroform-d) δ 10.02 (s, 1H), 8.44 (m, 1H), 7.99 (m, 1H), 7.88-7.74 (m, 2H), 4.64-4.54 (m, 2H), 4.03 (q, 2H), 1.43 (m, 3H).

Step ii: Hydrochloric acid (2 M, 1.04 mL, 2.08 mmol) was added to a solution of 4-(1-ethoxyvinyl)-2H-phthalazin-1-one (150 mg, 0.69 mmol) in IPA (4 mL) and EtOAc (0.5 mL). After 5 minutes a precipitate formed. The volatiles were removed in vacuo. The residue was re-suspended in IPA (4 mL) and toluene (1 mL), and the mixture was evaporated to dryness to provide 4-acetyl-2H-phthalazin-1-one (Va, 131 mg, 100% yield) as a white solid. ¹H NMR (400 MHz, Chloroform-d) δ 10.43 (s, 1H), 8.95 (m, 1H), 8.45 (m, 1H), 7.90 (m, 1H), 7.81 (m, 1H), 2.68 (s, 3H).

### 4-(Methylamino)ethyl]-2H-phthalazin-1-one (VIa)

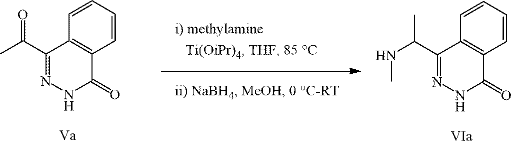

Tetraisopropoxytitanium (0.57 mL, 1.91 mmol) was added to a solution of 4-acetyl-2H-phthalazin-1-one (Va, 60 mg, 0.32 mmol) in a methylamine solution (2 M in THF, 1.28 mL, 2.56 mmol). The mixture was irradiated at 80° C. for 20 minutes, and then at 85° C. for an additional 10 minutes in a Biotage Initiator Plus microwave. The reaction mixture was diluted with 0.7 mL anhydrous methanol and cooled in an ice bath. Sodium borohydride (24 mg, 0.64 mmol) was added in one portion. The reaction mixture was stirred for 10 min, and the ice bath was removed. After an additional 20 min, the reaction mixture was slowly added to a rapidly stirred brine solution (0.5 mL) and diluted with 20 mL of 9:1 (v/v) EtOAc/MeCN. The mixture was filtered through CELITE®, and the filter cake was washed with an additional portion of EtOAc (15 mL). The combined filtrate was evaporated to dryness to provide crude racemic 4-[1-(methylamino)ethyl]-2H-phthalazin-1-one (VIa, 73 mg). LCMS: m/z found 204.2 [M+H]⁺, RT = 3.15 min, (Method D); ¹H NMR (400 MHz, DMSO-d₆) δ 12.55 (s, 1H), 8.40-8.33 (m, 1H), 8.26 (m, 1H), 7.98-7.82 (m, 1H), 7.86-7.78 (m, 1H), 4.16 (m, 1H), 2.23 (s, 3H), 1.38 (d, 3H).

### 3-chloro-4-fluorophenyl)-1-methyl-1-(1-(4-oxo-3,4-dihydrophthalazin-1-yl)ethyl)urea (Compounds 1, 3, 4)

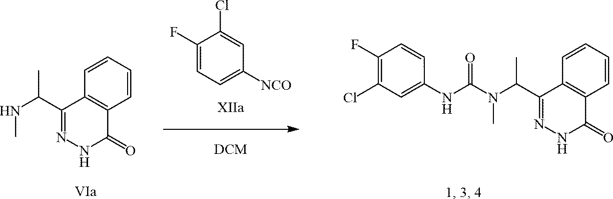

A solution of 2-chloro-l-fluoro-4-isocyanato-benzene (XIIa, 31 µL, 0.25 mmol) in DCM (0.7 mL) was added slowly to a 0° C. mixture of crude racemic 4-[1-(methylamino)ethyl]-2H-phthalazin-1-one (VIa, 64 mg, 0.31 mmol) in DCM (2 mL). After 5 minutes, two drops of MeOH were added and the crude reaction mixture was directly loaded on a preconditioned silica gel column. The product was isolated by flash-chromatography (silica gel, EtOAc/hexanes 5 - 60% gradient). The material was repurified by flash-chromatography (silica gel, acetone/hexanes 5 - 30% gradient)) to provide racemic 3-(3-chloro-4-fluoro-phenyl)-1-methyl-1-[1-(4-oxo-3H-phthalazin-1-yl)ethyl]urea (Compound 1, 62 mg, 52% yield). LCMS: m/z found 375.3/377.3 [M+H]⁺, RT = 7.08 min, (Method A); ¹H NMR (400 MHz, Methanol-d₄) δ 8.39 (m, 1H), 8.10 (m, 1H), 7.97-7.88 (m, 1H), 7.90-7.81 (m, 1H), 7.67 (m, 1H), 7.41-7.32 (m, 1H), 7.17 (t, 1H), 6.22 (m, 1H), 2.74 (s, 3H), 1.57 (d, 3H).

The enantiomers were subsequently separated by semi-preparative SFC: Method isocratic, Mobile phase (ACN:MeOH (1:1)): CO₂ - 20:80. Column: Chiralpak-AD (10 × 250 mm), 5 µm, flow rate: 9 g/min.

Enantiomer I (Compound 3): LCMS: m/z found 375.3/377.3 [M+H]⁺, RT = 7.33 min, (Method A); ¹H NMR (400 MHz, Methanol-d₄) δ 8.39 (m, 1H), 8.10 (m, 1H), 7.97-7.88 (m, 1H), 7.90-7.81 (m, 1H), 7.67 (m, 1H), 7.41-7.32 (m, 1H), 7.17 (t, 1H), 6.22 (m, 1H), 2.74 (s, 3H), 1.57 (d, 3H); Chiral analytical SFC: RT = 14.08 min, Column: Chiralpak AD (4.6 x 250 mm) 5 µm, 20% of (ACN:MeOH (1:1)), Flow rate: 3.0 g/min.

Enantiomer II (Compound 4): LCMS: m/z found 375.3/377.3 [M+H]⁺, RT = 7.33 min, (Method A); ¹H NMR (400 MHz, Methanol-d₄) δ 8.39 (m, 1H), 8.10 (m, 1H), 7.97-7.88 (m, 1H), 7.90-7.81 (m, 1H), 7.67 (m, 1H), 7.41-7.32 (m, 1H), 7.17 (t, 1H), 6.22 (m, 1H), 2.74 (s, 3H), 1.57 (d, 3H); Chiral analytical SFC: RT = 15.94 min, Column: Chiralpak AD (4.6 x 250 mm) 5 µm, 20% of (ACN:MeOH (1:1)), Flow rate: 3.0 g/min.

### 4-(Isobutylamino)ethyl]-2H-phthalazin-1-one (VIb)

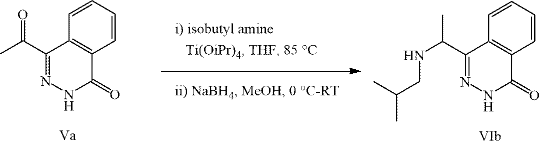

Tetraisopropoxytitanium (0.60 mL, 2.04 mmol) was added to a mixture of 4-acetyl-2H-phthalazin-1-one (Va, 64 mg, 0.34 mmol) and isobutylamine (67 µL, 0.68 mmol) in THF (1 mL). The mixture was irradiated to 85° C. for 30 min in a Biotage Initiator Plus microwave. The reaction mixture was diluted with anhydrous methanol (0.7 mL) and cooled in an ice bath. Sodium borohydride (19 mg, 0.51 mmol) was added in one portion. The reaction mixture was stirred for 10 minutes, and the ice bath was removed. After an additional 120 min, the reaction mixture was slowly added to a rapidly stirred brine solution (0.5 mL) and diluted with EtOAc/MeCN (20 mL, 9:1 v/v). The mixture was filtered through CELITE®, and the filter cake was washed with an additional portion of EtOAc (15 mL). The combined filtrate was evaporated to dryness to provide crude racemic 4-[1-(isobutylamino)ethyl]-2H-phthalazin-1-one (VIb, 83 mg). LCMS: m/z found 246.3 [M+H]⁺, RT = 3.48 min, (Method D); ¹H NMR (400 MHz, Methanol-d₄) δ 8.41 (m, 1H), 8.20-8.13 (m, 1H), 7.97 (m, 1H), 7.89 (m, 1H), 4.54 (m, 1H), 2.56 (m, 1H), 2.42 (m, 1H), 1.96-1.74 (m, 1H), 1.51 (d, 3H), 1.03-0.89 (m, 6H).

### 3-chloro-4-fluorophenyl)-1-isobutyl-1-(1-(4-oxo-3,4-dihydrophthalazin-1-yl)ethyl)urea (Compounds 2, 5, 6)

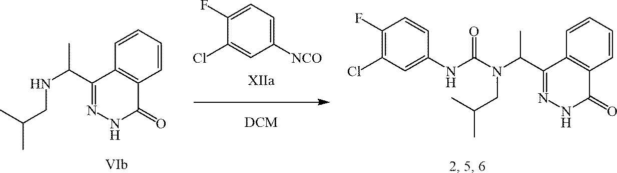

A solution of 2-chloro-l-fluoro-4-isocyanato-benzene (XIIa, 25 µL, 0.20 mmol) in 0.7 mL DCM (0.7 mL) was added slowly to a 0° C. mixture of crude racemic 4-[1-(isobutylamino)ethyl]-2H-phthalazin-1-one (VIb, 58 mg, 0.24 mmol) in DCM (2 mL). After 3 min, two drops MeOH were added and the crude reaction mixture was directly loaded on a preconditioned silica gel column. The product was isolated by flash-chromatography (silica gel, MeOH/DCM 1 - 4% gradient) to provide 3-(3-chloro-4-fluoro-phenyl)-1-isobutyl-1-[1-(4-oxo-3H-phthalazin-1-yl)ethyl]urea (Compound 2, 43 mg, 44% yield). LCMS: m/z found 417.3/419.3 [M+H]⁺, RT = 7.61 min, (Method A); ¹H NMR (400 MHz, Methanol-d₄) δ 8.38 (m, 1H), 8.26-8.19 (m, 1H), 7.96 (m, 1H), 7.91-7.82 (m, 1H), 7.64 (m, 1H), 7.34 (m 1H), 7.18 (t, 1H), 6.27 (m, 1H), 3.15-2.99 (m, 2H), 1.59 (d, 3H), 1.46 (m, 1H), 0.71 (d, 3H), 0.49 (d, 3H).

The enantiomers were subsequently separated by semi-preparative SFC: Method isocratic, Mobile phase: MeOH:CO₂ - 10:90. Column: Chiralpak-OJ (10 × 250 mm), 5 µm, flow rate: 9 g/min.

Enantiomer I (Compound 5): LCMS: m/z found 417.3/419.3 [M+H]⁺, RT = 7.65 min, (Method A); ¹H NMR (400 MHz, Methanol-d₄) δ 8.38 (m, 1H), 8.26-8.19 (m, 1H), 7.96 (m, 1H), 7.91-7.82 (m, 1H), 7.64 (m, 1H), 7.34 (m 1H), 7.18 (t, 1H), 6.27 (m, 1H), 3.15-2.99 (m, 2H), 1.59 (d, 3H), 1.46 (m, 1H), 0.71 (d, 3H), 0.49 (d, 3H). Chiral analytical SFC: RT = 6.35 min, Column: Chiralpak OJ (4.6*250 mm) 5 µm, 10% of MeOH, Flow rate: 3.0 g/min.

Enantiomer II (Compound 6): LCMS: m/z found 417.3/419.3 [M+H]⁺, RT = 7.62 min, (Method A); ¹H NMR (400 MHz, Methanol-d₄) δ 8.38 (m, 1H), 8.26-8.19 (m, 1H), 7.96 (m, 1H), 7.91-7.82 (m, 1H), 7.64 (m, 1H), 7.34 (m 1H), 7.18 (t, 1H), 6.27 (m, 1H), 3.15-2.99 (m, 2H), 1.59 (d, 3H), 1.46 (m, 1H), 0.71 (d, 3H), 0.49 (d, 3H). Chiral analytical SFC: RT = 8.53 min, Column: Chiralpak OJ (4.6 × 250 mm) 5 µm, 10% of MeOH, Flow rate: 3.0 g/min.

### N-methoxy-N,3-dimethyl-4-oxo-3,4-dihydrophthalazine-1-carboxamide (VIIIa)

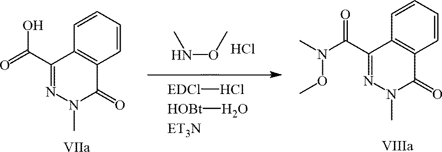

A mixture of 3-methyl-4-oxo-phthalazine-1-carboxylic acid (VIIa, 0.37 g, 1.81 mmol), N-methoxymethanamine hydrochloride (0.21 g, 2.17 mmol), HOBt monohydrate (0.24 g, 1.81 mmol), and triethylamine (0.38 mL, 2.72 mmol) was prepared in THF (10 mL). After cooling the mixture to 0° C., EDCI hydrochloride (0.42 g, 2.17 mmol) was added. The reaction mixture was allowed to warm to room temperature and stirred overnight. The volatiles were removed under reduced pressure and the product was isolated by flash-chromatography (silica gel, EtOAc/hexanes 20 - 80% gradient) to provide N-methoxy-N,3-dimethyl-4-oxo-phthalazine-1-carboxamide (VIIIa, 0.39 g, 86% yield). ¹H NMR (400 MHzChloroform-d) δ 8.51-8.42 (m, 1H), 7.79 (m, 2H), 7.70 (d, 1H), 3.87 (s, 3H), 3.67 (br s, 3H), 3.45 (br s, 3H).

### 4-acetyl-2-methylphthalazin-1(2H)-one (Vb)

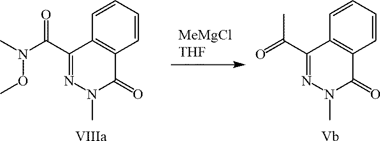

A solution of N-methoxy-N,3-dimethyl-4-oxo-phthalazine-1-carboxamide (VIIa, 0.39 g, 1.56 mmol) in anhydrous THF (14 mL) under nitrogen atmosphere was cooled in an ice bath. Methyl magnesium chloride solution (3 M in THF, 1.56 mL, 4.68 mmol) was added slowly. The reaction mixture was stirred at 0° C. for 1 h, then warmed to room temperature. After an additional 1 h, the mixture was cooled in an ice bath and quenched with saturated aqueous ammonium chloride solution (3 mL). The mixture was extracted with ethyl acetate (2 × 10 mL). The combined organics were then dried over sodium sulfate. The volatiles were removed under reduced pressure and the product was isolated by flash-chromatography (silica gel, MeOH/DCM 0 - 0% gradient) to provide 4-acetyl-2-methyl-phthalazin-1-one (Vb, 134 mg, 42% yield). ¹H NMR (400 MHz, DMSO-d₆) δ 8.78 (m, 1H), 8.31 (m, 1H), 7.97 (m, 1H), 7.89 (m, 1H), 3.83 (s, 3H), 2.62 (s, 3H).

### 4-(Isobutylamino)ethyl]-2-methyl-phthalazin-1-one (VIc)

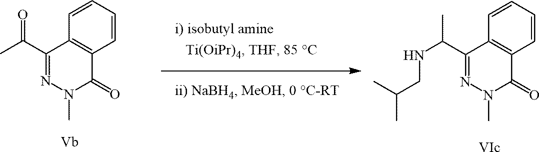

Tetraisopropoxytitanium (0.40 mL, 1.34 mmol) was added to a mixture of 4-acetyl-2-methyl-phthalazin-1-one (Vb, 68 mg, 0.33 mmol) and 2-methylpropan-1-amine (43 µL, 0.44 mmol) in THF (1.3 mL). The mixture was irradiated to 85° C. for 30 min in a Biotage Initiator Plus microwave. Upon cooling, the reaction mixture was diluted with anhydrous methanol (0.7 mL) and cooled in an ice bath. Sodium borohydride (19 mg, 0.50 mmol) was added in one portion. The reaction mixture was stirred for 10 minutes, and the ice bath was removed. After an additional 40 minutes, the reaction mixture was slowly added to a rapidly stirred brine solution (0.5 mL) and diluted with EtOAc/MeCN (20 mL, 9:1). The mixture was filtered through CELITE®, and the filter cake was washed with an additional portion of EtOAc (15 mL). The combined filtrate was evaporated to dryness to provide crude racemic 4-[1-(isobutylamino)ethyl]-2-methyl-phthalazin-1-one (VIc, 86 mg). LCMS: m/z found 260.3 [M+H]⁺, RT = 1.22 min, (Method B). ¹H NMR (400 MHz, Methanol-d₄) δ 8.42 (m, 1H), 8.15 (m, 1H), 8.00-7.82 (m, 2H), 4.59 (m, 1H), 3.85 (s, 3H), 2.61 (m, 1H), 2.46 (m, 1H), 1.86 (m, 1H), 1.53 (d, 3H), 1.11-0.91 (m, 6H).

### 3-chloro-4-fluorophenyl)-1-isobutyl-1-(1-methyl-4-oxo-3,4-dihydrophthalazin-1-yl)ethyl)urea (Compound 8)

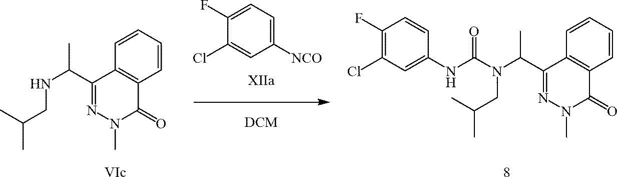

Racemic 3-(3-chloro-4-fluorophenyl)-1-isobutyl-1-(1-(3-methyl-4-oxo-3,4-dihydrophthalazin-1-yl)ethyl)urea was synthesized in an analogous manner as described above from racemic 4-(1-(isobutylamino)ethyl)-2-methylphthalazin-1(2H)-one (VIc) and 2-chloro-1-fluoro-4-isocyanatobenzene (XIIIa). LCMS: m/z found 431.3/433.4 [M+H]⁺, RT = 7.91 min, (Method A); ¹H NMR (400 MHz, DMSO-d₆) δ 8.47 (s, 1H), 8.30 (m, 1H), 8.12 (d, 1H), 7.96 (m, 1H), 7.90-7.76 (m, 2H), 7.48 (m, 1H), 7.33 (t, 1H), 6.20 (m, 1H), 3.77 (s, 3H), 3.11 (m, 1H), 2.96 (m, 1H), 1.48 (d, 3H), 1.35 (m, 1H), 0.63 (d, 3H), 0.39 (d, 3H).

### 3-fluorophenyl)-1-isobutyl-1-(1-(3-methyl-4-oxo-3,4-dihydrophthalazin-1-yl)ethyl)urea (Compound 9)

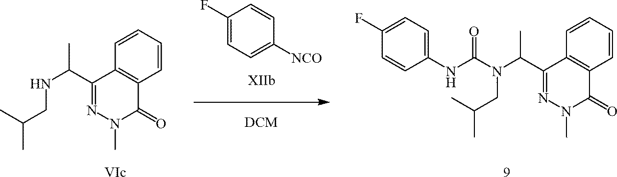

Racemic 3-(4-fluorophenyl)-1-isobutyl-1-(1-(3-methyl-4-oxo-3,4-dihydrophthalazin-1-yl)ethyl)urea was synthesized in an analogous manner as described above from racemic 4-(1-(isobutylamino)ethyl)-2-methylphthalazin-1(2H)-one (VIc) and 1-fluoro-4-isocyanatobenzene (XIIb). LCMS: m/z found 397.3. [M+H]⁺, RT = 7.51 min, (Method A); ¹H NMR (400 MHz, DMSO-d₆) δ 8.35-8.26 (m, 2H), 8.17 (d, 1H), 7.95 (m, 1H), 7.85 (m, 1H), 7.55-7.45 (m, 2H), 7.17-7.06 (m, 2H), 6.21 (m, 1H), 3.77 (d, 3H), 3.12 (m, 1H), 2.94 (m, 1H), 1.48 (d, 3H), 1.35 (m, 1H), 0.63 (d, 3H), 0.39 (d, 3H).

### 7-chloro-2H-phthalazin-1-one (IIa)

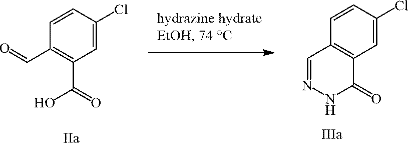

A solution of 5-chloro-2-formyl-benzoic acid (IIa, 0.83 g, 4.48 mmol) in ethanol (12 mL) was prepared. Hydrazine hydrate (0.70 mL, 22.38 mmol) was added over several minutes. The mixture was then heated to 74° C. A thick white precipitate formed within 30 minutes. After 2 h, the reaction mixture was cooled to room temperature and the resulting off-white precipitate was collected by vacuum filtration and washed with cold EtOH (6 mL). The collected precipitate was dried under high vacuum to provide 7-chloro-2H-phthalazin-1-one (IIIa, 0.64 g, 79% yield). ¹H NMR (400 MHz, DMSO- d₆) δ 12.82 (s, 1H), 8.41 (d, 1H), 8.17 (m, 1H), 8.00 (d, J = 1.4 Hz, 2H).

### 4-bromochlorophthalazin-1(2H)-one (IVb)

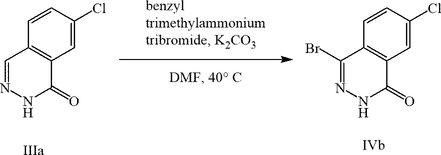

A suspension of 7-chloro-2H-phthalazin-1-one (IIIa, 0.57 g, 3.13 mmol) in DMF (6.7 mL) was prepared. Potassium carbonate (0.87 g, 6.27 mmol) was added, and the mixture was stirred for 10 min at room temperature. Benzyl trimethylammonium tribromide (2.44 g, 6.27 mmol) was added, and the mixture was heated in a 40° C. heating block for 5 h. After cooling to room temperature, the mixture was filtered through a pad of CELITE®. To the filtrate was added water (10 mL) and EtOAc (20 mL). The organic layer was washed with water (3 × 10 mL) and brine (10 mL), dried over sodium sulfate, and evaporated to dryness. The product was isolated by flash chromatography (silica gel, EtOAc/hexanes 5 - 60% gradient) to provide 4-bromo-7-chloro-2H-phthalazin-1-one (IVb, 0.73 g, 90% yield). ¹H NMR (400 MHz, DMSO-d₆) δ 13.09 (s, 1H), 8.24-8.14 (m, 1H), 8.08 (m, 1H), 7.95 (m, 1H).

### 4-acetyl-7-chlorophthalazin-1(2H)-one (Vc)

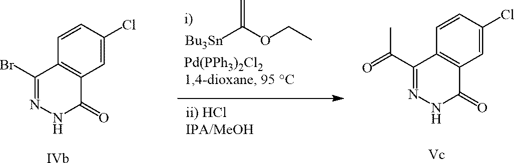

Step i: A mixture of 4-bromo-7-chloro-2H-phthalazin-1-one (IVb, 0.39 g, 1.48 mmol) in 1,4-dioxane (6 mL) in a pressure vessel was degassed with nitrogen. Tributyl(1-ethoxyvinyl)stannane (0.65 mL, 1.93 mmol) and dichlorobis(triphenylphosphine)palladium(II) (83 mg, 0.12 mmol) were added. The mixture was then heated in a 95° C. heating block for 2.5 h. The reaction mixture was diluted with EtOAc (25 mL) and filtered through a pad of CELITE®. The filter cake was washed with an additional portion of EtOAc (15 mL). The combined filtrate was evaporated to dryness. The product was isolated by flash-chromatography (silica gel, EtOAc/hexanes 5 - 55% gradient) to provide 7-chloro-4-(1-ethoxyvinyl)-2H-phthalazin-1-one (0.25 g, 67% yield). ¹H NMR (400 MHz, DMSO-d₆) δ 12.89 (s, 1H), 8.20 (m, 1H), 8.03-7.92 (m, 2H), 4.60 (d, 1H), 4.54 (d, 1H), 3.99 (q, 2H), 1.32 (t, 3H).

Step ii: Hydrochloric acid (2 M, 1.50 mL, 3.00 mmol) was added to a suspension of 7-chloro-4-(1-ethoxyvinyl)-2H-phthalazin-1-one (0.25 g, 1.00 mmol) in IPA (6 mL). MeOH (2 mL) was added and the mixture was stirred at room temperature for 2 h. The volatiles were removed in vacuo, and the resulting white solid was dried under high vacuum to provide 4-acetyl-7-chloro-2H-phthalazin-1-one (Vc, 211 mg, 95.0% yield). ¹H NMR (400 MHz, DMSO-d₆) δ 13.37 (s, 1H), 8.83 (m, 1H), 8.24-8.19 (m, 1H), 8.04 (m, 1H), 2.59 (s, 3H).

### 7-chloro-4-(1-(isobutylamino)ethyl)phthalazin-1(2H)-one (VId)

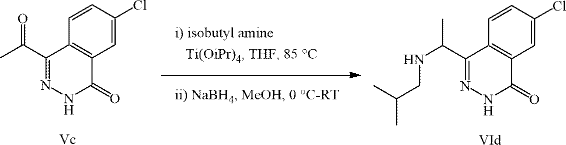

Tetraisopropoxytitanium (0.37 mL, 1.26 mmol) was added to a mixture of 4-acetyl-7-chloro-2H-phthalazin-1-one (Vc, 70 mg, 0.31 mmol) and 2-methylpropan-1-amine (37 µL, 0.38 mmol) in 1.5 mL THF. The mixture was irradiated to 85° C. for 30 min in a Biotage Initiator Plus microwave. The reaction mixture was then diluted with anhydrous MeOH (0.7 mL) and cooled in an ice bath. Sodium borohydride (18 mg, 0.47 mmol) was added in one portion. The reaction mixture was stirred for 20 minutes, and the ice bath was removed. After an additional 40 min, the reaction mixture was added slowly to a rapidly stirred brine solution (0.5 mL) and diluted with 20 mL of 9:1 (v/v) EtOAc/MeCN. The mixture was filtered through a pad of CELITE®, and the filter cake was washed with an additional portion of EtOAc (15 mL). The combined filtrate was evaporated to dryness to provide crude racemic 7-chloro-4-[1-(isobutylamino)ethyl]-2H-phthalazin-1-one (VId, 92 mg). LCMS: m/z found 280.3/282.3 [M+H]⁺, RT = 1.27 min, (Method B). ¹H NMR (400 MHz, Methanol-d₄) δ 8.35 (m, 1H), 8.25 (d, 1H), 7.93 (m, 1H), 4.40 (m, 1H), 2.49 (m, 1H), 2.33 (m, 1H), 1.84-1.69 (m, 1H), 1.47 (d, 3H), 0.91 (m, 6H).

### 3-chloro-4-fluorophenyl)-1-(1-(6-chloro-4-oxo-3,4-dihydrophthalazin-1-yl)ethyl)-1-isobutylurea (Compound 10)

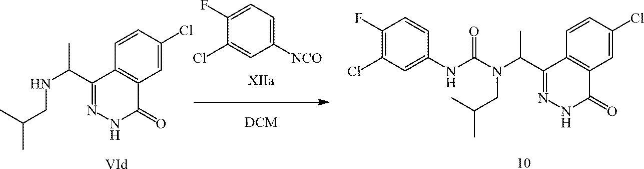

Racemic 3-(3-chloro-4-fluorophenyl)-1-(1-(6-chloro-4-oxo-3,4-dihydrophthalazin-1-yl)ethyl)-1-isobutylurea was synthesized in an analogous manner as described above from racemic 7-chloro-4-(1-(isobutylamino)ethyl)phthalazin-1(2H)-one (VId) and 2-chloro-1-fluoro-4-isocyanatobenzene (XIIa). LCMS: m/z found 451.3/453.3 [M+H]⁺, RT = 8.09 min, (Method A); ¹H NMR (400 MHz, DMSO-d₆) δ 12.91 (s, 1H), 8.47 (s, 1H), 8.24-8.11 (m, 2H), 8.08 (m, 1H), 7.80 (m, 1H), 7.48 (m, 1H), 7.33 (t, 1H), 6.17 (m, 1H), 3.11 (m, 1H), 2.91 (m, 1H), 1.46 (d, 3H), 1.33 (m, 1H), 0.62 (d, 3H), 0.40 (d, 3H).

### 1-(6-chloro-4-oxo-3,4-dihydrophthalazin-1-yl)ethyl)-3-(4-fluorophenyl)-1-isobutylurea (Compound 11)

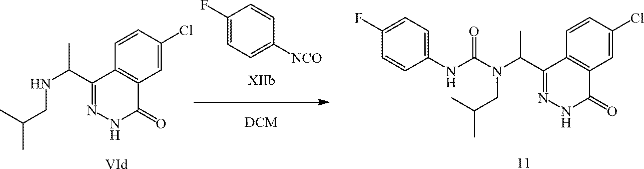

Racemic 1-(1-(6-chloro-4-oxo-3,4-dihydrophthalazin-1-yl)ethyl)-3-(4-fluorophenyl)-1-isobutylurea was synthesized in an analogous manner as described above from racemic 7-chloro-4-(1-(isobutylamino)ethyl)phthalazin-1(2H)-one (VId) and 1-fluoro-4-isocyanatobenzene (XIIb). LCMS: m/z found 417.3/419.2 [M+H]⁺, RT = 7.93 min, (Method A); ¹H NMR (400 MHz, DMSO-d₆) δ 12.89 (s, 1H), 8.33 (s, 1H), 8.24-8.16 (m, 2H), 8.07 (m, 1H), 7.55-7.44 (m, 2H), 7.17-7.06 (m, 2H), 6.18 (m, 1H), 3.12 (m, 1H), 2.89 (m, 1H), 1.45 (d, 3H), 1.35 (m, 1H), 0.62 (d, 3H), 0.40 (d, 3H).

### 7-chloro-4-(1-(methylamino)ethyl)phthalazin-1(2H)-one (VIe)

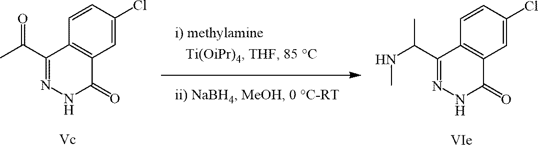

Tetraisopropoxytitanium (0.35 mL, 1.17 mmol) was added to a solution of 4-acetyl-7-chloro-2H-phthalazin-1-one (Vc, 65 mg, 0.29 mmol) in a methylamine solution (2 M in THF, 1.46 mL, 2.92 mmol). The mixture was irradiated to 85° C. for 30 min in a Biotage Initiator Plus microwave. The reaction mixture was diluted with anhydrous methanol (0.7 mL) and cooled in an ice bath. Sodium borohydride (17 mg, 0.44 mmol) was added in one portion. The reaction mixture was stirred for 20 min, and the ice bath was removed. After an additional 40 min, the reaction mixture was slowly added to a rapidly stirred brine solution (0.5 mL) and diluted with 20 mL of 9:1 (v/v) EtOAc/MeCN. The mixture was filtered through a pad of CELITE®, and the filter cake was washed with an additional portion of EtOAc (15 mL). The combined filtrate was evaporated to dryness to provide crude racemic 7-chloro-4-(1-(methylamino)ethyl)phthalazin-1(2H)-one (VIe, 45 mg). LCMS: m/z found 238.2/240.2 [M+H]⁺, RT = 1.30 min, (Method B); ¹H NMR (400 MHz, Methanol-d₄) δ 8.36 (m, 1H), 8.14 (m, 1H), 7.95 (m, 1H), 4.44 (m, 1H), 2.44 (s, 3H), 1.49 (d, 3H).

### 3-chloro-4-fluorophenyl)-1-(1-(6-chloro-4-oxo-3,4-dihydrophthalazin-1-yl)ethyl)-1-methylurea (Compound 12)

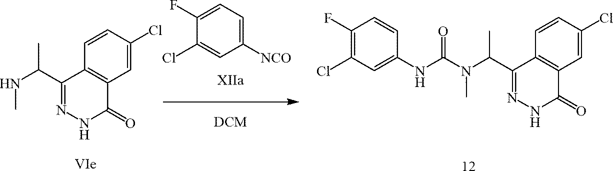

Racemic 3-(3-chloro-4-fluorophenyl)-1-(1-(6-chloro-4-oxo-3,4-dihydrophthalazin-1-yl)ethyl)-1-methylurea was synthesized in an analogous manner as described above from racemic 7-chloro-4-(1-(methylamino)ethyl)phthalazin-1(2H)-one (VIe) and 2-chloro-1-fluoro-4-isocyanatobenzene (XIIa). LCMS: m/z found 409.2/411.2 [M+H]⁺, RT = 7.92 min, (Method A); ¹H NMR (400 MHz, DMSO-d₆) δ 12.89 (s, 1H), 8.53 (s, 1H), 8.21 (d, 1H), 8.09-7.97 (m, 2H), 7.85 (m, 1H), 7.50 (m, 1H), 7.32 (t, 1H), 6.10 (m, 1H), 2.66 (s, 3H), 1.43 (d, 3H).

### 7-chloro-4-(1-((3-hydroxypropyl)amino)ethyl)phthalazin-1(2H)-one (VIf)

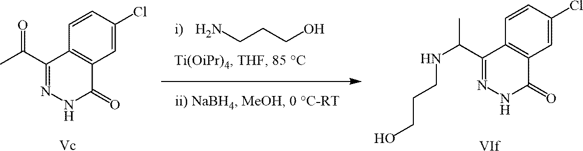

Tetraisopropoxytitanium (0.36 mL, 1.23 mmol) was added to a mixture of 4-acetyl-7-chloro-2H-phthalazin-1-one (Vc, 68 mg, 0.31 mmol) and 3-aminopropan-1-ol (28 µL, 0.37 mmol) in THF (1.5 mL). The mixture was irradiated to 85° C. for 30 min in a Biotage Initiator Plus microwave. The reaction mixture was then diluted with anhydrous methanol (0.7 mL) and cooled in an ice bath. Sodium borohydride (17 mg, 0.46 mmol) was added in one portion. The reaction mixture was stirred for 20 minutes, and the ice bath was removed. After an additional 30 min, the reaction mixture was slowly added to a rapidly stirred brine solution (0.5 mL) and diluted with EtOAc/MeCN (9:1 v/v, 20 mL). The mixture was filtered through a pad of CELITE®, and the filter cake was washed with an additional portion of EtOAc (15 mL). The combined filtrate was evaporated to dryness to provide crude racemic 7-chloro-4-(1-((3-hydroxypropyl)amino)ethyl)phthalazin-1(2H)-one (VIr, 53 mg). LCMS: m/z found 282.3/248.2 [M+H]⁺, RT = 1.33 min, (Method B); ¹H NMR (400 MHz, Methanol-d₄) δ 8.35 (m, 1H), 8.20 (d, 1H), 7.94 (m, 1H), 4.45 (m, 1H), 3.61 (t, 2H), 2.80-2.61 (m, 2H), 1.74 (m, 2H), 1.55-1.36 (m, 3H).

### 3-chloro-4-fluorophenyl)-1-(1-(6-chloro-4-oxo-3,4-dihydrophthalazin-1-yl)ethyl)-1-hydroxypropyl)urea (Compound 13)

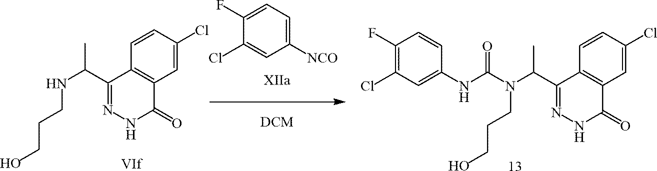

Racemic 3-(3-chloro-4-fluorophenyl)-1-(1-(6-chloro-4-oxo-3,4-dihydrophthalazin-1-yl)ethyl)-1-(3-hydroxypropyl)urea was synthesized in an analogous manner as described above from racemic 7-chloro-4-(1-((3-hydroxypropyl)amino)ethyl)phthalazin-1(2H)-one (VIf) and 2-chloro-1-fluoro-4-isocyanatobenzene (XIIa). LCMS: m/z found 453.2/455.2 [M+H]⁺, RT = 7.96 min, (Method A); ¹H NMR (400 MHz, DMSO-d₆) δ 12.90 (s, 1H), 8.78 (s, 1H), 8.21 (d, 1H), 8.06 (m, 1H), 7.98 (d, 1H), 7.80 (m, 1H), 7.43 (m, 1H), 7.34 (t, 1H), 6.13 (m, 1H), 5.02 (t, 1H), 3.33-3.08 (m, 4H), 1.46 (d, 3H), 1.14 (t, 2H).

### 1-(6-chloro-4-oxo-3,4-dihydrophthalazin-1-yl)ethyl)-3-(4-fluorophenyl)-1-(3-hydroxypropyl)urea (Compound 14)

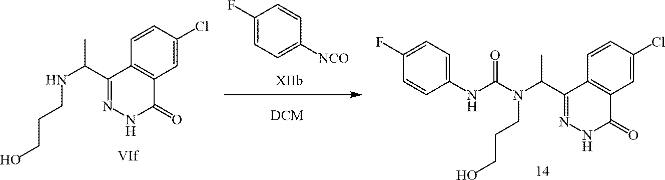

Racemic 1-(1-(6-chloro-4-oxo-3,4-dihydrophthalazin-1-yl)ethyl)-3-(4-fluorophenyl)-1-(3-hydroxypropyl)urea was synthesized in an analogous manner as described above from racemic 7-chloro-4-(1-((3-hydroxypropyl)amino)ethyl)phthalazin-1(2H)-one (VIf) and 1-fluoro-4-isocyanatobenzene (XIIb). LCMS: m/z found 419.2/421.2 [M+H]⁺, RT = 7.53 min, (Method A); ¹H NMR (400 MHz, DMSO-d₆) δ 12.89 (s, 1H), 8.63 (s, 1H), 8.21 (d, 1H), 8.09-7.97 (m, 2H), 7.54-7.44 (m, 2H), 7.17-7.06 (m, 2H), 6.15 (m, 1H), 4.99 (t, 1H), 3.33-3.08 (m, 4H), 1.46 (d, 3H), 1.15 (t, 2H).

### 7-fluoro-2H-phthalazin-1-one (IIIb)

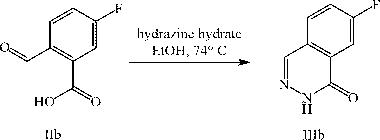

A solution of 5-fluoro-2-formyl-benzoic acid (IIb, 0.81 g, 4.84 mmol) in ethanol (12 mL) was prepared. Hydrazine hydrate (0.94 mL, 19.37 mmol) was added over several min and the mixture was heated to 74° C. A thick white precipitate formed within 30 min. After 2 h, the mixture was cooled to room temperature. The resulting white precipitate was collected by vacuum filtration, washed with ethanol (6 mL) and dried under high vacuum to provide 7-fluoro-2H-phthalazin-1-one (IIIb, 0.39 g, 49% yield). ¹H NMR (400 MHz, DMSO-d₆) δ 12.77 (s, 1H), 8.39 (d, 1H), 8.07 (m, 1H), 7.94-7.78 (m, 2H).

### 4-bromofluorophthalazin-1(2H)-one (IVc)

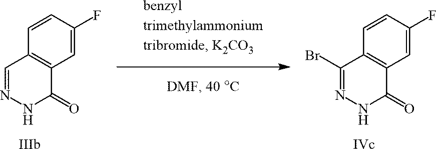

A solution of 7-fluoro-2H-phthalazin-1-one (IIIb, 0.32 g, 1.95 mmol) in anhydrous DMF (4 mL) was prepared. Potassium carbonate (0.54 g, 3.90 mmol) was added, and the mixture stirred for 10 min. Benzyl trimethylammonium tribromide (1.52 g, 3.90 mmol) was added, and the reaction was heated to 40° C. for 9 h. After cooling to room temperature, the mixture was filtered through a pad of CELITE®. To the filtrate, water (10 mL) and EtOAc (20 mL) were added. The organic layer was washed with water (3 × 10 mL) and brine (10 mL), dried over sodium sulfate and evaporated to dryness to provide crude 4-bromo-7-fluorophthalazin-1(2H)-one (IVc, 0.35 g). ¹H NMR (400 MHz, DMSO-d₆) δ 13.04 (s, 1H), 8.08-7.86 (m, 3H).

### 4-acetyl-7-fluorophthalazin-1(2H)-one (Vd)

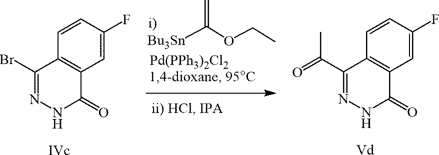

Step i: A mixture of 4-bromo-7-fluoro-2H-phthalazin-1-one (IVc, 0.25 g, 1.03 mmol) in 1,4-dioxane (4.7 mL) in a pressure vessel was degassed with nitrogen. Tributyl(1-ethoxyvinyl)stannane (0.45 mL, 1.34 mmol) and dichlorobis(triphenylphosphine) palladium(II) (60 mg, 0.08 mmol) were added. The mixture was heated in a 95° C. heating block for 2 h. Upon cooling, the reaction mixture was diluted with EtOAc (25 mL) and filtered through a pad of CELITE®. The filter cake was washed with an additional portion of EtOAc (15 mL). The combined filtrates were evaporated to dryness. The product was isolated by flash chromatography (silica gel, EtOAc/hexanes 5 - 75% gradient) to provide 4-(1-ethoxyvinyl)-7-fluoro-2H-phthalazin-1-one (0.19 g, 80% yield). ¹H NMR (400 MHz, DMSO-d₆) δ 12.84 (s, 1H), 8.04 (m, 1H), 7.94 (m, 1H), 7.81 (m, 1H), 4.60 (d, 1H), 4.53 (d, 1H), 3.99 (q, 2H), 1.33 (t, 3H).

Step ii: To a solution was prepared of 4-(1-ethoxyvinyl)-7-fluoro-2H-phthalazin-1-one (0.19 g, 0.82 mmol) in IPA (6 mL) was added hydrochloric acid (2 M, 1.24 mL, 2.48 mmol). After stirring for 30 min, the volatiles were removed in vacuo, and the off-white residue was dried under high vac overnight to provide 4-acetyl-7-fluoro-2H-phthalazin-1-one (Vd, 167 mg, 98% yield). ¹H NMR (400 MHz, DMSO-d₆) δ 13.32 (s, 1H), 8.90 (m, 1H), 7.96 (m, 1H), 7.88 (m, 1H), 2.59 (s, 3H).

### 7-fluoro-4-(1-(isobutylamino)ethyl)phthalazin-1(2H)-one (VIg)

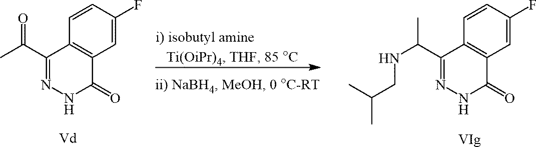

Tetraisopropoxytitanium, (0.32, 1.07 mmol) was added to a mixture of 4-acetyl-7-fluoro-2H-phthalazin-1-one (Vd, 55 mg, 0.27 mmol) and 2-methylpropan-1-amine (32 µL, 0.32 mmol) in THF (1.3 mL). The mixture was irradiated to 85° C. for 30 min in a Biotage Initiator Plus microwave. The reaction mixture was diluted with anhydrous methanol (0.7 mL) and cooled in an ice bath. Sodium borohydride (15 mg, 0.40 mmol) was added in one portion. The reaction mixture was stirred for 10 min, and the ice bath was removed. After an additional 20 min, the reaction mixture was added slowly to a rapidly stirred brine solution (0.5 mL) and diluted with 20 mL of 9:1 (v/v) EtOAc/MeCN. The mixture was filtered through a pad of CELITE®, and the filter cake was washed with an additional portion of EtOAc (15 mL). The combined filtrate was evaporated to dryness to provide crude racemic 7-fluoro-4-[1-(isobutylamino)ethyl]-2H-phthalazin-1-one (VIg, 73 mg). LCMS: m/z found 264.3 [M+H]⁺, RT = 1.36 min, (Method B); ¹H NMR (400 MHz, Methanol-d₄) δ 8.28 (m, 1H), 8.04 (m, 1H), 7.75 (m, 1H), 4.58 (m, 1H), 2.61 (m, 1H), 2.48 (m, 1H), 1.94-1.78 (m, 1H), 1.53 (d, 3H), 1.11-0.91 (m, 6H).

### 3-chloro-4-fluorophenyl)-1-(1-(6-fluoro-4-oxo-3,4-dihydrophthalazin-1-yl)ethyl)-1-isobutylurea (Compound 15)

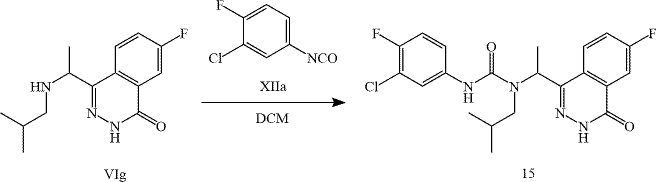

Racemic 3-(3-chloro-4-fluorophenyl)-1-(1-(6-fluoro-4-oxo-3,4-dihydrophthalazin-1 -yl)ethyl)-1-isobutylurea was synthesized in an analogous manner as described above from racemic 7-fluoro-4-(1-(isobutylamino)ethyl)phthalazin-1(2H)-one (VIg) and 2-chloro-1-fluoro-4-isocyanatobenzene (XIIa). LCMS: m/z found 435.3/437.3 [M+H]⁺, RT = 4.71 min, (Method D); ¹H NMR (400 MHz, Methanol-d₄) δ 8.34 (m, 1H), 8.01 (m, 1H), 7.74 (m, 1H), 7.64 (m, 1H), 7.38-7.29 (m, 1H), 7.18 (t, 1H), 6.26 (m, 1H), 3.08 (m, 2H), 1.58 (d, 3H), 1.46 (m, 1H), 0.72 (d, 3H), 0.48 (d, 3H).

### 1-(6-fluoro-4-oxo-3,4-dihydrophthalazin-1-yl)ethyl)-3-(4-fluorophenyl)-1-isobutylurea (Compounds 16, 20, 21)

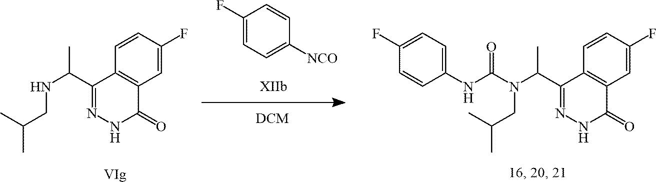

Racemic 1-(1-(6-fluoro-4-oxo-3,4-dihydrophthalazin-1-yl)ethyl)-3-(4-fluorophenyl)-1-isobutylurea was synthesized in an analogous manner as described above from racemic 7-fluoro-4-(1-(isobutylamino)ethyl)phthalazin-1(2H)-one (VIg) and 1-fluoro-4-isocyanatobenzene (XIIb). LCMS: m/z found 401.3 [M+H]⁺, RT = 8.01 min, (Method A); ¹H NMR (400 MHz, Methanol-d₄) δ 8.36 m, 1H), 8.01 (m, 1H), 7.73 (m, 1H), 7.46-7.34 (m, 2H), 7.11-7.00 (m, 2H), 6.27 (m, 1H), 3.17-2.98 (m, 2H), 1.57 (d, 3H), 1.48 (m, 1H), 0.73 (d, 3H), 0.48 (d, 3H).

The enantiomers were subsequently separated by semi-preparative SFC: Method isocratic, Mobile phase (ACN:MeOH (1:1)): CO₂ - 10:90. Column: Chiralpak-OJ (10 × 250 mm), 5 µm, flow rate: 9 g/min.

Enantiomer I (Compound 20): LCMS: m/z found 401.2 [M+H]⁺, RT = 7.95 min, (Method A); ¹H NMR (400 MHz, Methanol-d₄) δ 8.36 (m, 1H), 8.01 (m, 1H), 7.73 (m, 1H), 7.46-7.34 (m, 2H), 7.11-7.00 (m, 2H), 6.27 (m, 1H), 3.17-2.98 (m, 2H), 1.57 (d, 3H), 1.48 (m, 1H), 0.73 (d, 3H), 0.48 (d, 3H); Chiral analytical SFC: RT = 5.85 min, Column: Chiralpak OJ (10 × 250 mm) 5 µm, 10% of (ACN:MeOH (1:1)), Flow rate: 9.0 g/min.

Enantiomer II (Compound 21): LCMS: m/z found 401.3 [M+H]⁺, RT = 7.93 min, (Method A); ¹H NMR (400 MHz, Methanol-d₄) δ 8.36 (m, 1H), 8.01 (m, 1H), 7.73 (m, 1H), 7.46-7.34 (m, 2H), 7.11-7.00 (m, 2H), 6.27 (m, 1H), 3.17-2.98 (m, 2H), 1.57 (d, 3H), 1.48 (m, 1H), 0.73 (d, 3H), 0.48 (d, 3H); Chiral analytical SFC: RT = 8.34 min, Column: Chiralpak OJ (10 × 250 mm) 5 µm, 10% of (ACN:MeOH (1:1)), Flow rate: 9.0 g/min.

### 7-fluoro-4-(1-(methylamino)ethyl)phthalazin-1(2H)-one (VIh)

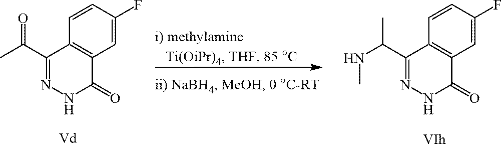

Tetraisopropoxytitanium (0.29 mL, 0.97 mmol) was added to a solution of 4-acetyl-7-fluoro-2H-phthalazin-1-one (Vd, 50 mg, 0.24 mmol) in a methylamine solution (2 M in THF, 1.46 mL, 2.92 mmol). The mixture was irradiated to 85° C. for 30 min in a Biotage Initiator Plus microwave. The reaction mixture was diluted with anhydrous methanol (0.7 mL) and cooled in an ice bath. Sodium borohydride (13 mg, 0.36 mmol) was added in one portion. The reaction mixture was stirred for 20 min, and the ice bath was removed. After an additional 40 min, the reaction mixture was slowly added to a rapidly stirred brine solution (0.5 mL) and diluted with 20 mL of 9:1 (v/v) EtOAc/MeCN. The mixture was filtered through a pad of CELITE®, and the filter cake was washed with an additional portion of EtOAc (15 mL). The combined filtrate was evaporated to dryness to provide crude racemic 7-fluoro-4-(1-(methylamino)ethyl)phthalazin-1(2H)-one (VIh, 60 mg). LCMS: m/z found 222.2 [M+H]⁺, RT = 1.33 min, (Method B); ¹H NMR (400 MHz, Methanol-d₄) δ 8.24 (m, 1H), 8.03 (m, 1H), 7.74 (m, 1H), 4.42 (m, 1H), 2.42 (s, 3H), 1.48 (d, J = 6.7 Hz, 3H).

### 3-chloro-4-fluorophenyl)-1-(1-(6-fluoro-4-oxo-3,4-dihydrophthalazin-1-yl)ethyl)-1-methylurea (Compound 17)

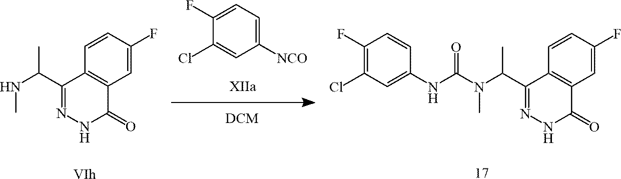

Racemic 3-(3-chloro-4-fluorophenyl)-1-(1-(6-fluoro-4-oxo-3,4-dihydrophthalazin-1-yl)ethyl)-1-methylurea was synthesized in an analogous manner as described above from racemic 7-fluoro-4-(1-(methylamino)ethyl)phthalazin-1(2H)-one (VIh) and 2-chloro-1-fluoro-4-isocyanatobenzene (XIIa). LCMS: m/z found 392.3/395.2 [M+H]⁺, RT = 7.69 min, (Method A); ¹H NMR (400 MHz, DMSO-d₆) δ 12.85 (s, 1H), 8.54 (s, 1H), 8.09 (m, 1H), 7.94 (m, 1H), 7.93-7.82 (m, 2H), 7.50 (m, 1H), 7.32 (t, 1H), 6.11 (m, 1H), 2.66 (s, 3H), 1.43 (d, 3H).

### 1-(6-fluoro-4-oxo-3,4-dihydrophthalazin-1-yl)ethyl)-3-(4-fluorophenyl)-1-methylurea (Compound 18)

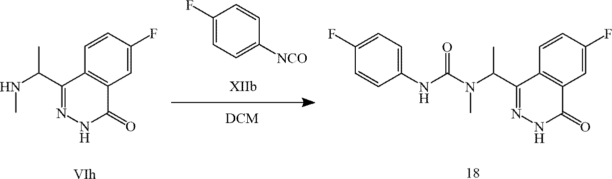

Racemic 1-(1-(6-fluoro-4-oxo-3,4-dihydrophthalazin-1-yl)ethyl)-3-(4-fluorophenyl)-1-methylurea was synthesized in an analogous manner as described above from racemic 7-fluoro-4-(1-(methylamino)ethyl)phthalazin-1(2H)-one (VIh) and 1-fluoro-4-isocyanatobenzene (XIIb). LCMS: m/z found 359.2 [M+H]⁺, RT = 7.37 min, (Method A); ¹H NMR (400 MHz, DMSO-d₆) δ 12.84 (s, 1H), 8.39 (s, 1H), 8.13 (m, 1H), 7.94 (m, 1H), 7.88 (m, 1H), 7.59-7.49 (m, 2H), 7.16-7.05 (m, 2H), 6.12 (m, 1H), 2.66 (s, 3H), 1.43 (d, 3H).

### 3-fluorophenyl)-1-isobutyl-1-(1-oxo-3,4-dihydrophthalazin-1-yl)ethyl)urea (Compound 19)

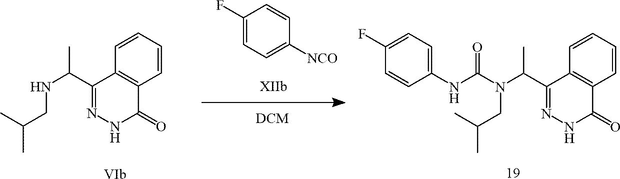

Racemic 3-(4-fluorophenyl)-1-isobutyl-1-(1-(4-oxo-3,4-dihydrophthalazin-1-yl)ethyl)urea was synthesized in an analogous manner as described above from racemic 4-[1-(isobutylamino)ethyl]-2H-phthalazin-1-one (VIb) and 1-fluoro-4-isocyanatobenzene (XIIb). LCMS: m/z found 383.3 [M+H]⁺, RT = 7.83 min, (Method A); ¹H NMR (400 MHz, Methanol-d₄) δ 8.38 (m, 1H), 8.28-8.21 (m, 1H), 7.95 (m, 1H), 7.86 (m, 1H), 7.44-7.34 (m, 2H), 7.11-7.00 (m, 2H), 6.28 (m, 1H), 3.15-2.99 (m, 2H), 1.59 (d, 3H), 1.48 (m, 1H), 0.72 (d, 3H), 0.49 (d, 3H).

### 6,7-difluoro-3-methyl-2H-phthalazine-1,4-dione (Xa)

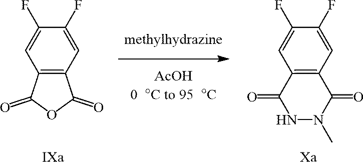

Methylhydrazine (0.47 mL, 8.96 mmol) was added slowly to a 0° C. solution of 5,6-difluoroisobenzofuran-1,3-dione (IXa, 1.50 g, 8.15 mmol) in 15 mL glacial acetic acid. After the hydrazine addition was complete, the reaction mixture was warmed to room temperature. A white solid slowly formed. The mixture was then heated to 95° C. for 3 h. An additional amount of white solid formed upon cooling. After standing at room temperature overnight, the white solid was collected by vacuum filtration, washed with 40 mL water, then dried under high vacuum. Additional material precipitated out of the filtrate. The material was collected by vacuum filtration, washed with water (10 mL), dried under high vacuum, and combined with the first precipitate to provide 6,7-difluoro-3-methyl-2H-phthalazine-1,4-dione (Xa, 1.38 g, 80%). LCMS: m/z found 213.1 [M+H]⁺, RT = 0.76 min, (Method B); ¹H NMR (400 MHz, DMSO-d₆) δ 11.96 (s, 1H), 8.13 (m, 1H), 7.93 (m, 1H), 3.56 (s, 3H).

### 6,7-difluoro-3-methyl-4-oxo-3,4-dihydrophthalazin-1-yl trifluoromethanesulfonate (XIa)

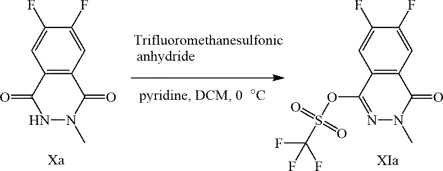

A suspension of pyridine (1.26 mL, 15.63 mmol) and 6,7-difluoro-3-methyl-2H-phthalazine-1,4-dione (Xa, 663 mg, 3.13 mmol) in DCM (32 mL) was cooled in an ice bath. A solution of trifluoromethanesulfonic anhydride (1 M in DCM, 3.44 mL, 3.44 mmol) was added slowly. The reaction mixture became a yellow solution. After 20 min at 0° C., the reaction mixture was diluted with DCM (30 mL) and washed hydrochloric acid (0.2 M, 2 × 50 mL). The organics were dried over sodium sulfate, filtered, and evaporated to dryness. The product was isolated by flash-chromatography (silica gel, EtOAc/hexanes 0 - 30% gradient) to provide (6,7-difluoro-3-methyl-4-oxo-phthalazin-1-yl) trifluoromethanesulfonate (XIa, 1.00 g, 93% yield). ¹H NMR (400 MHz, Chloroform-d) δ 8.25 (m, 1H), 7.62 (m, 1H), 3.80 (s, 3H).

### 4-acetyl-6,7-difluoro-2-methyl-phthalazin-1-one (Ve)

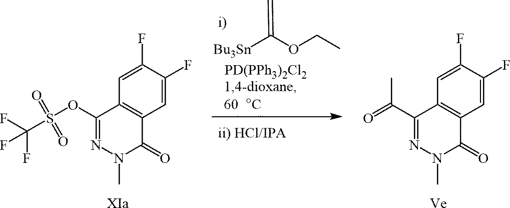

A solution of (6,7-difluoro-3-methyl-4-oxo-phthalazin-1-yl) trifluoromethanesulfonate (XIa, 0.97 g, 2.82 mmol) in 1,4-dioxane (12 mL) in a pressure tube was degassed with nitrogen. Tributyl(1-ethoxyvinyl)stannane (1.23 mL, 3.66 mmol) and dichlorobis(triphenylphosphine)palladium(II) (158 mg, 0.23 mmol) were added. The vessel was sealed, and the mixture heated in a 60° C. heating block for 45 min. Upon cooling, the reaction mixture was diluted with EtOAc (25 mL) and filtered through a pad of CELITE®. The filter cake was washed with an additional portion of EtOAc (25 mL). The combined filtrate was evaporated to dryness. The product was isolated by flash-chromatography (silica gel, EtOAc/hexanes 0 - 50% gradient) to provide 4-(1-ethoxyvinyl)-6,7-difluoro-2-methyl-phthalazin-1-one and the corresponding ketone in a 1:3 mixture (674 mg). Hydrochloric acid (2 M aqueous, 3.80 mL, 7.60 mmol) was added to a suspension of the above mixture in 20 mL IPA. After 80 min, the volatiles were removed in vacuo to provide 4-acetyl-6,7-difluoro-2-methyl-phthalazin-1-one (Ve, 0.67 g), which was carried forward crude. ¹H NMR (400 MHz, DMSO-d₆) δ 8.80-8.69 (m, 1H), 8.26 (m, 1H), 3.83 (s, 3H), 2.61 (s, 3H).

### 6,7-difluoro-2-methyl-4-(1-(methylamino)ethyl)phthalazin-1(2H)-one (VIt)

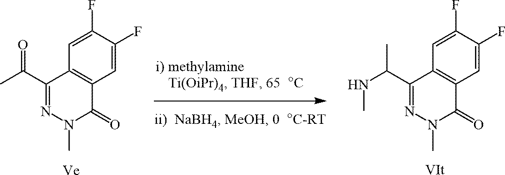

Tetraisopropoxytitanium (230 µL, 0.78 mmol) was added to a solution of 4-acetyl-6,7-difluoro-2-methyl-phthalazin-1-one (Ve, 110 mg, 0.46 mmol) in a methylamine solution (2 M in THF, 1.56 mL, 3.12 mmol) in a pressure vessel. The vessel was sealed, and the mixture was heated to 75° C. for 30 min in a Biotage Initiator Plus microwave. The reaction mixture was diluted with anhydrous methanol (0.7 mL) and cooled in an ice bath. Sodium borohydride (15 mg, 0.39 mmol) was added in one portion. The reaction mixture was stirred for 10 min, and the ice bath was removed. After an additional 40 min, the reaction mixture was slowly added to a rapidly stirred brine solution (0.5 mL) and diluted with 20 mL of 9:1 (v/v) EtOAc/MeCN. After stirring an additional 20 min, the mixture was filtered through a pad of CELITE°, and the filter cake was washed with an additional portion of EtOAc (15 mL). The combined filtrate was evaporated to dryness to provide crude racemic 6,7-difluoro-2-methyl-4-(1-(methylamino)ethyl)phthalazin-1(2H)-one (VIt, 150 mg, 40% pure). LCMS: m/z found 254.2 [M+H]⁺, RT = 0.58 min, (Method B).

### 3-chloro-4-fluorophenyl)-1-(1-(6,7-difluoro-3-methyl-4-oxo-3,4-dihydrophthalazin-1-yl)ethyl)-1-methylurea (Compound 22)

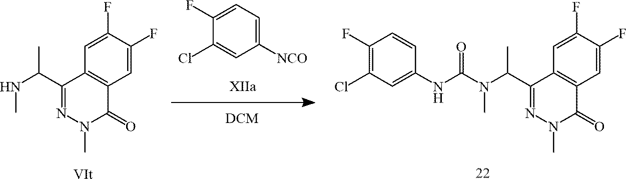

A mixture of crude racemic 6,7-difluoro-2-methyl-4-[1-(methylamino)ethyl]phthalazin-1-one (VIt, 70 mg, 0.28 mmol) in 2 mL DCM was cooled in an ice bath. A solution of 2-chloro-1-fluoro-4-isocyanato-benzene (XIIa, 24 µL, 0.19 mmol) in 0.5 mL DCM was added slowly. After 5 min, the reaction mixture was loaded directly on a pre-equilibrated silica gel column and the product was isolated by flash-chromatography (silica gel, MeOH/DCM 0.5-5% gradient). The product was re-purified by flash-chromatography (silica gel, EtOAc/DCM 0-30% gradient) to provide racemic 3-(3-chloro-4-fluoro-phenyl)-1-[1-(6,7-difluoro-3-methyl-4-oxo-phthalazin-1-yl)ethyl]-1-methyl-urea (22, 45 mg, 39% yield). LCMS: m/z found 425.1/427.2 [M+H]⁺, RT = 5.22 min, (Method A); ¹H NMR (400 MHz, DMSO-d₆) δ 8.56 (s, 1H), 8.23 (m, 1H), 8.08 (m, 1H), 7.81 (m, 1H), 7.51 (m, 1H), 7.34 (t, 1H), 6.06 (m, 1H), 3.76 (s, 3H), 2.70 (s, 3H), 1.44 (d, 3H).

### 6,7-difluoro-4-(1-(isobutylamino)ethyl)-2-methylphthalazin-1(2H)-one (VIn)

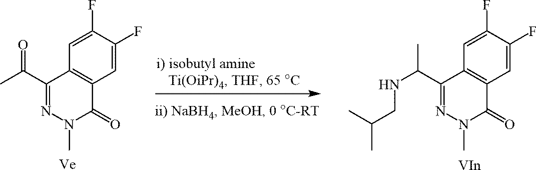

Tetraisopropoxytitanium (410 µL, 1.38 mmol) was added to a solution of 2-methylpropan-1-amine (55 µL, 0.55 mmol) and 4-acetyl-6,7-difluoro-2-methyl-phthalazin-1-one (Ve, 110 mg, 0.46 mmol) in anhydrous THF (1 mL) in a pressure vessel. The vessel was sealed, and the mixture was heated to 65° C. for 2.5 h in a heating block. The reaction mixture was diluted with anhydrous methanol (4 mL) and cooled in an ice bath. Sodium borohydride (26 mg, 0.69 mmol) was added in one portion. The reaction mixture was stirred for 10 min, and the ice bath was removed. After an additional 40 min, the reaction mixture was slowly added to a rapidly stirred brine solution (0.5 mL) and diluted with 20 mL of 9:1 (v/v) EtOAc/MeCN. After stirring an additional 20 min, the mixture was filtered through a pad of CELITE®, and the filter cake was washed with an additional portion of EtOAc (15 mL). The combined filtrate was evaporated to dryness to provide crude racemic 6,7-difluoro-4-(1-(isobutylamino)ethyl)-2-methylphthalazin-1(2H)-one (VIn, 150 mg). LCMS: m/z found 296.3 [M+H]⁺, RT = 0.76 min, (Method B); ¹H NMR (400 MHz, Methanol-d₄) δ 8.33 (m, 1H), 8.27-8.14 (m, 1H), 4.33 (m, 1H), 3.82 (s, 3H), 2.50 (m, 1H), 2.31 (m, 1H), 1.84-1.69 (m, 1H), 1.47 (d, 3H), 0.92 (m, 6H).

### 3-chloro-4-fluorophenyl)-1-(1-(6,7-difluoro-3-methyl-4-oxo-3,4-dihydrophthalazin-1-yl)ethyl)-1-isobutylurea (Compound 23)

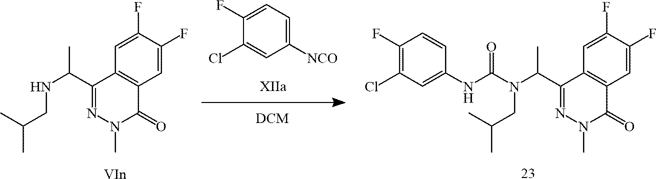

Racemic 3-(3-chloro-4-fluorophenyl)-1-(1-(6,7-difluoro-3-methyl-4-oxo-3,4-dihydrophthalazin-1-yl)ethyl)-1-isobutylurea was synthesized in an analogous manner as described above from racemic 6,7-difluoro-4-(1-(isobutylamino)ethyl)-2-methylphthalazin-1(2H)-one (VIn) and 2-chloro-1-fluoro-4-isocyanatobenzene (XIIa). LCMS: m/z found 467.1/469.2 [M+H]⁺, RT = 6.06 min, (Method A); ¹H NMR (400 MHz, Chloroform-d) δ 8.21 (m, 1H), 8.07 (m, 1H), 7.57 (m, 1H), 7.28-7.19 (m, 1H), 7.10 (t, 1H), 6.36 (s, 1H), 6.23 (m, 1H), 3.87 (s, 3H), 3.09-2.93 (m, 2H), 1.58-1.43 (m, 4H), 0.83 (d, 3H), 0.42 (d, 3H).

### 1-(6,7-difluoro-3-methyl-4-oxo-3,4-dihydrophthalazin-1-yl)ethyl)-3-(4-fluorophenyl)-1-isobutylurea (Compound 24)

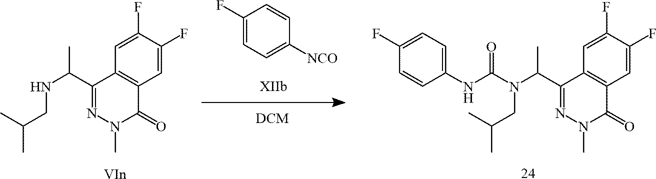

Racemic 1-(1-(6,7-difluoro-3-methyl-4-oxo-3,4-dihydrophthalazin-1-yl)ethyl)-3-(4-fluorophenyl)-1-isobutylurea was synthesized in an analogous manner as described above from racemic 6,7-difluoro-4-(1-(isobutylamino)ethyl)-2-methylphthalazin-1(2H)-one (VIn) and 1-fluoro-4-isocyanatobenzene (XIIb). LCMS: m/z found 433.2 [M+H]⁺, RT = 5.46 min, (Method A); ¹H NMR (400 MHz, Chloroform-d) δ 8.21 (m, 1H), 8.12 (m, 1H), 7.41-7.28 (m, 2H), 7.09-6.98 (m, 2H), 6.35 (s, 1H), 6.26 (m, 1H), 3.87 (s, 3H), 3.08-2.93 (m, 2H), 1.54 (m, 4H), 0.83 (d, 3H), 0.41 (d, 3H).

### 6,7-difluoro-4-(1-((3-hydroxypropyl)amino)ethyl)-2-methylphthalazin-1(2H)-one (VIo)

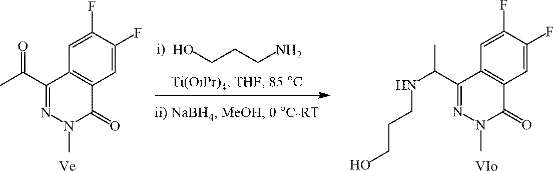

Tetraisopropoxytitanium (228 µL, 0.77 mmol) was added to a solution of 4-acetyl-6,7-difluoro-2-methyl-phthalazin-1-one (Ve, 61 mg, 0.26 mmol) and 3-aminopropan-1-ol (22 µL, 0.28 mmol) in anhydrous THF (1 mL) in a pressure vessel. The vessel was sealed, and the mixture heated to 65° C. for 2 h in a heating block. Upon cooling, the reaction mixture was diluted with anhydrous methanol (1 mL) and further cooled in an ice bath. Sodium borohydride 15 mg, 0.39 mmol) was added in one portion. The reaction mixture was stirred for 10 min, and the ice bath was removed. After an additional 40 min, the reaction mixture was added slowly to a rapidly stirred brine solution (0.5 mL) and diluted with 20 mL of 9:1 (v/v) EtOAc/MeCN. The mixture was filtered a pad of CELITE°, and the filter cake was washed with an additional portion of EtOAc (15 mL). The combined filtrate was evaporated to dryness to provide crude racemic 6,7-difluoro-4-(1-((3-hydroxypropyl)amino)ethyl)-2-methylphthalazin-1(2H)-one (VIo, 71 mg). LCMS: m/z found 298.2 [M+H]⁺, RT = 0.60 min, (Method B); ¹H NMR (400 MHz, Methanol-d₄) δ 8.30-8.13 (m, 2H), 4.36 (q, J= 6.7 Hz, 1H), 3.83 (s, 3H), 3.61 (t, J= 6.2 Hz, 2H), 2.78-2.61 (m, 2H), 1.79-1.68 (m, 2H), 1.47 (d, J= 6.7 Hz, 3H).

### 3-chloro-4-fluorophenyl)-1-(1-(6,7-difluoro-3-methyl-4-oxo-3,4-dihydrophthalazin-1-yl)ethyl)-1-hydroxypropyl)urea (Compound 25)

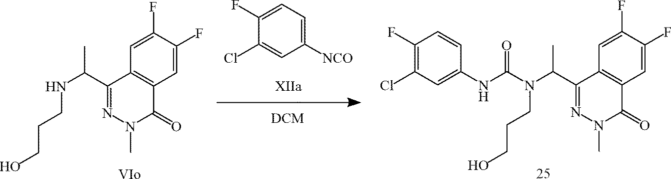

Racemic 3-(3-chloro-4-fluorophenyl)-1-(1-(6,7-difluoro-3-methyl-4-oxo-3,4-dihydrophthalazin-1-yl)ethyl)-1-(3-hydroxypropyl)urea was synthesized in an analogous manner as described above from racemic 6,7-difluoro-4-(1-((3-hydroxypropyl)amino)ethyl)-2-methylphthalazin-1(2H)-one (VIo) and 2-chloro-1-fluoro-4-isocyanatobenzene (XIIa). LCMS: m/z found 469.1/471.2 [M+H]⁺, RT = 5.06 min, (Method A); ¹H NMR (400 MHz, DMSO-d₆) δ 8.74 (d, 1H), 8.23 (m, 1H), 8.06 (m, 1H), 7.79-7.71 (m, 1H), 7.44 (m, 1H), 7.35 (t, 1H), 6.10 (m, 1H), 4.93 (t, 1H), 3.76 (s, 3H), 3.32-3.17 (m, 4H), 1.47 (d, 3H), 1.35-1.27 (m, 1H), 1.13 (m, 1H).

### 1-(6,7-difluoro-3-methyl-4-oxo-3,4-dihydrophthalazin-1-yl)ethyl)-3-(4-fluorophenyl)-1-(3-hydroxypropyl)urea (Compound 26)

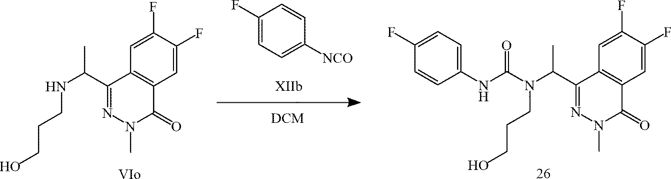

Racemic 1-(1-(6,7-difluoro-3-methyl-4-oxo-3,4-dihydrophthalazin-1-yl)ethyl)-3-(4-fluorophenyl)-1-(3-hydroxypropyl)urea was synthesized in an analogous manner as described above from racemic 6,7-difluoro-4-(1-((3-hydroxypropyl)amino)ethyl)-2-methylphthalazin-1(2H)-one (VIo) and 1-fluoro-4-isocyanatobenzene (XIIb). LCMS: m/z found 435.2 [M+H]⁺, RT = 4.39 min, (Method A); ¹H NMR (400 MHz, Chloroform-d) δ 8.20 (m, 1H), 8.10 (s, 1H), 8.01 (m, 1H), 7.50-7.39 (m, 2H), 6.98 (m, 2H), 6.24 (m, 1H), 3.87 (d, 3H), 3.60 (m, 1H), 3.56-3.44 (m, 1H), 3.34 (t, 2H), 1.96 (t, 1H), 1.56 (d, 3H), 1.47 (m, 1H), 1.28 (m, 1H).

### 6,7-difluoro-2,3-dihydrophthalazine-1,4-dione (IXb)

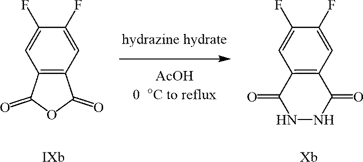

Hydrazine hydrate (0.58 mL, 12.01 mmol) was added slowly to a 0° C. solution of 5,6-difluoroisobenzofuran-1,3-dione (IXb, 1.52 g, 8.26 mmol) in glacial acetic acid (20 mL). After half the hydrazine was added, a thick white precipitate formed. After the hydrazine addition was complete, the reaction mixture was allowed to warm to room temperature, then heated to 85° C. for 3 h. A white solid formed upon cooling. After standing at room temperature for 72 h white crystalline material was collected by vacuum filtration, and washed with petroleum ether (20 mL) and water (50 mL). The material was dried under high vacuum to provide 6,7-difluoro-2,3-dihydrophthalazine-1,4-dione (Xb, 1.80 g, 83% yield) as a white solid. ¹H NMR (400 MHz, DMSO-d₆) δ 11.79 (br s, 2H), 8.02 (br s, 2H).

### 6,7-difluoro-4-oxo-3,4-dihydrophthalazin-1-yl Trifluoromethanesulfonate (XIb)

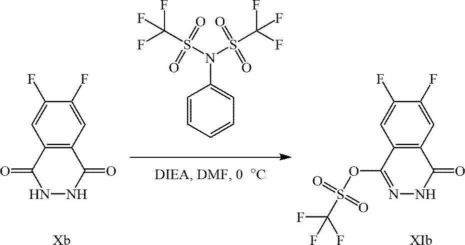

DIEA (3.09 mL, 17.77 mmol) was added to a 0° C. solution of 6,7-difluoro-2,3-dihydrophthalazine-1,4-dione (Xb, 1.76 g, 8.88 mmol) in anhydrous DMF (50 mL). N-Phenyl-bis(trifluoromethanesulfonimide) (3.49 g, 9.77 mmol) was added in one portion. After 15 min at 0° C., the reaction mixture was diluted with EtOAc (70 mL) and washed hydrochloric acid (0.2 M, 2 × 40 mL), water (30 mL), and brine (30 mL). The organics were dried over sodium sulfate, filtered, and evaporated to dryness to provide a white solid. The material was dissolved in DCM and absorbed on CELITE®. The product was isolated by flash-chromatography (silica gel, EtOAc/hexanes 5 - 60% gradient) to provide (6,7-difluoro-4-oxo-3H-phthalazin-1-yl) trifluoromethanesulfonate (XIb, 2.51 g, 86% yield) as a white solid. ¹H NMR (400 MHz, Chloroform- d) δ 9.97 (s, 1H), 8.27 (m, 1H), 7.67 (m, 1H).

### 4-acetyl-6,7-difluoro-2H-phthalazin-1-one (Vf)

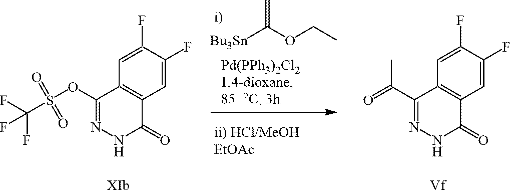

A solution of (6,7-difluoro-4-oxo-3H-phthalazin-1-yl) trifluoromethanesulfonate (XIb, 796 mg, 2.41 mmol) in 1,4-dioxane (10 mL) was degassed with nitrogen. Tributyl(1-ethoxyvinyl)stannane (1.05 mL, 3.13 mmol) and dichlorobis(triphenylphosphine) palladium(II), (135 mg, 0.19 mmol) were added. The mixture was heated at 85° C. in a heating block for 3 h. Upon cooling, the reaction mixture was diluted with EtOAc (50 mL) and filtered through a pad of CELITE®. The filter cake was washed with an additional portion of EtOAc (20 mL). The combined filtrate was evaporated to dryness. The product was isolated by flash-chromatography (silica gel, EtOAc/hexanes 10 - 60% gradient) to provide 0.33 g of a 1:2 mixture of 4-(1-ethoxyvinyl)-6,7-difluoro-2H-phthalazin-1-one and 4-(1-ethoxyvinyl)-6,7-difluoro-2H-phthalazin-1-one. The material was dissolved in EtOAc (30 mL) and hydrogen chloride solution (3 M in MeOH, 1.32 mL, 3.97 mmol) was added. After 20 min, the volatiles were removed in vacuo to provide 4-acetyl-6,7-difluoro-2H-phthalazin-1-one (Vf, 312 mg, 58% yield). ¹H NMR (400 MHz, Chloroform-d) δ 10.70 (s, 1H), 8.93 (m, 1H), 8.26-8.16 (m, 1H), 2.68 (s, 3H).

### 6,7-difluoro-4-(1-(isobutylamino)ethyl)phthalazin-1(2H)-one (VIj)

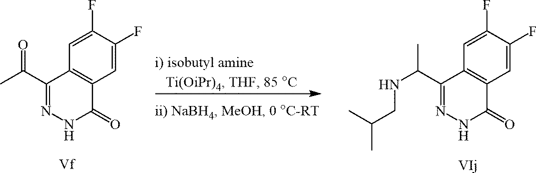

Tetraisopropoxytitanium (0.22 mL, 0.74 mmol) was added to a solution of 4-acetyl-6,7-difluoro-2H-phthalazin-1-one (Vf, 55 mg, 0.25 mmol) and 2-methylpropan-1-amine (27 µL, 0.27 mmol) in anhydrous THF (1 mL). The mixture was heated at 65° C. for 2 h in a heating block. The reaction mixture was then diluted with anhydrous methanol (1 mL) and cooled in an ice bath. Sodium borohydride (14 mg, 0.37 mmol) was added in one portion. The reaction mixture was stirred for 10 min, and the ice bath was removed. After an additional 40 min, the reaction mixture was slowly added to a rapidly stirred brine solution (0.5 mL) and diluted with 20 mL of 9:1 (v/v) EtOAc/MeCN. After stirring for 10 min, the mixture was filtered through a pad of CELITE®, and the filter cake was washed with an additional portion of EtOAc (15 mL). The combined filtrate was evaporated to dryness to provide crude racemic 6,7-difluoro-4-(1-(isobutylamino)ethyl)phthalazin-1(2H)-one (VIj, 75 mg). LCMS: m/z found 282.2 [M+H]⁺, RT = 0.67 min, (Method B); ¹H NMR (400 MHz, Methanol-d₄) δ 8.32 (m, 1H), 8.20 (m, 1H), 4.33 (m, 1H), 2.50 (m, 1H), 2.31 (m, 1H), 1.83-1.68 (m, 1H), 1.47 (d, 3H), 0.91 (m, 6H).

### 3-chloro-4-fluorophenyl)-1-(1-(6,7-difluoro-4-oxo-3,4-dihydrophthalazin-1-yl)ethyl)-1-isobutylurea (Compound 27)

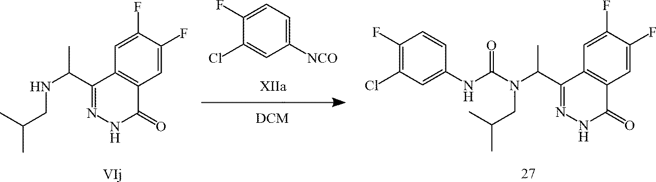

Racemic 3-(3-chloro-4-fluorophenyl)-1-(1-(6,7-difluoro-4-oxo-3,4-dihydrophthalazin-1-yl)ethyl)-1-isobutylurea was synthesized in an analogous manner as described above from 6,7-difluoro-4-(1-(isobutylamino)ethyl)phthalazin-1(2H)-one (VIj) and 2-chloro-1-fluoro-4-isocyanatobenzene (XIIa). LCMS: m/z found 453.1/455.2 [M+H]⁺, RT = 5.47 min, (Method A); ¹H NMR (400 MHz, DMSO-d₆) δ 12.94 (s, 1H), 8.51 (s, 1H), 8.28-8.17 (m, 2H), 7.75 (dd, 1H), 7.52-7.43 (m, 1H), 7.35 (t, 1H), 6.10 (m, 1H), 3.19 (m, 1H), 2.88 (m, 1H), 1.46 (d, 3H), 1.37 (m, 1H), 0.64 (d, 3H), 0.37 (d, 3H).

### 1-(6,7-difluoro-4-oxo-3,4-dihydrophthalazin-1-yl)ethyl)-1-isobutyl-3-phenylurea (Compounds 28, 39, 40)

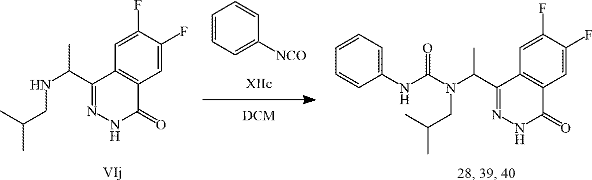

Racemic 1-(1-(6,7-difluoro-4-oxo-3,4-dihydrophthalazin-1-yl)ethyl)-1-isobutyl-3-phenylurea was synthesized in an analogous manner as described above 6,7-difluoro-4-(1-(isobutylamino)ethyl)phthalazin-1(2H)-one (VIj) and isocyanatobenzene (XIIc). LCMS: m/z found 401.2 [M+H]⁺, RT = 4.77 min, (Method A); ¹H NMR (400 MHz, DMSO-d₆) b 12.93 (s, 1H), 8.35-8.26 (m, 2H), 8.21 (m, 1H), 7.52-7.44 (m, 2H), 7.33-7.23 (m, 2H), 7.05-6.95 (m, 1H), 6.12 (m, 1H), 3.23 (m, 1H), 2.87 (m, 1H), 1.49-1.34 (m, 4H), 0.65 (d, 3H), 0.37 (d, 3H).

The enantiomers were subsequently separated by semi-preparative SFC: Method isocratic, Mobile phase (ACN:MeOH (1:1)): CO₂ - 40:60. Column: Chiralpak-AD (10 × 250 mm), 5 µm, flow rate: 9 g/min.

Enantiomer I (Compound 39): LCMS: m/z found 401.2 [M+H]⁺, RT = 4.71 min, (Method A); ¹H NMR (400 MHz, DMSO-d₆) δ 12.93 (s, 1H), 8.35-8.26 (m, 2H), 8.21 (m, 1H), 7.52-7.44 (m, 2H), 7.33-7.23 (m, 2H), 7.05-6.95 (m, 1H), 6.12 (m, 1H), 3.23 (m, 1H), 2.87 (m, 1H), 1.49-1.34 (m, 4H), 0.65 (d, 3H), 0.37 (d, 3H); Chiral analytical SFC: RT = 2.48 min, Column: Chiralpak AD (4.6 × 250 mm) 5 µm, 25% of (ACN:MeOH (1:1)), Flow rate: 3.0 g/min.

Enantiomer II (Compound 40): LCMS: m/z found 401.2 [M+H]⁺, RT = 4.72 min, (Method A); ¹H NMR (400 MHz, DMSO-d₆) δ 12.93 (s, 1H), 8.35-8.26 (m, 2H), 8.21 (m, 1H), 7.52-7.44 (m, 2H), 7.33-7.23 (m, 2H), 7.05-6.95 (m, 1H), 6.12 (m, 1H), 3.23 (m, 1H), 2.87 (m, 1H), 1.49-1.34 (m, 4H), 0.65 (d, 3H), 0.37 (d, 3H); Chiral analytical SFC: RT = 4.67 min, Column: Chiralpak AD (4.6 × 250 mm) 5 µm, 25% of (ACN:MeOH (1:1)), Flow rate: 3.0 g/min.

### 6,7-difluoro-4-(1-(methylamino)ethyl)phthalazin-1(2H)-one (VIk)

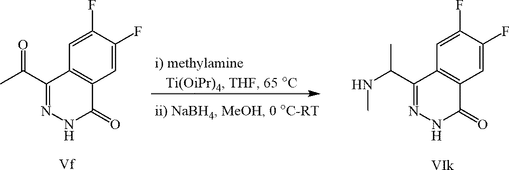

Tetraisopropoxytitanium (1.0 mL, 3.39 mmol) was added to a solution of 4-acetyl-6,7-difluoro-2H-phthalazin-1-one (Vf, 253 mg, 1.13 mmol) and a methylamine solution (2 M in THF, 0.62 mL, 1.24 mmol) in anhydrous THF (0.8 mL) in a pressure vessel. The vessel was capped and the mixture was heated at 65° C. for 2 h in a heating block. An additional portion of a methylamine solution (2 M in THF, 0.15 mL, 0.30 mmol) was added and the mixture was heated for an additional 1h. The reaction mixture was diluted with anhydrous methanol (5 mL) and cooled in an ice bath. Sodium borohydride (64 mg, 1.69 mmol) was added in one portion. The reaction mixture was stirred for 10 min, and the ice bath was removed. After an additional 25 min, the reaction mixture was slowly added to a rapidly stirred brine solution (0.5 mL) and diluted with 20 mL of 9:1 (v/v) EtOAc/MeCN. After stirring for 15 min, the mixture was filtered through a pad of CELITE®, and the filter cake was washed with an additional portion of EtOAc (15 mL). The combined filtrate was evaporated to dryness to provide crude racemic 6,7-difluoro-4-[1-(methylamino)ethyl]-2H-phthalazin-1-one (VIk, 271 mg). LCMS: m/z found 240.2 [M+H]⁺, RT = 0.51 min, (Method B); ¹H NMR (400 MHz, Methanol-d₄) δ 8.26-8.14 (m, 2H), 4.28 (m, 1H), 2.38 (s, 3H), 1.45 (d, 3H).

### 3-chloro-4-fluorophenyl)-1-(1-(6,7-difluoro-4-oxo-3,4-dihydrophthalazin-1-yl)ethyl)-1-methylurea (Compounds 29, 31, 32)

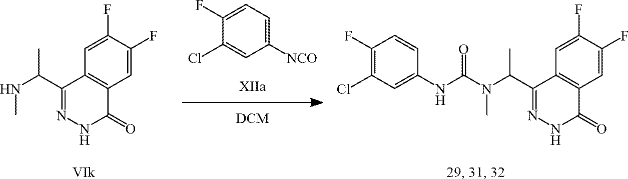

3-Chloro-4-fluorophenyl)-1-(1-(6,7-difluoro-4-oxo-3,4-dihydrophthalazin-1-yl)ethyl)-1-methylurea was synthesized in an analogous manner as described above from racemic 6,7-difluoro-4-(1-(methylamino)ethyl)phthalazin-1(2H)-one (VIk) and 2-chloro-1-fluoro-4-isocyanatobenzene (XIIa). LCMS: m/z found 411.1/413.1 [M+H]⁺, RT = 4.72 min, (Method A); ¹H NMR (400 MHz, DMSO-d₆) δ 12.93 (s, 1H), 8.56 (s, 1H), 8.20 (m, 1H), 8.06 (m, 1H), 7.81 (m, 1H), 7.51 (m, 1H), 7.34 (t, 1H), 6.05 (m, 1H), 2.68 (s, 3H), 1.43 (d, 3H).

The enantiomers were subsequently separated by semi-preparative SFC: Method isocratic, Mobile phase (ACN:MeOH (1:1)): CO₂ - 40:60. Column: Chiralpak-AD (10 × 250 mm), 5 µm, flow rate: 9 g/min.

Enantiomer I (Compound 31): LCMS: m/z found 411.1/413.1 [M+H]⁺, RT = 4.74 min, (Method A); ¹H NMR (400 MHz, Chloroform-d) δ 10.45 (s, 1H), 8.22 (m, 1H), 8.13 (m, 1H), 7.60 (m, 1H), 7.25 (m, 1H), 7.10 (t, 1H), 6.34 (s, 1H), 6.20 (m, 1H), 2.79 (s, 3H), 1.54 (d, 3H); Chiral analytical SFC: RT = 7.68 min, Column: Chiralpak AD (4.6 × 250 mm) 5 µm, 20% of (ACN:MeOH (1:1)), Flow rate: 3.0 g/min.

Enantiomer II (Compound 32): LCMS: m/z found 411.1/413.1 [M+H]⁺, RT = 4.74 min, (Method A); ¹H NMR (400 MHz, Chloroform-d) δ 10.30 (s, 1H), 8.22 (m, 1H), 8.13 (m, 1H), 7.60 (m, 1H), 7.30-7.21 (m, 1H), 7.10 (t, 1H), 6.33 (s, 1H), 6.20 (m, 1H), 2.79 (s, 3H), 1.54 (d, 3H); Chiral analytical SFC: RT = 9.96 min, Column: Chiralpak AD (4.6 × 250 mm) 5 µm, 20% of (ACN:MeOH (1:1)), Flow rate: 3.0 g/min. Enantiomer II (Compound 32) was also independently prepared by chiral synthesis starting from enantiomerically pure (R)-6,7-difluoro-4-(1-(methylamino)ethyl)phthalazin-1(2H)-one hydrochloride (VIr).

### 2-chloro-4-fluorophenyl)-1-(1-(6,7-difluoro-3-methyl-4-oxo-3,4-dihydrophthalazin-1-yl)ethyl)ureido)-N-(chloro-4-fluorophenyl)carbamoyl)ethane-1-sulfonamide (Compound 30)

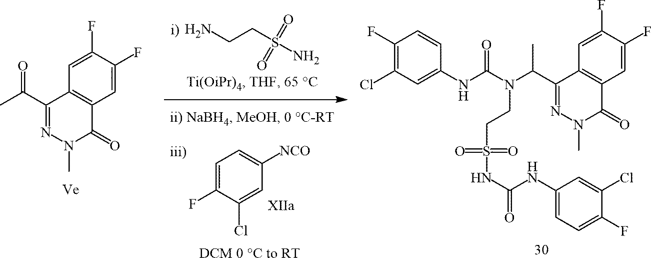

Tetraisopropoxytitanium (248 µL, 0.84 mmol) was added to a solution of 4-acetyl-6,7-difluoro-2-methyl-phthalazin-1-one (Ve, 67 mg, 0.28 mmol) and 2-aminoethanesulfonamide (36 µL, 0.28 mmol) in anhydrous THF (1 mL) in a pressure vessel. The vessel was capped, and the mixture was heated to 65° C. for 2 h in a heating block. Upon cooling to room temperature, the reaction mixture was diluted with anhydrous methanol (1 mL) and cooled in an ice bath. Sodium borohydride (16 mg, 0.42 mmol) was added in one portion. The reaction mixture was stirred for 10 minutes, and the ice bath was removed. After an additional 40 min, the reaction mixture was slowly added to a rapidly stirred brine solution (0.5 mL) and diluted with 20 mL of 9:1 (v/v) EtOAc/MeCN. The mixture was filtered through a pad of CELITE®, and the filter cake was washed with EtOAc (15 mL) and MeOH/DCM (1:4 (v/v), 15 mL). The combined filtrate was evaporated to dryness. The crude product mixture in DCM (2 mL) was cooled in an ice bath. A solution of 2-chloro-1-fluoro-4-isocyanato-benzene (XIIa, 26 µL, 0.21 mmol) in DCM (0.5 mL) was added slowly. After 5 min, the reaction mixture was loaded directly on a pre-equilibrated silica gel column and isolated by flash-chromatography (silica gel, MeOH/DCM 0.5 - 3% gradient). The material was repurified by flash-chromatography (silica gel, MeOH/DCM 1 - 9% gradient) to provide to provide racemic 3-(3-chloro-4-fluoro-phenyl)-1-[2-[(3-chloro-4-fluorophenyl)carbamoylsulfamoyl]ethyl]-1-[1-(6,7-difluoro-3-methyl-4-oxo-phthalazin-1-yl)ethyl]urea (30, 16 mg, 8% yield). LCMS: m/z found 689.1/691.1 [M+H]⁺, RT = 6.05 min, (Method A); ¹H NMR (400 MHz, Methanol-d₄) δ 8.20 (m, 1H), 8.03 (m, 1H), 7.64 (m, 2H), 7.39 (m, 1H), 7.23 (m, 1H), 7.14 (m, 2H), 6.13 (m, 1H), 3.87-3.64 (m, 5H), 3.56 (m, 1H), 3.03 (m, 1H), 1.62 (d, 3H).

### 3-cyclopropyl-1-(1-(6,7-difluoro-3-methyl-4-oxo-3,4-dihydrophthalazin-1-yl)ethyl)-1-isobutylurea (Compound 33)

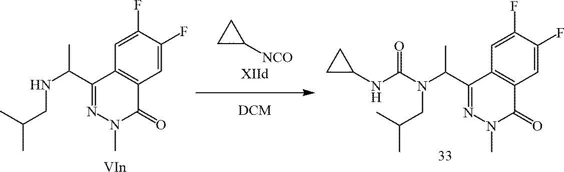

Racemic 3-cyclopropyl-1-(1-(6,7-difluoro-3-methyl-4-oxo-3,4-dihydrophthalazin-1-yl)ethyl)-1-isobutylureawas synthesized in an analogous manner as described above from racemic 6,7-difluoro-4-(1-(isobutylamino)ethyl)-2-methylphthalazin-l(2H)-one (VIn) and isocyanatocyclopropane (XIId). LCMS: m/z found 379.3 [M+H]⁺, RT = 4.48 min, (Method A); ¹H NMR (400 MHz, Chloroform-d) δ 8.16 (m, 2H), 6.22 (m, 1H), 4.67 (s, 1H), 3.84 (s, 3H), 2.85-2.66 (m, 3H), 1.46 (d, 3H), 1.46-1.31 (m, 1H), 0.86-0.72 (m, 2H), 0.71 (d, 3H), 0.54-0.42 (m, 2H), 0.33 (d, 3H).

### 1-(6,7-difluoro-3-methyl-4-oxo-3,4-dihydrophthalazin-1-yl)ethyl)-1-isobutyl-3-phenylurea (Compound 34)

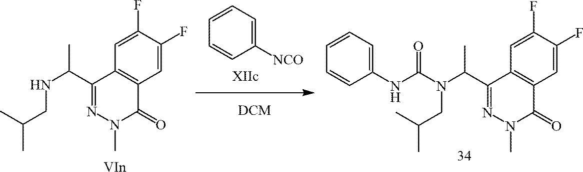

Racemic 1-(1-(6,7-difluoro-3-methyl-4-oxo-3,4-dihydrophthalazin-1-yl)ethyl)-1-isobutyl-3-phenylurea was synthesized in an analogous manner as described above from racemic 6,7-difluoro-4-(1-(isobutylamino)ethyl)-2-methylphthalazin-1(2H)-one (VIn) and isocyanatobenzene (XIIc). LCMS: m/z found 415.2 [M+H]⁺, RT = 5.36 min, (Method A); ¹H NMR (400 MHz, Chloroform-d) δ 8.17 (m, 2H), 7.47-7.38 (m, 2H), 7.39-7.29 (m, 2H), 7.14-7.05 (m, 1H), 6.40 (s, 1H), 6.27 (m, 1H), 3.87 (s, 3H), 3.07-2.96 (m, 2H), 1.60-1.47 (m, 4H), 0.84 (d, 3H), 0.41 (d, 3H).

### 3-cyclopentyl-1-(1-(6,7-difluoro-3-methyl-4-oxo-3,4-dihydrophthalazin-1-yl)ethyl)-1-isobutylurea (Compound 35)

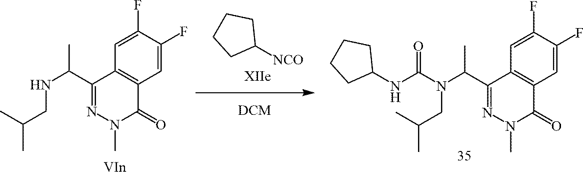

Racemic 3-cyclopentyl-1-(1-(6,7-difluoro-3-methyl-4-oxo-3,4-dihydrophthalazin-1-yl)ethyl)-1-isobutylurea was synthesized in an analogous manner as described above from racemic 6,7-difluoro-4-(1-(isobutylamino)ethyl)-2-methylphthalazin-1(2H)-one (VIn) and isocyanatocyclopentane (XIIe). LCMS: m/z found 407.2 [M+H]⁺, RT = 5.36 min, (Method A); ¹H NMR (400 MHz, Chloroform-d) δ 8.23-8.12 (m, 2H), 6.21 (m, 1H), 4.34 (d, 1H), 4.20 (m, 1H), 3.85 (s, 3H), 2.86-2.69 (m, 2H), 2.11-1.96 (m, 2H), 1.72-1.59 (m, 4H), 1.50-1.38 (m, 3H), 1.41-1.28 (m, 3H), 0.73 (d, 3H), 0.36 (d, 3H).

### (S)-N-((R)-1-(6,7-difluoro-3-methyl-4-oxo-3,4-dihydrophthalazin-1-yl)ethyl)-2-methylpropane-2-sulfinamide (XIVa) (XIVa)

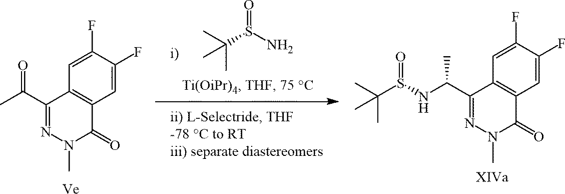

A mixture of 4-acetyl-6,7-difluoro-2-methyl-phthalazin-1-one (Ve, 49 mg, 0.21 mmol) and (S)-2-methylpropane-2-sulfinamide (30 mg, 0.25 mmol) was prepared in a pressure vessel. Anhydrous THF (0.25 mL) was added, and the mixture was stirred until a solution formed. Tetraisopropoxytitanium (0.12 mL, 0.41 mmol) was added, the vessel was sealed, and the reaction mixture was heated to 75° C. overnight in a heating block. The cooled reaction mixture was diluted with anhydrous THF (0.4 mL) and further cooled in a dry ice/acetone bath under nitrogen. L-Selectride (1 M in THF, 0.27 mL, 0.27 mmol) was added slowly and the reaction mixture was allowed to warm to room temperature over 3 h. The mixture was re-cooled to -40° C., quenched by the addition of MeOH (0.3 mL), and allowed to warm to room temperature. The reaction mixture was added dropwise into a rapidly stirred brine solution (0.5 mL) and diluted with EtOAc (20 mL). After stirring for 10 minutes, the mixture was filtered through a pad of CELITE®. The filter cake was washed with an additional portion of EtOAc (5 mL), and the combined filtrate was evaporated to dryness. The major diastereoisomer was isolated by flash-chromatography (silica gel, EtOAc/hexanes 5-95% gradient) to provide (S)-N-((R)-1-(6,7-Difluoro-3-methyl-4-oxo-3,4-dihydrophthalazin-1-yl)ethyl)-2-methylpropane-2-sulfinamide (XIVa, 41 mg, 58% yield). LCMS: m/z found 344.3 [M+H]⁺, RT = 2.35 min, (Method D); ¹H NMR (400 MHz, Chloroform-d) δ 8.24 (m, 1H), 7.70 (m, 1H), 4.94-4.82 (m, 1H), 3.83 (s, 3H), 3.62 (d, 1H), 1.74 (d, 3H), 1.20 (s, 9H).

### (S)-N-((R)-1-(6,7-difluoro-3-methyl-4-oxo-3,4-dihydrophthalazin-1-yl)ethyl)-N,2-dimethylpropane-2-sulfinamide (XVa)

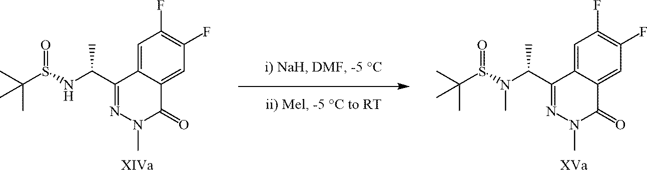

A solution of (S)-N-((R)-1-(6,7-difluoro-3-methyl-4-oxo-3,4-dihydrophthalazin-1-yl)ethyl)-2-methylpropane-2-sulfinamide (XIVa, 83 mg, 0.24 mmol) in anhydrous DMF (4 mL) under nitrogen was cooled to -5° C. in a brine/ice bath. Sodium hydride (60% in mineral oil, 14 mg, 0.34 mmol) was added in one portion. The reaction mixture turned light-yellow and a precipitate gradually formed, then re-dissolved. After 25 min at -5° C., iodomethane (26 µL, 0.41 mmol) was added. After 20 min, the reaction mixture was quenched by the slow addition of water (20 mL). The mixture was diluted with an additional 20 mL of water and extracted with EtOAc (3 × 20 mL). The combined organics were washed with water (3 × 10 mL), brine (15 mL), dried over sodium sulfate, filtered, and evaporated to dryness. The product was isolated by flash-chromatography (silica gel, EtOAc/hexanes 5-85% gradient) to provide (S)-N-((R)-1-(6,7-difluoro-3-methyl-4-oxo-3,4-dihydrophthalazin-1-yl)ethyl)-N,2-dimethylpropane-2-sulfinamide (XVa, 65 mg, 75% yield) as a single diastereomer. LCMS: m/z found 358.2 [M+H]⁺, RT = 4.09 min, (Method A); ¹H NMR (400 MHz, Chloroform- d) δ 8.23 (m, 1H), 7.76 (m, 1H), 5.01 (m, 1H), 3.85 (s, 3H), 2.46 (s, 3H), 1.66 (d, 3H), 1.24 (s, 9H).

### (R)-6,7-difluoro-2-methyl-4-(1-(methylamino)ethyl)phthalazin-1(2H)-one Hydrochloride (VIp)

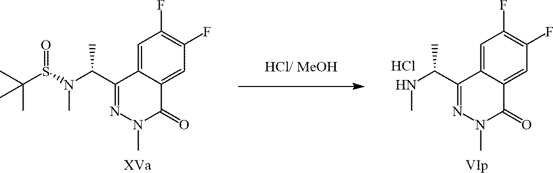

Hydrogen chloride (3 M in MeOH, 0.21 mL, 0.63 mmol) was added to solution of (S)-N-((R)-1-(6,7-difluoro-3-methyl-4-oxo-3,4-dihydrophthalazin-1-yl)ethyl)-N,2-dimethylpropane-2-sulfinamide (XVa, 57 mg, 0.16 mmol) in MeOH (4 mL). After stirring for 30 min at room temperature, the volatiles were removed in vacuo. The residue was triturated with diethyl ether (2 × 10 mL). Drying under high vacuum overnight provided enantiomerically pure (R)-6,7-difluoro-2-methyl-4-(1-(methylamino)ethyl)phthalazin-1(2H)-one hydrochloride (VIp, 46 mg). LCMS: m/z found 254.1 [M+H]⁺, RT = 0.73 min, (Method B); ¹H NMR (400 MHz, Methanol-d₄) δ 8.28 (m, 1H), 8.04 (m, 1H), 4.98 (m, 1H), 3.86 (s, 3H), 2.83 (s, 3H), 1.67 (d, 3H).

### (R)(3-chloro-4-fluorophenyl)-1-(1-(6,7-difluoromethyl-4-oxo-3,4-dihydrophthalazin-1-yl)ethyl)-1-methylurea (Compound 36)

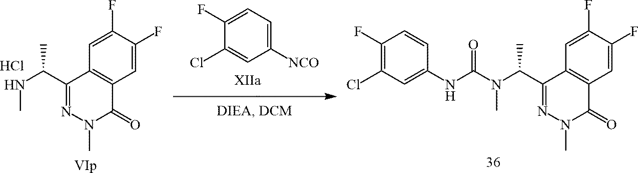

A mixture of DIEA (55 µL, 0.32 mmol) and enantiomerically pure (R)-6,7-difluoro-2-methyl-4-[1-(methylamino)ethyl]phthalazin-1-one hydrochloride (VIp, 46 mg, 0.16 mmol) in 2 mL DCM was cooled in an ice bath. A solution of 2-chloro-1-fluoro-4-isocyanato-benzene (XIIa, 20 µL, 0.16 mmol) in 0.5 mL DCM was added and the mixture was stirred for 5 min. The crude reaction mixture was loaded directly on a pre-equilibrated silica gel column. The product was isolated by flash chromatography (silica gel, EtOAc/DCM 0-30% gradient) to provide enantiomerically pure (R)-3-(3-chloro-4-fluorophenyl)-1-[1-(6,7-difluoro-3-methyl-4-oxo-phthalazin-1-yl)ethyl]-1-methyl-urea (54 mg, 80%). LCMS: m/z found 425.1/427.0 [M+H]⁺, RT = 5.18 min, (Method A); ¹H NMR (400 MHz, DMSO- d ₆ ) δ 8.56 (s, 1H), 8.22 (m, 1H), 8.08 (m, 1H), 7.81 (m, 1H), 7.51 (m, 1H), 7.34 (t, 1H), 6.06 (m, 1H), 3.76 (s, 3H), 2.70 (s, 3H), 1.44 (d, 3H).

### (S)-6,7-difluoro-2-methyl-4-(1-(methylamino)ethyl)phthalazin-1(2H)-one Hydrochloride (VIq)

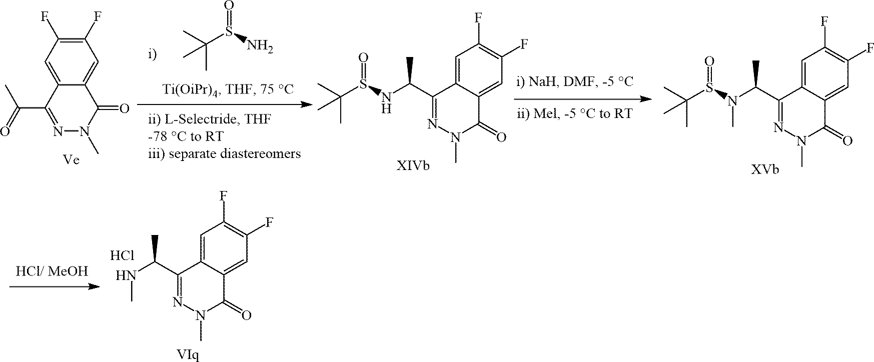

Enantiomerically pure (S)-6,7-difluoro-2-methyl-4-(1-(methylamino)ethyl)phthalazin-1(2H)-one hydrochloride (VIq) was synthesized in an analogous manner as described above from 4-acetyl-6,7-difluoro-2-methyl-phthalazin-1-one (Ve) and (R)-2-methylpropane-2-sulfinamide. LCMS: m/z found 254.2 [M+H]⁺, RT = 0.74 min, (Method B); ¹H NMR (400 MHz, Methanol-d₄) δ 8.28 (m, 1H), 8.05 (m, 1H), 4.98 (m, 1H), 3.86 (s, 3H), 2.83 (s, 3H), 1.67 (d, 3H).

### (S)(3-chloro-4-fluorophenyl)-1-(1-(6,7-difluoromethyl-4-oxo-3,4-dihydrophthalazin-1-yl)ethyl)-1-methylurea (Compound 37)

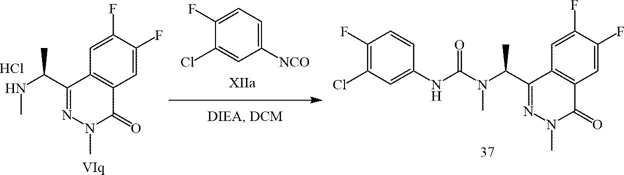

Enantiomerically pure (S)-3-(3-chloro-4-fluorophenyl)-1-(1-(6,7-difluoro-3-methyl-4-oxo-3,4-dihydrophthalazin-1-yl)ethyl)-1-methylurea was synthesized in an analogous manner as described above from enantiomerically pure (S)-6,7-difluoro-2-methyl-4-(1-(methylamino)ethyl)phthalazin-1(2H)-one hydrochloride (VIq), DIEA, and 1-fluoro-4-isocyanatobenzene (XIIa). LCMS: m/z found 425.1/427.1 [M+H]⁺, RT = 5.17 min, (Method A); ¹H NMR (400 MHz, DMSO- d ₆ ) δ 8.56 (s, 1H), 8.22 (m, 1H), 8.08 (m, 1H), 7.81 (m, 1H), 7.51 (m, 1H), 7.34 (t, 1H), 6.06 (m, 1H), 3.76 (s, 3H), 2.70 (s, 3H), 1.44 (d, 3H).

### (S)(1-(6,7-difluoro-3-methyl-4-oxo-3,4-dihydrophthalazinyl)ethyl)-3-(4-fluorophenyl)methylurea (Compound 38)

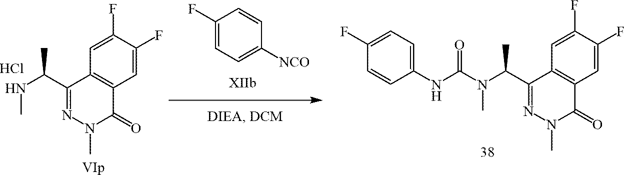

(S)(1-(6,7-Difluoro-3-methyl-4-oxo-3,4-dihydrophthalazinyl)ethyl)-3-(4-fluorophenyl)methylurea was synthesized in an analogous manner as described above from enantiomerically pure (S)-6,7-difluoro-2-methyl-4-(1-(methylamino)ethyl)phthalazine-1(2H)-one hydrochloride (VIq), DIEA, and 1-fluoro-4-isocyanatobenzene (XIIb). LCMS: m/z found 391.2 [M+H]⁺, RT = 4.60 min, (Method A); ¹H NMR (400 MHz, Chloroform- d) δ 8.21 (m, 1H), 8.10 (m, 1H), 7.43-7.34 (m, 2H), 7.08-6.99 (m, 2H), 6.31 (s, 1H), 6.20 (m, 1H), 3.87 (s, 3H), 2.77 (s, 3H), 1.54 (d, 3H).

### 4-acetyl-6,7-difluoro-2-((2-(trimethylsilyl)ethoxy)methyl)phthalazin-1(2H)-one (XVIIa)

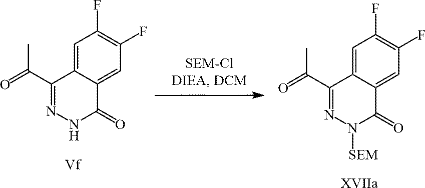

DIEA (0.45 mL, 2.61 mmol) was added to a mixture of 4-acetyl-6,7-difluoro-2H-phthalazin-1-one (Vf, 365 mg, 1.63 mmol) in DCM (35 mL). 2-(Chloromethoxy)ethyl-trimethyl-silane (350 µL, 1.95 mmol) was added in one portion. After stirring overnight at room temperature, the reaction mixture was diluted with 1 mL hexanes and loaded on a pre-equilibrated silica gel column. The product was isolated by flash chromatography (silica gel, EtOAc/hexanes 0-60% gradient) to provide 4-acetyl-6,7-difluoro-2-(2-trimethylsilylethoxymethyl)phthalazin-1-one (XVIIa, 0.44 g, 77% yield). ¹H NMR (400 MHz, Chloroform-d) δ 8.88 (m, 1H), 8.27-8.18 (m, 1H), 5.61 (s, 2H), 3.84-3.74 (m, 2H), 2.69 (d, 3H), 1.04-0.95 (m, 2H), -0.00 (s, 9H).

### (S)-N-((R)-1-(6,7-difluoro-4-oxo-3-((2-(trimethylsilyl)ethoxy)methyl)-3,4-dihydrophthalazin-1-yl)ethyl)-2-methylpropane-2-sulfinamide (XIVc)

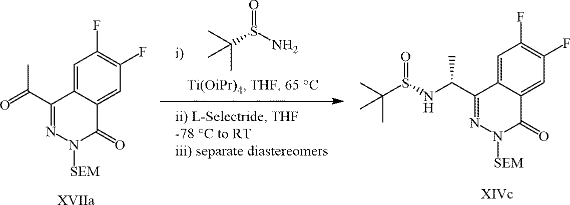

Anhydrous THF (0.4 mL) and tetraisopropoxytitanium (0.57 mL, 1.91 mmol) were added to a pressure tube containing 4-acetyl-6,7-difluoro-2-(2-trimethylsilylethoxymethyl)phthalazin-1-one (XVIIa, 339 mg, 0.96 mmol) and (S)-2-methylpropane-2-sulfinamide (151 mg, 1.24 mmol). The vessel was sealed and heated at 65° C. in a heating block overnight. The reaction mixture was diluted with anhydrous THF (0.8 mL) and cooled in a dry ice/acetone bath. L-Selectride solution (1 M in THF, 1.00 mL, 1.00 mmol) was added slowly. After 20 minutes, an additional 0.5 mL THF was added. After 2.25 h total, the reaction was quenched by the addition of MeOH (1.2 mL). The cooling bath was removed and the reaction mixture was added dropwise into a rapidly stirred brine solution (2 mL) and diluted with 60 mL EtOAc (60 mL). After stirring for 10 min, the mixture was filtered through a pad of CELITE®. The filter cake was washed with an additional portion of EtOAc (20 mL) and the combined filtrate was evaporated to dryness. LCMS showed a 93:7 ration of diastereomers. The major diastereomer was isolated by flash-chromatography (silica gel, EtOAc/hexanes 5-70% gradient) to provide (S)-N-((R)-1-(6,7-difluoro-4-oxo-3-((2-(trimethylsilyl)ethoxy)methyl)-3,4-dihydrophthalazin-1-yl)ethyl)-2-methylpropane-2-sulfinamide (XIVc, 288 mg, 63% yield). LCMS: m/z found 460.3 [M+H]⁺, RT = 5.42 min, (Method A); ¹H NMR (400 MHz, Chloroform-d) δ 8.26 (m, 1H), 7.69 (m, 1H), 5.53 (s, 2H), 4.89 (m, 1H), 3.80-3.66 (m, 2H), 3.63 (d, 1H), 1.75 (d, 3H), 1.19 (s, 9H), 1.02-0.89 (m, 2H), -0.01 (s, 9H).

### (S)-N-((R)-1-(6,7-difluoro-4-oxo-3-((2-(trimethylsilyl)ethoxy)methyl)-3,4-dihydrophthalazin-1-yl)ethyl)-N,2-dimethylpropane-2-sulfinamide (XVc)

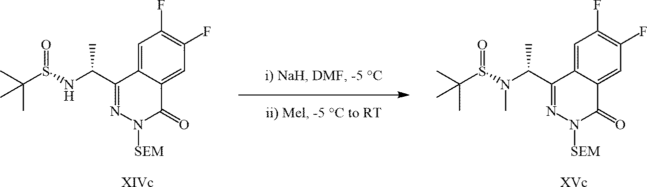

A solution of (S)-N-((R)-1-(6,7-difluoro-4-oxo-3-((2-(trimethylsilyl)ethoxy)methyl)-3,4-dihydrophthalazin-1-yl)ethyl)-2-methylpropane-2-sulfinamide (XIVc, 284 mg, 0.62 mmol) in anhydrous DMF (8 mL) under nitrogen atmosphere was cooled to -5° C. in a brine/ice bath. Sodium hydride (60% in mineral oil, 44 mg, 1.11 mmol) was added. The mixture turned light-yellow. After 25 min, iodomethane (70 µL, 1.11 mmol) was added. After an additional 15 min at -5° C., the reaction was quenched by the addition of 15 mL water. The mixture was extracted with EtOAc (2 × 25 mL). The combined organics were washed with water (2 × 15 mL), and brine (15 mL). The organics were dried over sodium sulfate, filtered, and evaporated to dryness. The crude material was absorbed on CELITE® and the product was isolated by flash chromatography (silica gel, MeOH/DCM 0-5% gradient) to provide (S)-N-((R)-1-(6,7-difluoro-4-oxo-3-((2-(trimethylsilyl)ethoxy)methyl)-3,4-dihydrophthalazin-1-yl)ethyl)-N,2-dimethylpropane-2-sulfinamide (XVc) 164 mg, 56% yield) as a single diastereomer. LCMS: m/z found 474.2 [M+H]⁺, RT = 5.93 min, (Method A); ¹H NMR (400 MHz, Chloroform-d) δ 8.26 (m, 1H), 7.78 (m, 1H), 5.55 (s, 2H), 5.03 (m, 1H), 3.79-3.70 (m, 2H), 2.47 (s, 3H), 1.67 (d, 3H), 1.24 (s, 9H), 1.03-0.91 (m, 2H), -0.02 (s, 9H).

### (R)-6,7-difluoro-4-(1-(methylamino)ethyl)phthalazin-1(2H)-one Hydrochloride (VIr)

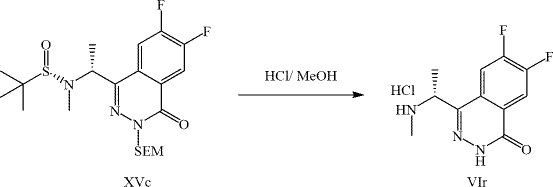

A solution (S)-N-((R)-1-(6,7-difluoro-4-oxo-3-((2-(trimethylsilyl)ethoxy)methyl)-3,4-dihydrophthalazin-1-yl)ethyl)-N,2-dimethylpropane-2-sulfinamide (XVc, 164 mg, 0.35 mmol) in hydrogen chloride (3 M in MeOH, 6.9 mL, 20.7 mmol) was prepared in a pressure vessel. The vessel was sealed and heated to 70° C. overnight. Upon cooling, the volatiles were removed in vacuo. The resulting residue was triturated with diethyl ether (2 × 10 mL) to provide (R)-6,7-difluoro-4-[1-(methylamino)ethyl]-2H-phthalazin-1-one hydrochloride (VIr, 90 mg, 94% yield) as a single enantiomer. LCMS: m/z found 240.2 [M+H]⁺, RT = 0.70 min, (Method B); ¹H NMR (400 MHz, Methanol- d ₄) δ 8.27 (m, 1H), 8.05 (m, 1H), 4.98 (m, 1H), 2.82 (s, 3H), 1.67 (d, 3H).

### (R)(3-chloro-4-fluorophenyl)-1-(1-(6,7-difluoro-4-oxo-3,4-dihydrophthalazin-1-yl)ethyl)-1-methylurea (Compound 32)

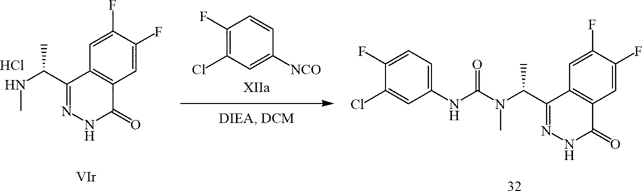

A mixture of DIEA (31 µL, 0.18 mmol) and enantiomerically pure (R)-6,7-difluoro-4-(1-(methylamino)ethyl)phthalazin-1(2H)-one hydrochloride (VIr, 20 mg, 73 µmol) in 2 mL of DCM was cooled in an ice bath. A solution of 2-chloro-1-fluoro-4-isocyanato-benzene (XIIa, 9 µL, 73 µmol) in 0.5 mL DCM was added and the mixture was stirred for 5 min. The crude reaction mixture was loaded directly on a pre-equilibrated silica gel column. The product was isolated by flash chromatography (silica gel, EtOAc/DCM 10-70% gradient) to provide enantiomerically pure (R)-3-(3-chloro-4-fluorophenyl)-1-(1-(6,7-difluoro-4-oxo-3,4-dihydrophthalazin-1-yl)ethyl)-1-methylurea (28 mg, 95 %). The product co-eluted by SFC with previously prepared Compound 32 (3-(3-chloro-4-fluorophenyl)-1-(1-(6,7-difluoro-4-oxo-3,4-dihydrophthalazin-1-yl)ethyl)-1-methylurea, enantiomer II). LCMS: m/z found 411.1/413.1 [M+H]⁺, RT = 4.74 min, (Method A); ¹H NMR (400 MHz, Chloroform-d) δ 10.07 (s, 1H), 8.22 (m, 1H), 8.13 (m, 1H), 7.60 (m, 1H), 7.30-7.21 (m, 1H), 7.10 (t, 1H), 6.33 (s, 1H), 6.20 (m, 1H), 2.78 (s, 3H), 1.54 (d, 3H); Chiral analytical SFC: RT = 7.97 min, Column: Chiralpak AD (4.6 × 250 mm) 5 µm, 30% of (ACN:MeOH (1:1)), Flow rate: 3.0 g/min.

Compound 32 was also independently prepared from racemic 6,7-difluoro-4-(1-(methylamino)ethyl)phthalazine-1(2H)-one (Vik) and isolated as the second eluting enantiomer (enantiomer II) under the chiral SFC conditions described elsewhere herein. The absolute configuration of Compound 32 was unambiguously determined by X-ray crystallography.

### X-ray Structure Determination of Compound 32

Crystals of Compound 32 were grown by vapor diffusion, using 3:2 ethyl acetate/hexanes as the solvent and hexanes as the anti-solvent. Compound 32, C₁₈H₁₄CIF₃N₄O₂, crystallizes in the tetragonal space group P4₁2₁2 (systematic absences h00: h=odd and 001: 1≠4n) with a=11.3172(1)Å, c=28.3100(2)Å, V=3625.92(7)Å³, Z=8, and d_{calc}=1.505 g/cm³ . X-ray intensity data were collected on a Rigaku XtaLAB Synergy-S diffractometer equipped with an HPC area detector (HyPix-6000HE) and employing confocal multilayer optic-monochromated Cu-Kα radiation (λ=1.54184 Å) at a temperature of 100 K. Preliminary indexing was performed from a series of sixty 0.5° rotation frames with exposures of 0.25 sec. for θ = ±47.20° and 1 sec. for θ = 107.75°. A total of 13606 frames (140 runs) were collected employing ω scans with a crystal to detector distance of 34.0 mm, rotation widths of 0.5° and exposures of 0.5 sec. for θ = ±47.20°, ±54.0°, +58.0°, +62.0°, ±66.0° and +70.0° and 2 sec. for θ = -74.0°, -78.0°, -82.0°, -86.25°, 90.0°, 94.0°, 98.0°, 102.0°, 106.0° and 107.75°.

Rotation frames were integrated using CrysAlisPro, producing a listing of unaveraged F² and σ(F²) values. A total of 175896 reflections were measured over the ranges 8.41 ≤ 2θ ≤ 148.91°, -14 ≤ h ≤ 13, -14 ≤ k ≤ 14, -35 ≤ 1 ≤ 35 yielding 3706 unique reflections (Rᵢₙₜ = 0.0435). The intensity data were corrected for Lorentz and polarization effects and for absorption using SCALE3 ABSPACK (minimum and maximum transmission 0.6779, 1.0000). The structure was solved by direct methods - ShelXT. Refinement was by full-matrix least squares based on F² using SHELXL-2018. All reflections were used during refinement. The weighting scheme used was w=1/[σ²(Fₒ² )+ (0.0275P)² + 2.0715P] where P = (Fₒ² + 2F_{c}²)/3. Non-hydrogen atoms were refined anisotropically and hydrogen atoms were refined using a riding model. Refinement converged to R1=0.0305 and wR2=0.0732 for 3690 observed reflections for which F > 4σ(F) and R1=0.0306 and wR2=0.0733 and GOF =1.075 for all 3706 unique, non-zero reflections and 255 variables. The maximum Δ/σ in the final cycle of least squares was 0.001 and the two most prominent peaks in the final difference Fourier were +0.52 and -0.47 e/Å³. The Hooft absolute structure parameter y was calculated using PLATON The resulting value was y = 0.000(1) indicating that the absolute structure has been assigned correctly. The Flack parameter refined to a similar value of 0.003(2). If these parameters are equal to 0 (within 3 standard deviations) then the absolute structure has been assigned correctly; if they are 1, the opposite enantiomer has been modeled.

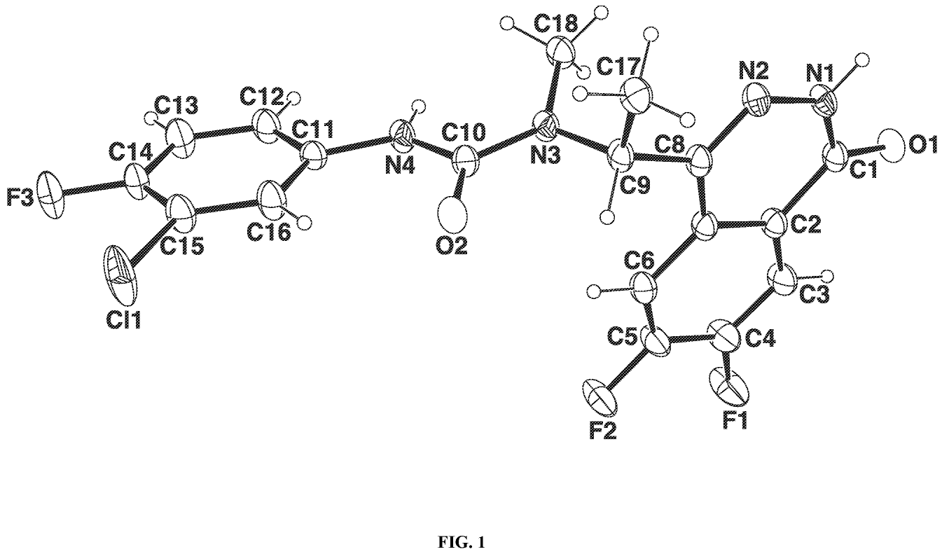

Table 1 lists cell information, data collection parameters, and refinement data. Final positional and equivalent isotropic thermal parameters are provided in Tables 2-3. FIG. 1 provides the ORTEP representation of Compound 32 with 50% probability thermal ellipsoids displayed, defining the absolute configuration of Compound 32 as (R)-3-(3-chloro-4-fluorophenyl)-1 -(1 -(6,7-difluoro-4-oxo-3,4-dihydrophthalazin-1 -yl)ethyl)-1 -methylurea.

TABLE 1

Summary of Structure Determination of Compound 32

| Empirical formula | C₁₈H₁₄ClF₃N₄O₂ |
|---|---|
| Formula weight | 410.78 |
| Diffractometer | Rigaku XtaLAB Synergy-S |
| Temperature (K) | 100 |
| Crystal system | tetragonal |
| Space group | P4₁2₁2 |
| a | 11.3172(1)Å |
| c | 28.3100(2)Å |
| Volume | 3625.92(7)Å³ |
| Z | 8 |
| d_{calc} | 1.505 g/cm³ |
| µ | 2.352 mm⁻¹ |
| F(000) | 1680.0 |
| Crystal size, mm | 0.22 × 0.14 × 0.08 |
| 2θ range for data collection | 8.414 - 148.912° |
| Index ranges | -14 ≤ h ≤ 13, -14 ≤ k ≤ 14, -35 ≤1 ≤ 35 |
| Reflections collected | 175896 |
| Independent reflections | 3706[R(int) = 0.0435] |
| Data/restraints/parameters | 3706/0/255 |
| Goodness-of-fit on F² | 1.075 |
| Final R indexes [I>=2σ (I)] | R₁ = 0.0305, wR₂ = 0.0732 |
| Final R indexes [all data] | R₁ = 0.0306, wR₂ = 0.0733 |
| Largest diff. peak/hole | 0.52/-0.47 eÅ⁻³ |
| Flack parameter | 0.003(2) |
| Hooft parameter | 0.000(1) |

TABLE 2

Refined Positional Parameters for Compound 32

| Atom | × | y | z | U(eq) |
|---|---|---|---|---|
| C1 | 0.1766(2) | 0.4866(2) | 0.80849(7) | 0.0221(4) |
| C2 | 0.2611(2) | 0.4270(2) | 0.77767(7) | 0.0223(4) |
| C3 | 0.3529(2) | 0.3619(2) | 0.79768(8) | 0.0287(5) |
| C4 | 0.4348(2) | 0.3124(2) | 0.76840(8) | 0.0317(5) |
| C5 | 0.4259(2) | 0.3266(2) | 0.71927(8) | 0.0289(5) |
| C6 | 0.3365(2) | 0.3896(2) | 0.69899(7) | 0.0246(5) |
| C7 | 0.2506(2) | 0.44166(19) | 0.72834(7) | 0.0216(4) |
| C8 | 0.1527(2) | 0.5109(2) | 0.71067(7) | 0.0215(4) |
| C9 | 0.1355(2) | 0.5339(2) | 0.65822(7) | 0.0222(4) |
| C10 | 0.2756(2) | 0.6005(2) | 0.59853(7) | 0.0222(4) |
| C11 | 0.4184(2) | 0.6839(2) | 0.54042(7) | 0.0222(4) |
| C12 | 0.5115(2) | 0.7632(2) | 0.53652(8) | 0.0272(5) |
| C13 | 0.5668(2) | 0.7819(2) | 0.49355(8) | 0.0328(5) |
| C14 | 0.5296(2) | 0.7202(2) | 0.45440(8) | 0.0271(5) |
| C15 | 0.4391(2) | 0.6406(2) | 0.45797(8) | 0.0291(5) |
| C16 | 0.3810(2) | 0.6215(2) | 0.50071(8) | 0.0290(5) |
| C17 | 0.0124(2) | 0.5778(2) | 0.64595(8) | 0.0260(5) |
| C18 | 0.2462(2) | 0.7225(2) | 0.67132(8) | 0.0258(5) |
| N1 | 0.09011(17) | 0.54710(18) | 0.78588(6) | 0.0230(4) |
| N2 | 0.07675(17) | 0.56186(17) | 0.73835(6) | 0.0228(4) |
| N3 | 0.22854(17) | 0.61696(16) | 0.64252(6) | 0.0223(4) |
| N4 | 0.36564(18) | 0.67458(17) | 0.58579(6) | 0.0234(4) |
| C11 | 0.39819(7) | 0.56199(8) | 0.40802(2) | 0.0508(2) |
| F1 | 0.52555(15) | 0.24988(16) | 0.78554(5) | 0.0463(5) |
| F2 | 0.51050(14) | 0.27684(15) | 0.69249(5) | 0.0401(4) |
| F3 | 0.58477(14) | 0.73702(15) | 0.41266(5) | 0.0389(4) |
| O1 | 0.18394(15) | 0.48792(15) | 0.85215(5) | 0.0253(3) |
| O2 | 0.23877(15) | 0.52125(14) | 0.57213(5) | 0.0268(4) |

TABLE 3

Positional Parameters for Hydrogens in Compound 32

| Atom | x | y | z | U(eq) |
|---|---|---|---|---|
| H3 | 0.358314 | 0.352331 | 0.830946 | 0.034 |
| H6 | 0.332277 | 0.39828 | 0.665651 | 0.03 |
| H9 | 0.148083 | 0.457463 | 0.641233 | 0.027 |
| H12 | 0.537609 | 0.805141 | 0.563662 | 0.033 |
| H13 | 0.629883 | 0.836945 | 0.491106 | 0.039 |
| H16 | 0.317331 | 0.56703 | 0.502722 | 0.035 |
| H17a | -0.003208 | 0.651852 | 0.662761 | 0.039 |
| H17b | -0.04613 | 0.518402 | 0.655339 | 0.039 |
| H17c | 0.007007 | 0.591374 | 0.611827 | 0.039 |
| H18a | 0.302307 | 0.704956 | 0.696746 | 0.039 |
| H18b | 0.170576 | 0.747113 | 0.684985 | 0.039 |
| H18c | 0.277675 | 0.786253 | 0.651554 | 0.039 |
| H1 | 0.036445 | 0.580725 | 0.803921 | 0.028 |
| H4 | 0.39374 | 0.721254 | 0.607963 | 0.028 |

### (R)(1-(6,7-difluoro-4-oxo-3,4-dihydrophthalazinyl)ethyl)methyl-3-(3,4-difluorophenyl)urea (Compound 41)

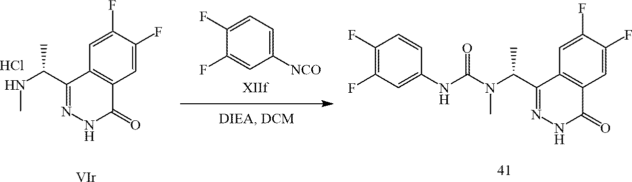

(R)-1 -(1 -(6, 7-Difluoro-4-oxo-3,4-dihy drophthal azin- 1 -yl)ethyl)-1 -m ethyl-3 -(3,4-difluorophenyl)urea was synthesized in an analogous manner as described above from enantiomerically pure (R)-6,7-difluoro-4-(1-(methylamino)ethyl)phthalazin-1(2H)-one hydrochloride (VIr) and 1,2-difluoro-4-isocyanatobenzene (XIIf). LCMS: m/z found 395.2 [M+H]⁺, RT = 4.11 min, (Method A); ¹H NMR (400 MHz, Chloroform-d) δ 10.18 (s, 1H), 8.26-8.17 (m, 1H), 8.14 (m, 1H), 7.58-7.47 (m, 1H), 7.10 (m, 1H), 7.05-6.95 (m, 1H), 6.34 (s, 1H), 6.20 (m, 1H), 2.79 (s, 3H), 1.59-1.50 (d, 3H).

### (R)(1-(6,7-difluoro-4-oxo-3,4-dihydrophthalazinyl)ethyl)methyl-3-(3,4,5-trifluorophenyl)urea (Compound 42)

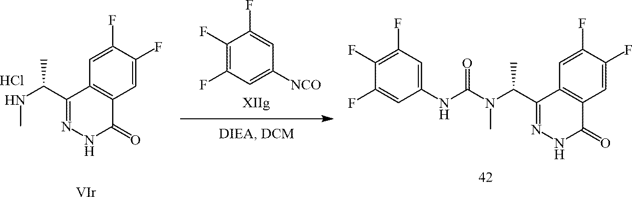

(R)(1-( 6, 7 -Difluoro-4-oxo-3,4-dihydrophthalazinyl)ethyl)methyl-3-(3,4,5-trifluorophenyl)urea was synthesized in an analogous manner as described above from enantiomerically pure (R)-6,7-difluoro-4-(1-(methylamino)ethyl)phthalazin-1(2H)-one hydrochloride (VIr) and 1,2,3-trifluoro-5-isocyanatobenzene (XIIg). LCMS: m/z found 413.2 [M+H]⁺, RT = 4.55 min, (Method A); ¹H NMR (400 MHz, Chloroform-d) δ 10.40 (s, 1H), 8.22 (m, 1H), 8.10 (m, 1H), 7.17 (m, 2H), 6.35 (s, 1H), 6.18 (m, 1H), 2.79 (s, 3H), 1.60-1.51 (m, 3H).

### (R)(1-(6,7-difluoro-4-oxo-3,4-dihydrophthalazinyl)ethyl)-3-(4-fluorophenyl)methylurea (Compound 43)

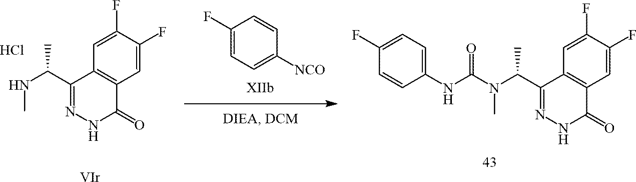

(R)-1 -(1 -(6,7-Difluoro-4-oxo-3,4-dihydrophthalazin-1 -yl)ethyl)-3 -(4-fluorophenyl)-1 -methylurea was synthesized in an analogous manner as described above enantiomerically pure (R)-6,7-difluoro-4-(1-(methylamino)ethyl)phthalazin-1(2H)-one hydrochloride (VIr) and 1-fluoro-4-isocyanatobenzene (XIIb). LCMS: m/z found 377.3 [M+H]⁺, RT = 3.73 min, (Method A); ¹H NMR (400 MHz, Chloroform-d) δ 10.02 (s, 1H), 8.19 (m, 2H), 7.42-7.34 (m, 2H), 7.08-6.99 (m, 2H), 6.31 (s, 1H), 6.21 (m, 1H), 2.78 (s, 3H), 1.58-1.50 (d, 3H).

### 5-fluoro-2,3-dihydrophthalazine-1,4-dione (Xc)

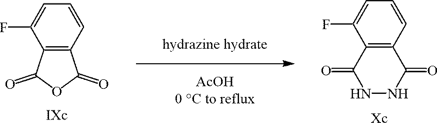

Hydrazine hydrate (1.29 mL, 26.49 mmol) was added slowly to a 0° C. solution of 4-fluoroisobenzofuran-1,3-dione (IXc, 4.00 g, 24.08 mmol) in glacial acetic acid (30 mL). After the hydrazine addition was complete, the reaction mixture was allowed to warm to room temperature, then heated at 90° C. for 90 min. Upon cooling and standing overnight at room temperature, a white crystalline material was collected by vacuum filtration, washed with 100 mL of water, and dried under high vacuum in a 50° C. vacuum oven to provide 5-fluoro-2,3-dihydrophthalazine-1,4-dione (Xc) (3.58 g, 83% yield). LCMS: m/z found 181.1 [M+H]⁺, RT = 0.77 min, (Method B); ¹H NMR (400 MHz, DMSO-d₆) δ 11.57 br s, 2H), 7.89 (m, 2H), 7.71-7.61 (m, 1H).

### 5-fluoro-4-oxo-3,4-dihydrophthalazin-1-yl Trifluoromethanesulfonate (XIc)

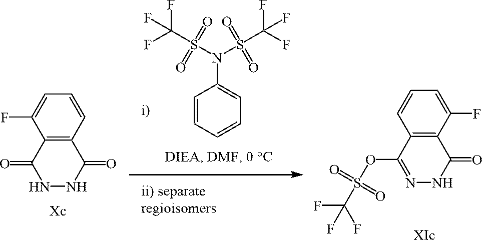

DIEA (2.13 mL, 12.21 mmol) was added to a 0° C. suspension of 5-fluoro-2,3-dihydrophthalazine-1,4-dione (Xc, 1.10 g, 6.11 mmol) in anhydrous DMF (30 mL). The mixture turned light-yellow. An additional 5 mL of DMF was added followed by 1,1,1-trifluoro-N-phenyl-N-(trifluoromethylsulfonyl)methanesulfonamide (2.40 g, 6.72 mmol) in one portion. After 5 min, the ice bath was removed, and a solution gradually formed. LCMS showed 2 product peaks in a 6:1 ratio. The reaction mixture was diluted with EtOAc (100 mL) and washed with hydrochloric acid (0.2 M, 2 × 50 mL), water (40 mL), and brine (40 mL). The organics were dried over sodium sulfate, filtered, and evaporated to dryness. The major regioisomer was isolated by flash chromatography (silica gel, EtOAc/hexanes 5-60% gradient) to provide 5-fluoro-4-oxo-3,4-dihydrophthalazin-1-yl trifluoromethanesulfonate (XIc, 0.89 g, 47% yield). ¹H NMR (400 MHz, DMSO-d₆) δ 12.84 (s, 1H), 8.11 (m, 1H), 7.83 (m, 1H), 7.67 (m, 1H).

### 4-acetyl-8-fluorophthalazin-1(2H)-one (Vh)

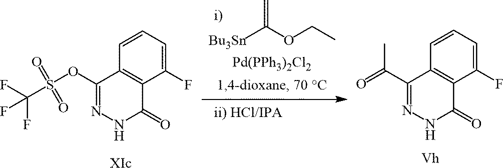

Step i: A solution of (5-fluoro-4-oxo-3H-phthalazin-1-yl) trifluoromethanesulfonate (XIc, 439 mg, 1.41 mmol) in 1,4-dioxane (4 mL) in a pressure vessel was degassed with nitrogen. Tributyl(1-ethoxyvinyl)stannane (0.61 mL, 1.83 mmol) and dichlorobis(triphenylphosphine)palladium(II) (79 mg, 0.11 mmol) were added. The vessel was sealed, and the mixture was heated in a 70° C. heating block for 3 h. Upon cooling, the reaction mixture was diluted with EtOAc (50 mL) and filtered through a pad of CELITE®. The filter cake was washed with an additional portion of EtOAc (20 mL). The combined filtrate was evaporated to dryness. The major product was isolated by flash chromatography (silica gel, EtOAc/hexanes 10-60% gradient) to provide 4-(1-ethoxyvinyl)-8-fluoro-2H-phthalazin-1-one (260 mg, 79% yield). ¹H NMR (400 MHz, Chloroform-d) δ 9.93 (s, 1H), 7.84-7.71 (m, 2H), 7.47-7.37 (m, 1H), 4.62-4.54 (m, 2H), 4.02 (q, 2H), 1.42 (t, 3H).

Step ii: Hydrochloric acid (2 M, 0.54 mL, 1.08 mmol) was added to a mixture of 4-(1-ethoxyvinyl)-5-fluoro-2H-phthalazin-1-one (85 mg, 0.36 mmol) in 10 mL IPA. After 3 h at room temperature, the volatiles were removed in vacuo to provide 4-acetyl-8-fluoro-2H-phthalazin-1-one (Vh, 72 mg, 96% yield). ¹H NMR (400 MHz, DMSO-d₆) δ 13.17 (s, 1H), 8.66-8.59 (m, 1H), 7.98 (m, 1H), 7.70-7.55 (m, 1H), 2.58 (s, 3H).

### 8-fluoro-4-(1-(methylamino)ethyl)phthalazin-1(2H)-one (VIs)

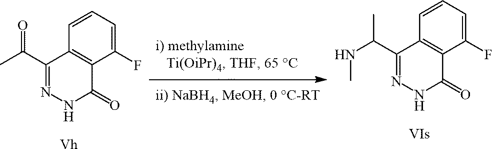

Tetraisopropoxytitanium (0.28 mL, 0.96 mmol) and methylamine (2 M in THF, 0.19 mL, 0.38 mmol) were added to a suspension of 4-acetyl-8-fluoro-2H-phthalazin-1-one (Vh, 66 mg, 0.32 mmol) in THF (0.5 mL) in a pressure vessel. The vessel was sealed and heated in a 65° C. heating block for 2 hours. After cooling to room temperature, the mixture was diluted with MeOH (0.7 mL) and cooled in an ice bath. Sodium borohydride (18 mg, 0.48 mmol) was added. After 30 min, LCMS showed desired product. The ice bath was removed, and after an additional 20 minutes, the reaction mixture was slowly added to a rapidly stirred brine solution (0.5 mL) and diluted with 20 mL of 9:1 (v/v) EtOAc/MeCN. The mixture was filtered through a pad of CELITE®, and the filter cake was washed with an additional portion of EtOAc (15 mL). The combined filtrate was evaporated to dryness to provide crude racemic 8-fluoro-4-(1-(methylamino)ethyl)phthalazin-1(2H)-one (VIs, 75 mg). ¹H NMR (400 MHz, Methanol-d₄) δ 8.02-7.80 (m, 2H), 7.56 (m, 1H), 4.42-4.28 (m, 1H), 2.40 (s, 3H), 1.46 (d, 3H).

### 3-chloro-4-fluorophenyl)-1-(1-(5-fluoro-4-oxo-3,4-dihydrophthalazin-1-yl)ethyl)-1-methylurea (Compound 44)

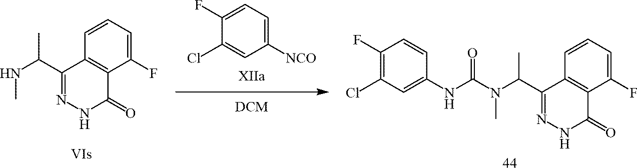

3-Chloro-4-fluorophenyl)-1-(1-(5-fluoro-4-oxo-3,4-dihydrophthalazin-1-yl)ethyl)-1-methylurea was synthesized in an analogous manner as described above from racemic 8-fluoro-4-(1-(methylamino)ethyl)phthalazin-1(2H)-one (VIs) and 2-chloro-1-fluoro-4-isocyanatobenzene (XIIa). LCMS: m/z found 393.2/395.2 [M+H]⁺, RT = 3.88 min, (Method A); ¹H NMR (400 MHz, DMSO-d₆) δ 12.71 (s, 1H), 8.54 (s, 1H), 7.96 (m, 1H), 7.85 (m, 1H), 7.78 (m, 1H), 7.65-7.55 (m, 1H), 7.50 (m, 1H), 7.32 (t, 1H), 6.06 (m, 1H), 2.66 (s, 3H), 1.42 (d, 3H).

### 3-(3,4-difluorophenyl)-1-(1-(5-fluoro-4-oxo-3,4-dihydrophthalazin-1-yl)ethyl)-1-methylurea (Compound 45)

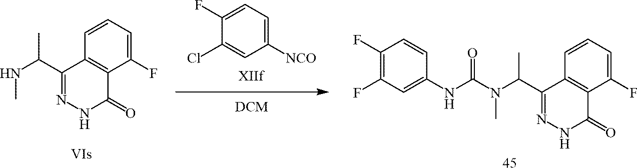

3 -(3,4-Difluorophenyl)-1 -(1 -(5 -fluoro-4-oxo-3,4-dihydrophthalazin-1 -yl)ethyl)-1 -methylurea was synthesized in an analogous manner as described above from racemic 8-fluoro-4-(1-(methylamino)ethyl)phthalazin-1(2H)-one (VIs) and 1,2-difluoro-4-isocyanatobenzene (XIIf). LCMS: m/z found 377.2 [M+H]⁺, RT = 3.53 min, (Method A); ¹H NMR (400 MHz, DMSO-d₆) δ 12.71 (s, 1H), 8.55 (s, 1H), 7.96 (m, 1H), 7.79 (d, 1H), 7.78-7.67 (m, 1H), 7.60 (m, 1H), 7.39-7.28 (m, 2H), 6.06 (m, 1H), 2.66 (s, 3H), 1.42 (d, 3H).

### (R)-N-(6,7-difluoro-4-oxo-3,4-dihydrophthalazin-1-yl)ethyl)-N-methyl-1H-indole-2-carboxamide (Compound 46)

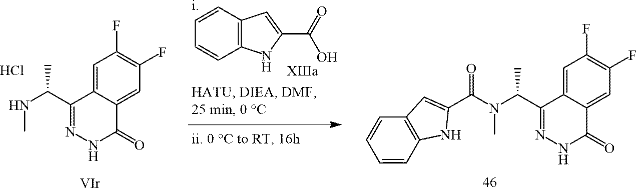

HATU (18 mg, 0.05 mmol) and DIEA (16 µL, 0.09 mmol) were added to a 0° C. solution of 1H-indole-2-carboxylic acid (XIIIa, 7.4 mg, 0.05 mmol) in anhydrous DMF (1 mL). After 10 min, a solution of (R)-6,7-difluoro-4-[1-(methylamino)ethyl]-2H-phthalazin-1-one hydrochloride (VIr, 13 mg, 0.05 mmol) and DIEA (9 µL, 0.05 mmol) in DMF (1 mL) was added. The cooling bath was removed. After 2 h, the reaction mixture was diluted with EtOAc (30 mL) and washed with water (2 × 15 mL) and brine (2 × 15 mL). The organics were dried over sodium sulfate and evaporated to dryness. The product was isolated by flash-chromatography (silica gel, EtOAc/heaxanes 5-60% gradient) to provide (R)-N-(1-(6,7-difluoro-4-oxo-3,4-dihydrophthalazin-1-yl)ethyl)-N-methyl-1H-indole-2-carboxamide (47, 13 mg, 72% yield). LCMS: m/z found 383.3 [M+H]⁺, RT = 4.07 min, (Method A); ¹H NMR (400 MHz, Chloroform-d) δ 10.44 (s, 1H), 9.49 (s, 1H), 8.23 (m, 1H), 8.08 (m, 1H), 7.69-7.62 (m, 1H), 7.48 (d, 1H), 7.33 (m, 1H), 7.14 (t, 1H), 6.88 (s, 1H), 6.53 (m, 1H), 3.18 (s, 3H), 1.64 (d, 3H).

### 6,7-difluoro-4-(1-(ethylamino)ethyl)phthalazin-1(2H)-one (VIm)

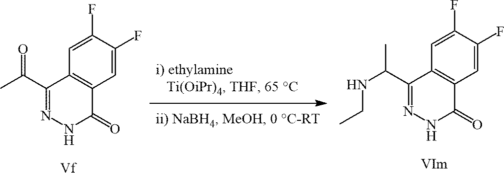

Tetraisopropoxytitanium (320 µL, 1.07 mmol) was added to a solution of 4-acetyl-6,7-difluoro-2H-phthalazin-1-one (Vf, 80 mg, 0.36 mmol) and an ethylamine solution (2 M in THF, 0.20 mL, 0.40 mmol) in 0.8 mL anhydrous THF in a pressure vessel. The vessel was sealed, and the mixture was heated to 65° C. for 2 h in a heating block. The reaction mixture was diluted with anhydrous methanol (4 mL) and cooled in an ice bath. Sodium borohydride (20 mg, 0.54 mmol) was added in one portion. The reaction mixture was stirred for 10 min, and the ice bath was removed. After an additional 25 min, the reaction mixture was added dropwise to a rapidly stirred brine solution (0.5 mL), and diluted with 20 mL of 9:1 (v/v) EtOAc/MeCN. After stirring for 15 min, the mixture was filtered through a pad of CELITE®, and the filter cake was washed with an additional portion of EtOAc (15 mL). The combined filtrate was evaporated to dryness to provide crude racemic 4-[1-(ethylamino)ethyl]-6,7-difluoro-2H-phthalazin-1-one (VIm, 90 mg). LCMS: m/z found 254.2 [M+H]⁺, RT = 0.52 min, (Method B); ¹H NMR (400 MHz, Methanol-d₄) δ 8.21 (m, 2H), 4.42 (m, 1H), 2.68 (m, 2H), 1.47 (d, 3H), 1.15 (t, 3H).

### N-(6,7-difluoro-4-oxo-3,4-dihydrophthalazin-1-yl)ethyl)-5-fluoro-N-ethyl-1H-indole-2-carboxamide (Compound 47)

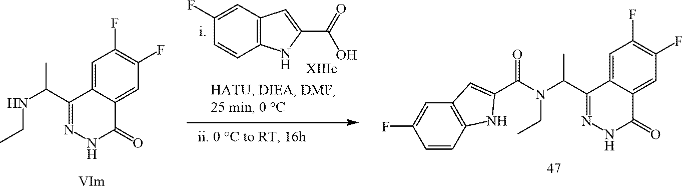

Racemic N-(1-(6,7-difluoro-4-oxo-3,4-dihydrophthalazin-1-yl)ethyl)-5-fluoro-N-methyl-1H-indole-2-carboxamide was synthesized in an analogous manner as described above from 6,7-difluoro-4-(1-(ethylamino)ethyl)phthalazin-1(2H)-one (VIm) and 5-fluoro-1H-indole-2-carboxylic acid (XIIIc). LCMS: m/z found 415.2 [M+H]⁺, RT = 4.71 min, (Method A); ¹H NMR (400 MHz, Chloroform-d) δ 10.56 (s, 1H), 9.60 (s, 1H), 8.24 (m, 1H), 7.97 (s, 1H), 7.41 (m, 1H), 7.32-7.23 (m, 1H), 7.09 (m, 1H), 6.82 (s, 1H), 6.58 (m, 1H), 3.87-3.55 (m, 2H), 1.65 (s, 3H), 0.98 (t, 3H).

### N-(6,7-difluoro-4-oxo-3,4-dihydrophthalazin-1-yl)ethyl)-N-ethyl-1H-indole-2-carboxamide (Compound 48)

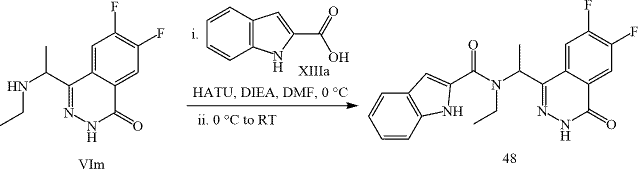

Racemic N-(1-(6,7-difluoro-4-oxo-3,4-dihydrophthalazin-1-yl)ethyl)-N-ethyl-1H-indole-2-carboxamide was synthesized in an analogous manner as described above from racemic 6,7-difluoro-4-(1-(ethylamino)ethyl)phthalazin-1(2H)-one (VIm) and 1H-indole-2-carboxylic acid (XIIIa). LCMS: m/z found 397.3 [M+H]⁺, RT = 4.62 min, (Method A); ¹H NMR (400 MHz, Chloroform-d) δ 10.41 (s, 1H), 9.53 (s, 1H), 8.23 (m, 1H), 7.98 (s, 1H), 7.66 (d, 1H), 7.48 (d, 1H), 7.33 (t, 1H), 7.15 (t, 1H), 6.87 (s, 1H), 6.59 (s, 1H), 3.79-3.56 (m, 2H), 1.66 (s, 3H), 0.99 (t, 3H).

### N-(6,7-difluoro-4-oxo-3,4-dihydrophthalazin-1-yl)ethyl)-N-ethyl-4,5-difluoro-1H-indole-2-carboxamide (Compound 49)

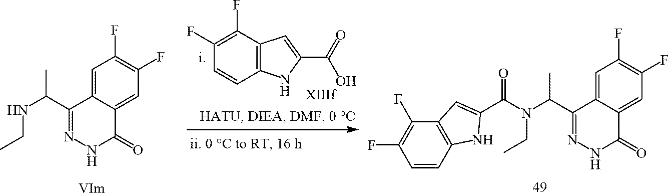

Racemic N-(1-(6,7-difluoro-4-oxo-3,4-dihydrophthalazin-1-yl)ethyl)-N-ethyl-4,5-difluoro-1H-indole-2-carboxamide was synthesized in an analogous manner as described above from racemic 6,7-difluoro-4-(1-(ethylamino)ethyl)phthalazin-1(2H)-one (VIm) and 4,5 -difluoro-1H-indole-2-carboxylic acid (XIIIf). LCMS: m/z found 433.2 [M+H]⁺, RT = 4.96 min, (Method A); ¹H NMR (400 MHz, Chloroform-d) δ 10.31 (s, 1H), 9.62 (s, 1H), 8.23 (m, 1H), 7.95 (s, 1H), 7.16 (q, 2H), 6.94 (s, 1H), 6.57 (s, 1H), 3.80 - 3.67 (m, 2H), 1.65 (s, 3H), 1.00 (t, 3H).

### N-(6,7-difluoro-4-oxo-3,4-dihydrophthalazin-1-yl)ethyl)-5,6-difluoro-N-methyl-1H-indole-2-carboxamide (Compound 50)

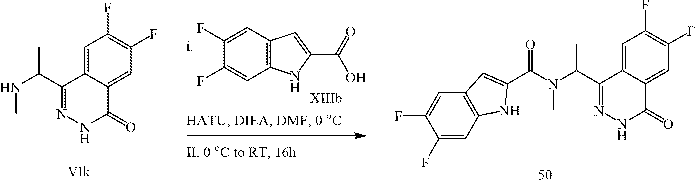

HATU (61 mg, 0.16 mmol) and DIEA (63 µL, 0.36 mmol) were added to a 0° C. solution of 5,6-difluoro-1H-indole-2-carboxylic acid (XIIIb, 29 mg, 0.15 mmol) in DMF (0.75 mL). After 25 min, a solution of 6,7-difluoro-4-[1-(methylamino)ethyl]-2H-phthalazin-1-one (VIk, 35 mg, 0.15 mmol) in DMF (0.75 mL) was added. The reaction mixture was allowed to warm to room temperature and stirred overnight. The reaction mixture was diluted with EtOAc (30 mL) and washed with 0.1 M hydrochloric acid (2 × 8 mL), followed by saturated sodium bicarbonate solution (10 mL). The organics were dried over sodium sulfate and evaporated to dryness. The product was isolated by flash chromatography (silica gel, MeOH/DCM 0.5 - 8% gradient) to provide to provide racemic N-(1-(6,7-difluoro-4-oxo-3,4-dihydrophthalazin-1-yl)ethyl)-5,6-difluoro-N-methyl-1H-indole-2-carboxamide (38 mg, 63% yield). LCMS: m/z found 419.2 [M+H]⁺, RT = 4.32 min, (Method A); ¹H NMR (400 MHz, DMSO-d₆) δ 12.99 (s, 1H), 11.92 (d, 1H), 8.21 (m, 1H), 8.01 (s, 1H), 7.61 (m 1H), 7.37 (m, 1H), 6.97-6.91 (m, 1H), 6.36 (d, 1H), 3.03 (s, 3H), 1.54 (d, 3H).

### N-(6,7-difluoro-4-oxo-3,4-dihydrophthalazin-1-yl)ethyl)-5-fluoro-N-methyl-1H-indole-2-carboxamide (Compound 51)

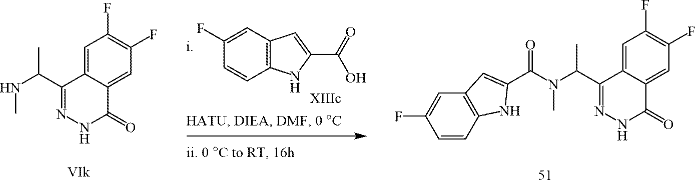

Racemic N-(1-(6,7-difluoro-4-oxo-3,4-dihydrophthalazin-1-yl)ethyl)-5-fluoro-N-methyl-1H-indole-2-carboxamide was synthesized in an analogous manner as described above from racemic 6,7-difluoro-4-(1-(methylamino)ethyl)phthalazin-1(2H)-one (VIk) and 5-fluoro-1H-indole-2-carboxylic acid (XIIIc). LCMS: m/z found 401.2 [M+H]⁺, RT = 4.13 min, (Method A); ¹H NMR (400 MHz, DMSO-d6) δ 13.00 (s, 1H), 11.85 (s, 1H), 8.22 (m, 1H), 8.01 (s, 1H), 7.45 (m, 1H), 7.35 (m, 1H), 7.08 (m, 1H), 6.91 (m, 1H), 6.37 (d, 1H), 3.03 (s, 3H), 1.54 (d, 3H).

### N-(6,7-difluoro-4-oxo-3,4-dihydrophthalazin-1-yl)ethyl)-6-fluoro-N-methyl-1H-indole-2-carboxamide (Compound 52)

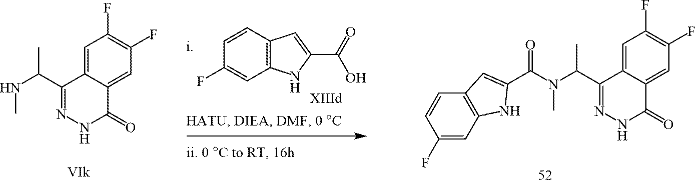

Racemic N-(1-(6,7-difluoro-4-oxo-3,4-dihydrophthalazin-1-yl)ethyl)-6-fluoro-N-methyl-1H-indole-2-carboxamide was synthesized in an analogous manner as described above from racemic 6,7-difluoro-4-(1-(methylamino)ethyl)phthalazin-1(2H)-one (VIk) and 6-fluoro-1H-indole-2-carboxylic acid (XIIId). LCMS: m/z found 401.2 [M+H]⁺, RT = 4.18 min, (Method A); ¹H NMR (400 MHz, DMSO-d₆) δ 13.00 (s, 1H), 11.82 (s, 1H), 8.22 (m, 1H), 8.02 (s, 1H), 7.62 (m, 1H), 7.17 (m, 1H), 7.00-6.87 (m, 2H), 6.38 (d, 1H), 3.04 (s, 3H), 1.54 (d, 3H).

### N-(6,7-difluoro-4-oxo-3,4-dihydrophthalazin-1-yl)ethyl)-4-fluoro-N-methyl-1H-indole-2-carboxamide (Compound 53)

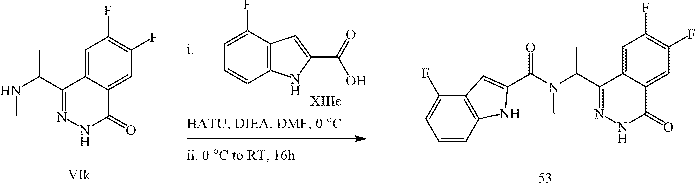

Racemic N-(1-(6,7-difluoro-4-oxo-3,4-dihydrophthalazin-1-yl)ethyl)-4-fluoro-N-methyl-1H-indole-2-carboxamide was synthesized in an analogous manner as described above from racemic 6,7-difluoro-4-(1-(methylamino)ethyl)phthalazin-1(2H)-one (VIk) and 4-fluoro-1H-indole-2-carboxylic acid (XIIIe). LCMS: m/z found 401.2 [M+H]⁺, RT = 4.21 min, (Method A); ¹H NMR (400 MHz, DMSO-d₆) δ 13.00 (s, 1H), 12.07 (s, 1H), 8.22 (m, 1H), 8.01 (s, 1H), 7.29 (m, 1H), 7.19 (m, 1H), 6.99-6.94 (m, 1H), 6.87-6.77 (m, 1H), 6.37 (d, 1H), 3.05 (s, 3H), 1.55 (d, 3H).

### N-(6,7-difluoro-4-oxo-3,4-dihydrophthalazin-1-yl)ethyl)-4,5-difluoro-N-methyl-1H-indole-2-carboxamide (Compound 54)

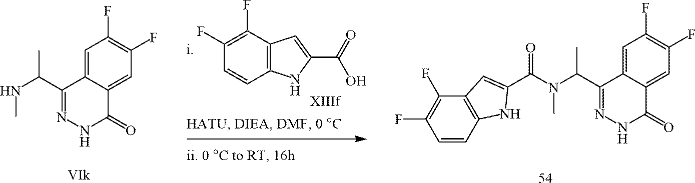

Racemic N-(1-(6,7-difluoro-4-oxo-3,4-dihydrophthalazin-1-yl)ethyl)-4,5-difluoro-N-methyl-1H-indole-2-carboxamide was synthesized in an analogous manner as described above from racemic 6,7-difluoro-4-(1-(methylamino)ethyl)phthalazin-1(2H)-one (VIk) and 4,5-difluoro-1H-indole-2-carboxylic acid (XIIIf). LCMS: m/z found 419.2 [M+H]⁺, RT = 4.36 min, (Method A); ¹H NMR (400 MHz, DMSO-d₆) δ 13.00 (s, 1H), 12.14 (s, 1H), 8.22 (m, 1H), 8.00 (s, 1H), 7.27 (m, 2H), 7.03 (s, 1H), 6.36 (d, 1H), 3.05 (s, 3H), 1.54 (d, 3H).

### N-(6,7-difluoro-4-oxo-3,4-dihydrophthalazin-1-yl)ethyl)-N,3-dimethyl-1H-indole-2-carboxamide (Compound 55)

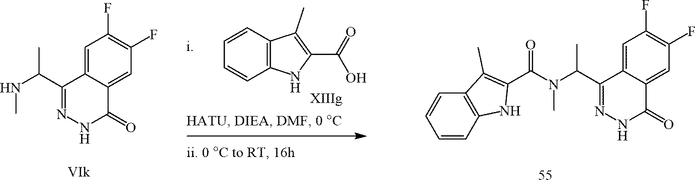

Racemic N-(1-(6,7-difluoro-4-oxo-3,4-dihydrophthalazin-1-yl)ethyl)-N,3-dimethyl-1H-indole-2-carboxamide was synthesized in an analogous manner as described above from racemic 6,7-difluoro-4-(1-(methylamino)ethyl)phthalazin-1(2H)-one (VIk) and 3-methyl-1H-indole-2-carboxylic acid (XIIIg). LCMS: m/z found 397.3 [M+H]⁺, RT = 4.11 min, (Method A); ¹H NMR (400 MHz, DMSO-d₆) δ 12.99 (s, 1H), 11.24 (s, 1H), 8.25 (m, 1H), 8.03 (s, 1H), 7.52 (d, 1H), 7.33 (m, 1H), 7.16 (m, 1H), 7.03 (m, 1H), 6.32 (s, 1H), 2.71 (s, 3H), 2.15 (s, 3H), 1.58 (d, 3H).

### N-(6,7-difluoro-4-oxo-3,4-dihydrophthalazin-1-yl)ethyl)-N-isobutyl-1H-indole-2-carboxamide (Compound 56)

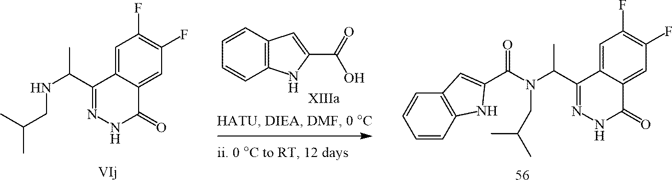

HATU (56 mg, 0.15 mmol) and DIEA (59 µL, 0.34 mmol) were added to a stirred 0° C. solution of 1H-indole-2-carboxylic acid (XIIIa, 22 mg, 0.13 mmol) in DMF (0.75 mL). After 25 min, a solution of 6,7-difluoro-4-[1-(isobutylamino)ethyl]-2H-phthalazin-l-one (VIj, 38 mg, 0.13 mmol) in DMF (0.75 mL) was added. The reaction mixture was stirred at room temperature for 12 days. The reaction mixture was diluted with EtOAc (30 mL) and washed with hydrochloric acid (0.1 M, 2 × 8 mL), followed by saturated NaHCO₃ (8 mL). The organics were dried over sodium sulfate and evaporated to dryness. The product was isolated by flash chromatography (silica gel, MeOH/DCM 0.5-7% gradient) to provide racemic N-[1-(6,7-difluoro-4-oxo-3H-phthalazin-1-yl)ethyl]-N-isobutyl-1H-indole-2-carboxamide (11 mg, 19%). LCMS: m/z found 425.3 [M+H]⁺, RT = 4.72 min, (Method A); ¹H NMR (400 MHz, DMSO-d₆) δ 13.01 (s, 1H), 11.76 (s, 1H), 8.23 (m, 1H), 7.61 (d, 1H), 7.45 (d, 1H), 7.25-7.16 (m, 1H), 7.05 (t, 1H), 6.90 (s, 1H), 6.37 (d, 1H), 3.28 - 3.13 (m, 2H), 1.63 (br s, 3H), 1.47 (m, 1H), 0.58 (br s, 3H), 0.39 (br s, 3H).

### (R)-N-(6,7-difluoro-4-oxo-3,4-dihydrophthalazin-1-yl)ethyl)-4,5-difluoro-N-methyl-1H-indole-2-carboxamide (Compound 57)

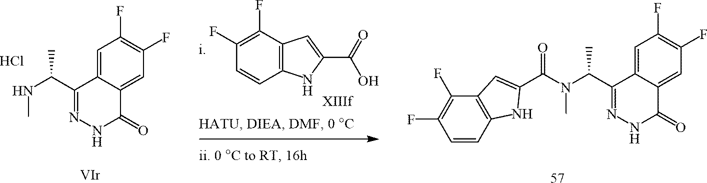

(R)-N-(6,7-difluoro-4-oxo-3,4-dihydrophthalazin-1-yl)ethyl)-4,5-difluoro-N-methyl-1H-indole-2-carboxamide was synthesized in an analogous manner as described above from enantiomerically pure (R)-6,7-difluoro-4-(methylamino)ethyl)phthalazin-1(2H)-one hydrochloride (VIr) and 4,5-difluoro-1H-indole-2-carboxylic acid (XIIIf). LCMS: m/z found 419.3 [M+H]⁺, RT = 4.39 min, (Method A); ¹H NMR (400 MHz, DMSO-d₆) δ 13.01 (s, 1H), 12.15 (s, 1H), 8.22 (m, 1H), 8.00 (s, 1H), 7.26 (m, 2H), 7.03 (s, 1H), 6.36 (d, 1H), 3.05 (s, 3H), 1.54 (d, 3H).

### (R)-N-(6,7-difluoro-4-oxo-3,4-dihydrophthalazin-1-yl)ethyl)-N-methyl-4,5,6,7-tetrahydro-1H-indole-2-carboxamide (Compound 58)

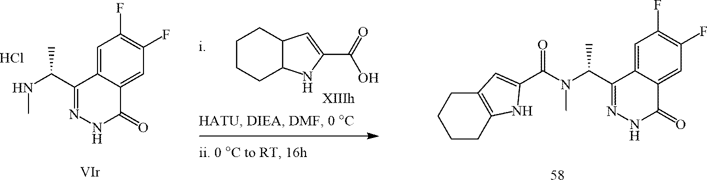

(R)-N-(6,7-Difluoro-4-oxo-3,4-dihydrophthalazin-1-yl)ethyl)-N-methyl-1H-indole-2-carboxamide was synthesized in an analogous manner as described above enantiomerically pure (R)-6,7-difluoro-4-(methylamino)ethyl)phthalazin-1(2H)-one hydrochloride (VIr) and 4,5,6,7-tetrahydro-1H-indole-2-carboxylic acid (XIIIh). LCMS: m/z found 387.3 [M+H]⁺, RT = 4.33 min, (Method A); ¹H NMR (400 MHz, DMSO-d₆) δ 12.95 (s, 1H), 11.16 (d, 1H), 8.19 (m, 1H), 8.00 (s, 1H), 6.37-6.28 (m, 2H), 2.87 (s, 3H), 2.54 (m, 2H), 2.41-2.36 (m, 2H), 1.73-1.61 (m, 4H), 1.45 (d, 3H).

### N-(6,7-difluoro-4-oxo-3,4-dihydrophthalazin-1-yl)ethyl)-N-methylindolizine-2-carboxamide (Compound 59)

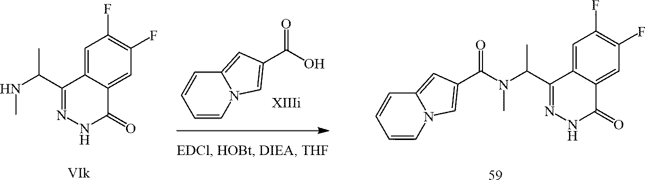

To a stirred solution of indolizine-2-carboxylic acid (XIIIi, 34 mg, 0.21 mmol) in THF (0.5 mL) at room temperature were added DIEA (110 µL, 0.63 mmol), EDCI-HCl (60 mg, 0.32 mmol) and HOBt monohydrate (42 mg, 0.32 mmol) and the reaction mixture was stirred at room temperature for 15 min. To this mixture was added racemic 6,7-difluoro-4-(1-(methylamino)ethyl)isoquinolin-1(2H)-one (VIk, 51 mg, 0.21 mmol) and the resulting reaction mixture was stirred at room temperature for 16 h. The mixture was then poured on to ice water/saturated sodium bicarbonate solution (10 mL) and the precipitated solid was collected by filtration. The solid was washed with water and triturated with diethyl ether (5 mL) and n-pentane (5 mL) to afford racemic N-(1-(6,7-difluoro-4-oxo-3,4-dihydrophthalazin-1-yl)ethyl)-N-methylindolizine-2-carboxamide (26 mg, 32% yield). LCMS: m/z found 383.4 [M+H]⁺, RT = 1.74 min, (Method E); ¹H NMR (400 MHz, DMSO-d₆): δ 12.95 (s, 1H), 8.23-8.19 (m, 2H), 7.99 (br s, 1H), 7.90 (s, 1H), 7.41 (d, 1H), 6.73 (t, 1H), 6.62-6.69 (m, 2H), 6.64 (br s, 1H), 2.89 (br s, 3H), 1.52 (br s, 3H).

### N-(6,7-difluoro-4-oxo-3,4-dihydrophthalazin-1-yl)ethyl)-4,6-difluoro-N-methyl-1H-indole-2-carboxamide (Compound 60)

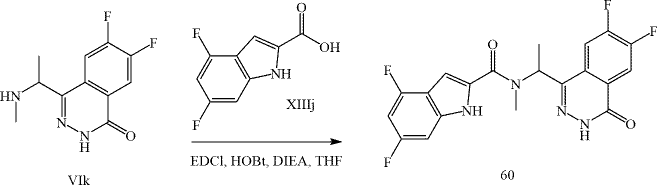

Racemic N-(1-(6,7-difluoro-4-oxo-3,4-dihydrophthalazin-1-yl)ethyl)-4,6-difluoro-N-methyl-1H-indole-2-carboxamide was synthesized in an analogous manner as described above from racemic 6,7-difluoro-4-(1-(methylamino)ethyl)phthalazin-1(2H)-one (VIk) and 4,6-difluoro-1H-indole-2-carboxylic acid (XIIIj). LCMS: m/z found 419.3 [M+H]⁺, RT = 1.98 min, (Method E); ¹H NMR (400 MHz, DMSO-d₆): δ 12.99 (s, 1H), 12.14 (s, 1H), 8.21 (t, 1H), 8.01 (br s, 1H), 7.06 (d, 1H), 6.99 (s, 1H), 6.89 (t, 1H), 6.37 (q, 1H), 3.04 (s, 3H), 1.54 (d, 3H).

### N-(6,7-difluoro-4-oxo-3,4-dihydrophthalazin-1-yl)ethyl)-3-fluoro-N-methyl-4-(trifluoromethyl)benzamide (Compound 62)

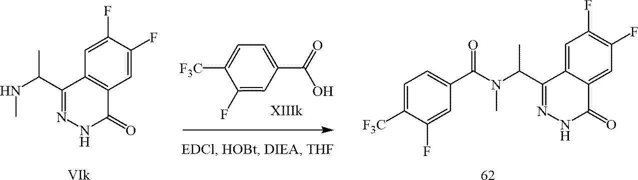

Racemic N-(1-(6,7-difluoro-4-oxo-3,4-dihydrophthalazin-1-yl)ethyl)-3-fluoro-N-methyl-4-(trifluoromethyl)benzamide was synthesized in an analogous manner as described above from racemic 6,7-difluoro-4-(1-(methylamino)ethyl)phthalazin-1(2H)-one (VIk) and 3-fluoro-4-(trifluoromethyl)benzoic acid (XIIIk). LCMS: m/z found 430.4 [M+H]⁺, RT = 2.12 min, (Method E); ¹H NMR (400 MHz, DMSO-d₆): δ 12.96 (s, 1H), 8.24 (t, 1H), 7.99-7.95 (m, 1H), 7.85 (t, 1H), 7.60 (d, 1H), 7.37 (d, 1H), 6.29 (q, 1H), 2.58 (s, 3H), 1.55 (d, 3H).

### 4-bromo-N-(1-(6,7-difluoro-4-oxo-3,4-dihydrophthalazin-1-yl)ethyl)-3-fluoro-N-methylbenzamide (Compound 63)

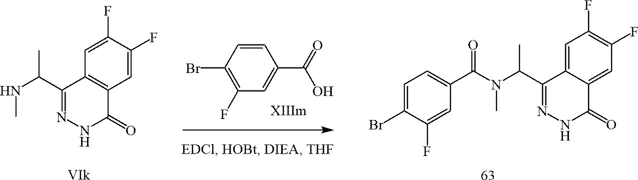

Racemic 4-bromo-N-(1-(6,7-difluoro-4-oxo-3,4-dihydrophthalazin-1-yl)ethyl)-3-fluoro-N-methylbenzamide was synthesized in an analogous manner as described above from racemic 6,7-difluoro-4-(1-(methylamino)ethyl)phthalazin-1(2H)-one (VIk) and 4-bromo-3-fluorobenzoic acid (XIIIm). LCMS: m/z found 440.2/442.2 [M+H]⁺, RT = 2.08 min, (Method E); ¹H NMR (400 MHz, DMSO-d₆): δ 12.95 (s, 1H), 8.23 (t, 1H), 7.97-7.93 (m, 1 H), 7.77 (t, 1H), 7.44 (d, 1H), 7.15-7.13 (dd, 1H), 6.27 (q, 1H), 2.58 (s, 3H), 1.53 (d, 3H).

### 4-Chloro-N-(1-(6,7-difluoro-4-oxo-3,4-dihydrophthalazin-1-yl)ethyl)-3-fluoro-N-methylbenzamide (Compound 64)

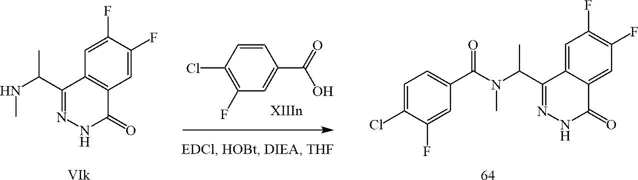

Racemic 4-chloro-N-(1-(6,7-difluoro-4-oxo-3,4-dihydrophthalazin-1-yl)ethyl)-3-fluoro-N-methylbenzamide was synthesized in an analogous manner as described above from racemic 6,7-difluoro-4-(1-(methylamino)ethyl)phthalazin-1(2H)-one (VIk) and 4-chloro-3-fluorobenzoic acid (XIIIn). LCMS: m/z found 396.3 [M+H]⁺, RT = 2.04 min, (Method E); ¹H NMR (400 MHz, DMSO-d₆): δ 12.95 (s, 1 H), 8.23 (t, 1 H), 7.97-7.93 (m, 1 H), 7.66 (t, 1 H), 7.49 (d, 1H), 7.21 (d, 1H), 6.27 (q, 1H), 2.58 (s, 3H), 1.53 (d, 3H).

### 4-bromo-N-(1-(6,7-difluoro-4-oxo-3,4-dihydrophthalazin-1-yl)ethyl)-N-methylbenzamide (Compound 65)

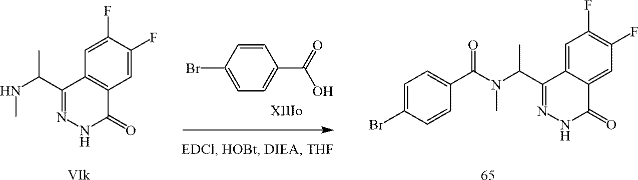

Racemic 4-bromo-N-(1-(6,7-difluoro-4-oxo-3,4-dihydrophthalazin-1-yl)ethyl)-N-methylbenzamide was synthesized in an analogous manner as described above from racemic 6,7-difluoro-4-(1-(methylamino)ethyl)phthalazin-1(2H)-one (VIk) and 4-bromobenzoic acid (XIIIo). LCMS: m/z found 422.3/424.3 [M+H]⁺, RT = 2.04 min, (Method E); ¹H NMR (400 MHz, DMSO-d₆): δ 12.94 (s, 1H), 8.23 (t, 1H), 7.98-7.93 (m, 1H), 7.64 (d, 2H), 7.31 (d, 2H), 6.28 (q, 1H), 2.56 (s, 3H), 1.53 (d, 3H).

### N-(6,7-difluoro-4-oxo-3,4-dihydrophthalazin-1-yl)ethyl)-N-methyl-4-(trifluoromethyl)benzamide (Compound 66)

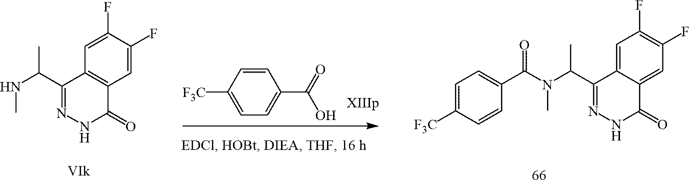

Racemic N-(1-(6,7-difluoro-4-oxo-3,4-dihydrophthalazin-1-yl)ethyl)-N-methyl-4-(trifluoromethyl)benzamide was synthesized in an analogous manner as described above from racemic 6,7-difluoro-4-(1-(methylamino)ethyl)phthalazin-1(2H)-one (VIk) and 4-(trifluoromethyl)benzoic acid (XIIIp). LCMS: m/z found 412.37 [M+H]⁺, RT = 2.07 min, (Method E); ¹H NMR (400 MHz, DMSO-d₆): δ 12.96 (s, 1H), 8.26 (t, 1H), 8.00-7.96 (d, 1H), 7.81-7.79 (d, 2H), 7.58-7.56 (d, 2H), 6.32 (q, 1H), 2.55 (s, 3H), 1.55 (d, 3H).

### N-(6,7-difluoro-4-oxo-3,4-dihydrophthalazin-1-yl)ethyl)-3,4,5-trifluoro-N-methylbenzamide (Compound 67)

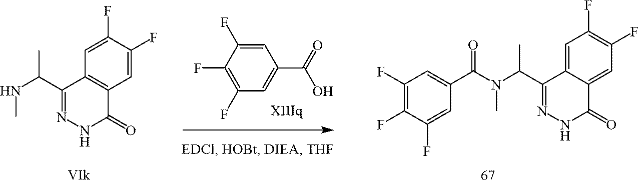

Racemic N-(1-(6,7-difluoro-4-oxo-3,4-dihydrophthalazin-1-yl)ethyl)-3,4,5-trifluoro-N-methylbenzamide was synthesized in an analogous manner as described above from racemic 6,7-difluoro-4-(1-(methylamino)ethyl)phthalazin-1(2H)-one (VIk) and 3,4,5-trifluorobenzoic acid (XIIIq). LCMS: m/z found 398.4 [M+H]⁺, RT = 2.03 min, (Method E); ¹H NMR (400 MHz, DMSO-d₆): δ 12.94 (s, 1H), 8.23 (t, 1H), 7.96-7.91 (m, 1H), 7.42 (t, 2H), 6.25 (q, 1H), 2.60 (s, 3H), 1.53 (d, 3H).

### 3-chloro-N-(1-(6,7-difluoro-4-oxo-3,4-dihydrophthalazin-1-yl)ethyl)-4-fluoro-N-methylbenzamide (Compound 68)

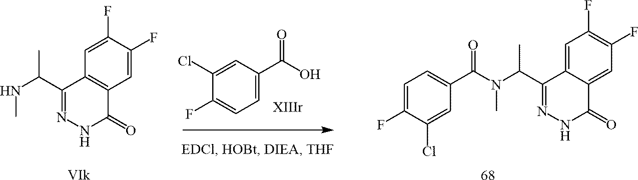

Racemic 3-chloro-N-(1-(6,7-difluoro-4-oxo-3,4-dihydrophthalazin-1-yl)ethyl)-4-fluoro-N-methylbenzamide was synthesized in an analogous manner as described above from racemic 6,7-difluoro-4-(1-(methylamino)ethyl)phthalazin-1(2H)-one (VIk) and 3-chloro-4-fluorobenzoic acid (XIIIr). LCMS: m/z found 396.3 [M+H]⁺, RT = 2.04 min, (Method E); ¹H NMR (400 MHz, DMSO-d₆): δ 12.94 (s, 1H), 8.23 (t, 1H), 7.99-7.94 (m, 1H), 7.66 (d, 1H), 7.47 (t, 1H), 7.40-7.37 (m, 1H), 6.28 (q, 1H), 2.59 (s, 3H), 1.53 (d, 3H).

### 4-chloro-N-(1-(6,7-difluoro-4-oxo-3,4-dihydrophthalazin-1-yl)ethyl)-N-methylbenzamide (Compound 69)

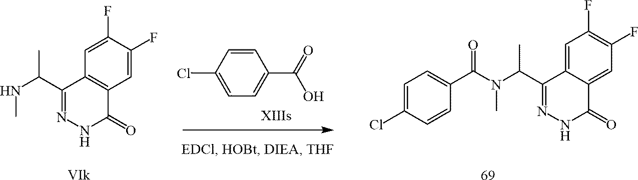

Racemic 4-chloro-N-(1-(6,7-difluoro-4-oxo-3,4-dihydrophthalazin-1-yl)ethyl)-N-methylbenzamide was synthesized in an analogous manner as described above from racemic 6,7-difluoro-4-(1-(methylamino)ethyl)phthalazin-1(2H)-one (VIk) and 4-chlorobenzoic acid (XIIIs). LCMS: m/z found 378.2 [M+H]⁺, RT = 1.83 min, (Method F); ¹H NMR (400 MHz, DMSO-d₆): δ 12.94 (s, 1H), 8.23 (t, 1H), 7.95 (t, 1H), 7.50 (d, 2H), 7.38 (d, 2H), 6.28 (q, 1H), 2.57 (s, 3H), 1.53 (d, 3H).

### N-(6,7-difluoro-4-oxo-3,4-dihydrophthalazin-1-yl)ethyl)-3,4-difluoro-N-methylbenzamide (Compound 70)

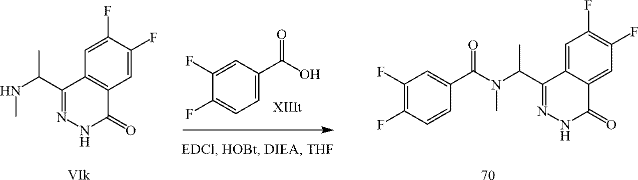

Racemic N-(1-(6,7-difluoro-4-oxo-3,4-dihydrophthalazin-1-yl)ethyl)-3,4-difluoro-N-methylbenzamide was synthesized in an analogous manner as described above from racemic 6,7-difluoro-4-(1-(methylamino)ethyl)phthalazin-1(2H)-one (VIk) and 3,4-difluorobenzoic acid (XIIIt). LCMS: m/z found 380.3 [M+H]⁺, RT = 1.79 min, (Method F); ¹H NMR (400 MHz, DMSO-d₆): δ 12.94 (s, 1H), 8.22 (t, 1H), 7.97-7.29 (m, 1H), 7.55-7.46 (m, 2H), 7.23-7.22 (m, 1H), 6.26 (m, 1H), 2.59 (s, 3H), 1.53 (d, 3H).

### N-(6,7-difluoro-4-oxo-3,4-dihydrophthalazin-1-yl)ethyl)-3-(difluoromethyl)-N-methylbenzamide (Compound 71)

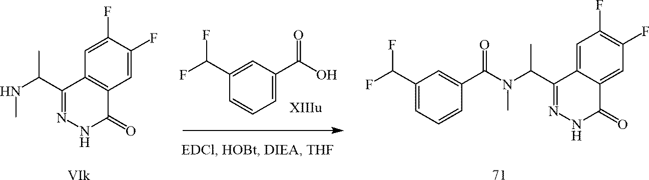

Racemic N-(1-(6,7-difluoro-4-oxo-3,4-dihydrophthalazin-1-yl)ethyl)-3-(difluoromethyl)-N-methylbenzamide was synthesized in an analogous manner as described above from racemic 6,7-difluoro-4-(1-(methylamino)ethyl)phthalazin-1(2H)-one (VIk) and 3-(difluoromethyl)benzoic acid (XIIIu). LCMS: m/z found 394.4 [M+H]⁺, RT = 1.88 min, (Method E); ¹H NMR (400 MHz, DMSO-d₆): δ 12.95 (s, 1H), 8.23 (t, 1H), 7.99 (t, 1H), 7.67-7.53 (m, 4H), 7.06 (t, 1H), 6.33-6.30 (m, 1H), 2.58 (s, 3H), 1.53 (d, 3H).

### N-(6,7-difluoro-4-oxo-3,4-dihydrophthalazin-1-yl)ethyl)-3-(difluoromethyl)-4-fluoro-N-methylbenzamide (Compound 72)

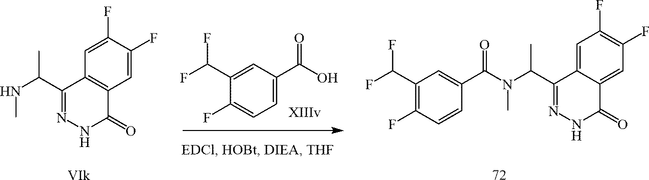

Racemic N-(1-(6,7-difluoro-4-oxo-3,4-dihydrophthalazin-1-yl)ethyl)-3-(difluoromethyl)-4-fluoro-N-methylbenzamidewas synthesized in an analogous manner as described above from racemic 6,7-difluoro-4-(1-(methylamino)ethyl)phthalazin-1(2H)-one (VIk) and 3-(difluoromethyl)-4-fluorobenzoic acid (XIIIv). LCMS: m/z found 412.3 [M+H]⁺, RT = 1.80 min, (Method F); ¹H NMR (400 MHz, DMSO-d₆): δ 12.95 (s, 1H), 8.22 (t, 1H), 7.97 (t, 1H), 7.66-7.62 (m, 2H), 7.44 (t, 1H), 7.21 (t, 1H), 6.30-6.28 (m, 1H), 2.60 (s, 3H), 1.55 (d, 3H).

### Phenyl (difluoromethyl)-4-fluorophenyl)carbamate (XIIh)

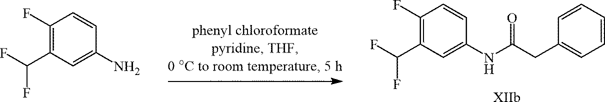

To a 0° C. stirred solution of 3-(difluoromethyl)-4-fluoroaniline (1.00 g, 6.21 mmol) in THF (10 mL) was added pyridine (1.4 mL, 18.63 mmol) and phenyl chloroformate (1.00 mL, 6.83 mmol). The reaction was stirred at room temperature for 5 h. The mixture was then diluted with EtOAc (50 mL) and washed with water (10 mL), brine solution (10 mL), dried over sodium sulfate and evaporated to dryness. The residue was purified by flash chromatography (100-20 mesh silica gel, eluting with a linear gradient of 0-20% EtOAc/pet-ether) to provide phenyl (3-(difluoromethyl)-4-fluorophenyl)carbamate (XIIh, 800 mg, 46%). LCMS: m/z found 282.3 [M+H]⁺, RT = 1.99 min, (Method E); ¹H NMR (400 MHz, CDCl₃): δ 7.65-7.60 (m, 2H), 7.42-7.38 (m, 2H), 7.27-7.24 (m, 1H), 7.20-7.18 (m, 2H), 7.15 (d, 1H), 7.10-6.73 (m, 2H).

### 1-(6,7-difluoro-4-oxo-3,4-dihydrophthalazin-1-yl)ethyl)-3-(3-(difluoromethyl)-4-fluorophenyl)-1-methylurea (Compound 73)

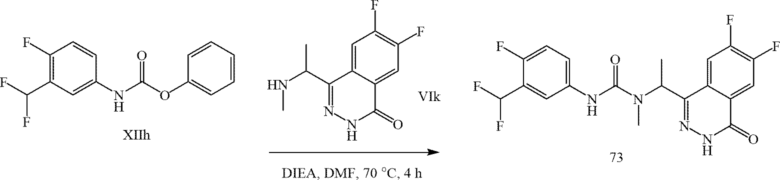

To a stirred solution of racemic 6,7-difluoro-4-(1-(methylamino)ethyl)phthalazin-1(2H)-one (VIk, 50 mg, 0.21 mmol) in DMF (0.5 mL) was added 0.07 ml (0.42 mmol, 2.0 eq.) of DIEA (70 µL, 0.42 mmol) and phenyl (3-(difluoromethyl)-4-fluorophenyl)carbamate (XIIh, 54 mg, 0.23 mmol). The mixture was heated to 70° C. for 4 h. The mixture was then diluted with water (20 mL) and stirred for 5 min at 0° C. The precipitated solids werecollected by filtration and purified by trituration using diethyl ether to obtain racemic 1-(1-(6,7-difluoro-4-oxo-3,4-dihydrophthalazin-1-yl)ethyl)-3-(3-(difluoromethyl)-4-fluorophenyl)-1-methylurea (32 mg, 42% yield) as an off-white solid. LCMS: m/z found 427.2 [M+H]⁺, RT = 1.87 min, (Method F); ¹H NMR (400 MHz, DMSO-d₆): δ 12.91 (s, 1H), 8.60 (s, 1H), 8.20 (t, 1H), 8.10-8.05 (m, 1H), 7.84-7.83 (m, 1H), 7.73-7.70 (m, 1H), 7.34-7.07 (m, 2H), 6.06 (m, 1H), 2.69 (s, 3H), 1.43 (d, 3H).

### N-(6,7-difluoro-4-oxo-3,4-dihydrophthalazin-1-yl)ethyl)-N-methylbenzamide (Compound 74)

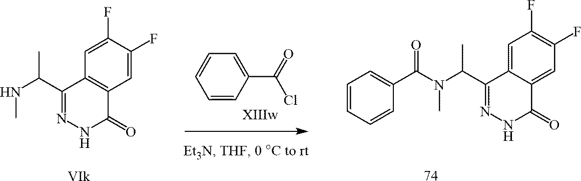

To a stirred solution of racemic 6,7-difluoro-4-(1-(methylamino)ethyl) isoquinolin-1(2H)-one (VIk, 50 mg, 0.21 mmol) in THF (2 mL) at 0° C. was added triethylamine (33 µL, 0.42 mmol), followed by benzoyl chloride (XIIIw, 35 mg, 0.25 mmol). The mixture was allowed to warm to room temperature and stirred for 2 h. The organic volatiles were then removed under reduced pressure and the obtained residue was stirred with saturated sodium bicarbonate solution (10 mL). The precipitated solid was collected by filtration and dried under vacuum. The crude compound was triturated with acetone (5 mL) to afford racemic N-(1-(6,7-difluoro-4-oxo-3,4-dihydrophthalazin-1-yl)ethyl)-N-methylbenzamide (74, 24 mg, 33% yield). LCMS: m/z found 344.3 [M+H]⁺, RT = 1.68 min, (Method F); ¹H NMR (400 MHz, DMSO-d₆): δ 12.94 (s, 1H), 8.23 (t, 1H), 7.99-7.91 (m, 1H), 7.44-7.34 (m, 5H), 6.30 (q, 1H), 2.56 (s, 3H), 1.54 (d, 3H).

### 8-chloro-N-(1-(6,7-difluoro-4-oxo-3,4-dihydrophthalazin-1-yl)ethyl)-N-methylindolizine-2-carboxamide (Compound 75)

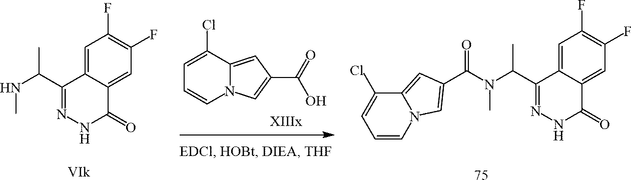

Racemic 8-chloro-N-(1-(6,7-difluoro-4-oxo-3,4-dihydrophthalazin-1-yl)ethyl)-N-methylindolizine-2-carboxamide was synthesized in an analogous manner as described above from racemic 6,7-difluoro-4-(1-(methylamino)ethyl)phthalazin-1(2H)-one (VIk) and 8-chloroindolizine-2-carboxylic acid (XIIIx). LCMS: m/z found 417.3/419.3 [M+H]⁺, RT = 3.71 min, (Method F); ¹H NMR (400 MHz, DMSO-d₆): δ 12.90 (s, 1H), 8.27-8.19 (m, 2H), 8.05 (s, 1H), 7.99 (m, 1H), 6.96 (d, 1H), 6.71 (m, 1H), 6.64 (t, 1H), 6.35 (m, 1H), 2.89 (br s, 3H), 1.55 (br s, 3H).

### Phenyl cyano-4-fluorophenyl)carbamate (XIIi)

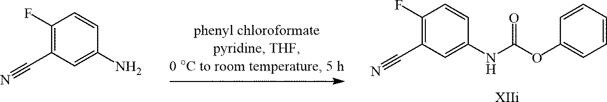

To a 0° C. stirred solution of 5-amino-2-fluorobenzonitrile (1.00 g, 7.35 mmol) in THF (10 mL) was added pyridine (2.4 mL, 29.40 mmol) and phenyl chloroformate (1.27 g, 8.08 mmol) and the mixture was stirred at room temperature for 16 h. The mixture was then diluted with EtOAc (50 mL) and washed with water (10 mL), brine solution (10 mL), dried over sodium sulfate and evaporated to dryness. The residue was purified by flash chromatography (100-20 mesh silica gel, eluting with a linear gradient of 0-20% EtOAc/pet-ether) to provide phenyl (3-cyano-4-fluorophenyl)carbamate (XIIi, 850 mg, 45% yield). LCMS: m/z found 257.2 [M+H]⁺, RT = 1.88 min, (Method D).

### 3-cyano-4-fluorophenyl)-1-(1-(6,7-difluoro-4-oxo-3,4-dihydrophthalazin-1-yl)ethyl)-1-methylurea (Compound 76)

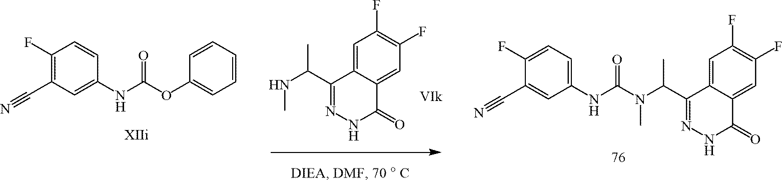

To a stirred solution of racemic 6,7-difluoro-4-(1-(methylamino)ethyl)phthalazin-1(2H)-one (VIk, 50 mg, 0.21 mmol) in DMF (0.5 mL) was added DIEA (70 µL, 0.42 mmol) and phenyl (3-cyano-4-fluorophenyl)carbamate (XIIi, 54 mg, 0.23 mmol) and the mixture was heated at 70° C. for 4 h. The mixture was cooled, diluted with water (20 mL) and stirred for 5 min at 0° C. The precipitated solids were collected by filtration and purified by trituration using diethyl ether to provide racemic 3-(3-cyano-4-fluorophenyl)-1-(1-(6,7-difluoro-4-oxo-3,4-dihydrophthalazin-1-yl)ethyl)-1-methylurea (32 mg, 42% yield). LCMS: m/z found 402.4 [M+H]⁺, RT = 1.77 min, (Method E); ¹H NMR (400 MHz, DMSO-d₆): δ 12.92 (s, 1H), 8.72 (s, 1H), 8.20 (t, 1H), 8.07-8.02 (m, 2H), 7.90-7.86 (m, 1H), 7.47 (t, 1H), 6.05 (q, 1H), 2.70 (s, 3H), 1.44 (d, 3H).

### N-(6,7-difluoro-4-oxo-3,4-dihydrophthalazin-1-yl)ethyl)-8-fluoro-N-methylindolizine-2-carboxamide (Compound 77)

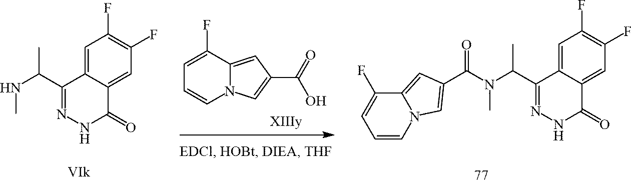

Racemic N-(1-(6,7-difluoro-4-oxo-3,4-dihydrophthalazin-1-yl)ethyl)-8-fluoro-N-methylindolizine-2-carboxamide was synthesized in an analogous manner as described above from racemic 6,7-difluoro-4-(1-(methylamino)ethyl)phthalazin-1(2H)-one (VIk) and 8-fluoroindolizine-2-carboxylic acid (XIIIy). LCMS: m/z found 401.3 [M+H]⁺, RT = 6.06 min, (Method A); ¹H NMR (400 MHz, DMSO-d₆): δ 12.95 (s, 1H), 8.21 (t, 1H), 8.12 (bs, 1H), 8.07 (s, 1H), 8.1-7.9 (br s, 1H), 6.75 (s, 1H), 6.62 (d, 2H), 6.34 (br s, 1H), 2.89 (br s, 3H), 1.52 (br s, 3H).

### (2R)-N-(6,7-difluoro-4-oxo-3,4-dihydrophthalazin-1-yl)ethyl)-N-methylindoline-2-carboxamide (Compound 78)

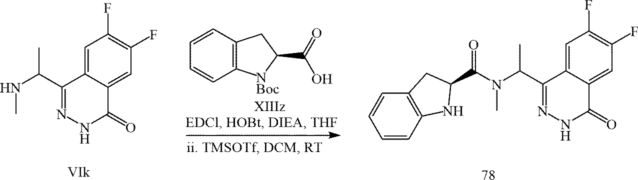

Step i: tert-Butyl (2S)-2-((1-(6,7-difluoro-4-oxo-3,4-dihydrophthalazin-1-yl)ethyl)(methyl)carbamoyl)indoline-1-carboxylate was synthesized as a mixture of diastereomers in an analogous manner as described above from racemic 6,7-difluoro-4-(1-(methylamino)ethyl)phthalazin-1(2H)-one (VIk) and (S)-1-(tert-butoxycarbonyl)indoline-2-carboxylic acid (XIIIz).

Step ii: To a stirred solution of tert-butyl (2S)-2-((1-(6,7-difluoro-4-oxo-3,4-dihydrophthalazin-1-yl)ethyl)(methyl)carbamoyl)indoline-1-carboxylate (55 mg, 0.113 mmol) in DCM (5 mL) was added trimethylsilyl trifluoromethanesulfonate (40 µL, 0.22 mmol) at 0° C. The reaction mixture was allowed to warm to room temperature and stirred for 2 h. The volatiles were removed under reduced pressure. The resulting residue was diluted with a saturated NaHCO₃ solution (10 mL). The resulting precipitate was collected by filtration and washed with water (10 mL) followed by n-pentane (10 mL). The residue was purified by flash chromatography (silica gel, 14-30% MeOH in DCM gradient) to afford (2R)-N-(1-(6,7-Difluoro-4-oxo-3,4-dihydrophthalazin-1-yl)ethyl)-N-methylindoline-2-carboxamide (13 mg, 29% yield) as a mixture of diastereomers. LCMS: m/z found 385.3 [M+H]⁺, RT = 5.24 min, (Method A); ¹H NMR (400 MHz, DMSO-d₆): δ 12.93 (s, 1H), 8.20 (t, 1H), 7.58 (dd, 1H), 6.95-6.87 (m, 2H), 6.59-6.52 (m, 2H), 6.15-6.10 (m, 1H) 5.77 (s, 1H), 4.67-4.63 (m, 1H), 3.31-3.20 (m, 1H), 2.80-2.70 (m, 4H), 1.41 (d, 3H).

### (R)chloro-N-(1-(6,7-difluoro-4-oxo-3,4-dihydrophthalazin-1-yl)ethyl)-N-methyl-4H-thieno[3,2-b]pyrrole-5-carboxamide (Compound 79)

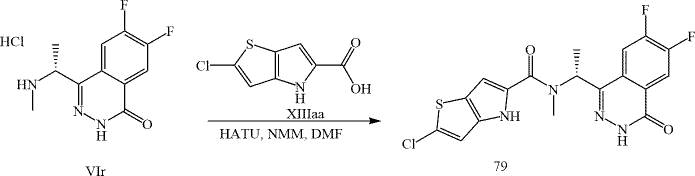

(R)Chloro-N-(1-(6,7-difluoro-4-oxo-3,4-dihydrophthalazin-1-yl)ethyl)-N-methyl-4H-thieno[3,2-b]pyrrole-5-carboxamide was synthesized in an analogous manner as described above from (R)-6,7-difluoro-4-(1-(methylamino)ethyl)phthalazin-1(2H)-one hydrochloride (VIr) and 2-chloro-4H-thieno[3,2-b]pyrrole-5-carboxylic acid (XIIIaa). LCMS: m/z found 423.0 [M+H]⁺, RT = 4.63 min, (Method A); ¹H NMR (400 MHz, DMSO-d₆): δ 12.98 (s, 1H), 12.04 - 11.99 (m, 1H), 8.21 (dd, 1H), 8.01 (s, 1H), 7.08 (s, 1H), 6.91 (d, 1H), 6.34 (q, 1H), 2.97 (s, 3H), 1.51 (d, 3H).

### N-(6,7-difluoro-4-oxo-3,4-dihydrophthalazin-1-yl)ethyl)-N-methyl-4H-thieno [3,2-b]pyrrole-5-carboxamide (Compounds 80 and 81)

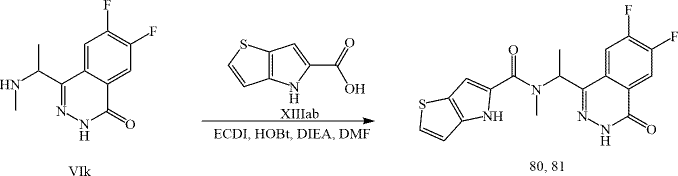

Racemic N-(1-(6,7-difluoro-4-oxo-3,4-dihydrophthalazin-1-yl)ethyl)-N-methyl-4H-thieno[3,2-b]pyrrole-5-carboxamide was synthesized in an analogous manner as described above from racemic 6,7-difluoro-4-(1-(methylamino)ethyl)phthalazin-1(2H)-one (VIk) and 4H-thieno[3,2-b]pyrrole-5-carboxylic acid (XIIIab). The enantiomers were subsequently separated by preparative SFC: Method isocratic, Mobile phase 1:1 Methanol/Acetonitrile: CO₂ - 60:40. Column: Chiralpak IA (30 × 250 mm), 5 µm, flow rate: 110 g/min.

Enantiomer I (Compound 80): LCMS: m/z found 389.0 [M+H]⁺, RT = 3.85 min, (Method A); ¹H NMR (400 MHz, DMSO-d₆): δ 13.1 (bs, 1H), 11.85 (bs, 1 H), 8.2 (t, 1H), 8.0 (bs, 1H), 7.43 (d, 1H), 6.98 (d, 1H), 6.94 (s, 1H), 6.36 (q, 1H), 2.98 (s, 3H), 1.52 (d, 3H); Chiral analytical SFC: RT = 2.88 min, Column: Chiralpak IA (4.6 × 150 mm) 3 µm, 50% (0.2% 7 M Methanolic Ammonia in Acetonitrile:Methanol[1:1]), Flow rate: 3.0 g/min.

Enantiomer II (Compound 81): LCMS: m/z found 389.0 [M+H]⁺, RT = 3.85 min, (Method A); ¹H NMR (400 MHz, DMSO-d₆): δ 12.95 (bs, 1H), 11.94 (bs, 1 H), 8.2 (t, 1H), 8.0 (bs, 1H), 7.42 (d, 1H), 6.98 (d, 1H), 6.93 (s, 1H), 6.36 (m, 1H), 2.98 (bs, 3H), 1.52 (d, 3H); Chiral analytical SFC: RT = 4.38 min, Column: Chiralpak IA (4.6 × 150 mm) 3 µm, 50% (0.2% 7 M Methanolic Ammonia in Acetonitrile:Methanol)[1:1]), Flow rate: 3.0 g/min.

### N-(6,7-difluoro-4-oxo-3,4-dihydrophthalazin-1-yl)ethyl)-N-methyl-2,3-dihydro-1H-indene-5-carboxamide (Compounds 82 and 83)

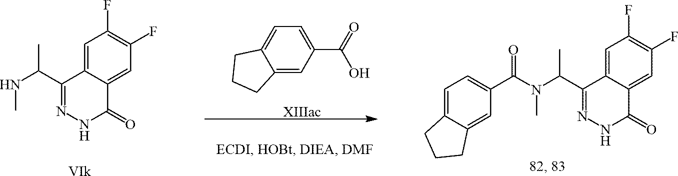

Racemic N-(1-(6,7-difluoro-4-oxo-3,4-dihydrophthalazin-1-yl)ethyl)-N-methyl-2,3-dihydro-1H-indene-5-carboxamide was synthesized in an analogous manner as described above from racemic 6,7-difluoro-4-(1-(methylamino)ethyl)phthalazin-1(2H)-one (VIk) and 2,3-dihydro-1H-indene-5-carboxylic acid (XIIIac). The enantiomers were subsequently separated by preparative SFC: Method isocratic, Mobile phase Methanol: CO₂ - 40:60. Column: Chiralpak IC (30 × 250 mm), 5 µm, flow rate: 110 g/min.

Enantiomer I (Compound 82): LCMS: m/z found 384.1 [M+H]⁺, RT = 4.38 min, (Method A); ¹H NMR (400 MHz, DMSO-d₆): δ 12.93 (bs, 1H), 8.2 (t, 1H), 7.98 (t, 1H), 7.26 (d, 1H), 7.19 (s, 1H), 7.07 (d, 1H), 6.29 (m, 1H), 2.86 (t, 4H), 2.57 (bs, 3H), 2.05-1.97 (m, 2H), 1.53 (d, 3H); Chiral analytical SFC: RT = 4.83 min, Column: Chiralpak IC (4.6 × 150 mm) 3 µm, 30 % Methanol, Flow rate: 3.0 g/min.

Enantiomer II (Compound 83): LCMS: m/z found 384.1 [M+H]⁺, RT = 4.38 min, (Method A); ¹H NMR (400 MHz, DMSO-d₆): δ 12.93 (bs, 1 H), 8.2 (t, 1H), 7.98 (t, 1H), 7.26 (d, 1H), 7.19 (s, 1H), 7.07 (d, 1H), 6.29 (m, 1H), 2.86 (t, 4H), 2.57 (bs, 3H), 2.05-1.97 (m, 2H), 1.53 (d, 3H); Chiral analytical SFC: RT = 7.37 min, Column: Chiralpak IC (4.6 x 150 mm) 3 µm, 30% Methanol, Flow rate: 3.0 g/min.

### N-(6,7-difluoro-4-oxo-3,4-dihydrophthalazin-1-yl)ethyl)-N-methylbenzo[d]thiazole-5-carboxamide (Compounds 84 and 85)

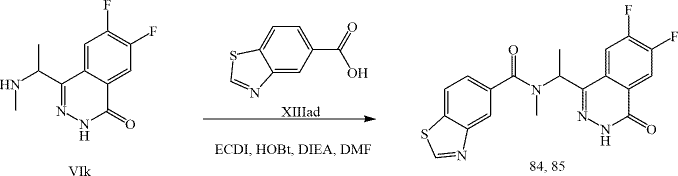

Racemic N-(1-(6,7-difluoro-4-oxo-3,4-dihydrophthalazin-1-yl)ethyl)-N-methylbenzo[d]thiazole-5-carboxamide was synthesized in an analogous manner as described above from racemic 6,7-difluoro-4-(1-(methylamino)ethyl)phthalazin-1(2H)-one (VIk) and benzo[d]thiazole-5-carboxylic acid (XIIIad). The enantiomers were subsequently separated by preparative SFC: Method isocratic, Mobile phase Methanol: CO₂ - 40:60. Column: Chiralpak IC (30 × 250 mm), 5 µm, flow rate: 110 g/min.

Enantiomer I (Compound 84): LCMS: m/z found 401.1 [M+H]⁺, RT = 3.13 min, (Method A); ¹H NMR (400 MHz, DMSO-d₆): δ 12.91 (bs, 1H), 9.48 (s, 1H), 8.24 (m, 2H), 8.06-8.01 (m, 2H), 7.45 (d, 1H), 6.35 (d, 1H), 2.62 (bs, 3H), 1.58 (d, 3H); Chiral analytical SFC: RT = 4.74 min, Column: Chiralpak IG-3 (4.6 × 150 mm) 3 µm, 50% (0.2% 7N Methanolic Ammonia in Acetonitrile:Methanol)(1:1), Flow rate: 3.0 g/min.

Enantiomer II (Compound 85): LCMS: m/z found 401.0 [M+H]⁺, RT = 3.13 min, (Method A); ¹H NMR (400 MHz, DMSO-d₆): δ 12.87 (bs, 1H), 9.48 (s, 1H), 8.24 (m, 2H), 8.06-8.01 (m, 2H), 7.45 (d, 1H), 6.35 (d, 1H), 2.62 (bs, 3H), 1.58 (d, 3H); Chiral analytical SFC: RT = 6.94 min, Column: Chiralpak IG-3 (4.6 × 150 mm) 3 µm, 50% (0.2% 7N Methanolic Ammonia in Acetonitrile:Methanol)(1:1), Flow rate: 3.0 g/min.

### N-(6,7-difluoro-4-oxo-3,4-dihydrophthalazin-1-yl)ethyl)-N-methylbenzo[d]thiazole-6-carboxamide (Compounds 86 and 87)

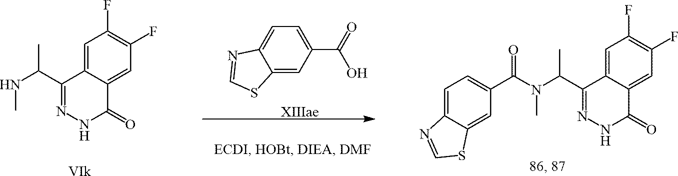

Racemic N-(1-(6,7-difluoro-4-oxo-3,4-dihydrophthalazin-1-yl)ethyl)-N-methylbenzo[d]thiazole-6-carboxamide was synthesized in an analogous manner as described above from racemic 6,7-difluoro-4-(1-(methylamino)ethyl)phthalazin-1(2H)-one (VIk) and benzo[d]thiazole-6-carboxylic acid (XIIIae). The enantiomers were subsequently separated by preparative SFC: Method isocratic, Mobile phase methanol: CO₂ - 40:60. Column: Chiralpak IA (30 × 250 mm), 5 µm, flow rate: 110 g/min.

Enantiomer I (Compound 86): LCMS: m/z found 401.0 [M+H]⁺, RT = 3.06 min, (Method A); ¹H NMR (400 MHz, DMSO-d₆): δ 12.96 (bs, 1H), 9.48 (s, 1H), 8.24 (s, 2H), 8.13 (d, 1H), 8.03 (t, 1H), 7.50 (d, 1H), 6.34 (d, 1H), 2.61 (bs, 3H), 1.57 (d, 3H); Chiral analytical SFC: RT = 1.99 min, Column: Chiralpak IA (4.6 × 150 mm) 3 µm, 40 % Methanol, Flow rate: 3.0 g/min.

Enantiomer II (Compound 87): LCMS: m/z found 401.1 [M+H]⁺, RT = 3.06 min, (Method A); ¹H NMR (400 MHz, DMSO-d₆): δ 12.96 (bs, 1H), 9.48 (s, 1H), 8.24 (s, 2H), 8.13 (d, 1H), 8.03 (t, 1H), 7.50 (d, 1H), 6.34 (d, 1H), 2.61 (bs, 3H), 1.57 (d, 3H); Chiral analytical SFC: RT = 3.08 min, Column: Chiralpak IA (4.6 × 150 mm) 3 µm, 40 % Methanol, Flow rate: 3.0 g/min.

### N-(6,7-difluoro-4-oxo-3,4-dihydrophthalazin-1-yl)ethyl)-N-methylbenzo[d]oxazole-5-carboxamide (Compounds 88 and 89)

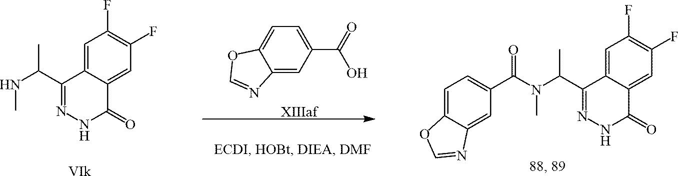

Racemic N-(1-(6,7-difluoro-4-oxo-3,4-dihydrophthalazin-1-yl)ethyl)-N-methylbenzo[d]oxazole-5-carboxamide was synthesized in an analogous manner as described above from racemic 6,7-difluoro-4-(1-(methylamino)ethyl)phthalazin-1(2H)-one (VIk) and benzo[d]oxazole-5-carboxylic acid (Xlllaf). The enantiomers were subsequently separated by preparative SFC: Method isocratic, Mobile phase Methanol: CO₂ - 40:60. Column: Chiralpak IC (30 × 250 mm), 5 µm, flow rate: 110 g/min.

Enantiomer I (Compound 88): LCMS: m/z found 385.0 [M+H]⁺, RT = 2.88 min, (Method A); ¹H NMR (400 MHz, DMSO-d₆): δ 12.94 (bs, 1 H), 8.83 (s, 1H), 8.26 (m, 1H), 8.02 (m, 1H), 7.85 (t, 2H), 7.43 (d, 1H), 6.33 (d, 1H), 2.60 (bs, 3H), 1.57 (d, 3H); Chiral analytical SFC: RT = 2.65 min, Column: Chiralpak IG-3 (4.6 × 150 mm) 3 µm, 50 % Methanol, Flow rate: 3.0 g/min.

Enantiomer II (Compound 89): LCMS: m/z found 385.0 [M+H]⁺, RT = 2.88 min, (Method A); ¹H NMR (400 MHz, DMSO-d₆): δ 12.95 (bs, 1H), 8.83 (s, 1H), 8.26 (m, 1H), 8.02 (m, 1H), 7.85 (t, 2H), 7.43 (d, 1H), 6.33 (d, 1H), 2.60 (bs, 3H), 1.57 (d, 3H); Chiral analytical SFC: RT = 3.99 min, Column: Chiralpak IG-3 (4.6 × 150 mm) 3 µm, 50% Methanol, Flow rate: 3.0 g/min.

### N-(6,7-difluoro-4-oxo-3,4-dihydrophthalazin-1-yl)ethyl)-N-methylbenzo[d]oxazole-6-carboxamide (Compounds 90 and 91)

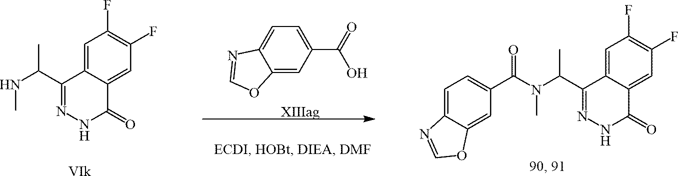

Racemic N-(1-(6,7-difluoro-4-oxo-3,4-dihydrophthalazin-1-yl)ethyl)-N-methylbenzo[d]oxazole-6-carboxamide was synthesized in an analogous manner as described above from racemic 6,7-difluoro-4-(1-(methylamino)ethyl)phthalazin-1(2H)-one (VIk) and benzo[d]oxazole-6-carboxylic acid (XIIIag). The enantiomers were subsequently separated by preparative SFC: Method isocratic, Mobile phase methanol: CO₂ - 40:60. Column: Chiralpak IG (30 × 250 mm), 5 µm, flow rate: 110 g/min.

Enantiomer I (Compound 90): LCMS: m/z found 385.1 [M+H]⁺, RT = 2.85 min, (Method A); ¹H NMR (400 MHz, DMSO-d₆): δ 12.95 (bs, 1H), 8.84 (s, 1H), 8.24 (t, 1H), 8.01 (t, 1H), 7.86-7.84 (m, 2H), 7.37 (d, 1H), 6.33 (m, 1H), 2.60 (s, 3H), 1.57 (d, 3H); Chiral analytical SFC: RT = 2.79 min, Column: Chiralpak IG-3 (4.6 × 150 mm) 3 µm, 40 % Methanol, Flow rate: 3.0 g/min.

Enantiomer II (Compound 91): LCMS: m/z found 385.0 [M+H]⁺, RT = 2.85 min, (Method A); ¹H NMR (400 MHz, DMSO-d₆): δ 12.95 (bs, 1H), 8.84 (s, 1H), 8.24 (t, 1H), 8.01 (t, 1H), 7.86-7.84 (m, 2H), 7.37 (d, 1H), 6.33 (m, 1H), 2.60 (s, 3H), 1.55 (d, 3H); Chiral analytical SFC: RT = 4.39 min, Column: Chiralpak IG-3 (4.6 × 150 mm) 3 µm, 40 % Methanol, Flow rate: 3.0 g/min.

### 5-chloro-N-(1-(6,7-difluoro-4-oxo-3,4-dihydrophthalazin-1-yl)ethyl)-N-methyl-6-(trifluoromethyl)nicotinamide (Compounds 92 and 93)

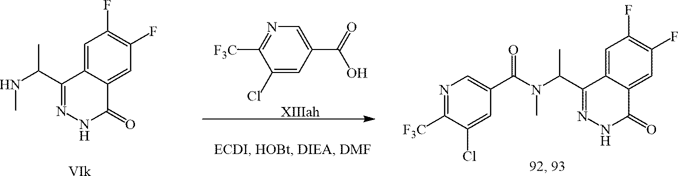

Racemic 5-chloro-N-(1-(6,7-difluoro-4-oxo-3,4-dihydrophthalazin-1-yl)ethyl)-N-methyl-6-(trifluoromethyl)nicotinamide was synthesized in an analogous manner as described above from racemic 6,7-difluoro-4-(1-(methylamino)ethyl)phthalazin-1(2H)-one (VIk) and 5-chloro-6-(trifluoromethyl)nicotinic acid (XIIIah). The enantiomers were subsequently separated by preparative SFC: Method isocratic, Mobile phase Methanol: CO₂ - 40:60. Column: Chiralpak IC (30 × 250 mm), 5 µm, flow rate: 110 g/min.

Enantiomer I (Compound 92): LCMS: m/z found 447.0 [M+H]⁺, RT = 4.45 min, (Method A); ¹H NMR (400 MHz, DMSO-d₆): δ 12.72 (bs, 1H), 8.70 (s, 1H), 8.38 (s, 1H), 8.24 (t, 1H), 8.0 (t, 1H), 6.32-6.27 (m, 1H), 2.65 (s, 3H), 1.57 (d, 3H); Chiral analytical SFC: RT = 1.06 min, Column: Chiralpak IA (4.6 × 150 mm) 3 µm, 40 % Methanol, Flow rate: 3.0 g/min.

Enantiomer II (Compound 93): LCMS: m/z found 447.0 [M+H]⁺, RT = 4.46 min, (Method A); ¹H NMR (400 MHz, DMSO-d₆): δ 12.97 (bs, 1H), 8.70 (s, 1H), 8.38 (s, 1H), 8.24 (t, 1H), 8.0 (t, 1H), 6.32-6.27 (m, 1H), 2.65 (bs, 3H), 1.57 (d, 3H); Chiral analytical SFC: RT = 2.09 min, Column: Chiralpak IA (4.6 × 150 mm) 3 µm, 30 % Methanol, Flow rate: 3.0 g/min.

### 4-chloro-N-(1-(6,7-difluoro-4-oxo-3,4-dihydrophthalazin-1-yl)ethyl)-3,5-difluoro-N-methylbenzamide (Compounds 94 and 95)

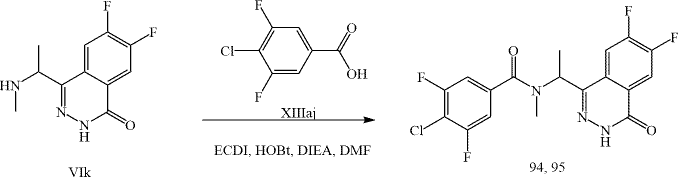

Racemic 4-chloro-N-(1-(6,7-difluoro-4-oxo-3,4-dihydrophthalazin-1-yl)ethyl)-3,5-difluoro-N-methylbenzamide was synthesized in an analogous manner as described above from racemic 6,7-difluoro-4-(1-(methylamino)ethyl)phthalazin-1(2H)-one (VIk) and 4-chloro-3,5-difluorobenzoic acid (XIIIaj). The enantiomers were subsequently separated by preparative SFC: Method isocratic, Mobile phase Methanol: CO₂ - 35:65. Column: Chiralpak IC-3 (30 × 250 mm), 5 µm, flow rate: 110 g/min.

Enantiomer I (Compound 94): LCMS: m/z found 414.0 [M+H]⁺, RT = 4.49 min, (Method A); ¹H NMR (400 MHz, DMSO-d₆): δ 12.95 (bs, 1H), 8.25 (t, 1H), 7.97 (m, 1H), 7.41 (d, 2H), 6.27 (m, 1H), 2.60 (bs, 3H), 1.57 (d, 3H); Chiral analytical SFC: RT = 1.61 min, Column: Chiralpak IC-3 (4.6 × 150 mm) 3 µm, 30 % Methanol, Flow rate: 3.0 g/min.

Enantiomer II (Compound 95): LCMS: m/z found 414.0 [M+H]⁺, RT = 4.48 min, (Method A); ¹H NMR (400 MHz, DMSO-d₆): δ 12.94 (bs, 1H), 8.25 (t, 1H), 7.97 (m, 1H), 7.41 (d, 2H), 6.27 (m, 1H), 2.60 (bs, 3H), 1.57 (d, 3H); Chiral analytical SFC: RT = 1.99 min, Column: Chiralpak IC-3 (4.6 × 150 mm) 3 µm, 30 % Methanol, Flow rate: 3.0 g/min.

### N-(6,7-difluoro-4-oxo-3,4-dihydrophthalazin-1-yl)ethyl)-4-(difluoromethyl)-6-fluoro-N-methyl-1H-indole-2-carboxamide (Compounds 96 and 97)

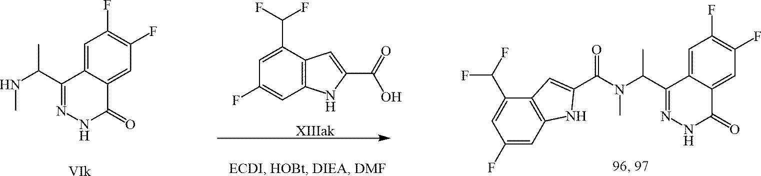

Racemic N-(1-(6,7-difluoro-4-oxo-3,4-dihydrophthalazin-1-yl)ethyl)-4-(difluoromethyl)-6-fluoro-N-methyl-1H-indole-2-carboxamide was synthesized in an analogous manner as described above from racemic 6,7-difluoro-4-(1-(methylamino)ethyl)phthalazin-1(2H)-one (VIk) and 4-(difluoromethyl)-6-fluoro-1H-indole-2-carboxylic acid (XIIIak). The enantiomers were subsequently separated by preparative SFC: Method isocratic, Mobile phase Methanol: CO₂ - 40:60. Column: Chiralpak IC (30 x 250 mm), 5 µm, flow rate: 110 g/min.

Enantiomer I (Compound 96): LCMS: m/z found 451.0 [M+H]⁺, RT = 4.46 min, (Method A); ¹H NMR (400 MHz, DMSO-d₆): δ 12.98 (bs, 1H), 12.16 (s, 1H), 8.22 (t, 1H), 8.02 (bs, 1H), 7.42-7.14 (m, 3H), 7.04 (bs, 1H), 6.37 (d, 1H), 3.04 (bs, 3H), 1.55 (d, 3H); Chiral analytical SFC: RT = 3.41 min, Column: Chiralpak IG-3 (4.6 × 150 mm) 3 µm, 30 % Methanol, Flow rate: 3.0 g/min.

Enantiomer II (Compound 97): LCMS: m/z found 451.0 [M+H]⁺, RT = 4.46 min, (Method A); ¹H NMR (400 MHz, DMSO-d₆): δ 12.98 (bs, 1 H), 12.16 (s, 1H), 8.22 (t, 1H), 8.02 (bs, 1H), 7.42-7.14 (m, 3H), 7.04 (bs, 1H), 6.37 (d, 1H), 3.04 (bs, 3H), 1.55 (d, 3H); Chiral analytical SFC: RT = 4.49 min, Column: Chiralpak IG-3 (4.6 × 150 mm) 3 µm, 30 % Methanol, Flow rate: 3.0 g/min.

### N-(6,7-difluoro-4-oxo-3,4-dihydrophthalazin-1-yl)ethyl)-4-(difluoromethyl)-N-methyl-1H-indole-2-carboxamide (Compounds 98 and 99)

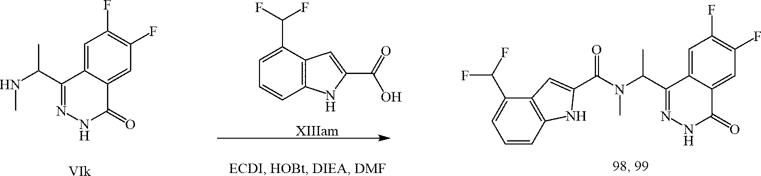

Racemic N-(1-(6,7-difluoro-4-oxo-3,4-dihydrophthalazin-1-yl)ethyl)-4-(difluoromethyl)-N-methyl-1H-indole-2-carboxamide was synthesized in an analogous manner as described above from racemic 6,7-difluoro-4-(1-(methylamino)ethyl)phthalazin-1(2H)-one (VIk) and 4-(difluoromethyl)-1H-indole-2-carboxylicacid (XIIIam). The enantiomers were subsequently separated by preparative SFC: Method isocratic, Mobile phase Methanol: CO₂ - 30:70. Column: Chiralpak IC-3 (30 × 250 mm), 5 µm, flow rate: 110 g/min.

Enantiomer I (Compound 98): LCMS: m/z found 433.1 [M+H]⁺, RT = 3.87 min, (Method A); ¹H NMR (400 MHz, DMSO-d₆): δ 12.95 (bs, 1H), 8.34 (bs, 1H), 8.24 (t, 1H), 8.04 (m, 2H), 7.17 (m, 2H), 6.77 (m, 2H), 6.36 (bs, 1H), 2.89 (bs, 3H), 1.53 (d, 3H); Chiral analytical SFC: RT = 3.90 min, Column: Chiralpak IC-3 (4.6 × 150 mm) 3 µm, 30 % Methanol, Flow rate: 3.0 g/min.

Enantiomer II (Compound 99): LCMS: m/z found 433.0 [M+H]⁺, RT = 4.46 min, (Method A); ¹H NMR (400 MHz, DMSO-d₆): δ 12.95 (bs, 1H), 8.34 (bs, 1H), 8.24 (t, 1H), 8.04 (m, 2H), 7.17 (m, 2H), 6.77 (m, 2H), 6.36 (bs, 1H), 2.89 (bs, 3H), 1.53 (d, 3H); Chiral analytical SFC: RT = 5.13 min, Column: Chiralpak IC-3 (4.6 × 150 mm) 3 µm, 30% Methanol, Flow rate: 3.0 g/min.

### 4-bromo-N-(1-(6,7-difluoro-4-oxo-3,4-dihydrophthalazin-1-yl)ethyl)-3,5-difluoro-N-methylbenzamide (Compounds 100 and 101)

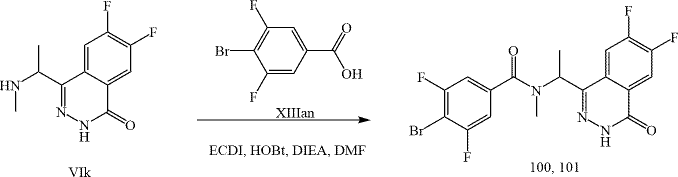

Racemic 4-bromo-N-(1-(6,7-difluoro-4-oxo-3,4-dihydrophthalazin-1-yl)ethyl)-3,5-difluoro-N-methylbenzamide was synthesized in an analogous manner as described above from racemic 6,7-difluoro-4-(1-(methylamino)ethyl)phthalazin-1(2H)-one (VIk) and 4-bromo-3,5-difluorobenzoic acid (XIIIan). The enantiomers were subsequently separated by preparative SFC: Method isocratic, Mobile phase Methanol: CO₂ - 35:65. Column: Chiralpak IG-3 (30 × 250 mm), 5 µm, flow rate: 110 g/min.

Enantiomer I (Compound 100): LCMS: m/z found 457.9/459.9 [M+H]⁺, RT = 4.52 min, (Method A); ¹H NMR (400 MHz, DMSO-d₆): δ 12.95 (bs, 1H), 8.25 (t, 1H), 7.97 (m, 1H), 7.35 (d, 2H), 6.36 (bs, 1H), 2.60 (bs, 3H), 1.54 (d, 3H); Chiral analytical SFC: RT = 3.33 min, Column: Chiralpak IG-3 (4.6 × 150 mm) 3 µm, 25 % Methanol, Flow rate: 3.0 g/min.

Enantiomer II (Compound 101): LCMS: m/z found 457.9/459.9 [M+H]⁺, RT = 4.52 min, (Method A); ¹H NMR (400 MHz, DMSO-d₆): δ 12.95 (bs, H), 8.25 (t, 1H), 7.97 (m, 1H), 7.35 (d, 2H), 6.36 (bs, 1H), 2.60 (bs, 3H), 1.54 (d, 3H); Chiral analytical SFC: RT = 4.65 min, Column: Chiralpak IG-3 (4.6 × 150 mm) 3 µm, 25 % Methanol, Flow rate: 3.0 g/min.

### 2-fluoro-4-((2-(trimethylsilyl)ethoxy)methyl)-4H-thieno[3,2-b]pyrrole-5-carboxylic Acid (XIIIao)

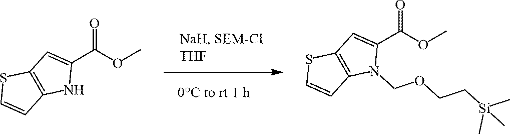

Step i: To a stirred 0° C. solution of methyl 4H-thieno[3,2-b]pyrrole-5-carboxylate (10 g, 52.2 mmol) in 100 mL of THF was added NaH (60% in mineral oil, 6.6 g, 166 mmol). After addition, the reaction was stirred at room temperature for 10 min followed by the addition of 2-(trimethylsilyl)ethoxymethyl chloride (14.6 mL, 82.3 mmol). The reaction mixture was allowed stir at RT for 1 h. The reaction mixture was quenched with water (100 mL). The aqueous layer was extracted with EtOAc (2 × 200 mL). The combined organics were washed with brine (200 mL), dried over anhydrous Na₂SO₄ and concentrated under reduced pressure. The obtained crude product was triturated with n-pentane (50 mL) at room temperature, filtered, dried under vacuum to afford methyl 4-((2-(trimethylsilyl)ethoxy)methyl)-4H-thieno[3,2-b]pyrrole-5-carboxylate (10 g, 75% yield) as an off-white solid. LCMS: m/z found 312.28 [M+H]⁺.

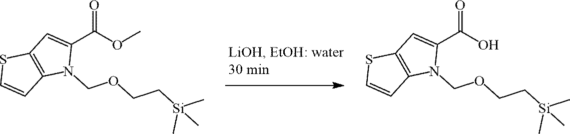

Step ii: To a stirred solution of methyl 4-((2-(trimethylsilyl)ethoxy)methyl)-4H-thieno[3,2-b]pyrrole-5-carboxylate (10 g, 32.1 mmol) in 100 mL of EtOH was added LiOH (3.8 g, 160.5 mmol). After addition, the reaction was stirred at room temperature for 30 min. The reaction mixture was neutralized with 1 M HCl (50 mL) and the aqueous layer was extracted with 10% methanol in dichloromethane (2 × 20 mL). The combined organic layers were washed with brine (20 mL), dried over anhydrous Na₂SO₄ and concentrated under reduced pressure. The obtained crude product was triturated with n-pentane (50 mL) at room temperature, filtered, dried under vacuum to afford (4-((2-(trimethylsilyl)ethoxy)methyl)-4H-thieno[3,2-b]pyrrole-5-carboxylic acid (8 g, 83% yield) as an off-white solid. LCMS: m/z found 298.4 [M+H]⁺.

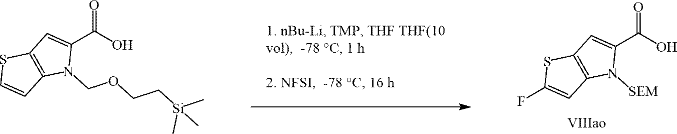

Step iii: To a stirred solution of 4-((2-(trimethylsilyl)ethoxy)methyl)-4H-thieno[3,2-b]pyrrole-5-carboxylic acid (6.0 g, 20.17 mmol) and 2,2,6,6-tetramethylpiperidine (7.6 mL, 40.34 mmol) in 60 mL of dry THF at -78° C. was added n-BuLi (2.5 M solution in hexane, 16.13 mmol, 40.34 mmol) dropwise. The reaction mixture was stirred at -78° C. for 30 min. To the reaction mixture was added N-fluorobenzenesulfonimide (7.6 g, 24.2 mmol) at -78° C. The mixture was allowed to warm to room temperature and stirred for 16 h. The reaction mixture was diluted with water (100 mL) and extracted with ethyl acetate (2 × 100 mL). The combined organic layers were dried over anhydrous Na₂SO₄ and concentrated under reduced pressure. The crude product was purified by reverse phase semi-prep MPLC to afford 2-fluoro-4-((2-(trimethylsilyl)ethoxy)methyl)-4H-thieno[3,2-b]pyrrole-5-carboxylic acid (VIIIao, 1.8 g, 28% yield). LCMS: m/z found 324.29 [M+H]+, ¹H NMR (400 MHz, DMSO-d₆): 12.55 (bs, 1H), 7.19 (s, 1H), 7.17 (d, 1H), 5.81 (s, 2H), 3.44 (t, 2H), 0.77 (t, 2H), 0.18 (s, 9H).

N-(6,7-Difluoro-4-oxo-3,4-dihydrophthalazin-1-yl)ethyl)-2-fluoro-N-methyl-4H-thieno[3,2-b]pyrrole-5-carboxamide (Compounds 102 and 103)

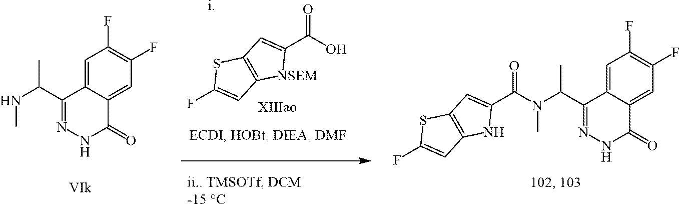

Step i: To a stirred solution of 2-fluoro-4-((2-(trimethylsilyl)ethoxy)methyl)-4H-thieno[3,2-b]pyrrole-5-carboxylic acid (XIIIao, 316 mg, 0.96 mmol) in 1 mL of DMF at room temperature were added DIEA (0.45 mL, 2.42 mmol), EDCI.HCl (230 mg, 1.20 mmol), and HOBt (164 mg, 1.20 mmol). After stirring at RT for 15 min, 6,7-difluoro-4-(1-(methylamino)ethyl)phthalazin-1(2H)-one (VIk, 200 mg, 0.81 mmol) was added. After stirring at RT overnight, the reaction mixture was poured into ice cold water (10 mL) and stirred for 30 min. A solid precipitate formed, which was collected, washed with water and dried it under vacuum to obtain N-(1-(6,7-difluoro-4-oxo-3,4-dihydrophthalazin-1-yl)ethyl)-2-fluoro-N-methyl-4-((2-(trimethylsilyl)ethoxy)methyl)-4H-thieno[3,2-b]pyrrole-5-carboxamide (150 mg, 35% yield). LCMS: m/z found 537.2 [M+H]+.

Step ii: To a -15° C. of solution of N-(1-(6,7-difluoro-4-oxo-3,4-dihydrophthalazin-1-yl)ethyl)-2-fluoro-N-methyl-4-((2-(trimethylsilyl)ethoxy)methyl)-4H-thieno[3,2-b]pyrrole-5-carboxamide (100 mg, 0.18 mmol) in DCM (1 mL) was added TMS-OTf (80 µL, 0.37 mmol). The resulting reaction mixture was stirred at -15° C. for 30 minutes. The reaction mixture was quenched with 10% saturated sodium carbonate solution (10 mL) and extracted with EtOAc (3 × 50 mL). The combined organics were dried over sodium sulfate and concentrated. The crude product was triturated with diethyl ether (10 mL) and filtered. The resulting solid was dried under vacuum and lyophilized to afford racemic N-(1-(6,7-difluoro-4-oxo-3,4-dihydrophthalazin-1-yl)ethyl)-2-fluoro-N-methyl-4H-thieno[3,2-b]pyrrole-5-carboxamide (70 mg, 56% yield). LCMS: m/z found 407.35 [M+H]+. The enantiomers were subsequently separated by preparative SFC: Method isocratic, Mobile phase Methanol: CO₂ -40:60. Column: Chiralpak IC-3 (30 × 250 mm), 5 µm, flow rate: 110 g/min.

Enantiomer I (Compound 102): LCMS: m/z found 407.0 [M+H]⁺, RT = 4.23 min, (Method A); ¹H NMR (400 MHz, DMSO-d₆): δ 12.96 (bs, 1H), 11.99 (bs, 1H), 8.2 (t, 1H), 8.0 (bs, 1H), 6.90 (s, 1H), 6.76 (bs, 1H), 6.34 (d, 1H), 2.96 (s, 3H), 1.51 (d, 3H); Chiral analytical SFC: RT = 4.20 min, Column: Chiralpak IC-3 (4.6 × 150 mm) 3 µm, 25 % (0.5 % DEA in Methanol), Flow rate: 3.0 g/min.

Enantiomer II (Compound 103): LCMS: m/z found 407.0 [M+H]⁺, RT = 4.23 min, (Method A); ¹H NMR (400 MHz, DMSO-d₆): δ 12.96 (bs, 1H), 11.99 (bs, 1H), 8.2 (t, 1H), 8.0 (bs, 1H), 6.90 (s, 1H), 6.76 (bs, 1H), 6.34 (d, 1H), 2.96 (s, 3H), 1.51 (d, 3H); Chiral analytical SFC: RT = 5.63 min, Column: Chiralpak IC-3 (4.6 × 150 mm) 3 µm, 25 % (0.5 % DEA in Methanol), Flow rate: 3.0 g/min.

### N-(6,7-difluoro-4-oxo-3,4-dihydrophthalazin-1-yl)ethyl)-N,1-dimethyl-1H-indole-6-carboxamide (Compounds 104 and 105)

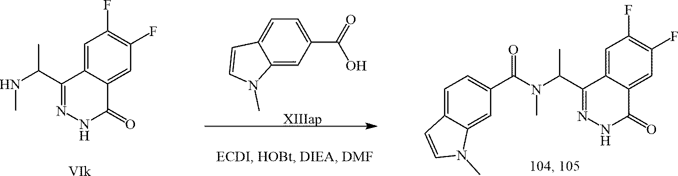

Racemic N-(1-(6,7-difluoro-4-oxo-3,4-dihydrophthalazin-1-yl)ethyl)-N,1-dimethyl-1H-indole-6-carboxamide was synthesized in an analogous manner as described above from racemic 6,7-difluoro-4-(1-(methylamino)ethyl)phthalazin-1(2H)-one (VIk) and 1-methyl-1H-indole-6-carboxylic acid (XIIIap). The enantiomers were subsequently separated by preparative SFC: Method isocratic, Mobile phase Methanol: CO₂ - 40:60. Column: Chiralpak IC (30 × 250 mm), 5 µm, flow rate: 110 g/min.

Enantiomer I (Compound 104): LCMS: m/z found 397.0 [M+H]⁺, RT = 3.84 min, (Method A); ¹H NMR (400 MHz, DMSO-d₆): δ 12.94 (bs, 1H), 8.23 (t, 1H), 8.05 (bs, 1H), 7.58-7.43 (m, 3H), 6.98 (s, 1H), 6.46 (d, 1H), 6.33 (bs, 1H), 3.79 (s, 3H), 2.65 (s, 3H), 1.57 (d, 3H); Chiral analytical SFC: RT = 3.68 min, Column: Chiralpak IC-3 (4.6 × 150 mm) 3 µm, 40 % Methanol, Flow rate: 3.0 g/min.

Enantiomer II (Compound 105): LCMS: m/z found 397.0 [M+H]⁺, RT = 3.84 min, (Method A); ¹H NMR (400 MHz, DMSO-d₆): δ 12.94 (bs, 1H), 8.23 (t, 1H), 8.05 (bs, 1H), 7.58-7.43 (m, 3H), 6.98 (s, 1H), 6.46 (d, 1H), 6.34 (bs, 1H), 3.79 (s, 3H), 2.65 (s, 3H), 1.57 (d, 3H); Chiral analytical SFC: RT = 5.53 min, Column: Chiralpak IC-3 (4.6 × 150 mm) 3 µm, 40 % Methanol, Flow rate: 3.0 g/min.

### N-(6,7-difluoro-4-oxo-3,4-dihydrophthalazin-1-yl)ethyl)-N,1-dimethyl-1H-indole-5-carboxamide (Compounds 106 and 107)

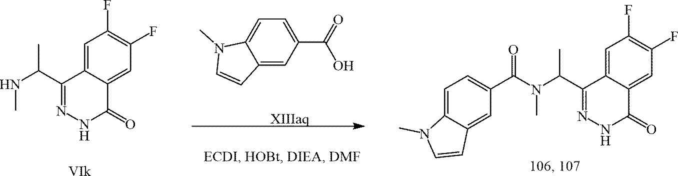

Racemic N-(1-(6,7-difluoro-4-oxo-3,4-dihydrophthalazin-1-yl)ethyl)-N,1-dimethyl-1H-indole-5-carboxamide was synthesized in an analogous manner as described above from racemic 6,7-difluoro-4-(1-(methylamino)ethyl)phthalazin-1(2H)-one (VIk) and 1-methyl-1H-indole-5-carboxylic acid (XIIIaq). The enantiomers were subsequently separated by preparative SFC: Method isocratic, Mobile phase Methanol: CO₂ - 40:60. Column: Chiralpak IC-3 (30 × 250 mm), 5 µm, flow rate: 110 g/min.

Enantiomer I (Compound 106): LCMS: m/z found 397.1 [M+H]⁺, RT = 3.69 min, (Method A); ¹H NMR (400 MHz, DMSO-d₆): δ 12.94 (bs, 1H), 8.23 (t, 1H), 8.05 (bs, 1H), 7.58-7.43 (m, 3H), 6.98 (s, 1H), 6.46 (d, 1H), 6.33 (bs, 1H), 3.80 (s, 3H), 2.69 (s, 3H), 1.57 (d, 3H); Chiral analytical SFC: RT = 2.13 min, Column: Chiralpak IC-3 (4.6 × 150 mm) 3 µm, 50 % (0.2% 7 N methanolic ammonia in Acetonitrile/Methanol [1:1]), Flow rate: 3.0 g/min.

Enantiomer II (Compound 107): LCMS: m/z found 397.1 [M+H]⁺, RT = 3.69 min, (Method A); ¹H NMR (400 MHz, DMSO-d₆): δ 12.94 (bs, 1H), 8.23 (t, 1H), 8.05 (bs, 1H), 7.58-7.43 (m, 3H), 6.98 (s, 1H), 6.46 (d, 1H), 6.34 (bs, 1H), 3.80 (s, 3H), 2.69 (s, 3H), 1.57 (d, 3H); Chiral analytical SFC: RT = 4.59 min, Column: Chiralpak IC-3 (4.6 × 150 mm) 3 µm, 50 % (0.2 % 7 N methanolic ammonia in Acetonitrile/Methanol [1:1]), Flow rate: 3.0 g/min.

### N-(6,7-difluoro-4-oxo-3,4-dihydrophthalazin-1-yl)ethyl)-4-(difluoromethyl)-3,5-difluoro-N-methylbenzamide (Compounds 108 and 109)

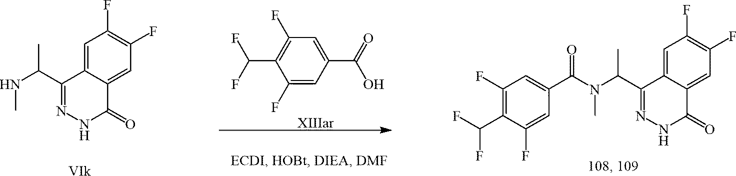

Racemic N-(1-(6,7-difluoro-4-oxo-3,4-dihydrophthalazin-1-yl)ethyl)-4-(difluoromethyl)-3,5-difluoro-N-methylbenzamide was synthesized in an analogous manner as described above from racemic 6,7-difluoro-4-(1-(methylamino)ethyl)phthalazin-1(2H)-one (VIk) and 4-(difluoromethyl)-3,5-difluorobenzoic acid (XIIIar). The enantiomers were subsequently separated by preparative SFC: Method isocratic, Mobile phase Methanol: CO₂ -40:60. Column: Chiralpak IG-3 (30 × 250 mm), 5 µm, flow rate: 110 g/min.

Enantiomer I (Compound 108): LCMS: m/z found 430.0 [M+H]⁺, RT = 4.20 min, (Method A); ¹H NMR (400 MHz, DMSO-d₆): δ 12.95 (bs, 1H), 8.23 (t, 1H), 7.98-7.93 (m, 1H), 7.44-7.18 (m, 3H), 6.28-6.23 (m, 1H), 2.59 (s, 3H), 1.54 (d, 3H); Chiral analytical SFC: RT = 1.76 min, Column: Chiralpak IG-3 (4.6 × 150 mm) 3 µm, 20% (0.5% DEA in Methanol), Flow rate: 3.0 g/min.

Enantiomer II (Compound 109): LCMS: m/z found 430.0 [M+H]⁺, RT = 4.20 min, (Method A); ¹H NMR (400 MHz, DMSO-d₆): δ 12.95 (bs, 1H), 8.23 (t, 1H), 7.98-7.93 (m, 1H), 7.44-7.18 (m, 3H), 6.28-6.23 (m, 1H), 2.59 (s, 3H), 1.54 (d, 3H); Chiral analytical SFC: RT = 2.29 min, Column: Chiralpak IG-3 (4.6 × 150 mm) 3 µm, 20% (0.5 % DEA in Methanol), Flow rate: 3.0 g/min.

### N-(6,7-difluoro-4-oxo-3,4-dihydrophthalazin-1-yl)ethyl)-N-methyl-1H-indazole-5-carboxamide (Compounds 110 and 111)

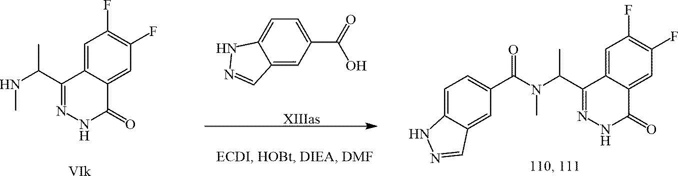

Racemic N-(1-(6,7-difluoro-4-oxo-3,4-dihydrophthalazin-1-yl)ethyl)-N-methyl-1H-indazole-5-carboxamide was synthesized in an analogous manner as described above from racemic 6,7-difluoro-4-(1-(methylamino)ethyl)phthalazin-1(2H)-one (VIk) and 1H-indazole-5-carboxylic acid (XIIIas). The enantiomers were subsequently separated by preparative SFC: Method isocratic, Mobile phase Methanol: CO₂ - 40:60. Column: Chiralpak IG (30 x 250 mm), 5 µm, flow rate: 110 g/min.

Enantiomer I (Compound 110): LCMS: m/z found 384.1 [M+H]⁺, RT = 2.61 min, (Method A); ¹H NMR (400 MHz, DMSO-d₆): δ 13.25 (bs, 1H), 12.95 (bs, 1H), 8.24 (t, 1H), 8.12 (t, 1H), 8.02 (bs, 1H), 7.82 (s, 1H), 7.58 (d, 1H), 7.33 (d, 1H), 6.33 (s, 1H), 2.63 (s, 3H), 1.56 (d, 3H); Chiral analytical SFC: RT = 3.84 min, Column: Chiralpak IG-3 (4.6 × 150 mm) 3 µm, 30 % (0.5% DEA in Methanol), Flow rate: 3.0 g/min.

Enantiomer II (Compound 111): LCMS: m/z found 384.0 [M+H]⁺, RT = 2.60 min, (Method A); ¹H NMR (400 MHz, DMSO-d₆): δ 12.95 (bs, 2H), 8.23 (t, 1H), 8.12 (t, 1H), 8.02 (bs, 1H), 7.82 (s, 1H), 7.58 (d, 1H), 7.33 (d, 1H), 6.33 (s, 1H), 2.63 (s, 3H), 1.57 (d, 3H); Chiral analytical SFC: RT = 5.07 min, Column: Chiralpak IC-3 (4.6 × 150 mm) 3 µm, 30% (0.5% DEA in Methanol), Flow rate: 3.0 g/min.

### N-(6,7-difluoro-4-oxo-3,4-dihydrophthalazin-1-yl)ethyl)-N-methyl-1H-indazole-6-carboxamide (Compounds 112 and 113)

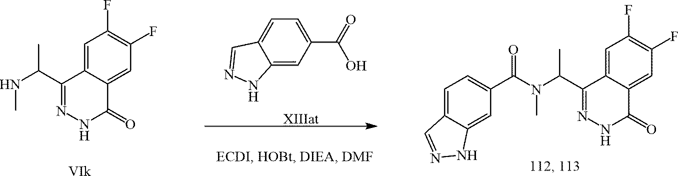

Racemic N-(1-(6,7-difluoro-4-oxo-3,4-dihydrophthalazin-1-yl)ethyl)-N-methyl-1H-indazole-6-carboxamide was synthesized in an analogous manner as described above from racemic 6,7-difluoro-4-(1-(methylamino)ethyl)phthalazin-1(2H)-one (VIk) and 1H-indazole-6-carboxylic acid (XIIIat). The enantiomers were subsequently separated by preparative SFC: Method isocratic, Mobile phase methanol: CO₂ - 40:60. Column: Chiralpak IC (30 x 250 mm), 5 µm, flow rate: 110 g/min.

Enantiomer I (Compound 112): LCMS: m/z found 384.0 [M+H]⁺, RT = 2.81 min, (Method A); ¹H NMR (400 MHz, DMSO-d₆): δ 12.99 (bs, 2H), 8.24 (t, 1H), 8.13 (s, 1H), 8.03 (t, 1H), 7.82 (d, 1H), 7.48 (s, 1H), 7.03 (d, 1H), 6.34 (d, 1H), 2.54 (s, 3H), 1.57 (d, 3H). Chiral analytical SFC: RT = 4.57 min, Column: Chiralpak IC-3 (4.6 × 150 mm) 3 µm, 40 % Methanol, Flow rate: 3.0 g/min.

Enantiomer II (Compound 113): LCMS: m/z found 384.1 [M+H]⁺, RT = 2.79 min, (Method A); ¹H NMR (400 MHz, DMSO-d₆): δ 13.0 (bs, 2H), 8.24 (t, 1H), 8.13 (s, 1H), 8.03 (t, 1H), 7.82 (d, 1H), 7.48 (s, 1H), 7.03 (d, 1H), 6.34 (d, 1H), 2.57 (s, 3H), 1.57 (d, 3H); Chiral analytical SFC: RT = 5.68 min, Column: Chiralpak IC (4.6 × 150 mm) 3 µm, 40 % Methanol, Flow rate: 3.0 g/min.

### N-(6,7-difluoro-4-oxo-3,4-dihydrophthalazin-1-yl)ethyl)-5-fluoro-N-methyl-6-(trifluoromethyl)nicotinamide (Compound 114)

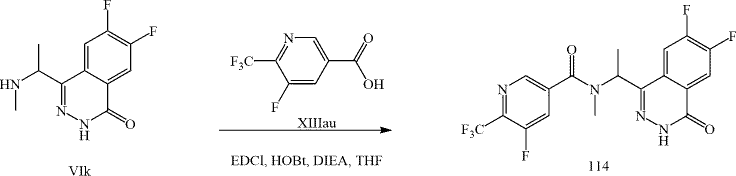

Racemic N-(1-(6,7-difluoro-4-oxo-3,4-dihydrophthalazin-1-yl)ethyl)-5-fluoro-N-methyl-6-(trifluoromethyl)nicotinamide was synthesized in an analogous manner as described above from racemic 6,7-difluoro-4-(1-(methylamino)ethyl)phthalazin-1(2H)-one (VIk) and 5-fluoro-6-(trifluoromethyl)nicotinic acid (XIIIau). LCMS: m/z found 431.0 [M+H]⁺, RT = 4.17 min, (Method A); ¹H NMR (400 MHz, DMSO-d₆): δ 12.95 (bs, 1H), 8.61 (s, 1H), 8.25-8.16 (m, 2H), 7.92 (t, 1H), 6.30-6.25 (m, 1H), 2.64 (bs, 3H), 1.57 (d, 3H).

### Example 2: Biological Results

Representative compounds of the disclosure were tested for their abilities to inhibit formation of relaxed circular DNA (rcDNA) in a HepDE19 assay, as described elsewhere herein. Results are illustrated in Table 4.

TABLE 4

| | | |
|---|---|---|
| Cmpd | Nomenclature | DE-19bDNA EC₅₀ (µM) |
| 1 | 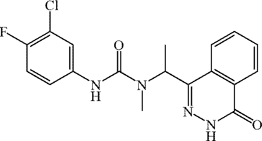 [SMILES: CC(c1n[nH]c(=O)c2ccccc12)N(C)C(=O)Nc1ccc(F)c(Cl)c1] | 0.24 |
| | 3-(3-chloro-4-fluorophenyl)-1-methyl-1-(1-(4-oxo-3,4-dihydrophthalazin-1-yl)ethyl)urea | |
| 2 | 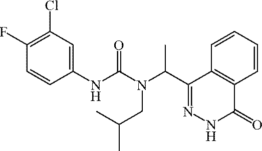 [SMILES: CC(C)CN(C(=O)Nc1ccc(F)c(Cl)c1)C(C)c1n[nH]c(=O)c2ccccc12] | 0.01 |
| | 3-(3-chloro-4-fluorophenyl)-1-isobutyl-1-(1-(4-oxo-3,4-dihydrophthalazin-1-yl)ethyl)urea | |
| 3 | 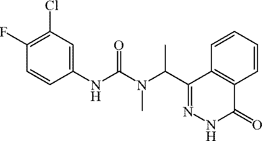 [SMILES: CC(c1n[nH]c(=O)c2ccccc12)N(C)C(=O)Nc1ccc(F)c(Cl)c1] | 14 |
| | 3-(3-chloro-4-fluorophenyl)-1-methyl-1-(1-(4-oxo-3,4-dihydrophthalazin-1-yl)ethyl)urea | |
| | enantiomer I | |
| 4 | 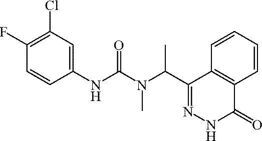 [SMILES: CC(c1n[nH]c(=O)c2ccccc12)N(C)C(=O)Nc1ccc(F)c(Cl)c1] | 0.20 |
| | 3-(3-chloro-4-fluorophenyl)-1-methyl-1-(1-(4-oxo-3,4-dihydrophthalazin-1-yl)ethyl)urea | |
| | enantiomer II | |
| 5 | 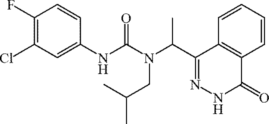 [SMILES: CC(C)CN(C(=O)Nc1ccc(F)c(Cl)c1)C(C)c1n[nH]c(=O)c2ccccc12] | 0.01 |
| | 3-(3-chloro-4-fluorophenyl)-1-isobutyl-1-(1-(4-oxo-3,4-dihydrophthalazin-1-yl)ethyl)urea | |
| | enantiomer I | |
| 6 | 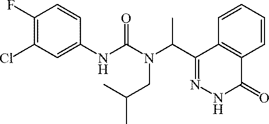 [SMILES: CC(C)CN(C(=O)Nc1ccc(F)c(Cl)c1)C(C)c1n[nH]c(=O)c2ccccc12] | 1.7 |
| | 3-(3-chloro-4-fluorophenyl)-1-isobutyl-1-(1-(4-oxo-3,4-dihydrophthalazin-1-yl)ethyl)urea | |
| | enantiomer II | |
| 8 | 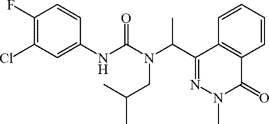 [SMILES: CC(C)CN(C(=O)Nc1ccc(F)c(Cl)c1)C(C)c1nn(C)c(=O)c2ccccc12] | 0.02 |
| | 3-(3-chloro-4-fluorophenyl)-1-isobutyl-1-(1-(3-methyl-4-oxo-3,4-dihydrophthalazin-1-yl)ethyl)urea | |
| 9 | 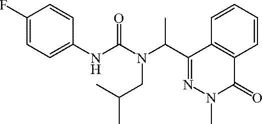 [SMILES: CC(C)CN(C(=O)Nc1ccc(F)cc1)C(C)c1nn(C)c(=O)c2ccccc12] | 0.03 |
| | 3-(4-fluorophenyl)-1-isobutyl-1-(1-(3-methyl-4-oxo-3,4-dihydrophthalazin-1-yl)ethyl)urea | |
| 10 | 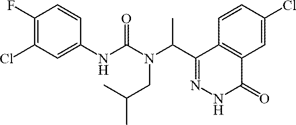 [SMILES: CC(C)CN(C(=O)Nc1ccc(F)c(Cl)c1)C(C)c1n[nH]c(=O)c2cc(Cl)ccc12] | 0.02 |
| | 3-(3-chloro-4-fluorophenyl)-1-(1-(6-chloro-4-oxo-3,4-dihydrophthalazin-1-yl)ethyl)-1-isobutylurea | |
| 11 | 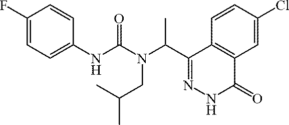 [SMILES: CC(C)CN(C(=O)Nc1ccc(F)cc1)C(C)c1n[nH]c(=O)c2cc(Cl)ccc12] | 0.04 |
| | 1-(1-(6-chloro-4-oxo-3,4-dihydrophthalazin-1-yl)ethyl)-3-(4-fluorophenyl)-1-isobutylurea | |
| 12 | 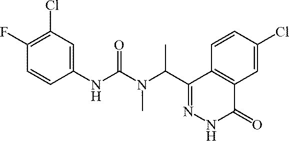 [SMILES: CC(c1n[nH]c(=O)c2cc(Cl)ccc12)N(C)C(=O)Nc1ccc(F)c(Cl)c1] | 0.26 |
| | 3-(3-chloro-4-fluorophenyl)-1-(1-(6-chloro-4-oxo-3,4-dihydrophthalazin-1-yl)ethyl)-1-methylurea | |
| 13 | 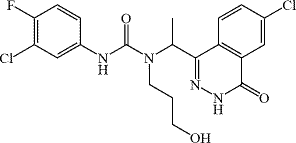 [SMILES: CC(c1n[nH]c(=O)c2cc(Cl)ccc12)N(CCCO)C(=O)Nc1ccc(F)c(Cl)c1] | 0.022 |
| | 3-(3-chloro-4-fluorophenyl)-1-(1-(6-chloro-4-oxo-3,4-dihydrophthalazin-1-yl)ethyl)-1-(3-hydroxypropyl)urea | |
| 14 | 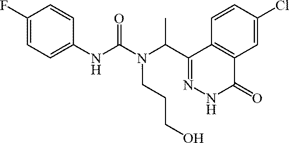 [SMILES: CC(c1n[nH]c(=O)c2cc(Cl)ccc12)N(CCCO)C(=O)Nc1ccc(F)cc1] | 0.11 |
| | 1-(1-(6-chloro-4-oxo-3,4-dihydrophthalazin-1-yl)ethyl)-3-(4-fluorophenyl)-1-(3-hydroxypropyl)urea | |
| 15 | 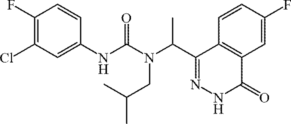 [SMILES: CC(C)CN(C(=O)Nc1ccc(F)c(Cl)c1)C(C)c1n[nH]c(=O)c2cc(F)ccc12] | 0.01 |
| | 3-(3-chloro-4-fluorophenyl)-1-(1-(6-fluoro-4-oxo-3,4-dihydrophthalazin-1-yl)ethyl)-1-isobutylurea | |
| 16 | 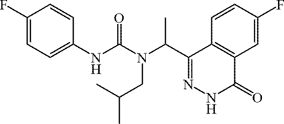 [SMILES: CC(C)CN(C(=O)Nc1ccc(F)cc1)C(C)c1n[nH]c(=O)c2cc(F)ccc12] | 0.016 |
| | 1-(1-(6-fluoro-4-oxo-3,4-dihydrophthalazin-1-yl)ethyl)-3-(4-fluorophenyl)-1-isobutylurea | |
| 17 | 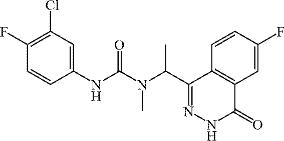 [SMILES: CC(c1n[nH]c(=O)c2cc(F)ccc12)N(C)C(=O)Nc1ccc(F)c(Cl)c1] | 0.081 |
| | 3-(3-chloro-4-fluorophenyl)-1-(1-(6-fluoro-4-oxo-3,4-dihydrophthalazin-1-yl)ethyl)-1-methylurea | |
| 18 | 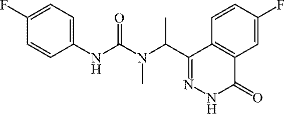 [SMILES: CC(c1n[nH]c(=O)c2cc(F)ccc12)N(C)C(=O)Nc1ccc(F)cc1] | 0.50 |
| | 1-(1-(6-fluoro-4-oxo-3,4-dihydrophthalazin-1-yl)ethyl)-3-(4-fluorophenyl)-1-methylurea | |
| 19 | 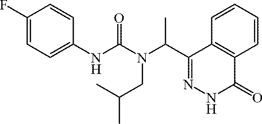 [SMILES: CC(C)CN(C(=O)Nc1ccc(F)cc1)C(C)c1n[nH]c(=O)c2ccccc12] | 0.048 |
| | 3-(4-fluorophenyl)-1-isobutyl-1-(1-(4-oxo-3,4-dihydrophthalazin-1-yl)ethyl)urea | |
| 20 | 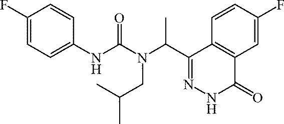 [SMILES: CC(C)CN(C(=O)Nc1ccc(F)cc1)C(C)c1n[nH]c(=O)c2cc(F)ccc12] | 0.008 |
| | 1-(1-(6-fluoro-4-oxo-3,4-dihydrophthalazin-1-yl)ethyl)-3-(4-fluorophenyl)-1-isobutylurea | |
| | enantiomer I | |
| 21 | 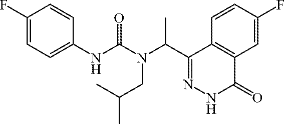 [SMILES: CC(C)CN(C(=O)Nc1ccc(F)cc1)C(C)c1n[nH]c(=O)c2cc(F)ccc12] | 2.4 |
| | 1-(1-(6-fluoro-4-oxo-3,4-dihydrophthalazin-1-yl)ethyl)-3-(4-fluorophenyl)-1-isobutylurea | |
| | enantiomer II | |
| 22 | 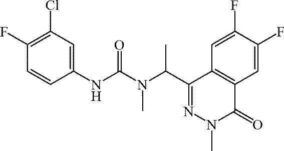 [SMILES: CC(c1nn(C)c(=O)c2cc(F)c(F)cc12)N(C)C(=O)Nc1ccc(F)c(Cl)c1] | 0.026 |
| | 3-(3-chloro-4-fluorophenyl)-1-(1-(6,7-difluoro-3-methyl-4-oxo-3,4-dihydrophthalazin-1-yl)ethyl)-1-methylurea | |
| 23 | 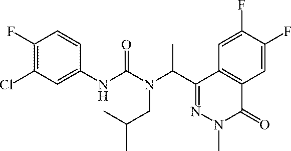 [SMILES: CC(C)CN(C(=O)Nc1ccc(F)c(Cl)c1)C(C)c1nn(C)c(=O)c2cc(F)c(F)cc12] | 0.003 |
| | 3-(3-chloro-4-fluorophenyl)-1-(1-(6,7-difluoro-3-methyl-4-oxo-3,4-dihydrophthalazin-1-yl)ethyl)-1-isobutylurea | |
| 24 | 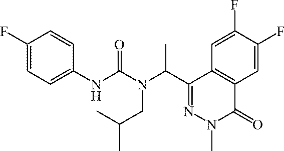 [SMILES: CC(C)CN(C(=O)Nc1ccc(F)cc1)C(C)c1nn(C)c(=O)c2cc(F)c(F)cc12] | 0.007 |
| | 1-(1-(6,7-difluoro-3-methyl-4-oxo-3,4-dihydrophthalazin-1-yl)ethyl)-3-(4-fluorophenyl)-1-isobutylurea | |
| 25 | 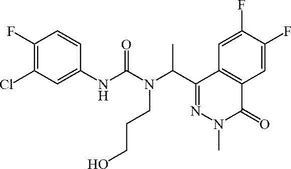 [SMILES: CC(c1nn(C)c(=O)c2cc(F)c(F)cc12)N(CCCO)C(=O)Nc1ccc(F)c(Cl)c1] | 0.01 |
| | 3-(3-chloro-4-fluorophenyl)-1-(1-(6,7-difluoro-3-methyl-4-oxo-3,4-dihydrophthalazin-1-yl)ethyl)-1-(3-hydroxypropyl)urea | |
| 26 | 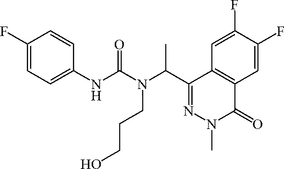 [SMILES: CC(c1nn(C)c(=O)c2cc(F)c(F)cc12)N(CCCO)C(=O)Nc1ccc(F)cc1] | 0.031 |
| | 3-(4-fluorophenyl)-1-(1-(6,7-difluoro-3 -methyl-4-oxo-3,4-dihydrophthalazin-1-yl)ethyl)-1-(3-hydroxypropyl)urea | |
| 27 | 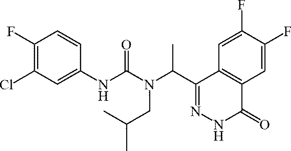 [SMILES: CC(C)CN(C(=O)Nc1ccc(F)c(Cl)c1)C(C)c1n[nH]c(=O)c2cc(F)c(F)cc12] | 0.002 |
| | 3-(3-chloro-4-fluorophenyl)-1-(1-(6,7-difluoro-4-oxo-3,4-dihydrophthalazin-1-yl)ethyl)-1-isobutylurea | |
| 28 | 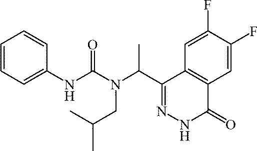 [SMILES: CC(C)CN(C(=O)Nc1ccccc1)C(C)c1n[nH]c(=O)c2cc(F)c(F)cc12] | 0.012 |
| | 1-(1-(6,7-difluoro-4-oxo-3,4-dihydrophthalazin-1-yl)ethyl)-1-isobutyl-3-phenylurea | |
| 29 | 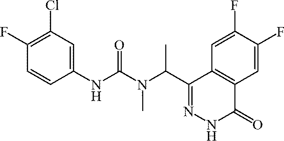 [SMILES: CC(c1n[nH]c(=O)c2cc(F)c(F)cc12)N(C)C(=O)Nc1ccc(F)c(Cl)c1] | 0.023 |
| | 3-(3-chloro-4-fluorophenyl)-1-(1-(6,7-difluoro-4-oxo-3,4-dihydrophthalazin-1-yl)ethyl)-1-methylurea | |
| 30 | 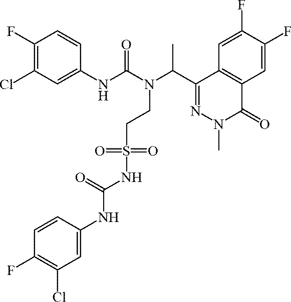 [SMILES: CC(c1nn(C)c(=O)c2cc(F)c(F)cc12)N(CCS(=O)(=O)NC(=O)Nc1ccc(F)c(Cl)c1)C(=O)Nc1ccc(F)c(Cl)c1] | 0.058 |
| | 2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide | |
| 31 | 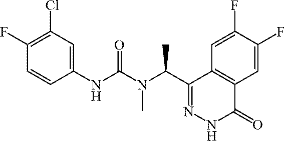 [SMILES: C[C@@H](c1n[nH]c(=O)c2cc(F)c(F)cc12)N(C)C(=O)Nc1ccc(F)c(Cl)c1] | 1.8 |
| | (S)-3-(3-chloro-4-fluorophenyl)-1-(1-(6,7-difluoro-4-oxo-3,4-dihydrophthalazin-1-yl)ethyl)-1-methylurea | |
| | enantiomer I | |
| 32 | 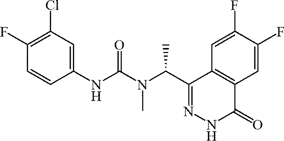 [SMILES: C[C@H](c1n[nH]c(=O)c2cc(F)c(F)cc12)N(C)C(=O)Nc1ccc(F)c(Cl)c1] | 0.021 |
| | (R)-3-(3-chloro-4-fluorophenyl)-1-(1-(6,7-difluoro-4-oxo-3,4- | |
| | dihydrophthalazin-1-yl)ethyl)-1-methylurea | |
| | enantiomer II | |
| 33 | 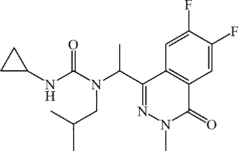 [SMILES: CC(C)CN(C(=O)NC1CC1)C(C)c1nn(C)c(=O)c2cc(F)c(F)cc12] | 6.3 |
| | 3-cyclopropyl-1-(1-(6,7-difluoro-3-methyl-4-oxo-3,4-dihydrophthalazin-1-yl)ethyl)-1-isobutylurea | |
| 34 | 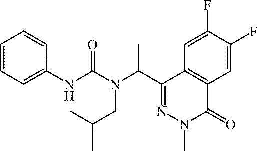 [SMILES: CC(C)CN(C(=O)Nc1ccccc1)C(C)c1nn(C)c(=O)c2cc(F)c(F)cc12] | 0.01 |
| | 1-(1-(6,7-difluoro-3-methyl-4-oxo-3,4-dihydrophthalazin-1-yl)ethyl)-1-isobutyl-3-phenylurea | |
| 35 | 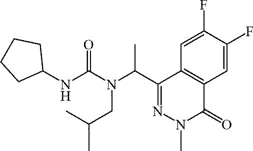 [SMILES: CC(C)CN(C(=O)NC1CCCC1)C(C)c1nn(C)c(=O)c2cc(F)c(F)cc12] | 6.9 |
| | 3-cyclopentyl-1-(1-(6,7-difluoro-3-methyl-4-oxo-3,4-dihydrophthalazin-1-yl)ethyl)-1-isobutylurea | |
| 36 | 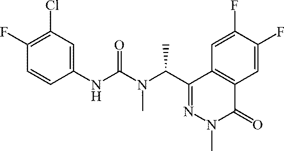 [SMILES: C[C@H](c1nn(C)c(=O)c2cc(F)c(F)cc12)N(C)C(=O)Nc1ccc(F)c(Cl)c1] | 0.013 |
| | (R)-3-(3-chloro-4-fluorophenyl)-1-(1-(6,7-difluoro-3-methyl-4-oxo-3,4-dihydrophthalazin-1-yl)ethyl)-1-methylurea | |
| 37 | 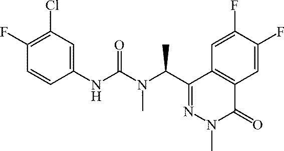 [SMILES: C[C@@H](c1nn(C)c(=O)c2cc(F)c(F)cc12)N(C)C(=O)Nc1ccc(F)c(Cl)c1] | 2.3 |
| | (S)-3-(3-chloro-4-fluorophenyl)-1-(1-(6,7-difluoro-3-methyl-4-oxo-3,4-dihydrophthalazin-1-yl)ethyl)-1-methylurea | |
| 38 | 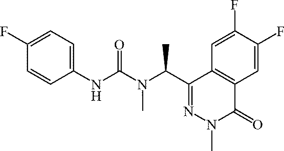 [SMILES: C[C@@H](c1nn(C)c(=O)c2cc(F)c(F)cc12)N(C)C(=O)Nc1ccc(F)cc1] | 8.2 |
| | (S)-1-(1-(6,7-difluoro-3-methyl-4-oxo-3,4-dihydrophthalazin-1-yl)ethyl)-3-(4-fluorophenyl)-1-methylurea | |
| 39 | 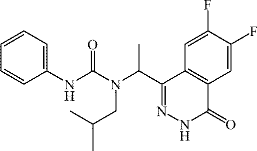 [SMILES: CC(C)CN(C(=O)Nc1ccccc1)C(C)c1n[nH]c(=O)c2cc(F)c(F)cc12] | 3.7 |
| | 1-(1-(6,7-difluoro-4-oxo-3,4-dihydrophthalazin-1-yl)ethyl)-1-isobutyl-3-phenylurea | |
| | enantiomer I | |
| 40 | 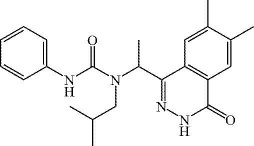 [SMILES: Cc1cc2c(C(C)N(CC(C)C)C(=O)Nc3ccccc3)n[nH]c(=O)c2cc1C] | 0.017 |
| | 1-(1-(6,7-difluoro-4-oxo-3,4-dihydrophthalazin-1-yl)ethyl)-1-isobutyl-3-phenylurea | |
| | enantiomer II | |
| 41 | 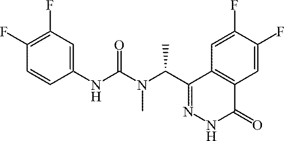 [SMILES: C[C@H](c1n[nH]c(=O)c2cc(F)c(F)cc12)N(C)C(=O)Nc1ccc(F)c(F)c1] | 0.024 |
| | (R)-1-(1-(6,7-difluoro-4-oxo-3,4-dihydrophthalazin-1-yl)ethyl)-3-(3,4-difluorophenyl)-1-methylurea | |
| 42 | 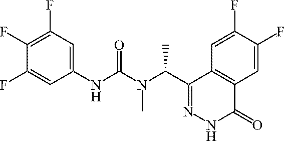 [SMILES: C[C@H](c1n[nH]c(=O)c2cc(F)c(F)cc12)N(C)C(=O)Nc1cc(F)c(F)c(F)c1] | 0.025 |
| | (R)-1-(1-(6,7-difluoro-4-oxo-3,4-dihydrophthalazin-1-yl)ethyl)-1-methyl-3-(3,4,5-trifluorophenyl)urea | |
| 43 | 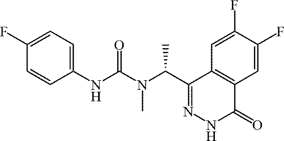 [SMILES: C[C@H](c1n[nH]c(=O)c2cc(F)c(F)cc12)N(C)C(=O)Nc1ccc(F)cc1] | 0.059 |
| | (R)-1-(1-(6,7-difluoro-4-oxo-3,4-dihydrophthalazin-1-yl)ethyl)-3-(4-fluorophenyl)-1-methylurea | |
| 44 | 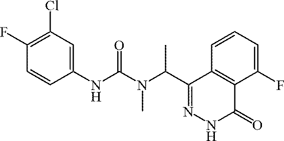 [SMILES: CC(c1n[nH]c(=O)c2c(F)cccc12)N(C)C(=O)Nc1ccc(F)c(Cl)c1] | 0.29 |
| | 3-(3-chloro-4-fluorophenyl)-1-(1-(5-fluoro-4-oxo-3,4-dihydrophthalazin-1-yl)ethyl)-1-methylurea | |
| 45 | 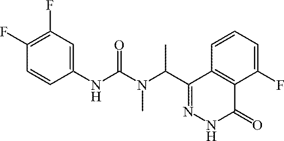 [SMILES: CC(c1n[nH]c(=O)c2c(F)cccc12)N(C)C(=O)Nc1ccc(F)c(F)c1] | 0.79 |
| | 3-(3,4-difluorophenyl)-1-(1-(5-fluoro-4-oxo-3,4-dihydrophthalazin-1-yl)ethyl)-1-methylurea | |
| 46 | 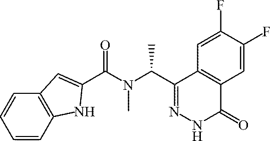 [SMILES: C[C@H](c1n[nH]c(=O)c2cc(F)c(F)cc12)N(C)C(=O)c1cc2ccccc2[nH]1] | 0.032 |
| | (R)-N-(1-(6,7-difluoro-4-oxo-3,4-dihydrophthalazin-1-yl)ethyl)-N-methyl-1H-indole-2-carboxamide | |
| 47 | 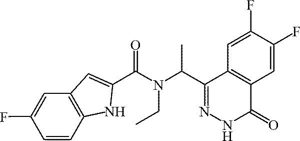 [SMILES: CCN(C(=O)c1cc2cc(F)ccc2[nH]1)C(C)c1n[nH]c(=O)c2cc(F)c(F)cc12] | 0.041 |
| | N-(1-(6,7-difluoro-4-oxo-3,4-dihydrophthalazin-1-yl)ethyl)-N-ethyl-5-fluoro-1H-indole-2-carboxamide | |
| 48 | 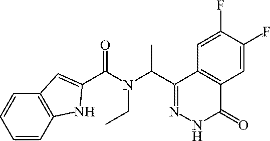 [SMILES: CCN(C(=O)c1cc2ccccc2[nH]1)C(C)c1n[nH]c(=O)c2cc(F)c(F)cc12] | 0.060 |
| | N-(1-(6,7-difluoro-4-oxo-3,4-dihydrophthalazin-1-yl)ethyl)-N-ethyl-1H-indole-2-carboxamide | |
| 49 | 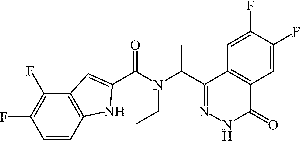 [SMILES: CCN(C(=O)c1cc2c(F)c(F)ccc2[nH]1)C(C)c1n[nH]c(=O)c2cc(F)c(F)cc12] | 0.025 |
| | N-(1-(6,7-difluoro-4-oxo-3,4-dihydrophthalazin-1-yl)ethyl)-N-ethyl-4,5-difluoro-1H-indole-2-carboxamide | |
| 50 | 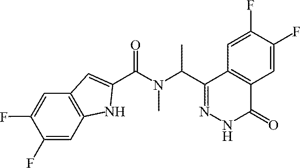 [SMILES: CC(c1n[nH]c(=O)c2cc(F)c(F)cc12)N(C)C(=O)c1cc2cc(F)c(F)cc2[nH]1] | 0.040 |
| | N-(1-(6,7-difluoro-4-oxo-3,4-dihydrophthalazin-1-yl)ethyl)-5,6-difluoro-N-methyl-1H-indole-2-carboxamide | |
| 51 | 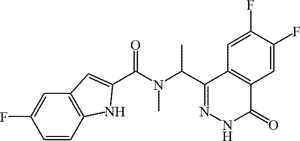 [SMILES: CC(c1n[nH]c(=O)c2cc(F)c(F)cc12)N(C)C(=O)c1cc2cc(F)ccc2[nH]1] | 0.039 |
| | N-(1-(6,7-difluoro-4-oxo-3,4-dihydrophthalazin-1-yl)ethyl)-5-fluoro-N-methyl-1H-indole-2-carboxamide | |
| 52 | 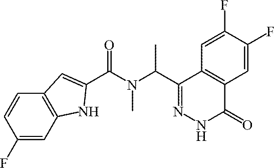 [SMILES: CC(c1n[nH]c(=O)c2cc(F)c(F)cc12)N(C)C(=O)c1cc2ccc(F)cc2[nH]1] | 0.043 |
| | N-(1-(6,7-difluoro-4-oxo-3,4-dihydrophthalazin-1-yl)ethyl)-6-fluoro-N-methyl-1H-indole-2-carboxamide | |
| 53 | 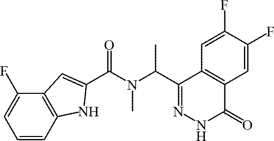 [SMILES: CC(c1n[nH]c(=O)c2cc(F)c(F)cc12)N(C)C(=O)c1cc2c(F)cccc2[nH]1] | 0.083 |
| | N-(1-(6,7-difluoro-4-oxo-3,4-dihydrophthalazin-1-yl)ethyl)-4-fluoro-N-methyl-1H-indole-2-carboxamide | |
| 54 | 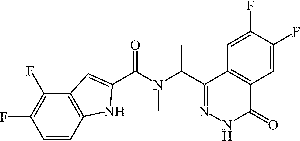 [SMILES: CC(c1n[nH]c(=O)c2cc(F)c(F)cc12)N(C)C(=O)c1cc2c(F)c(F)ccc2[nH]1] | 0.024 |
| | N-(1-(6,7-difluoro-4-oxo-3,4-dihydrophthalazin-1-yl)ethyl)-4,5-difluoro-N-methyl-1H-indole-2-carboxamide | |
| 55 | 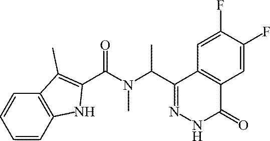 [SMILES: Cc1c(C(=O)N(C)C(C)c2n[nH]c(=O)c3cc(F)c(F)cc23)[nH]c2ccccc12] | 0.44 |
| | N-(1-(6,7-difluoro-4-oxo-3,4-dihydrophthalazin-1-yl)ethyl)-N,3-dimethyl-1H-indole-2-carboxamide | |
| 56 | 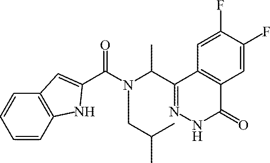 [SMILES: CC(C)CN(C(=O)c1cc2ccccc2[nH]1)C(C)c1n[nH]c(=O)c2cc(F)c(F)cc12] | 0.39 |
| | N-(1-(6,7-difluoro-4-oxo-3,4-dihydrophthalazin-l-yl)ethyl)-N-isobutyl-1H-indole-2-carboxamide | |
| 57 | 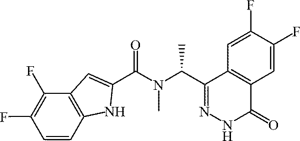 [SMILES: C[C@H](c1n[nH]c(=O)c2cc(F)c(F)cc12)N(C)C(=O)c1cc2c(F)c(F)ccc2[nH]1] | 0.012 |
| | (R)-N-(1-(6,7-difluoro-4-oxo-3,4-dihydrophthalazin-1-yl)ethyl)-4,5-difluoro-N-methyl-1H-indole-2-carboxamide | |
| 58 | 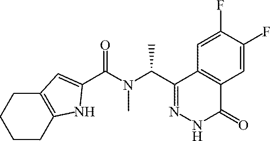 [SMILES: C[C@H](c1n[nH]c(=O)c2cc(F)c(F)cc12)N(C)C(=O)c1cc2c([nH]1)CCCC2] | 0.12 |
| | (R)-N-(1-(6,7-difluoro-4-oxo-3,4-dihydrophthalazin-1-yl)ethyl)-N-methyl-4,5,6,7-tetrahydro-1H-indole-2-carboxamide | |
| 59 | 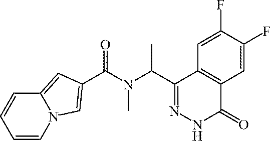 [SMILES: CC(c1n[nH]c(=O)c2cc(F)c(F)cc12)N(C)C(=O)c1cc2ccccn2c1] | 0.13 |
| | N-(1-(6,7-difluoro-4-oxo-3,4-dihydrophthalazin-1-yl)ethyl)-N-methylindolizine-2-carboxamide | |
| 60 | 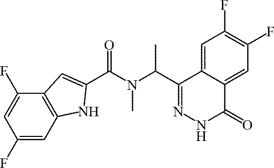 [SMILES: CC(c1n[nH]c(=O)c2cc(F)c(F)cc12)N(C)C(=O)c1cc2c(F)cc(F)cc2[nH]1] | 0.05 |
| | N-(1-(6,7-difluoro-4-oxo-3,4-dihydrophthalazin-1-yl)ethyl)-4,6-difluoro-N-methyl-1H-indole-2-carboxamide | |
| 62 | 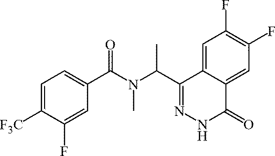 [SMILES: CC(c1n[nH]c(=O)c2cc(F)c(F)cc12)N(C)C(=O)c1ccc(C(F)(F)F)c(F)c1] | 0.10 |
| | N-(1-(6,7-difluoro-4-oxo-3,4-dihydrophthalazin-1-yl)ethyl)-3-fluoro- | |
| | N-methyl-4-(trifluoromethyl)benzamide | |
| 63 | 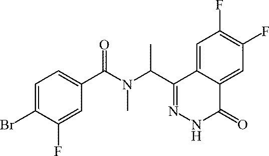 [SMILES: CC(c1n[nH]c(=O)c2cc(F)c(F)cc12)N(C)C(=O)c1ccc(Br)c(F)c1] | 0.06 |
| | 4-bromo-N-(1-(6,7-difluoro-4-oxo-3,4-dihydrophthalazin-1-yl)ethyl)-3-fluoro-N-methylbenzamide | |
| 64 | 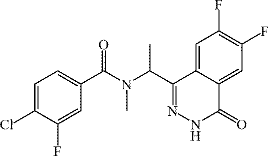 [SMILES: CC(c1n[nH]c(=O)c2cc(F)c(F)cc12)N(C)C(=O)c1ccc(Cl)c(F)c1] | 0.14 |
| | 4-chloro-N-(1-(6,7-difluoro-4-oxo-3,4-dihydrophthalazin-1-yl)ethyl)-3-fluoro-N-methylbenzamide | |
| 65 | 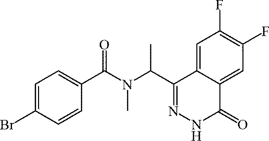 [SMILES: CC(c1n[nH]c(=O)c2cc(F)c(F)cc12)N(C)C(=O)c1ccc(Br)cc1] | 0.06 |
| | 4-bromo-N-(1-(6,7-difluoro-4-oxo-3,4-dihydrophthalazin-1-yl)ethyl)-N-methylbenzamide | |
| 66 | 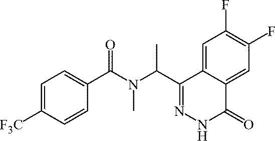 [SMILES: CC(c1n[nH]c(=O)c2cc(F)c(F)cc12)N(C)C(=O)c1ccc(C(F)(F)F)cc1] | 0.10 |
| | N-(1-(6,7-difluoro-4-oxo-3,4-dihydrophthalazin-1-yl)ethyl)-N-methyl-4-(trifluoromethyl)benzamide | |
| 67 | 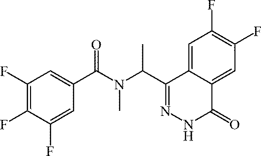 [SMILES: CC(c1n[nH]c(=O)c2cc(F)c(F)cc12)N(C)C(=O)c1cc(F)c(F)c(F)c1] | 0.74 |
| | N-(1-(6,7-difluoro-4-oxo-3,4-dihydrophthalazin-1-yl)ethyl)-3,4,5-trifluoro-N-methylbenzamide | |
| 68 | 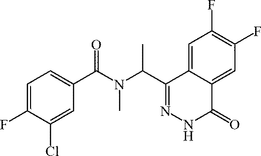 [SMILES: CC(c1n[nH]c(=O)c2cc(F)c(F)cc12)N(C)C(=O)c1ccc(F)c(Cl)c1] | 0.52 |
| | 3-chloro-N-(1-(6,7-difluoro-4-oxo-3,4-dihydrophthalazin-1-yl)ethyl)-4-fluoro-N-methylbenzamide | |
| 69 | 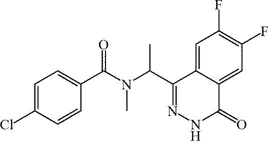 [SMILES: CC(c1n[nH]c(=O)c2cc(F)c(F)cc12)N(C)C(=O)c1ccc(Cl)cc1] | 0.20 |
| | 4-chloro-N-(1-(6,7-difluoro-4-oxo-3,4-dihydrophthalazin-1-yl)ethyl)-N-methylbenzamide | |
| 70 | 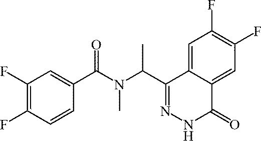 [SMILES: CC(c1n[nH]c(=O)c2cc(F)c(F)cc12)N(C)C(=O)c1ccc(F)c(F)c1] | 0.89 |
| | N-(1-(6,7-difluoro-4-oxo-3,4-dihydrophthalazin-1-yl)ethyl)-3,4-difluoro-N-methylbenzamide | |
| 71 | 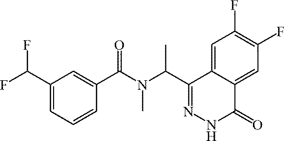 [SMILES: CC(c1n[nH]c(=O)c2cc(F)c(F)cc12)N(C)C(=O)c1cccc(C(F)F)c1] | 0.22 |
| | N-(1-(6,7-difluoro-4-oxo-3,4-dihydrophthalazin-1-yl)ethyl)-3-(difluoromethyl)-N-methylbenzamide | |
| 72 | 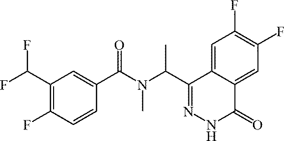 [SMILES: CC(c1n[nH]c(=O)c2cc(F)c(F)cc12)N(C)C(=O)c1ccc(F)c(C(F)F)c1] | 0.34 |
| | N-(1-(6,7-difluoro-4-oxo-3,4-dihydrophthalazin-1-yl)ethyl)-3-(difluoromethyl)-4-fluoro-N-methylbenzamide | |
| 73 | 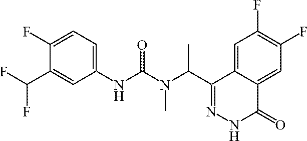 [SMILES: CC(c1n[nH]c(=O)c2cc(F)c(F)cc12)N(C)C(=O)Nc1ccc(F)c(C(F)F)c1] | 0.02 |
| | 1-(1-(6,7-difluoro-4-oxo-3,4-dihydrophthalazin-1-yl)ethyl)-3-(3-(difluoromethyl)-4-fluorophenyl)-1-methylurea | |
| 74 | 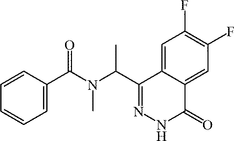 [SMILES: CC(c1n[nH]c(=O)c2cc(F)c(F)cc12)N(C)C(=O)c1ccccc1] | 17.68 |
| | N-(1-(6,7-difluoro-4-oxo-3,4-dihydrophthalazin-1-yl)ethyl)-N- | |
| | methylbenzamide | |
| 75 | 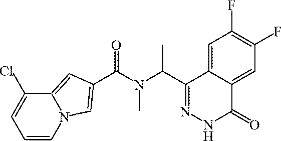 [SMILES: CC(c1n[nH]c(=O)c2cc(F)c(F)cc12)N(C)C(=O)c1cc2c(Cl)cccn2c1] | 0.06 |
| | 8-chloro-N-(1-(6,7-difluoro-4-oxo-3,4-dihydrophthalazin-1-yl)ethyl)-N-methylindolizine-2-carboxamide | |
| 76 | 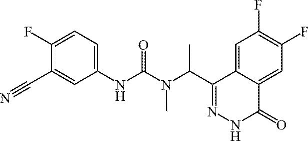 [SMILES: CC(c1n[nH]c(=O)c2cc(F)c(F)cc12)N(C)C(=O)Nc1ccc(F)c(C#N)c1] | 0.04 |
| | 3-(3-cyano-4-fluorophenyl)-1-(1-(6,7-difluoro-4-oxo-3,4-dihydrophthalazin-1-yl)ethyl)-1-methylurea | |
| 77 | 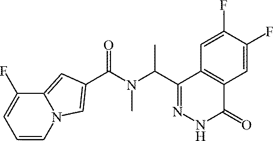 [SMILES: CC(c1n[nH]c(=O)c2cc(F)c(F)cc12)N(C)C(=O)c1cc2c(F)cccn2c1] | 8.38 |
| | N-(1-(6,7-difluoro-4-oxo-3,4-dihydrophthalazin-1-yl)ethyl)-8-fluoro-N-methylindolizine-2-carboxamide | |
| 78 | 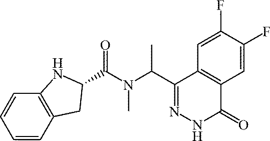 [SMILES: CC(c1n[nH]c(=O)c2cc(F)c(F)cc12)N(C)C(=O)[C@@H]1Cc2ccccc2N1] | 3.64 |
| | (2S)-N-(1-(6,7-difluoro-4-oxo-3,4-dihydrophthalazin-1-yl)ethyl)-N-methylindoline-2-carboxamide | |
| 79 | 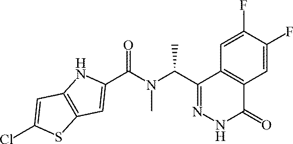 [SMILES: C[C@H](c1n[nH]c(=O)c2cc(F)c(F)cc12)N(C)C(=O)c1cc2sc(Cl)cc2[nH]1] | 0.03 |
| | (R)-2-chloro-N-(1-(6,7-difluoro-4-oxo-3,4-dihydrophthalazin-1-yl)ethyl)-N-methyl-4H-thieno[3,2-b]pyrrole-5-carboxamide | |
| 80 | 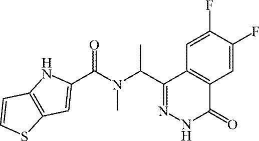 [SMILES: CC(c1n[nH]c(=O)c2cc(F)c(F)cc12)N(C)C(=O)c1cc2sccc2[nH]1] | 14.13 |
| | N-(1-(6,7-difluoro-4-oxo-3,4-dihydrophthalazin-1-yl)ethyl)-N-methyl-4H-thieno[3,2-b]pyrrole-5-carboxamide | |
| | enantiomer I | |
| 81 | 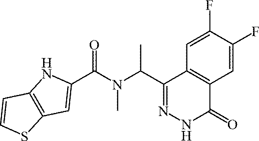 [SMILES: CC(c1n[nH]c(=O)c2cc(F)c(F)cc12)N(C)C(=O)c1cc2sccc2[nH]1] | 0.11 |
| | N-(1-(6,7-difluoro-4-oxo-3,4-dihydrophthalazin-1-yl)ethyl)-N-methyl-4H-thieno[3,2-b]pyrrole-5-carboxamide | |
| | enantiomer II | |
| 82 | 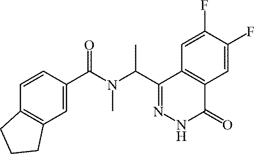 [SMILES: CC(c1n[nH]c(=O)c2cc(F)c(F)cc12)N(C)C(=O)c1ccc2c(c1)CCC2] | 9.01 |
| | N-(1-(6,7-difluoro-4-oxo-3,4-dihydrophthalazin-1-yl)ethyl)-N-methyl-2,3-dihydro-1H-indene-5-carboxamide | |
| | enantiomer I | |
| 83 | 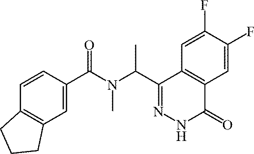 [SMILES: CC(c1n[nH]c(=O)c2cc(F)c(F)cc12)N(C)C(=O)c1ccc2c(c1)CCC2] | 0.08 |
| | N-(1-(6,7-difluoro-4-oxo-3,4-dihydrophthalazin-1-yl)ethyl)-N-methyl-2,3-dihydro-1H-indene-5-carboxamide | |
| | enantiomer II | |
| 84 | 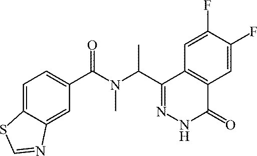 [SMILES: CC(c1n[nH]c(=O)c2cc(F)c(F)cc12)N(C)C(=O)c1ccc2scnc2c1] | >25 |
| | N-(1-(6,7-difluoro-4-oxo-3,4-dihydrophthalazin-1-yl)ethyl)-N-methylbenzo[d]thiazole-5-carboxamide | |
| | enantiomer I | |
| 85 | 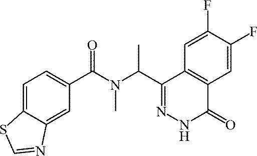 [SMILES: CC(c1n[nH]c(=O)c2cc(F)c(F)cc12)N(C)C(=O)c1ccc2scnc2c1] | 2.22 |
| | N-(1-(6,7-difluoro-4-oxo-3,4-dihydrophthalazin-1-yl)ethyl)-N-methylbenzo[d]thiazole-5-carboxamide | |
| | enantiomer II | |
| 86 | 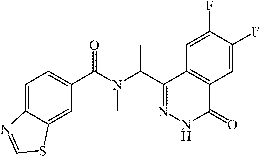 [SMILES: CC(c1n[nH]c(=O)c2cc(F)c(F)cc12)N(C)C(=O)c1ccc2ncsc2c1] | 25.0 |
| | N-(1-(6,7-difluoro-4-oxo-3,4-dihydrophthalazin-l-yl)ethyl)-N-methylbenzo[d]thiazole-6-carboxamide | |
| | enantiomer I | |
| 87 | 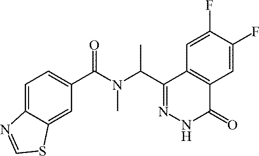 [SMILES: CC(c1n[nH]c(=O)c2cc(F)c(F)cc12)N(C)C(=O)c1ccc2ncsc2c1] | 5.57 |
| | N-(1-(6,7-difluoro-4-oxo-3,4-dihydrophthalazin-l-yl)ethyl)-N-methylbenzo[d]thiazole-6-carboxamide | |
| | enantiomer II | |
| 88 | 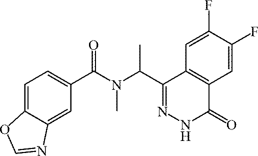 [SMILES: CC(c1n[nH]c(=O)c2cc(F)c(F)cc12)N(C)C(=O)c1ccc2ocnc2c1] | >25 |
| | N-(1-(6,7-difluoro-4-oxo-3,4-dihydrophthalazin-1-yl)ethyl)-N-methylbenzo[d]oxazole-5-carboxamide | |
| | enantiomer I | |
| 89 | 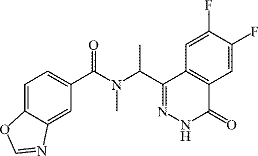 [SMILES: CC(c1n[nH]c(=O)c2cc(F)c(F)cc12)N(C)C(=O)c1ccc2ocnc2c1] | 2.42 |
| | N-(1-(6,7-difluoro-4-oxo-3,4-dihydrophthalazin-1-yl)ethyl)-N-methylbenzo[d]oxazole-5-carboxamide | |
| | enantiomer II | |
| 90 | 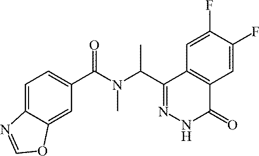 [SMILES: CC(c1n[nH]c(=O)c2cc(F)c(F)cc12)N(C)C(=O)c1ccc2ncoc2c1] | >25 |
| | N-(1-(6,7-difluoro-4-oxo-3,4-dihydrophthalazin-1-yl)ethyl)-N-methylbenzo[d]oxazole-6-carboxamide | |
| | enantiomer I | |
| 91 | 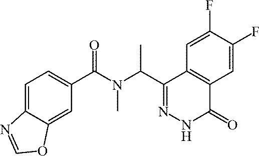 [SMILES: CC(c1n[nH]c(=O)c2cc(F)c(F)cc12)N(C)C(=O)c1ccc2ncoc2c1] | 3.86 |
| | N-(1-(6,7-difluoro-4-oxo-3,4-dihydrophthalazin-1-yl)ethyl)-N-methylbenzo[d]oxazole-6-carboxamide | |
| | enantiomer II | |
| 92 | 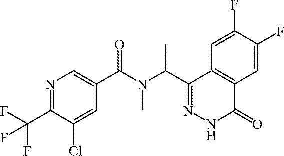 [SMILES: CC(c1n[nH]c(=O)c2cc(F)c(F)cc12)N(C)C(=O)c1cnc(C(F)(F)F)c(Cl)c1] | >25 |
| | 5-chloro-N-(1-(6,7-difluoro-4-oxo-3,4-dihydrophthalazin-1-yl)ethyl)-N-methyl-6-(trifluoromethyl)nicotinamide | |
| | enantiomer I | |
| 93 | 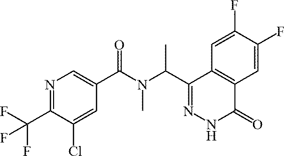 [SMILES: CC(c1n[nH]c(=O)c2cc(F)c(F)cc12)N(C)C(=O)c1cnc(C(F)(F)F)c(Cl)c1] | 1.66 |
| | 5-chloro-N-(1-(6,7-difluoro-4-oxo-3,4-dihydrophthalazin-1-yl)ethyl)-N-methyl-6-(trifluoromethyl)nicotinamide | |
| | enantiomer II | |
| 94 | 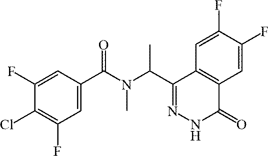 [SMILES: CC(c1n[nH]c(=O)c2cc(F)c(F)cc12)N(C)C(=O)c1cc(F)c(Cl)c(F)c1] | 13.30 |
| | 4-chloro-N-(1-(6,7-difluoro-4-oxo-3,4-dihydrophthalazin-1-yl)ethyl)-3,5-difluoro-N-methylbenzamide | |
| | enantiomer I | |
| 95 | 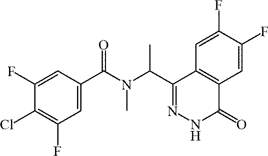 [SMILES: CC(c1n[nH]c(=O)c2cc(F)c(F)cc12)N(C)C(=O)c1cc(F)c(Cl)c(F)c1] | 0.08 |
| | 4-chloro-N-(1-(6,7-difluoro-4-oxo-3,4-dihydrophthalazin-1-yl)ethyl)-3,5-difluoro-N-methylbenzamide | |
| | enantiomer II | |
| 96 | 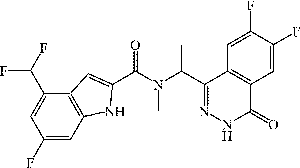 [SMILES: CC(c1n[nH]c(=O)c2cc(F)c(F)cc12)N(C)C(=O)c1cc2c(C(F)F)cc(F)cc2[nH]1] | 2.52 |
| | N-(1-(6,7-difluoro-4-oxo-3,4-dihydrophthalazin-1-yl)ethyl)-4-(difluoromethyl)-6-fluoro-N-methyl-1H-indole-2-carboxamide | |
| | enantiomer I | |
| 97 | 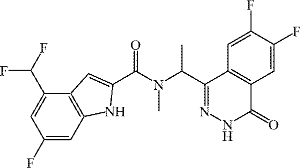 [SMILES: CC(c1n[nH]c(=O)c2cc(F)c(F)cc12)N(C)C(=O)c1cc2c(C(F)F)cc(F)cc2[nH]1] | 0.02 |
| | N-(1-(6,7-difluoro-4-oxo-3,4-dihydrophthalazin-1-yl)ethyl)-4-(difluoromethyl)-6-fluoro-N-methyl-1H-indole-2-carboxamide | |
| | enantiomer II | |
| 98 | 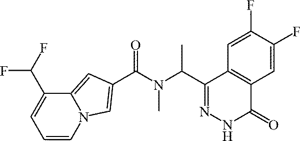 [SMILES: CC(c1n[nH]c(=O)c2cc(F)c(F)cc12)N(C)C(=O)c1cc2c(C(F)F)cccn2c1] | 7.44 |
| | N-(1-(6,7-difluoro-4-oxo-3,4-dihydrophthalazin-1-yl)ethyl)-8-(difluoromethyl)-N-methylindolizine-2-carboxamide | |
| | enantiomer I | |
| 99 | 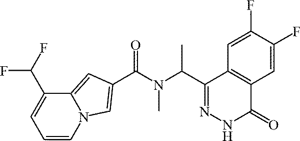 [SMILES: CC(c1n[nH]c(=O)c2cc(F)c(F)cc12)N(C)C(=O)c1cc2c(C(F)F)cccn2c1] | 0.01 |
| | N-(1-(6,7-difluoro-4-oxo-3,4-dihydrophthalazin-1-yl)ethyl)-8-(difluoromethyl)-N-methylindolizine-2-carboxamide | |
| | enantiomer II | |
| 100 | 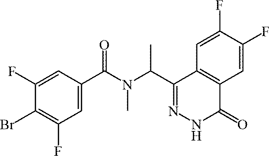 [SMILES: CC(c1n[nH]c(=O)c2cc(F)c(F)cc12)N(C)C(=O)c1cc(F)c(Br)c(F)c1] | 4.69 |
| | 4-bromo-N-(1-(6,7-difluoro-4-oxo-3,4-dihydrophthalazin-1-yl)ethyl)-3,5-difluoro-N-methylbenzamide | |
| | enantiomer I | |
| 101 | 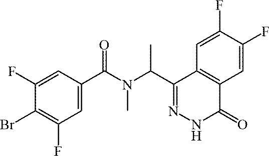 [SMILES: CC(c1n[nH]c(=O)c2cc(F)c(F)cc12)N(C)C(=O)c1cc(F)c(Br)c(F)c1] | 0.05 |
| | 4-bromo-N-(1-(6,7-difluoro-4-oxo-3,4-dihydrophthalazin-1-yl)ethyl)-3,5-difluoro-N-methylbenzamide | |
| | enantiomer II | |
| 102 | 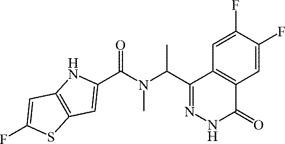 [SMILES: CC(c1n[nH]c(=O)c2cc(F)c(F)cc12)N(C)C(=O)c1cc2sc(F)cc2[nH]1] | 3.77 |
| | N-(1-(6,7-difluoro-4-oxo-3,4-dihydrophthalazin-1-yl)ethyl)-2-fluoro-N-methyl-4H-thieno[3,2-b]pyrrole-5-carboxamide | |
| | enantiomer I | |
| 103 | 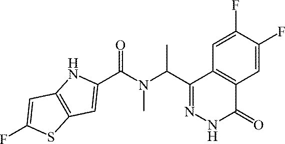 [SMILES: CC(c1n[nH]c(=O)c2cc(F)c(F)cc12)N(C)C(=O)c1cc2sc(F)cc2[nH]1] | 0.06 |
| | N-(1-(6,7-difluoro-4-oxo-3,4-dihydrophthalazin-1-yl)ethyl)-2-fluoro-N-methyl-4H-thieno[3,2-b]pyrrole-5-carboxamide | |
| | enantiomer II | |
| 104 | 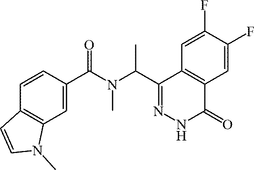 [SMILES: CC(c1n[nH]c(=O)c2cc(F)c(F)cc12)N(C)C(=O)c1ccc2ccn(C)c2c1] | >25 |
| | N-(1-(6,7-difluoro-4-oxo-3,4-dihydrophthalazin-1-yl)ethyl)-N,1-dimethyl-1H-indole-6-carboxamide | |
| | enantiomer I | |
| 105 | 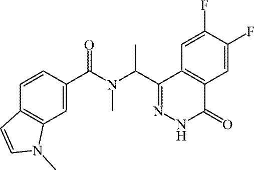 [SMILES: CC(c1n[nH]c(=O)c2cc(F)c(F)cc12)N(C)C(=O)c1ccc2ccn(C)c2c1] | 1.00 |
| | N-(1-(6,7-difluoro-4-oxo-3,4-dihydrophthalazin-1-yl)ethyl)-N,1-dimethyl-1H-indole-6-carboxamide | |
| | enantiomer II | |
| 106 | 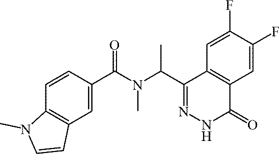 [SMILES: CC(c1n[nH]c(=O)c2cc(F)c(F)cc12)N(C)C(=O)c1ccc2c(ccn2C)c1] | 4.03 |
| | N-(1-(6,7-difluoro-4-oxo-3,4-dihydrophthalazin-1-yl)ethyl)-N,1-dimethyl-1H-indole-5-carboxamide | |
| | enantiomer I | |
| 107 | 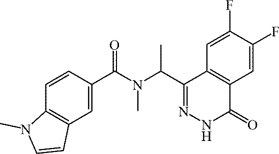 [SMILES: CC(c1n[nH]c(=O)c2cc(F)c(F)cc12)N(C)C(=O)c1ccc2c(ccn2C)c1] | 0.11 |
| | N-(1-(6,7-difluoro-4-oxo-3,4-dihydrophthalazin-1-yl)ethyl)-N,1-dimethyl-1H-indole-5-carboxamide | |
| | enantiomer II | |
| 108 | 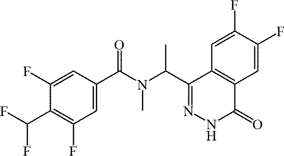 [SMILES: CC(c1n[nH]c(=O)c2cc(F)c(F)cc12)N(C)C(=O)c1cc(F)c(C(F)F)c(F)c1] | 9.95 |
| | N-(1-(6,7-difluoro-4-oxo-3,4-dihydrophthalazin-1-yl)ethyl)-4-(difluoromethyl)-3,5-difluoro-N-methylbenzamide | |
| | enantiomer I | |
| 109 | 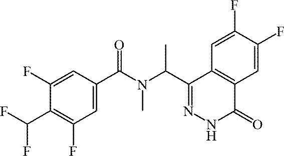 [SMILES: CC(c1n[nH]c(=O)c2cc(F)c(F)cc12)N(C)C(=O)c1cc(F)c(C(F)F)c(F)c1] | 0.11 |
| | N-(1-(6,7-difluoro-4-oxo-3,4-dihydrophthalazin-1-yl)ethyl)-4-(difluoromethyl)-3,5-difluoro-N-methylbenzamide | |
| | enantiomer II | |
| 110 | 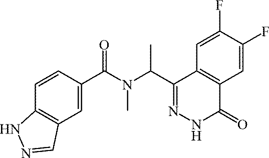 [SMILES: CC(c1n[nH]c(=O)c2cc(F)c(F)cc12)N(C)C(=O)c1ccc2[nH]ncc2c1] | >25 |
| | N-(1-(6,7-difluoro-4-oxo-3,4-dihydrophthalazin-1-yl)ethyl)-N-methyl-1H-indazole-5-carboxamide | |
| | enantiomer I | |
| 111 | 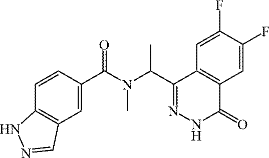 [SMILES: CC(c1n[nH]c(=O)c2cc(F)c(F)cc12)N(C)C(=O)c1ccc2[nH]ncc2c1] | 8.88 |
| | N-(1-(6,7-difluoro-4-oxo-3,4-dihydrophthalazin-1-yl)ethyl)-N-methyl-1H-indazole-5-carboxamide | |
| | enantiomer II | |
| 112 | 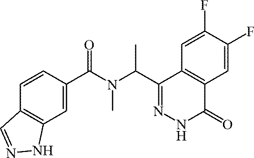 [SMILES: CC(c1n[nH]c(=O)c2cc(F)c(F)cc12)N(C)C(=O)c1ccc2cn[nH]c2c1] | >25 |
| | N-(1-(6,7-difluoro-4-oxo-3,4-dihydrophthalazin-1-yl)ethyl)-N-methyl-1H-indazole-6-carboxamide | |
| | enantiomer I | |
| 113 | 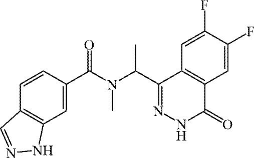 [SMILES: CC(c1n[nH]c(=O)c2cc(F)c(F)cc12)N(C)C(=O)c1ccc2cn[nH]c2c1] | 2.49 |
| | N-(1-(6,7-difluoro-4-oxo-3,4-dihydrophthalazin-1-yl)ethyl)-N-methyl-1H-indazole-6-carboxamide | |
| | enantiomer II | |
| 114 | 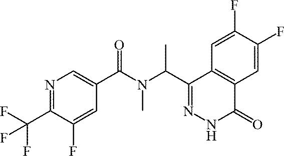 [SMILES: CC(c1n[nH]c(=O)c2cc(F)c(F)cc12)N(C)C(=O)c1cnc(C(F)(F)F)c(F)c1] | 4.18 |
| | N-(1-(6,7-difluoro-4-oxo-3,4-dihydrophthalazin-1-yl)ethyl)-5-fluoro-N-methyl-6-(trifluoromethyl)nicotinamide | |

### Enumerated Embodiments

The following exemplary embodiments are provided, the numbering of which is not to be construed as designating levels of importance:

Embodiment 1 provides a compound of formula (Ia) or (Ib), or a salt, solvate, prodrug, stereoisomer, tautomer, or isotopically labelled derivative thereof, or any mixtures thereof:

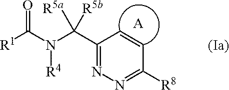

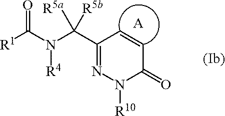

wherein in (Ia) or (Ib): ring A

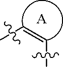

is selected from the group consisting of:

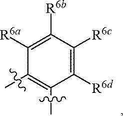

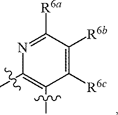

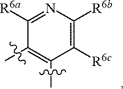

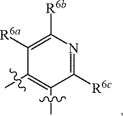

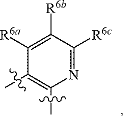

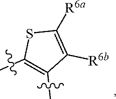

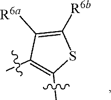

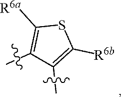

(wherein there is no bridgehead double bond in the bicyclic structure including ring A),

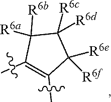

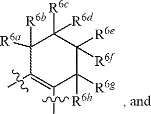

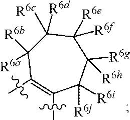

or ring A is absent and

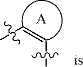

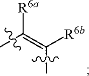

R¹ is selected from the group consisting of —NR²R³,

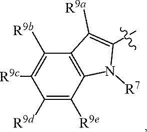

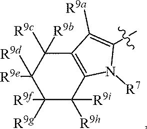

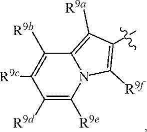

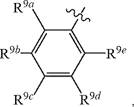

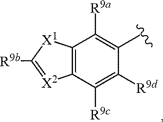

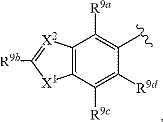

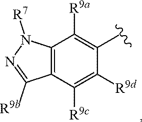

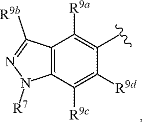

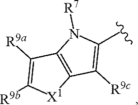

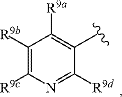

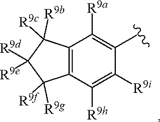

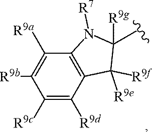

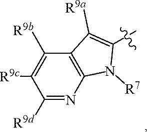

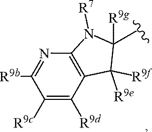

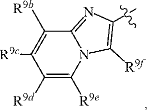

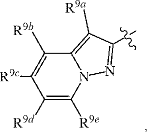

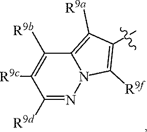

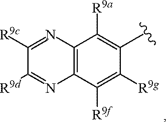

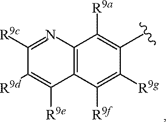

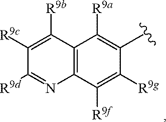

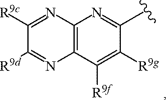

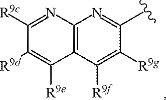

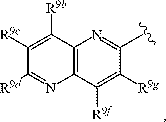

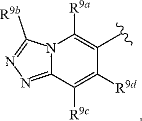

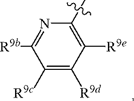

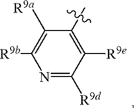

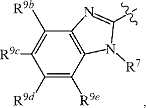

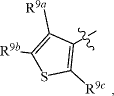

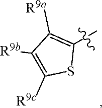

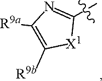

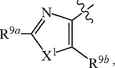

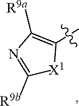

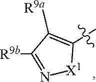

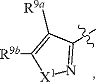

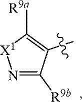

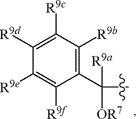

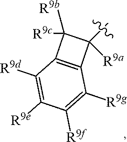

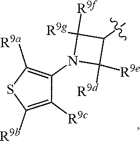

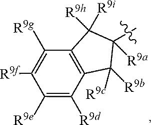

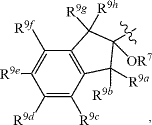

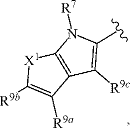

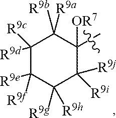

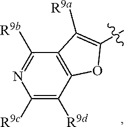

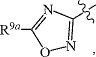

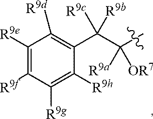

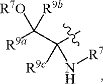

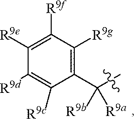

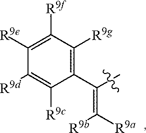

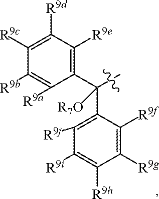

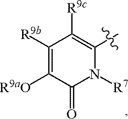

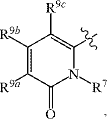

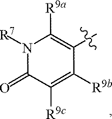

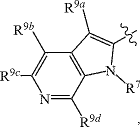

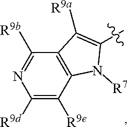

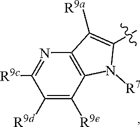

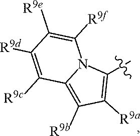

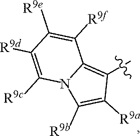

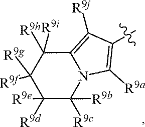

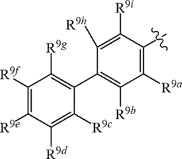

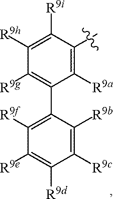

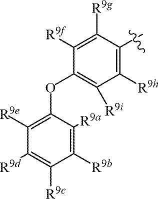

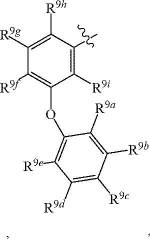

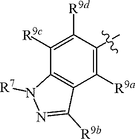

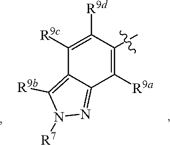

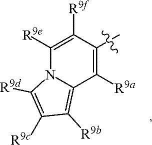

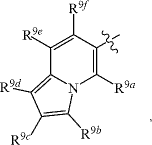

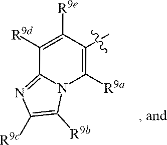

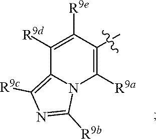

X¹ is selected from the group consisting of O, S, and N(R⁷);
X² is selected from the group consisting of N and CR^{9e};
R² is selected from the group consisting of optionally substituted C₃-C₈ cycloalkyl, optionally substituted phenyl, optionally substituted benzyl, optionally substituted heteroaryl, and —(CH₂)(optionally substituted heteroaryl);
R³ is selected from the group consisting of H and optionally substituted C₁-C₆ alkyl;
R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁷, —OC(═O)R⁷, —SR⁷, —S(═O)R⁷, —S(═O)₂R⁷, —S(═O)₂NR⁷R⁷, —S(═O)₂NHC(═O)NHR⁷, —N(R⁷)S(═O)₂R⁷, —N(R⁷)C(═O)R⁷, —C(═0)NR⁷R⁷, and —NR⁷R⁷;
R^{5a} is selected from the group consisting of H and optionally substituted C₁-C₆ alkyl;
R^{5b} is selected from the group consisting of H and optionally substituted C₁-C₆ alkyl;
each occurrence of R^{6a}, R^{6b}, R^{6c}, R^{6d}, R^{6e}, R^{6f}, R^{6g}, R^{6h}, R⁶ⁱ, and R^{6j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁷ is independently selected from the group consisting of H, optionally substituted C₁-C₆ alkyl, optionally substituted C₃-C₈ cycloalkyl, optionally substituted phenyl, and optionally substituted hetereoaryl;
R⁸ is selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃—Cs cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, R^{9d}, R^{9e}, R^{9f}, R^{9g}, R^{9h}, R⁹ⁱ, and R^{9j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, -S(optionally substituted C₁-C₆ alkyl), —SO(optionally substituted C₁-C₆ alkyl), -SO₂(optionally substituted C₁-C₆ alkoxy), —C(═O)OH, —C(═O)O(optionally substituted C₁-C₆ alkyl), —C(═O)O(optionally substituted C₃—Cs cycloalkyl), —O(optionally substituted C₁-C₆ alkyl), -O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
R¹⁰ is selected from the group consisting of H, C₁-C₆ alkyl, optionally substituted C₃-C₈ cycloalkyl, optionally substituted C₁-C₆ alkoxy, and optionally substituted C₃-C₈ cycloalkoxy.

Embodiment 2 provides the compound of Embodiment 1, which is at least one of:

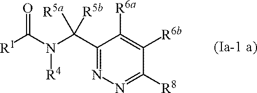

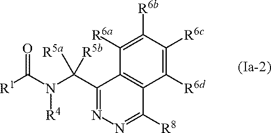

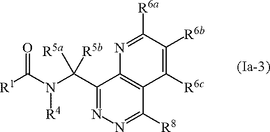

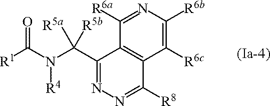

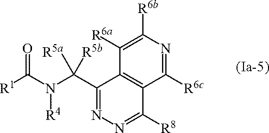

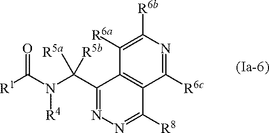

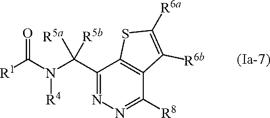

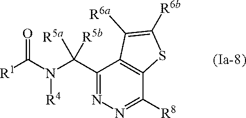

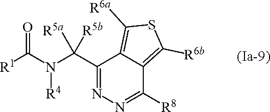

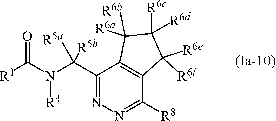

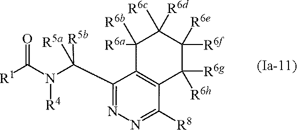

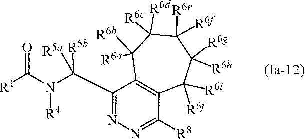

Embodiment 3 provides the compound of Embodiment 1, which is at least one of:

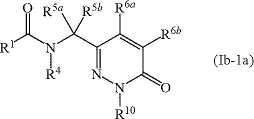

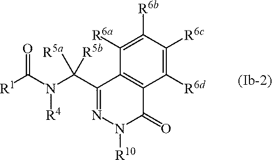

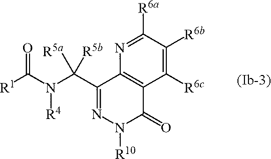

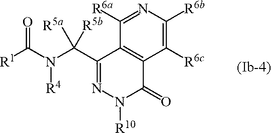

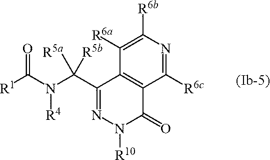

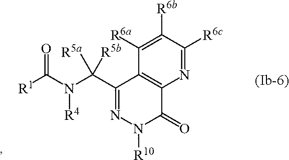

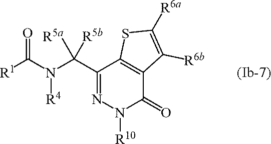

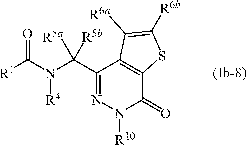

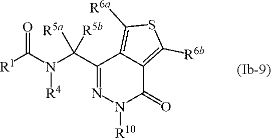

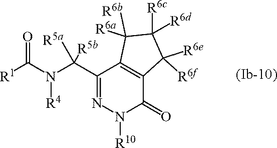

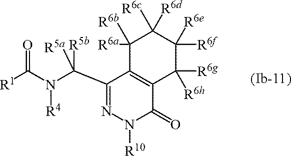

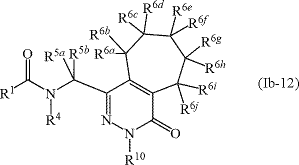

Embodiment 4 provides the compound of any of Embodiments 1-3, which is at least one of:

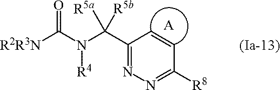

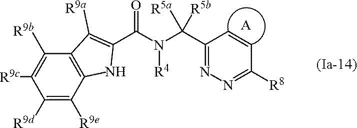

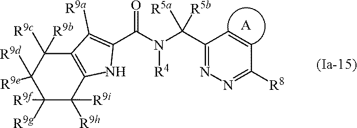

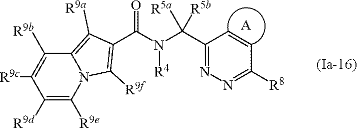

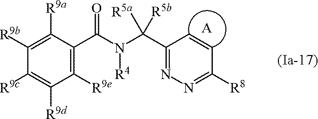

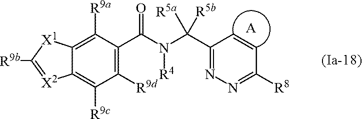

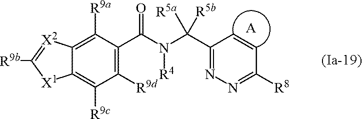

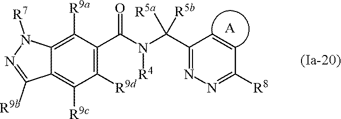

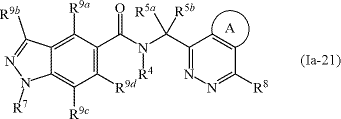

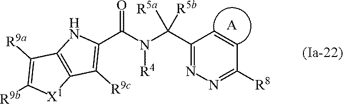

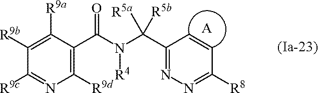

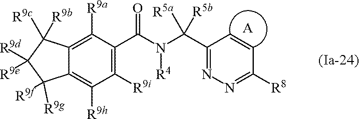

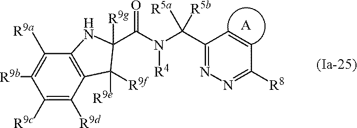

Embodiment 5 provides the compound of any of Embodiments 1-4, which is at least one of:

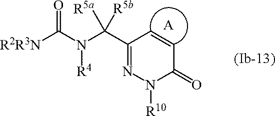

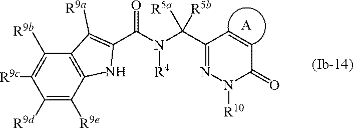

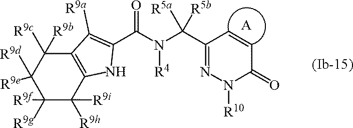

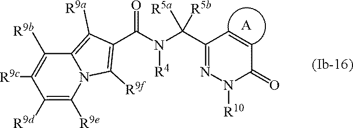

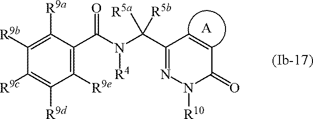

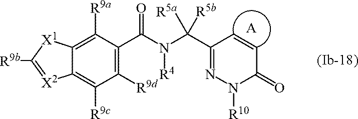

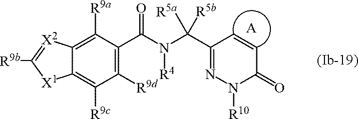

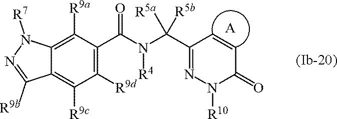

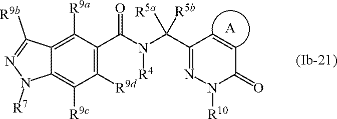

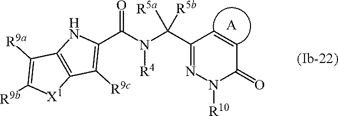

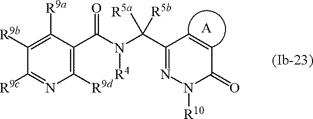

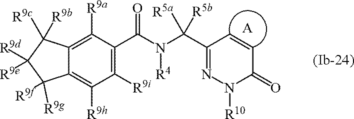

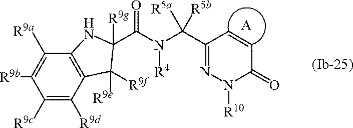

Embodiment 6 provides the compound of any of Embodiments 1-5, which is at least one of:

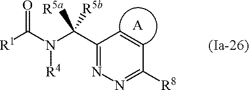

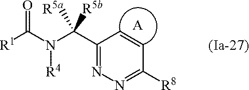

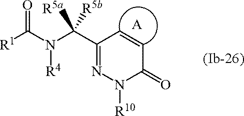

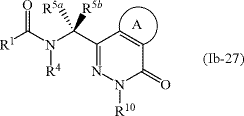

Embodiment 7 provides the compound of any of Embodiments 1-6, which is at least one of:

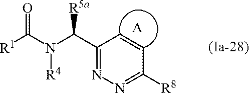

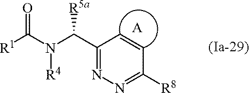

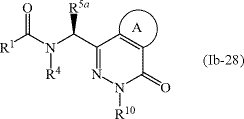

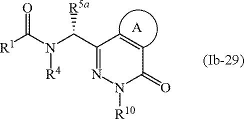

Embodiment 8 provides the compound of any of Embodiments 1-7, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

Embodiment 9 provides the compound of any of Embodiments 1-8, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 10 provides the compound of any of Embodiments 1-9, wherein ring A is selected from the group consisting of:

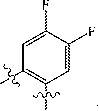

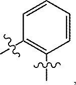

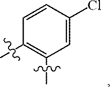

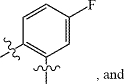

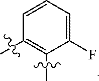

Embodiment 11 provides the compound of any of Embodiments 1-10, wherein R² is phenyl optionally substituted with at least one selected from the group consisting of C₁-C₆ alkyl, halo, C₁-C₃ haloalkyl, and —CN.

Embodiment 12 provides the compound of any of Embodiments 1-11, wherein R² is selected from the group consisting of phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, and 4-difluoromethyl-3-fluorophenyl.

Embodiment 13 provides the compound of any of Embodiments 1-12, wherein R³ is selected from the group consisting of H and methyl.

Embodiment 14 provides the compound of any of Embodiments 1-13, wherein R⁴ is selected from the group consisting of methyl, sec-butyl,

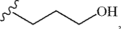

and

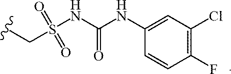

Embodiment 15 provides the compound of any of Embodiments 1-14, wherein R¹ is selected from the group consisting of:

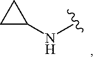

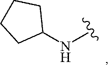

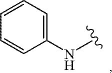

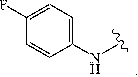

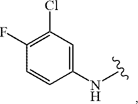

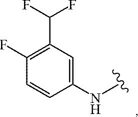

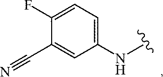

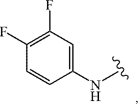

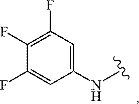

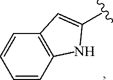

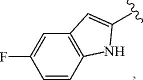

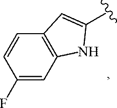

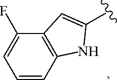

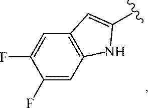

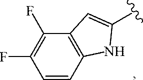

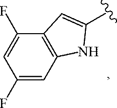

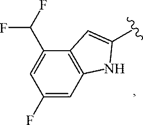

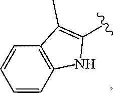

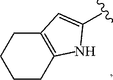

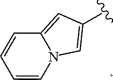

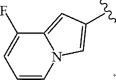

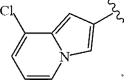

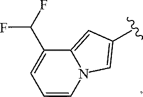

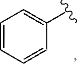

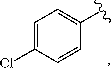

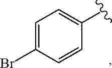

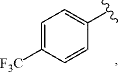

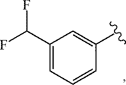

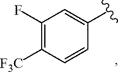

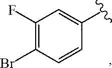

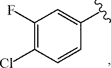

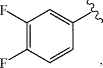

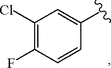

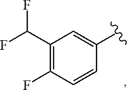

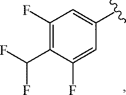

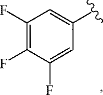

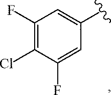

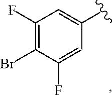

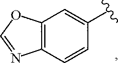

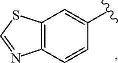

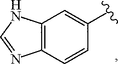

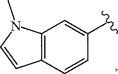

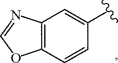

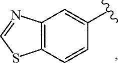

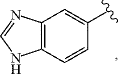

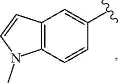

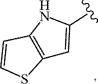

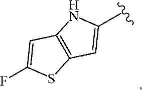

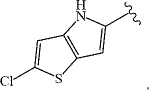

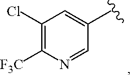

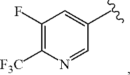

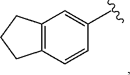

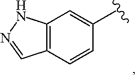

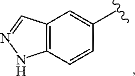

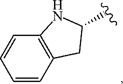

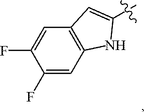

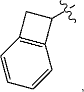

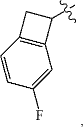

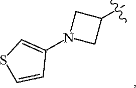

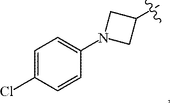

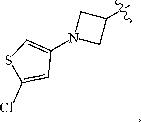

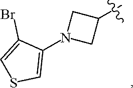

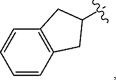

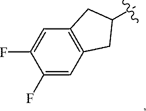

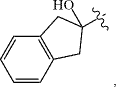

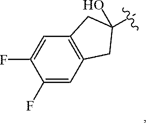

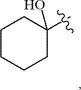

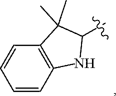

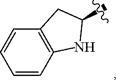

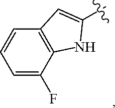

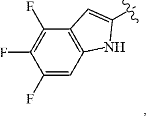

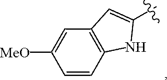

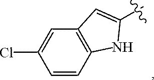

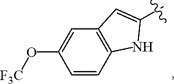

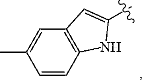

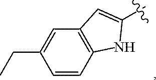

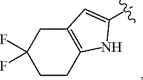

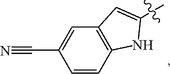

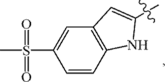

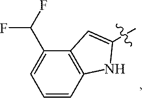

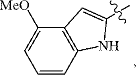

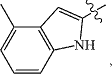

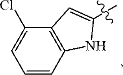

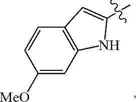

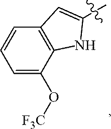

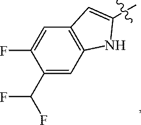

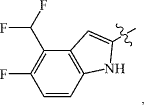

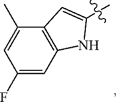

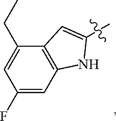

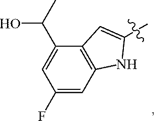

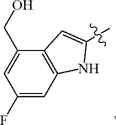

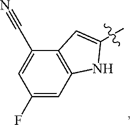

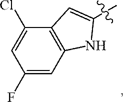

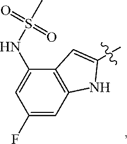

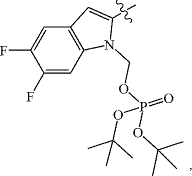

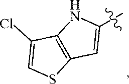

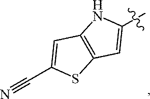

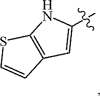

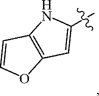

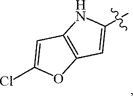

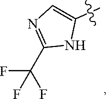

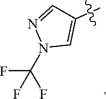

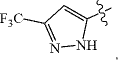

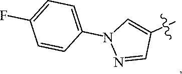

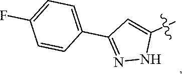

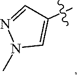

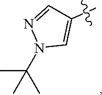

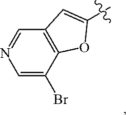

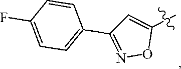

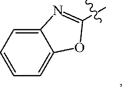

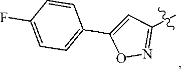

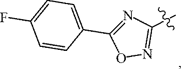

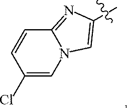

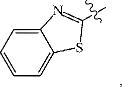

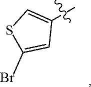

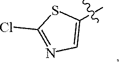

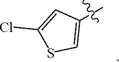

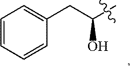

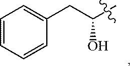

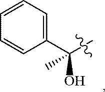

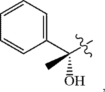

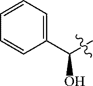

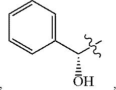

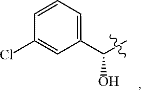

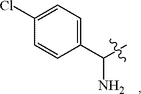

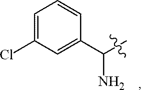

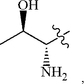

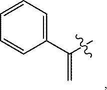

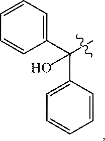

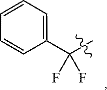

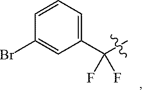

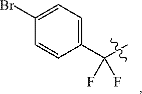

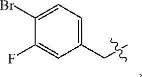

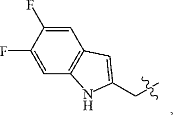

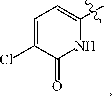

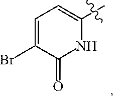

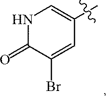

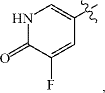

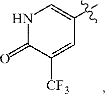

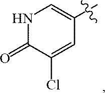

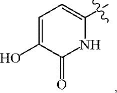

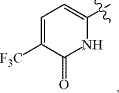

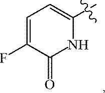

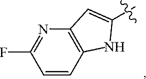

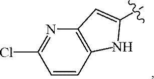

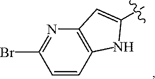

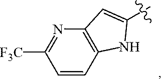

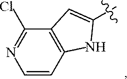

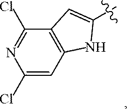

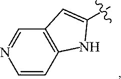

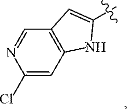

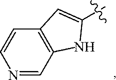

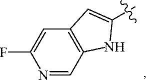

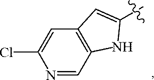

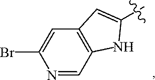

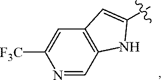

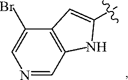

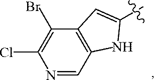

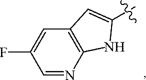

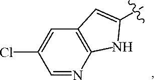

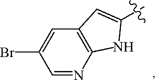

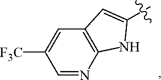

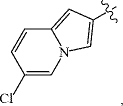

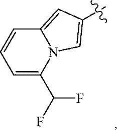

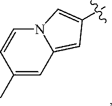

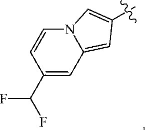

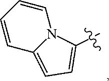

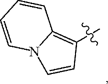

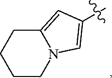

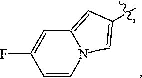

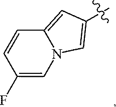

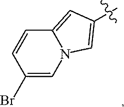

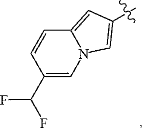

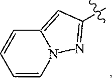

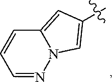

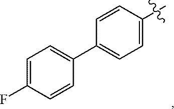

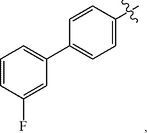

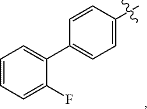

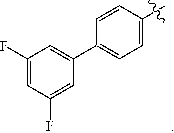

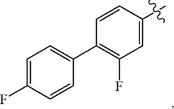

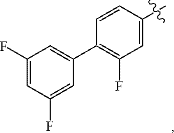

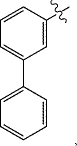

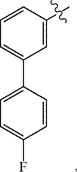

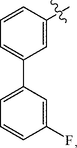

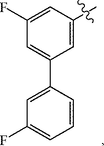

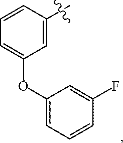

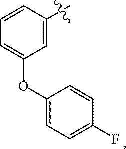

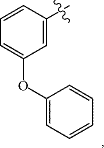

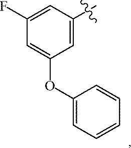

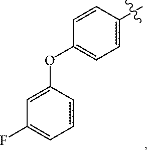

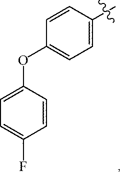

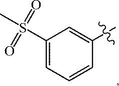

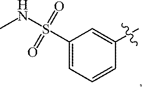

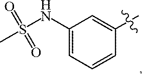

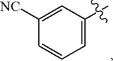

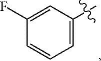

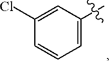

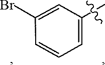

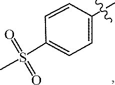

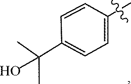

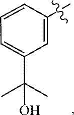

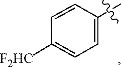

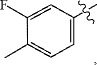

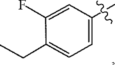

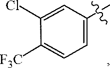

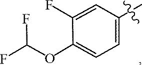

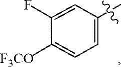

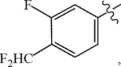

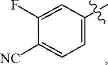

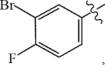

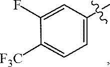

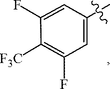

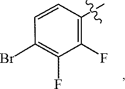

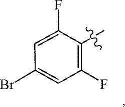

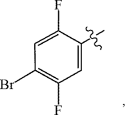

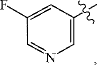

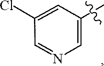

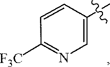

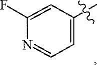

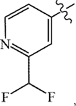

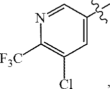

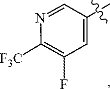

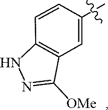

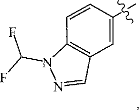

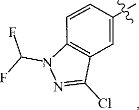

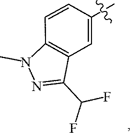

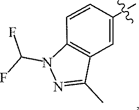

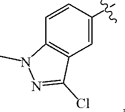

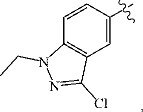

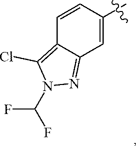

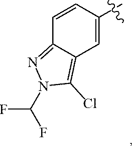

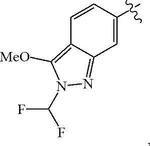

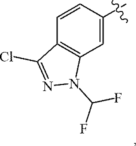

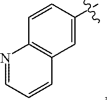

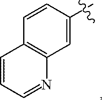

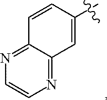

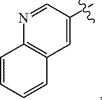

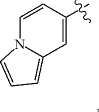

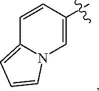

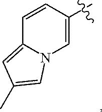

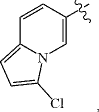

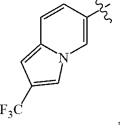

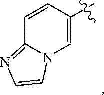

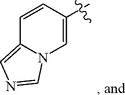

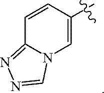

Embodiment 16 provides the compound of any of Embodiments 1-15, which is at least one selected from the group consisting of:
3-(3-chloro-4-fluorophenyl)-1-methyl-1-(1-(4-oxo-3,4-dihydrophthalazin-1-yl)ethyl)urea;
3-(3-chloro-4-fluorophenyl)-1-isobutyl-1-(1-(4-oxo-3,4-dihydrophthalazin-1-yl)ethyl)urea;
3-(3-chloro-4-fluorophenyl)-1-isobutyl-1-(1-(3-methyl-4-oxo-3,4-dihydrophthalazin-1-yl)ethyl)urea;
3-(4-fluorophenyl)-1-isobutyl-1-(1-(3-methyl-4-oxo-3,4-dihydrophthalazin-1-yl)ethyl)urea;
3-(3-chloro-4-fluorophenyl)-1-(1-(6-chloro-4-oxo-3,4-dihydrophthalazin-1-yl)ethyl)-1-isobutylurea;
1-(1-(6-chloro-4-oxo-3,4-dihydrophthalazin-1-yl)ethyl)-3-(4-fluorophenyl)-1-isobutylurea;
3-(3-chloro-4-fluorophenyl)-1-(1-(6-chloro-4-oxo-3,4-dihydrophthalazin-1-yl)ethyl)-1-methylurea;
3-(3-chloro-4-fluorophenyl)-1-(1-(6-chloro-4-oxo-3,4-dihydrophthalazin-1-yl)ethyl)-1-(3-hydroxypropyl)urea;
1-(1-(6-chloro-4-oxo-3,4-dihydrophthalazin-1-yl)ethyl)-3-(4-fluorophenyl)-1-(3-hydroxypropyl)urea;
3-(3-chloro-4-fluorophenyl)-1-(1-(6-fluoro-4-oxo-3,4-dihydrophthalazin-1-yl)ethyl)-1-isobutylurea;
1-(1-(6-fluoro-4-oxo-3,4-dihydrophthalazin-1-yl)ethyl)-3-(4-fluorophenyl)-1-isobutylurea;
3-(3-chloro-4-fluorophenyl)-1-(1-(6-fluoro-4-oxo-3,4-dihydrophthalazin-1-yl)ethyl)-1-methylurea;
1-(1-(6-fluoro-4-oxo-3,4-dihydrophthalazin-1-yl)ethyl)-3-(4-fluorophenyl)-1-methylurea;
3-(4-fluorophenyl)-1-isobutyl-1-(1-(4-oxo-3,4-dihydrophthalazin-1-yl)ethyl)urea;
3-(3-chloro-4-fluorophenyl)-1-(1-(6,7-difluoro-3-methyl-4-oxo-3,4-dihydrophthalazin-1-yl)ethyl)-1-methylurea;
3-(3-chloro-4-fluorophenyl)-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-3-(4-fluorophenyl)-1-isobutylurea;
3-(3-chloro-4-fluorophenyl)-1-(1-(6,7-difluoro-3-methyl-4-oxo-3,4-dihydrophthalazin-1-yl)ethyl)-1-(3-hydroxypropyl)urea;
3-(4-fluorophenyl)-1-(1-(6,7-difluoro-3-methyl-4-oxo-3,4-dihydrophthalazin-1-yl)ethyl)-1-(3-hydroxypropyl)urea;
3-(3-chloro-4-fluorophenyl)-1-(1-(6,7-difluoro-4-oxo-3,4-dihydrophthalazin-1-yl)ethyl)-1-isobutylurea;
1-(1-(6,7-difluoro-4-oxo-3,4-dihydrophthalazin-1-yl)ethyl)-1-isobutyl-3-phenylurea;
3-(3-chloro-4-fluorophenyl)-1-(1-(6,7-difluoro-4-oxo-3,4-dihydrophthalazin-1-yl)ethyl)-1-methylurea;
2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide;
3-cyclopropyl-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-1-isobutyl-3-phenylurea;
3 -cyclopentyl-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-3-(4-fluorophenyl)-1-methylurea;
1-(1-(6,7-difluoro-4-oxo-3,4-dihydrophthalazin-1-yl)ethyl)-3-(3,4-difluorophenyl)-1-methylurea;
1-(1-(6,7-difluoro-4-oxo-3,4-dihydrophthalazin-1-yl)ethyl)-1-methyl-3-(3,4,5-trifluorophenyl)urea;
1-(1-(6,7-difluoro-4-oxo-3,4-dihydrophthalazin-1-yl)ethyl)-3-(4-fluorophenyl)-1-methylurea;
3-(3-chloro-4-fluorophenyl)-1-(1-(5-fluoro-4-oxo-3,4-dihydrophthalazin-1-yl)ethyl)-1-methylurea;
3-(3,4-difluorophenyl)-1-(1-(5-fluoro-4-oxo-3,4-dihydrophthalazin-1-yl)ethyl)-1-methylurea;
N-(1-(6,7-difluoro-4-oxo-3,4-dihydrophthalazin-1-yl)ethyl)-N-methyl-1H-indole-2-carboxamide;
N-(1-(6,7-difluoro-4-oxo-3,4-dihydrophthalazin-1-yl)ethyl)-N-ethyl-5-fluoro-1H-indole-2-carboxamide;
N-(1-(6,7-difluoro-4-oxo-3,4-dihydrophthalazin-1-yl)ethyl)-N-ethyl-1H-indole-2-carboxamide;
N-(1-(6,7-difluoro-4-oxo-3,4-dihydrophthalazin-1-yl)ethyl)-N-ethyl-4,5-difluoro-1H-indole-2-carboxamide;
N-(1-(6,7-difluoro-4-oxo-3,4-dihydrophthalazin-1-yl)ethyl)-5,6-difluoro-N-methyl-1H-indole-2-carboxamide;
N-(1-(6,7-difluoro-4-oxo-3,4-dihydrophthalazin-1-yl)ethyl)-5-fluoro-N-methyl-1H-indole-2-carboxamide;
N-(1-(6,7-difluoro-4-oxo-3,4-dihydrophthalazin-1-yl)ethyl)-6-fluoro-N-methyl-1H-indole-2-carboxamide;
N-(1-(6,7-difluoro-4-oxo-3,4-dihydrophthalazin-1-yl)ethyl)-4-fluoro-N-methyl-1H-indole-2-carboxamide;
N-(1-(6,7-difluoro-4-oxo-3,4-dihydrophthalazin-1-yl)ethyl)-4,5-difluoro-N-methyl-1H-indole-2-carboxamide;
N-(1-(6,7-difluoro-4-oxo-3,4-dihydrophthalazin-1-yl)ethyl)-N,3-dimethyl-1H-indole-2-carboxamide;
N-(1-(6,7-difluoro-4-oxo-3,4-dihydrophthalazin-1-yl)ethyl)-N-isobutyl-1H-indole-2-carboxamide;
N-(1-(6,7-difluoro-4-oxo-3,4-dihydrophthalazin-1-yl)ethyl)-N-methyl-4,5,6,7-tetrahydro-1H-indole-2-carboxamide;
N-(1-(6,7-difluoro-4-oxo-3,4-dihydrophthalazin-1-yl)ethyl)-N-methylindolizine-2-carboxamide;
N-(1-(6,7-difluoro-4-oxo-3,4-dihydrophthalazin-1-yl)ethyl)-4,6-difluoro-N-methyl-1H-indole-2-carboxamide;
N-(1-(6,7-difluoro-4-oxo-3,4-dihydrophthalazin-1-yl)ethyl)-3-fluoro-N-methyl-4-(trifluoromethyl)benzamide;
4-bromo-N-(1-(6,7-difluoro-4-oxo-3,4-dihydrophthalazin-1-yl)ethyl)-3-fluoro-N-methylbenzamide;
4-chloro-N-(1-(6,7-difluoro-4-oxo-3,4-dihydrophthalazin-1-yl)ethyl)-3-fluoro-N-methylbenzamide;
4-bromo-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-N-methyl-4-(trifluoromethyl)benzamide;
N-(1-(6,7-difluoro-4-oxo-3,4-dihydrophthalazin-1-yl)ethyl)-3,4,5-trifluoro-N-methylbenzamide;
3-chloro-N-(1-(6,7-difluoro-4-oxo-3,4-dihydrophthalazin-1-yl)ethyl)-4-fluoro-N-methylbenzamide;
4-chloro-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-3,4-difluoro-N-methylbenzamide;
N-(1-(6,7-difluoro-4-oxo-3,4-dihydrophthalazin-1-yl)ethyl)-3-(difluoromethyl)-N-methylbenzamide;
N-(1-(6,7-difluoro-4-oxo-3,4-dihydrophthalazin-1-yl)ethyl)-3-(difluoromethyl)-4-fluoro-N-methylbenzamide;
1-(1-(6,7-difluoro-4-oxo-3,4-dihydrophthalazin-1-yl)ethyl)-3 -(3 -(difluoromethyl)-4-fluorophenyl)-1-methylurea;
N-(1-(6,7-difluoro-4-oxo-3,4-dihydrophthalazin-1-yl)ethyl)-N-methylbenzamide;
8-chloro-N-(1-(6,7-difluoro-4-oxo-3,4-dihydrophthalazin-1-yl)ethyl)-N-methylindolizine-2-carboxamide;
3-(3-cyano-4-fluorophenyl)-1-(1-(6,7-difluoro-4-oxo-3,4-dihydrophthalazin-1-yl)ethyl)-1-methylurea;
N-(1-(6,7-difluoro-4-oxo-3,4-dihydrophthalazin-1-yl)ethyl)-8-fluoro-N-methylindolizine-2-carboxamide;
N-(1-(6,7-difluoro-4-oxo-3,4-dihydrophthalazin-1-yl)ethyl)-N-methylindoline-2-carboxamide;
2-chloro-N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-2,3-dihydro-1H-indene-5-carboxamide;
N-(1-(6,7-difluoro-4-oxo-3,4-dihydrophthalazin-1-yl)ethyl)-N-methylbenzo[d]thiazole-5-carboxamide;
N-(1-(6,7-difluoro-4-oxo-3,4-dihydrophthalazin-1-yl)ethyl)-N-methylbenzo[d]thiazole-6-carboxamide;
N-(1-(6,7-difluoro-4-oxo-3,4-dihydrophthalazin-1-yl)ethyl)-N-methylbenzo[d]oxazole-5-carboxamide;
N-(1-(6,7-difluoro-4-oxo-3,4-dihydrophthalazin-1-yl)ethyl)-N-methylbenzo[d]oxazole-6-carboxamide;
5-chloro-N-(1-(6,7-difluoro-4-oxo-3,4-dihydrophthalazin-1-yl)ethyl)-N-methyl-6-(trifluoromethyl)nicotinamide;
4-chloro-N-(1-(6,7-difluoro-4-oxo-3,4-dihydrophthalazin-1-yl)ethyl)-3, 5-difluoro-N-methylbenzamide;
N-(1-(6,7-difluoro-4-oxo-3,4-dihydrophthalazin-1-yl)ethyl)-4-(difluoromethyl)-6-fluoro-N-methyl-1H-indole-2-carboxamide;
N-(1-(6,7-difluoro-4-oxo-3,4-dihydrophthalazin-1-yl)ethyl)-8-(difluoromethyl)-N-methylindolizine-2-carboxamide;
4-bromo-N-(1-(6,7-difluoro-4-oxo-3,4-dihydrophthalazin-1-yl)ethyl)-3,5-difluoro-N-methylbenzamide;
N-(1-(6,7-difluoro-4-oxo-3,4-dihydrophthalazin-1-yl)ethyl)-2-fluoro-N-methyl-4H-thieno[3,2-b]pyrrole-5-carboxamide;
N-(1-(6,7-difluoro-4-oxo-3,4-dihydrophthalazin-1-yl)ethyl)-N,1-dimethyl-1H-indole-6-carboxamide;
N-(1-(6,7-difluoro-4-oxo-3,4-dihydrophthalazin-1-yl)ethyl)-N,1-dimethyl-1H-indole-5-carboxamide;
N-(1-(6,7-difluoro-4-oxo-3,4-dihydrophthalazin-1-yl)ethyl)-4-(difluoromethyl)-3,5-difluoro-N-methylbenzamide;
N-(1-(6,7-difluoro-4-oxo-3,4-dihydrophthalazin-1-yl)ethyl)-N-methyl-1H-indazole-5-carboxamide;
N-(1-(6,7-difluoro-4-oxo-3,4-dihydrophthalazin-1-yl)ethyl)-N-methyl-1H-indazole-6-carboxamide;
N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled derivative, stereoisomer, or tautomer thereof, or any mixtures thereof.

Embodiment 17 provides the compound of any of Embodiments 1-16, which is at least one selected from the group consisting of:
(R)-3-(3-chloro-4-fluorophenyl)-1-methyl-1-(1-(4-oxo-3,4-dihydrophthalazin-1-yl)ethyl)urea;
(S)-3-(3-chloro-4-fluorophenyl)-1-methyl-1-(1-(4-oxo-3,4-dihydrophthalazin-1-yl)ethyl)urea;
(R)-3-(3-chloro-4-fluorophenyl)-1-isobutyl-1-(1-(4-oxo-3,4-dihydrophthalazin-1-yl)ethyl)urea;
(S)-3-(3-chloro-4-fluorophenyl)-1-isobutyl-1-(1-(4-oxo-3,4-dihydrophthalazin-1-yl)ethyl)urea;
(R)-3-(3-chloro-4-fluorophenyl)-1-isobutyl-1-(1-(3-methyl-4-oxo-3,4-dihydrophthalazin-1-yl)ethyl)urea;
(S)-3-(3-chloro-4-fluorophenyl)-1-isobutyl-1-(1-(3-methyl-4-oxo-3,4-dihydrophthalazin-1-yl)ethyl)urea;
(R)-3-(4-fluorophenyl)-1-isobutyl-1-(1-(3-methyl-4-oxo-3,4-dihydrophthalazin-1-yl)ethyl)urea;
(S)-3-(4-fluorophenyl)-1-isobutyl-1-(1-(3-methyl-4-oxo-3,4-dihydrophthalazin-1-yl)ethyl)urea;
(R)-3-(3-chloro-4-fluorophenyl)-1-(1-(6-chloro-4-oxo-3,4-dihydrophthalazin-1-yl)ethyl)-1-isobutylurea;
(S)-3-(3-chloro-4-fluorophenyl)-1-(1-(6-chloro-4-oxo-3,4-dihydrophthalazin-1-yl)ethyl)-1-isobutylurea;
(R)-1-(1-(6-chloro-4-oxo-3,4-dihydrophthalazin-1-yl)ethyl)-3-(4-fluorophenyl)-1-isobutylurea;
(S)-1-(1-(6-chloro-4-oxo-3,4-dihydrophthalazin-1-yl)ethyl)-3-(4-fluorophenyl)-1-isobutylurea;
(R)-3-(3-chloro-4-fluorophenyl)-1-(1-(6-chloro-4-oxo-3,4-dihydrophthalazin-1-yl)ethyl)-1-methylurea;
(S)-3-(3-chloro-4-fluorophenyl)-1-(1-(6-chloro-4-oxo-3,4-dihydrophthalazin-1-yl)ethyl)-1-methylurea;
(R)-3-(3-chloro-4-fluorophenyl)-1-(1-(6-chloro-4-oxo-3,4-dihydrophthalazin-1-yl)ethyl)-1-(3 -hydroxypropyl)urea;
(S)-3-(3-chloro-4-fluorophenyl)-1-(1-(6-chloro-4-oxo-3,4-dihydrophthalazin-1-yl)ethyl)-1-(3 -hydroxypropyl)urea;
(R)-1-(1-(6-chloro-4-oxo-3,4-dihydrophthalazin-1-yl)ethyl)-3 -(4-fluorophenyl)-1-(3-hydroxypropyl)urea;
(S)-1-(1-(6-chloro-4-oxo-3,4-dihydrophthalazin-1-yl)ethyl)-3-(4-fluorophenyl)-1-(3-hydroxypropyl)urea;
(R)-3-(3-chloro-4-fluorophenyl)-1-(1-(6-fluoro-4-oxo-3,4-dihydrophthalazin-1-yl)ethyl)-1-isobutylurea;
(S)-3-(3-chloro-4-fluorophenyl)-1-(1-(6-fluoro-4-oxo-3,4-dihydrophthalazin-1-yl)ethyl)-1-isobutylurea;
(R)-1-(1-(6-fluoro-4-oxo-3,4-dihydrophthalazin-1-yl)ethyl)-3-(4-fluorophenyl)-1-isobutylurea;
(S)-1-(1-(6-fluoro-4-oxo-3,4-dihydrophthalazin-1-yl)ethyl)-3-(4-fluorophenyl)-1-isobutylurea;
(R)-3-(3-chloro-4-fluorophenyl)-1-(1-(6-fluoro-4-oxo-3,4-dihydrophthalazin-1-yl)ethyl)-1-methylurea;
(S)-3-(3-chloro-4-fluorophenyl)-1-(1-(6-fluoro-4-oxo-3,4-dihydrophthalazin-1-yl)ethyl)-1-methylurea;
(R)-1-(1-(6-fluoro-4-oxo-3,4-dihydrophthalazin-1-yl)ethyl)-3-(4-fluorophenyl)-1-methylurea;
(S)-1-(1-(6-fluoro-4-oxo-3,4-dihydrophthalazin-1-yl)ethyl)-3-(4-fluorophenyl)-1-methylurea;
(R)-3-(4-fluorophenyl)-1-isobutyl-1-(1-(4-oxo-3,4-dihydrophthalazin-1-yl)ethyl)urea;
(S)-3-(4-fluorophenyl)-1-isobutyl-1-(1-(4-oxo-3,4-dihydrophthalazin-1-yl)ethyl)urea;
(R)-3-(3-chloro-4-fluorophenyl)-1-(1-(6,7-difluoro-3-methyl-4-oxo-3,4-dihydrophthalazin-1-yl)ethyl)-1-methylurea;
(S)-3-(3-chloro-4-fluorophenyl)-1-(1-(6,7-difluoro-3-methyl-4-oxo-3,4-dihydrophthalazin-1-yl)ethyl)-1-methylurea;
(R)-3-(3-chloro-4-fluorophenyl)-1-(1-(6,7-difluoro-3-methyl-4-oxo-3,4-dihydrophthalazin-1-yl)ethyl)-1-isobutylurea;
(S)-3-(3-chloro-4-fluorophenyl)-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthalazin-1-yl)ethyl)-3-(4-fluorophenyl)-1-isobutylurea;
(S)-1-(1-(6,7-difluoro-3-methyl-4-oxo-3,4-dihydrophthalazin-1-yl)ethyl)-3-(4-fluorophenyl)-1-isobutylurea;
(R)-3-(3-chloro-4-fluorophenyl)-1-(1-(6,7-difluoro-3-methyl-4-oxo-3,4-dihydrophthalazin-1-yl)ethyl)-1-(3-hydroxypropyl)urea;
(S)-3-(3-chloro-4-fluorophenyl)-1-(1-(6,7-difluoro-3-methyl-4-oxo-3,4-dihydrophthalazin-1-yl)ethyl)-1-(3-hydroxypropyl)urea;
(R)-3-(4-fluorophenyl)-1-(1-(6,7-difluoro-3-methyl-4-oxo-3,4-dihydrophthalazin-1-yl)ethyl)-1-(3-hydroxypropyl)urea;
(S)-3-(4-fluorophenyl)-1-(1-(6,7-difluoro-3-methyl-4-oxo-3,4-dihydrophthalazin-1-yl)ethyl)-1-(3-hydroxypropyl)urea;
(R)-3-(3-chloro-4-fluorophenyl)-1-(1-(6,7-difluoro-4-oxo-3,4-dihydrophthalazin-1-yl)ethyl)-1-isobutylurea;
(S)-3-(3-chloro-4-fluorophenyl)-1-(1-(6,7-difluoro-4-oxo-3,4-dihydrophthalazin-1-yl)ethyl)-1-isobutylurea;
(R)-1-(1-(6,7-difluoro-4-oxo-3,4-dihydrophthalazin-1-yl)ethyl)-1-isobutyl-3-phenylurea;
(S)-1-(1-(6,7-difluoro-4-oxo-3,4-dihydrophthalazin-1-yl)ethyl)-1-isobutyl-3-phenylurea;
(R)-3-(3-chloro-4-fluorophenyl)-1-(1-(6,7-difluoro-4-oxo-3,4-dihydrophthalazin-1-yl)ethyl)-1-methylurea;
(S)-3-(3-chloro-4-fluorophenyl)-1-(1-(6,7-difluoro-4-oxo-3,4-dihydrophthalazin-1-yl)ethyl)-1-methylurea;
(R)-2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide;
(S)-2-(3-(3-chloro-4-fluorophenyl)-1-(1-(6,7-difluoro-3-methyl-4-oxo-3,4-dihydrophthalazin-1-yl)ethyl)ureido)-N-((3-chloro-4-fluorophenyl)carbamoyl)ethane-1-sulfonamide;
(R)-3-cyclopropyl-1-(1-(6,7-difluoro-3-methyl-4-oxo-3,4-dihydrophthalazin-1-yl)ethyl)-1-isobutylurea;
(S)-3-cyclopropyl-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthalazin-1-yl)ethyl)-1-isobutyl-3-phenylurea;
(S)-1-(1-(6,7-difluoro-3-methyl-4-oxo-3,4-dihydrophthalazin-1-yl)ethyl)-1-isobutyl-3-phenylurea;
(R)-3-cyclopentyl-1-(1-(6,7-difluoro-3-methyl-4-oxo-3,4-dihydrophthalazin-1-yl)ethyl)-1-isobutylurea;
(S)-3-cyclopentyl-1-(1-(6,7-difluoro-3-methyl-4-oxo-3,4-dihydrophthalazin-1-yl)ethyl)-1-isobutylurea;
(R)-1-(1-(6,7-difluoro-3-methyl-4-oxo-3,4-dihydrophthalazin-1-yl)ethyl)-3-(4-fluorophenyl)-1-methylurea;
(S)-1-(1-(6,7-difluoro-3-methyl-4-oxo-3,4-dihydrophthalazin-1-yl)ethyl)-3-(4-fluorophenyl)-1-methylurea;
(R)-1-(1-(6,7-difluoro-4-oxo-3,4-dihydrophthalazin-1-yl)ethyl)-3-(3,4-difluorophenyl)-1-methylurea;
(S)-1-(1-(6,7-difluoro-4-oxo-3,4-dihydrophthalazin-1-yl)ethyl)-3-(3,4-difluorophenyl)-1-methylurea;
(R)-1-(1-(6,7-difluoro-4-oxo-3,4-dihydrophthalazin-1-yl)ethyl)-1-methyl-3-(3,4,5-trifluorophenyl)urea;
(S)-1-(1-(6,7-difluoro-4-oxo-3,4-dihydrophthalazin-1-yl)ethyl)-1-methyl-3-(3,4,5-trifluorophenyl)urea;
(R)-1-(1-(6,7-difluoro-4-oxo-3,4-dihydrophthalazin-1-yl)ethyl)-3-(4-fluorophenyl)-1-methylurea;
(S)-1-(1-(6,7-difluoro-4-oxo-3,4-dihydrophthalazin-1-yl)ethyl)-3-(4-fluorophenyl)-1-methylurea;
(R)-3-(3-chloro-4-fluorophenyl)-1-(1-(5-fluoro-4-oxo-3,4-dihydrophthalazin-1-yl)ethyl)-1-methylurea;
(S)-3-(3-chloro-4-fluorophenyl)-1-(1-(5-fluoro-4-oxo-3,4-dihydrophthalazin-1-yl)ethyl)-1-methylurea;
(R)-3-(3,4-difluorophenyl)-1-(1-(5-fluoro-4-oxo-3,4-dihydrophthalazin-1-yl)ethyl)-1-methylurea;
(S)-3-(3,4-difluorophenyl)-1-(1-(5-fluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-N-methyl-1H-indole-2-carboxamide;
(S)-N-(1-(6,7-difluoro-4-oxo-3,4-dihydrophthalazin-1-yl)ethyl)-N-methyl-1H-indole-2-carboxamide;
(R)-N-(1-(6,7-difluoro-4-oxo-3,4-dihydrophthalazin-1-yl)ethyl)-N-ethyl-5-fluoro-1H-indole-2-carboxamide;
(S)-N-(1-(6,7-difluoro-4-oxo-3,4-dihydrophthalazin-1-yl)ethyl)-N-ethyl-5-fluoro-1H-indole-2-carboxamide;
(R)-N-(1-(6,7-difluoro-4-oxo-3,4-dihydrophthalazin-1-yl)ethyl)-N-ethyl-1H-indole-2-carboxamide;
(S)-N-(1-(6,7-difluoro-4-oxo-3,4-dihydrophthalazin-1-yl)ethyl)-N-ethyl-1H-indole-2-carboxamide;
(R)-N-(1-(6,7-difluoro-4-oxo-3,4-dihydrophthalazin-1-yl)ethyl)-N-ethyl-4,5-difluoro-1H-indole-2-carboxamide;
(S)-N-(1-(6,7-difluoro-4-oxo-3,4-dihydrophthalazin-1-yl)ethyl)-N-ethyl-4,5-difluoro-1H-indole-2-carboxamide;
(R)-N-(1-(6,7-difluoro-4-oxo-3,4-dihydrophthalazin-1-yl)ethyl)-5,6-difluoro-N-methyl-1H-indole-2-carboxamide;
(S)-N-(1-(6,7-difluoro-4-oxo-3,4-dihydrophthalazin-1-yl)ethyl)-5,6-difluoro-N-methyl-1H-indole-2-carboxamide;
(R)-N-(1-(6,7-difluoro-4-oxo-3,4-dihydrophthalazin-1-yl)ethyl)-5-fluoro-N-methyl-1H-indole-2-carboxamide;
(S)-N-(1-(6,7-difluoro-4-oxo-3,4-dihydrophthalazin-1-yl)ethyl)-5-fluoro-N-methyl-1H-indole-2-carboxamide;
(R)-N-(1-(6,7-difluoro-4-oxo-3,4-dihydrophthalazin-1-yl)ethyl)-6-fluoro-N-methyl-1H-indole-2-carboxamide;
(S)-N-(1-(6,7-difluoro-4-oxo-3,4-dihydrophthalazin-1-yl)ethyl)-6-fluoro-N-methyl-1H-indole-2-carboxamide;
(R)-N-(1-(6,7-difluoro-4-oxo-3,4-dihydrophthalazin-1-yl)ethyl)-4-fluoro-N-methyl-1H-indole-2-carboxamide;
(S)-N-(1-(6,7-difluoro-4-oxo-3,4-dihydrophthalazin-1-yl)ethyl)-4-fluoro-N-methyl-1H-indole-2-carboxamide;
(R)-N-(1-(6,7-difluoro-4-oxo-3,4-dihydrophthalazin-1-yl)ethyl)-4,5-difluoro-N-methyl-1H-indole-2-carboxamide;
(S)-N-(1-(6,7-difluoro-4-oxo-3,4-dihydrophthalazin-1-yl)ethyl)-4,5-difluoro-N-methyl-1H-indole-2-carboxamide;
(R)-N-(1-(6,7-difluoro-4-oxo-3,4-dihydrophthalazin-1-yl)ethyl)-N,3-dimethyl-1H-indole-2-carboxamide;
(S)-N-(1-(6,7-difluoro-4-oxo-3,4-dihydrophthalazin-1-yl)ethyl)-N,3-dimethyl-1H-indole-2-carboxamide;
(R)-N-(1-(6,7-difluoro-4-oxo-3,4-dihydrophthalazin-1-yl)ethyl)-N-isobutyl-1H-indole-2-carboxamide;
(S)-N-(1-(6,7-difluoro-4-oxo-3,4-dihydrophthalazin-1-yl)ethyl)-N-isobutyl-1H-indole-2-carboxamide;
(R)-N-(1-(6,7-difluoro-4-oxo-3,4-dihydrophthalazin-1-yl)ethyl)-N-methyl-4,5,6,7-tetrahydro-1H-indole-2-carboxamide;
(S)-N-(1-(6,7-difluoro-4-oxo-3,4-dihydrophthalazin-1-yl)ethyl)-N-methyl-4,5,6,7-tetrahydro-1H-indole-2-carboxamide;
(R)-N-(1-(6,7-difluoro-4-oxo-3,4-dihydrophthalazin-1-yl)ethyl)-N-methylindolizine-2-carboxamide;
(S)-N-(1-(6,7-difluoro-4-oxo-3,4-dihydrophthalazin-1-yl)ethyl)-N-methylindolizine-2-carboxamide;
(R)-N-(1-(6,7-difluoro-4-oxo-3,4-dihydrophthalazin-1-yl)ethyl)-4,6-difluoro-N-methyl-1H-indole-2-carboxamide;
(S)-N-(1-(6,7-difluoro-4-oxo-3,4-dihydrophthalazin-1-yl)ethyl)-4,6-difluoro-N-methyl-1H-indole-2-carboxamide;
(R)-N-(1-(6,7-difluoro-4-oxo-3,4-dihydrophthalazin-1-yl)ethyl)-3-fluoro-N-methyl-4-(trifluoromethyl)benzamide;
(S)-N-(1-(6,7-difluoro-4-oxo-3,4-dihydrophthalazin-1-yl)ethyl)-3-fluoro-N-methyl-4-(trifluoromethyl)benzamide;
(R)-4-bromo-N-(1-(6,7-difluoro-4-oxo-3,4-dihydrophthalazin-1-yl)ethyl)-3-fluoro-N-methylbenzamide;
(S)-4-bromo-N-(1-(6,7-difluoro-4-oxo-3,4-dihydrophthalazin-1-yl)ethyl)-3-fluoro-N-methylbenzamide;
(R)-4-chloro-N-(1-(6,7-difluoro-4-oxo-3,4-dihydrophthalazin-1-yl)ethyl)-3-fluoro-N-methylbenzamide;
(S)-4-chloro-N-(1-(6,7-difluoro-4-oxo-3,4-dihydrophthalazin-1-yl)ethyl)-3-fluoro-N-methylbenzamide;
(R)-4-bromo-N-(1-(6,7-difluoro-4-oxo-3,4-dihydrophthalazin-1-yl)ethyl)-N-methylbenzamide;
(S)-4-bromo-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-N-methyl-4-(trifluoromethyl)benzamide;
(S)-N-(1-(6,7-difluoro-4-oxo-3,4-dihydrophthalazin-1-yl)ethyl)-N-methyl-4-(trifluoromethyl)benzamide;
(R)-N-(1-(6,7-difluoro-4-oxo-3,4-dihydrophthalazin-1-yl)ethyl)-3,4,5-trifluoro-N-methylbenzamide;
(S)-N-(1-(6,7-difluoro-4-oxo-3,4-dihydrophthalazin-1-yl)ethyl)-3,4,5-trifluoro-N-methylbenzamide;
(R)-3-chloro-N-(1-(6,7-difluoro-4-oxo-3,4-dihydrophthalazin-1-yl)ethyl)-4-fluoro-N-methylbenzamide;
(S)-3-chloro-N-(1-(6,7-difluoro-4-oxo-3,4-dihydrophthalazin-1-yl)ethyl)-4-fluoro-N-methylbenzamide;
(R)-4-chloro-N-(1-(6,7-difluoro-4-oxo-3,4-dihydrophthalazin-1-yl)ethyl)-N-methylbenzamide;
(S)-4-chloro-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-3,4-difluoro-N-methylbenzamide;
(S)-N-(1-(6,7-difluoro-4-oxo-3,4-dihydrophthalazin-1-yl)ethyl)-3,4-difluoro-N-methylbenzamide;
(R)-N-(1-(6,7-difluoro-4-oxo-3,4-dihydrophthalazin-1-yl)ethyl)-3-(difluoromethyl)-N-methylbenzamide;
(S)-N-(1-(6,7-difluoro-4-oxo-3,4-dihydrophthalazin-1-yl)ethyl)-3-(difluoromethyl)-N-methylbenzamide;
(R)-N-(1-(6,7-difluoro-4-oxo-3,4-dihydrophthalazin-1-yl)ethyl)-3-(difluoromethyl)-4-fluoro-N-methylbenzamide;
(S)-N-(1-(6,7-difluoro-4-oxo-3,4-dihydrophthalazin-1-yl)ethyl)-3-(difluoromethyl)-4-fluoro-N-methylbenzamide;
(R)-1-(1-(6,7-difluoro-4-oxo-3,4-dihydrophthalazin-1-yl)ethyl)-3-(3-(difluoromethyl)-4-fluorophenyl)-1-methylurea;
(S)-1-(1-(6,7-difluoro-4-oxo-3,4-dihydrophthalazin-1-yl)ethyl)-3-(3-(difluoromethyl)-4-fluorophenyl)-1-methylurea;
(R)-N-(1-(6,7-difluoro-4-oxo-3,4-dihydrophthalazin-1-yl)ethyl)-N-methylbenzamide;
(S)-N-(1-(6,7-difluoro-4-oxo-3,4-dihydrophthalazin-1-yl)ethyl)-N-methylbenzamide;
(R)-8-chloro-N-(1-(6,7-difluoro-4-oxo-3,4-dihydrophthalazin-1-yl)ethyl)-N-methylindolizine-2-carboxamide;
(S)-8-chloro-N-(1-(6,7-difluoro-4-oxo-3,4-dihydrophthalazin-1-yl)ethyl)-N-methylindolizine-2-carboxamide;
(R)-3-(3-cyano-4-fluorophenyl)-1-(1-(6,7-difluoro-4-oxo-3,4-dihydrophthalazin-1-yl)ethyl)-1-methylurea;
(S)-3-(3-cyano-4-fluorophenyl)-1-(1-(6,7-difluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-8-fluoro-N-methylindolizine-2-carboxamide;
(S)-N-(1-(6,7-difluoro-4-oxo-3,4-dihydrophthalazin-1-yl)ethyl)-8-fluoro-N-methylindolizine-2-carboxamide;
(2S)-N-(1-(6,7-difluoro-4-oxo-3,4-dihydrophthalazin-1-yl)ethyl)-N-methylindoline-2-carboxamide;
(S)-N-((S)-1-(6,7-difluoro-4-oxo-3,4-dihydrophthalazin-1-yl)ethyl)-N-methylindoline-2-carboxamide;
(S)-N-((R)-1-(6,7-difluoro-4-oxo-3,4-dihydrophthalazin-1-yl)ethyl)-N-methylindoline-2-carboxamide;
(2R)-N-(1-(6,7-difluoro-4-oxo-3,4-dihydrophthalazin-1-yl)ethyl)-N-methylindoline-2-carboxamide;
(R)-N-((R)-1-(6,7-difluoro-4-oxo-3,4-dihydrophthalazin-1-yl)ethyl)-N-methylindoline-2-carboxamide;
(R)-N-((S)-1-(6,7-difluoro-4-oxo-3,4-dihydrophthalazin-1-yl)ethyl)-N-methylindoline-2-carboxamide;
(R)-2-chloro-N-(1-(6,7-difluoro-4-oxo-3,4-dihydrophthalazin-1-yl)ethyl)-N-methyl-4H-thieno[3,2-b]pyrrole-5-carboxamide;
(S)-2-chloro-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-4H-thieno[3,2-b]pyrrole-5-carboxamide;
(S)-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-2,3-dihydro-1H-indene-5-carboxamide;
(S)-N-(1-(6,7-difluoro-4-oxo-3,4-dihydrophthalazin-1-yl)ethyl)-N-methyl-2,3-dihydro-1H-indene-5-carboxamide;
(R)-N-(1-(6,7-difluoro-4-oxo-3,4-dihydrophthalazin-1-yl)ethyl)-N-methylbenzo[d]thiazole-5-carboxamide;
(S)-N-(1-(6,7-difluoro-4-oxo-3,4-dihydrophthalazin-1-yl)ethyl)-N-methylbenzo[d]thiazole-5-carboxamide;
(R)-N-(1-(6,7-difluoro-4-oxo-3,4-dihydrophthalazin-1-yl)ethyl)-N-methylbenzo[d]thiazole-6-carboxamide;
(S)-N-(1-(6,7-difluoro-4-oxo-3,4-dihydrophthalazin-1-yl)ethyl)-N-methylbenzo[d]thiazole-6-carboxamide;
(R)-N-(1-(6,7-difluoro-4-oxo-3,4-dihydrophthalazin-1-yl)ethyl)-N-methylbenzo[d]oxazole-5-carboxamide;
(S)-N-(1-(6,7-difluoro-4-oxo-3,4-dihydrophthalazin-1-yl)ethyl)-N-methylbenzo[d]oxazole-5-carboxamide;
(R)-N-(1-(6,7-difluoro-4-oxo-3,4-dihydrophthalazin-1-yl)ethyl)-N-methylbenzo[d]oxazole-6-carboxamide;
(S)-N-(1-(6,7-difluoro-4-oxo-3,4-dihydrophthalazin-1-yl)ethyl)-N-methylbenzo[d]oxazole-6-carboxamide;
(R)-5-chloro-N-(1-(6,7-difluoro-4-oxo-3,4-dihydrophthalazin-1-yl)ethyl)-N-methyl-6-(trifluoromethyl)nicotinamide;
(S)-5-chloro-N-(1-(6,7-difluoro-4-oxo-3,4-dihydrophthalazin-1-yl)ethyl)-N-methyl-6-(trifluoromethyl)nicotinamide;
(R)-4-chloro-N-(1-(6,7-difluoro-4-oxo-3,4-dihydrophthalazin-1-yl)ethyl)-3,5-difluoro-N-methylbenzamide;
(S)-4-chloro-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-4-(difluoromethyl)-6-fluoro-N-methyl-1H-indole-2-carboxamide;
(S)-N-(1-(6,7-difluoro-4-oxo-3,4-dihydrophthalazin-1-yl)ethyl)-4-(difluoromethyl)-6-fluoro-N-methyl-1H-indole-2-carboxamide;
(R)-N-(1-(6,7-difluoro-4-oxo-3,4-dihydrophthalazin-1-yl)ethyl)-8-(difluoromethyl)-N-methylindolizine-2-carboxamide;
(S)-N-(1-(6,7-difluoro-4-oxo-3,4-dihydrophthalazin-1-yl)ethyl)-8-(difluoromethyl)-N-methylindolizine-2-carboxamide;
(R)-4-bromo-N-(1-(6,7-difluoro-4-oxo-3,4-dihydrophthalazin-1-yl)ethyl)-3,5-difluoro-N-methylbenzamide;
(S)-4-bromo-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(S)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(R)-N-(1-(6,7-difluoro-4-oxo-3,4-dihydrophthalazin-1-yl)ethyl)-N,1-dimethyl-1H-indole-6-carboxamide;
(S)-N-(1-(6,7-difluoro-4-oxo-3,4-dihydrophthalazin-1-yl)ethyl)-N,1-dimethyl-1H-indole-6-carboxamide;
(R)-N-(1-(6,7-difluoro-4-oxo-3,4-dihydrophthalazin-1-yl)ethyl)-N,1-dimethyl-1H-indole-5-carboxamide;
(S)-N-(1-(6,7-difluoro-4-oxo-3,4-dihydrophthalazin-1-yl)ethyl)-N,1-dimethyl-1H-indole-5-carboxamide;
(R)-N-(1-(6,7-difluoro-4-oxo-3,4-dihydrophthalazin-1-yl)ethyl)-4-(difluoromethyl)-3,5-difluoro-N-methylbenzamide;
(S)-N-(1-(6,7-difluoro-4-oxo-3,4-dihydrophthalazin-1-yl)ethyl)-4-(difluoromethyl)-3,5-difluoro-N-methylbenzamide;
(R)-N-(1-(6,7-difluoro-4-oxo-3,4-dihydrophthalazin-1-yl)ethyl)-N-methyl-1H-indazole-5-carboxamide;
(S)-N-(1-(6,7-difluoro-4-oxo-3,4-dihydrophthalazin-1-yl)ethyl)-N-methyl-1H-indazole-5-carboxamide;
(R)-N-(1-(6,7-difluoro-4-oxo-3,4-dihydrophthalazin-1-yl)ethyl)-N-methyl-1H-indazole-6-carboxamide;
(S)-N-(1-(6,7-difluoro-4-oxo-3,4-dihydrophthalazin-1-yl)ethyl)-N-methyl-1H-indazole-6-carboxamide;
(R)-N-(1-(6,7-difluoro-4-oxo-3,4-dihydrophthalazin-1-yl)ethyl)-5-fluoro-N-methyl-6-(trifluoromethyl)nicotinamide;
(S)-N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled derivative, stereoisomer, or tautomer thereof, or any mixtures thereof.

Embodiment 18 provides a pharmaceutical composition comprising at least one compound of any one of Embodiments 1-17 and a pharmaceutically acceptable carrier.

Embodiment 19 provides the pharmaceutical composition of Embodiment 18, further comprising at least one additional agent useful for treating hepatitis infection.

Embodiment 20 provides the pharmaceutical composition of Embodiment 19, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 21 provides the pharmaceutical composition of Embodiment 20, wherein the immunostimulator is a checkpoint inhibitor.

Embodiment 22 provides the pharmaceutical composition of Embodiment 21, wherein the checkpoint inhibitor is a PD-L1 inhibitor.

Embodiment 23 provides a method of treating, ameliorating, and/or preventing hepatitis B virus (HBV) infection in a subject, the method comprising administering to the subject in need thereof a therapeutically effective amount of at least one compound of any one of Embodiments 1-17 and/or at least one pharmaceutical composition of any one of Embodiments 18-22.

Embodiment 24 provides the method of Embodiment 23, wherein the subject is further infected with hepatitis D virus (HDV).

Embodiment 25 provides the method of any of Embodiments 23-24, wherein the at least one compound and/or composition is administered to the subject in a pharmaceutically acceptable composition.

Embodiment 26 provides the method of any of Embodiments 23-25, wherein the subject is further administered at least one additional agent useful for treating, ameliorating, and/or preventing the hepatitis B virus infection.

Embodiment 27 provides the method of Embodiment 26, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 28 provides the method of Embodiment 27, wherein the immunostimulator is a checkpoint inhibitor.

Embodiment 29 provides the method of Embodiment 28, wherein the checkpoint inhibitor is a PD-L1 inhibitor.

Embodiment 30 provides the method of any of Embodiments 26-29, wherein the subject is co-administered the at least one compound and/or composition and the at least one additional agent.

Embodiment 31 provides the method of any of Embodiments 26-30, wherein the at least one compound and/or composition and the at least one additional agent are coformulated.

Embodiment 32 provides a method of inhibiting expression and/or function of a viral capsid protein directly or indirectly in a heptatis B virus-infected subject, the method comprising administering to the subject in need thereof a therapeutically effective amount of at least one compound of any one of Embodiments 1-17 and/or at least one pharmaceutical composition of any one of Embodiments 18-22.

Embodiment 33 provides the method of Embodiment 32, wherein the subject is further infected with hepatitis D virus (HDV).

Embodiment 34 provides the method of any of Embodiments 32-33, wherein the at least one compound and/or composition is administered to the subject in a pharmaceutically acceptable composition.

Embodiment 35 provides the method of any of Embodiments 32-34, wherein the subject is further administered at least one additional agent useful for treating the hepatitis B viral infection.

Embodiment 36 provides the method of Embodiment 35, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript, and therapeutic vaccine.

Embodiment 37 provides the method of Embodiment 36, wherein the immunostimulator is a checkpoint inhibitor.

Embodiment 38 provides the method of Embodiment 37, wherein the checkpoint inhibitor is a PD-L1 inhibitor.

Embodiment 39 provides the method of any of Embodiments 32-38, wherein the subject is co-administered the at least one compound and/or composition and the at least one additional agent.

Embodiment 40 provides the method of any of Embodiments 32-39, wherein the at least one compound and/or composition and the at least one additional agent are coformulated.

Embodiment 41 provides the method of any of Embodiments 23-40, wherein the subject is a mammal.

Embodiment 42 provides the method of Embodiment 41, wherein the mammal is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A compound of formula (Ia) or (Ib), or a salt, solvate, prodrug, stereoisomer, tautomer, or isotopically labelled derivative thereof, or any mixtures thereof: 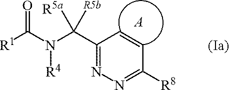 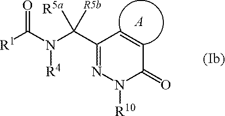
wherein in (Ia) or (Ib):
ring A 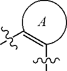
is selected from the group consisting of: 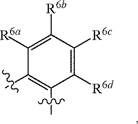 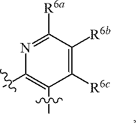 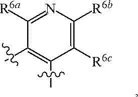 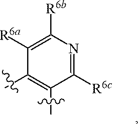 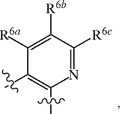 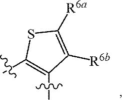 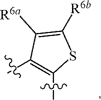 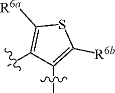
(wherein there is no bridgehead double bond in the bicyclic structure including ring A), 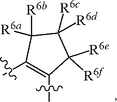 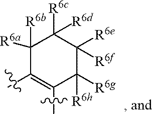 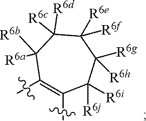
or ring A is absent and 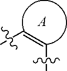 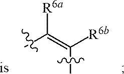
R¹ is selected from the group consisting of —NR²R³, 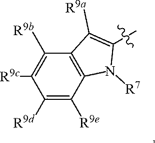 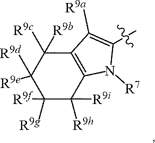 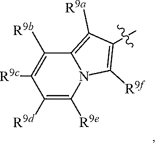 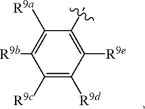 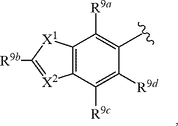 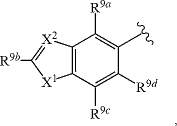 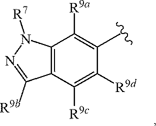 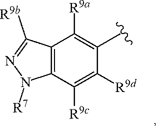 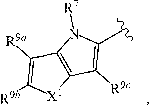 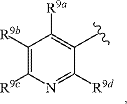 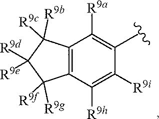 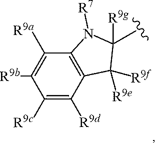 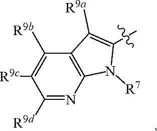 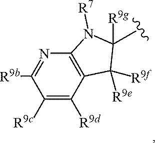 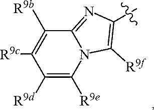 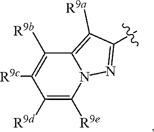 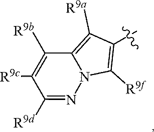 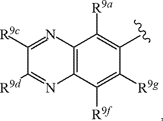 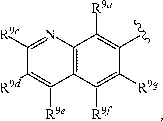 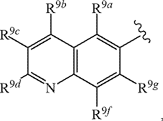 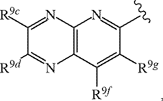 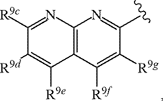 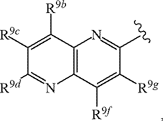 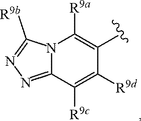 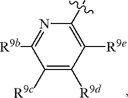 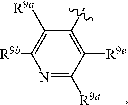 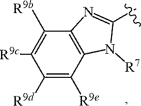 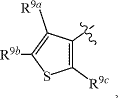 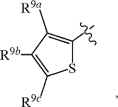 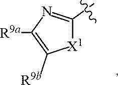 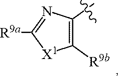 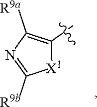 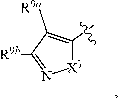 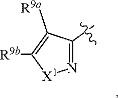 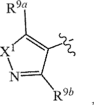 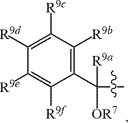 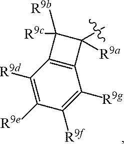 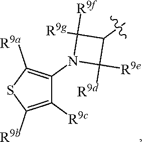 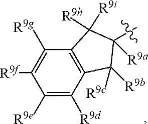 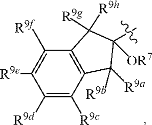 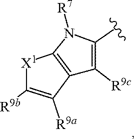 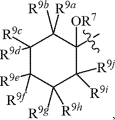 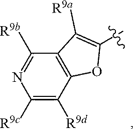 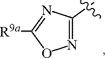 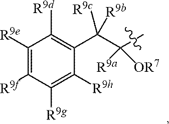 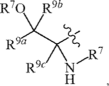 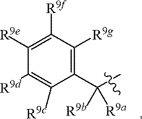 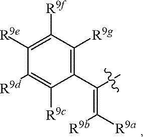 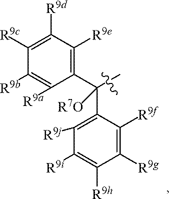 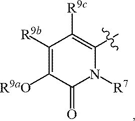 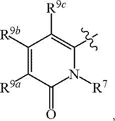 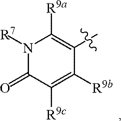 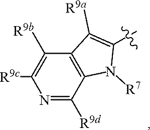 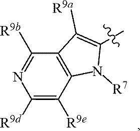 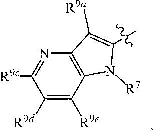 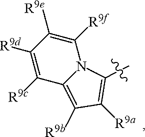 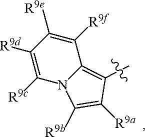 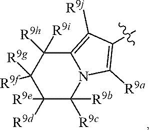 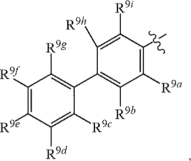 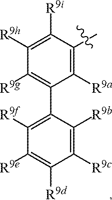 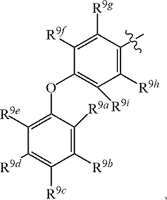 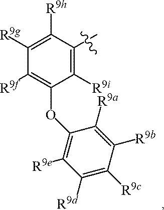 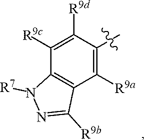 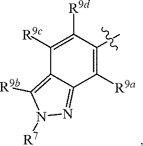 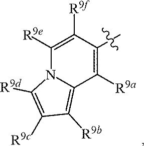 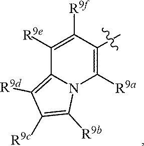 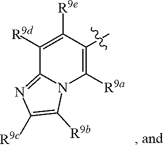 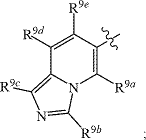
X¹ is selected from the group consisting of O, S, and N(R⁷);
X² is selected from the group consisting of N and CR^{9e};
R² is selected from the group consisting of optionally substituted C₃-C₈ cycloalkyl, optionally substituted phenyl, optionally substituted benzyl, optionally substituted heteroaryl, and -(CH₂)(optionally substituted heteroaryl);
R³ is selected from the group consisting of H and optionally substituted C₁-C₆ alkyl;
R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁷, —OC(═O)R⁷, —SR⁷, —S(═O)R⁷, —S(═ 0)₂R⁷, —S(═O)₂NR⁷R⁷, —S(═O)₂NHC(═O)NHR⁷, —N(R⁷)S(═O)₂R⁷, —N(R⁷)C(═O)R⁷, —C(═O)NR⁷R⁷, and —NR⁷R⁷;
R^{5a} and R^{5b} are each independently selected from the group consisting of H and optionally substituted C₁-C₆ alkyl;
each occurrence of R^{6a}, R^{6b}, R^{6c}, R^{6d}, R^{6e}, R^{6f}, R^{6g}, R^{6h}, R⁶ⁱ, and R^{6j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, -S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), -O(optionally substituted C₁-C₆ alkyl), —O(optionally substituted C₃-C₈ cycloalkyl), —NH₂, -NH(optionally substituted C₁-C₆ alkyl), —NH(optionally substituted C₃-C₈ cycloalkyl), -N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁷ is independently selected from the group consisting of H, optionally substituted C₁-C₆ alkyl, optionally substituted C₃-C₈ cycloalkyl, optionally substituted phenyl, and optionally substituted hetereoaryl;
R⁸ is selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), -O(optionally substituted C₁-C₆ alkyl), —O(optionally substituted C₃-C₈ cycloalkyl), —NH₂, -NH(optionally substituted C₁-C₆ alkyl), —NH(optionally substituted C₃-C₈ cycloalkyl), -N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R^{9a}, R^{9b}, R^{9c}, R^{9d}, R^{9e}, R^{9f}, R^{9g}, R^{9h}, R⁹ⁱ, and R^{9j} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl; and
R¹⁰ is selected from the group consisting of H, C₁-C₆ alkyl, optionally substituted C₃-C₈ cycloalkyl, optionally substituted C₁-C₆ alkoxy, and optionally substituted C₃-C₈ cycloalkoxy.

2. The compound of claim 1, which is at least one selected from the group consisting of: 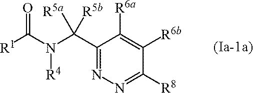 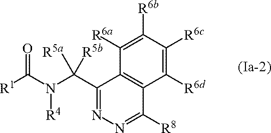 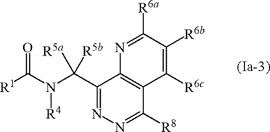 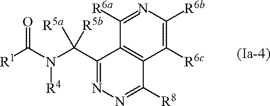 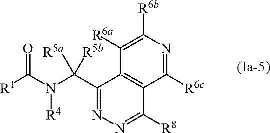 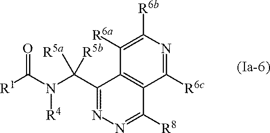 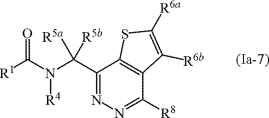 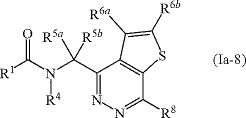 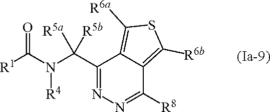 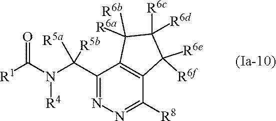 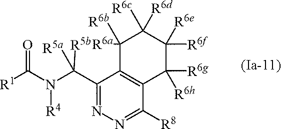 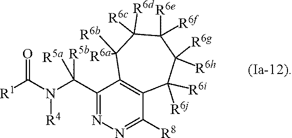 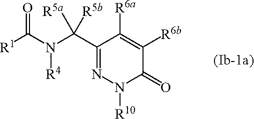 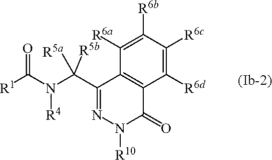 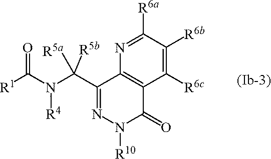 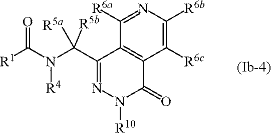 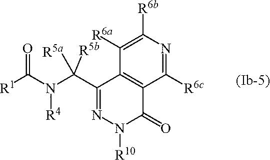 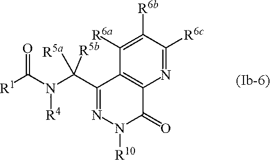 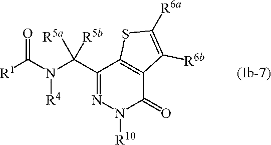 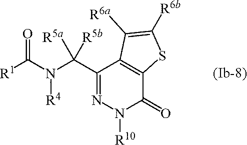 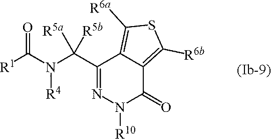 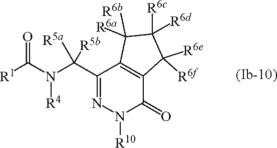 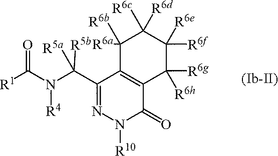
and 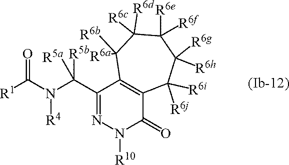
.

3. (canceled)

4. The compound of claim 1, which is at least one selected from the group consisting of: 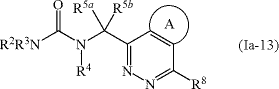 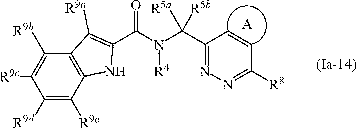 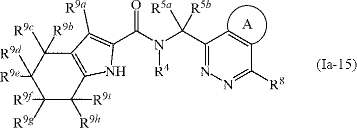 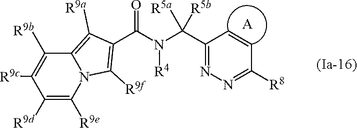 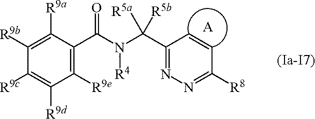 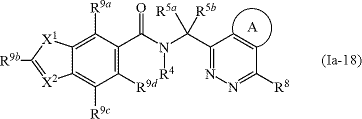 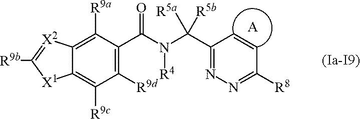 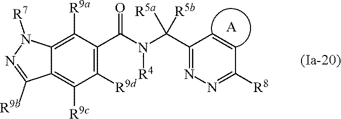 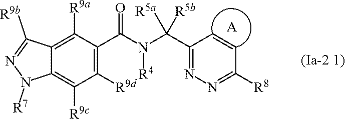 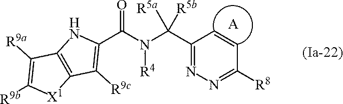 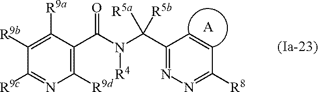 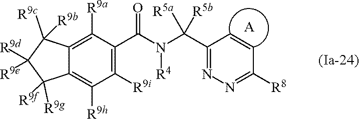 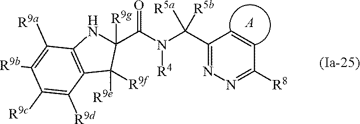 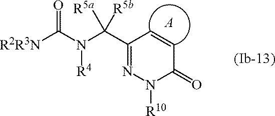 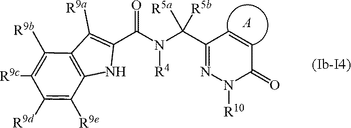 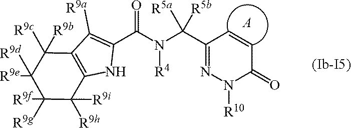 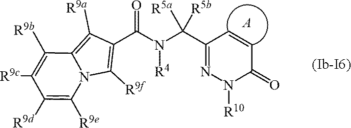 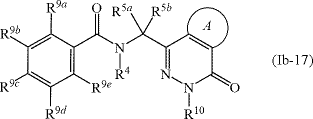 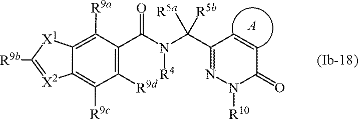 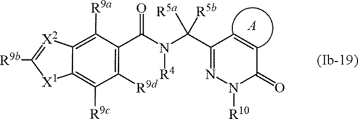 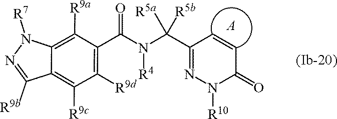 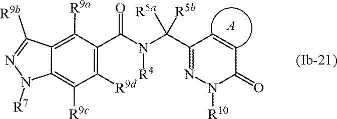 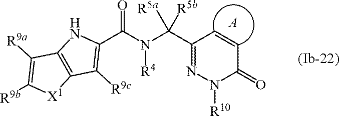 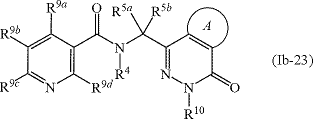 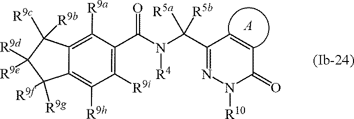
and 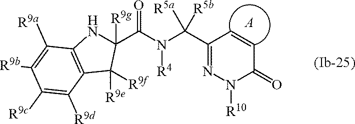
.

5. (canceled)

6. The compound of claim 1, which is at least one selected from the group consisting of: 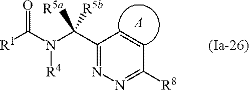 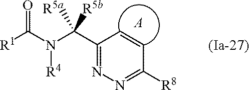 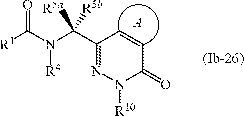
and 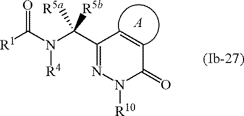
.

7. The compound of claim 1, which is at least one selected from the group consisting of: 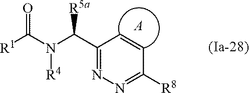 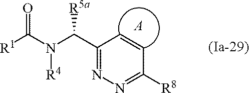 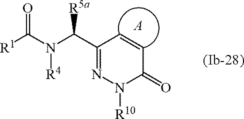
and 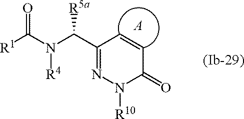
.

8. The compound of claim 1, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)zRb, N(R^{b})S(═0)₂R^{b}, —S(═0)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁—₃O—.

9. The compound of claim 1, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═0)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

10. The compound of claim 1, wherein ring A is selected from the group consisting of: 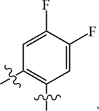 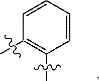 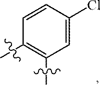 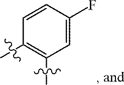 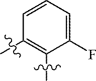
.

11. The compound of claim 1, wherein R² is phenyl optionally substituted with at least one selected from the group consisting of C₁-C₆ alkyl, halo, C₁-C₃ haloalkyl, and —CN.

12. The compound of claim 1, wherein R² is selected from the group consisting of phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, and 4-difluoromethyl-3-fluorophenyl.

13. The compound of claim 1, wherein R³ is selected from the group consisting of H and methyl.

14. The compound of claim 1, wherein R⁴ is selected from the group consisting of methyl, sec-butyl, 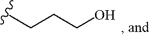 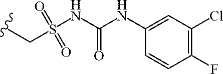
.

15. The compound of claim 1, wherein R¹ is selected from the group consisting of: 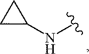 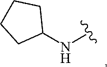 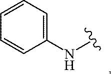 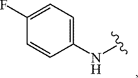 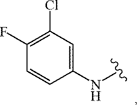 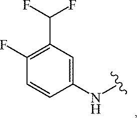 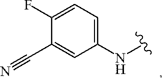 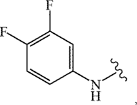 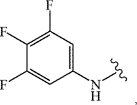 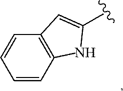 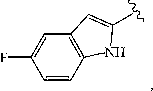 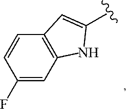 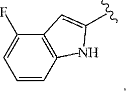 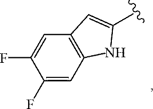 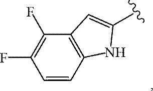 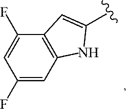 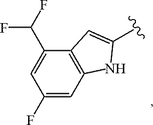 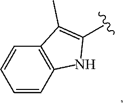 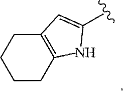 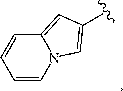 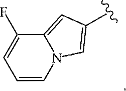 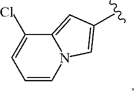 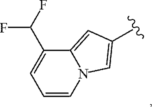 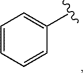 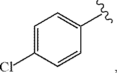 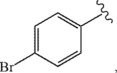 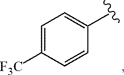 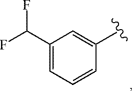 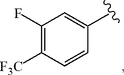 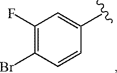 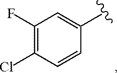 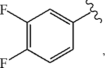 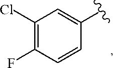 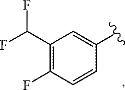 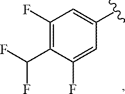 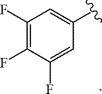 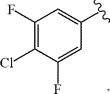 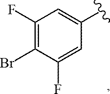 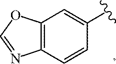 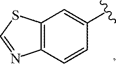 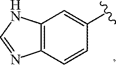 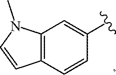 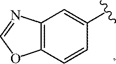 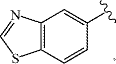 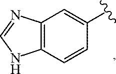 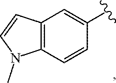 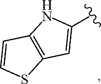 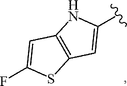 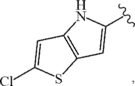 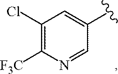 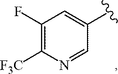 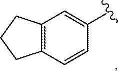 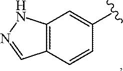 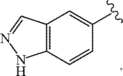 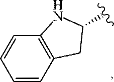 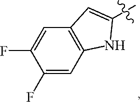 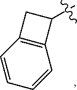 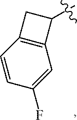 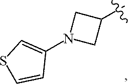 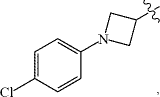 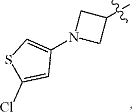 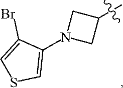 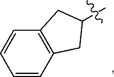 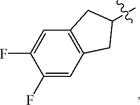 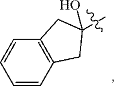 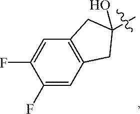 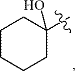 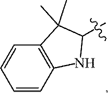 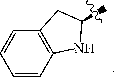 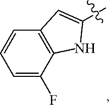 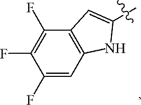 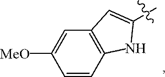 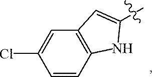 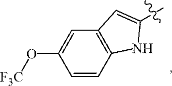 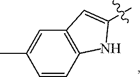 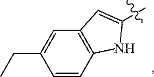 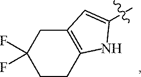 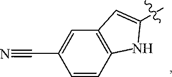 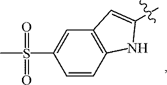 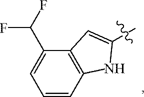 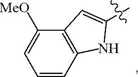 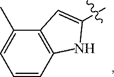 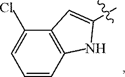 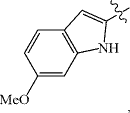 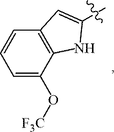 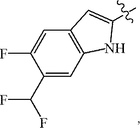 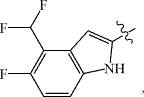 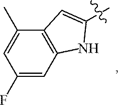 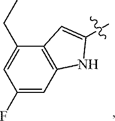 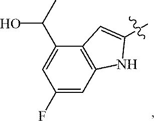 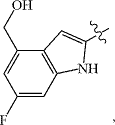 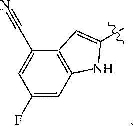 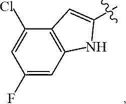 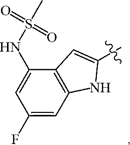 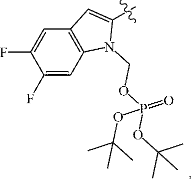 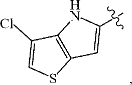 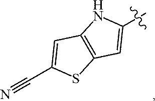 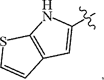 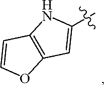 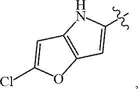 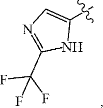 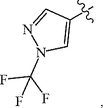 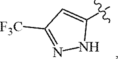 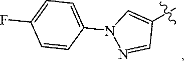 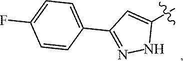 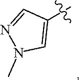 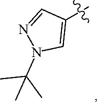 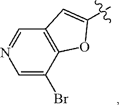 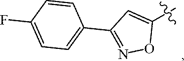 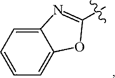 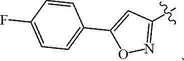 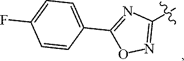 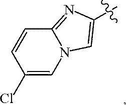 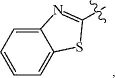 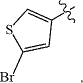 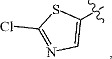 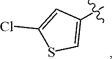 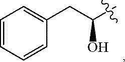 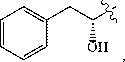 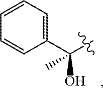 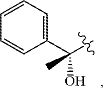 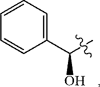 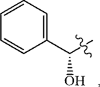 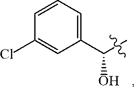 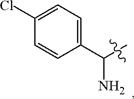 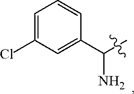 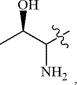 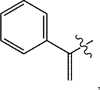 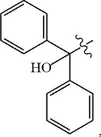 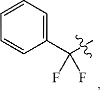 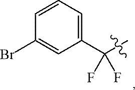 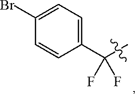 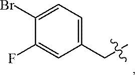 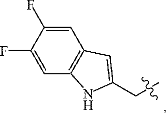 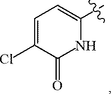 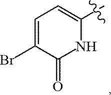 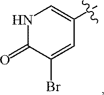 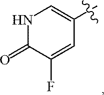 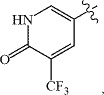 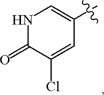 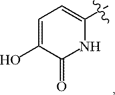 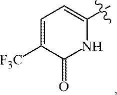 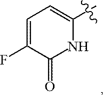 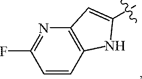 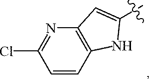 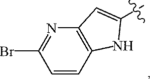 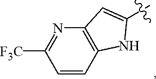 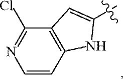 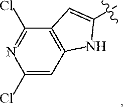 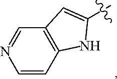 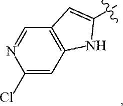 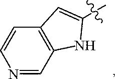 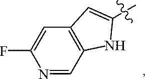 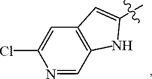 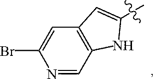 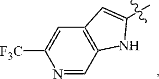 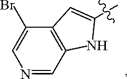 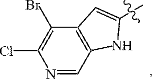 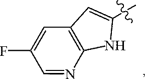 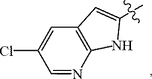 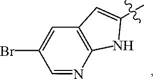 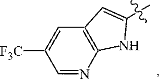 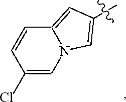 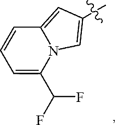 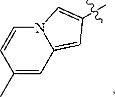 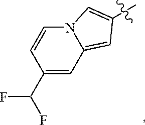 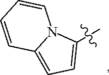 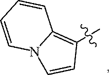 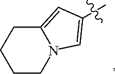 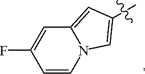 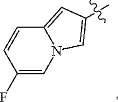 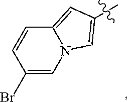 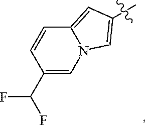 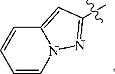 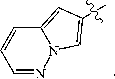 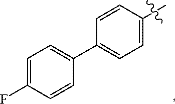 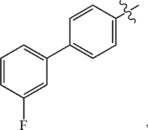 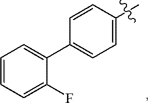 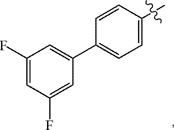 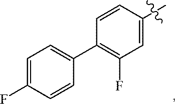 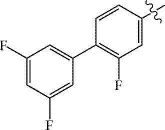 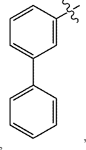 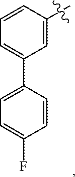 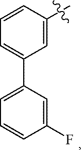 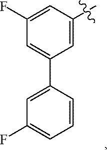 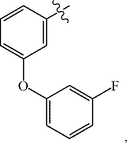 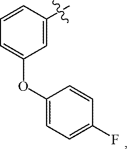 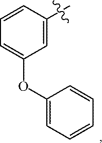 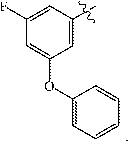 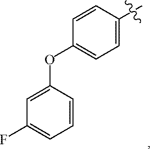 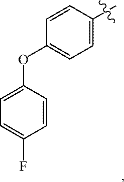 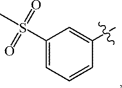 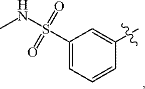 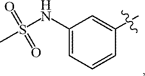 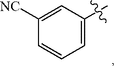 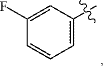 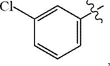 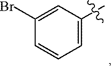 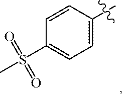 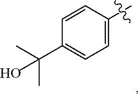 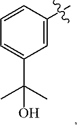 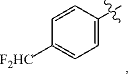 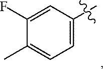 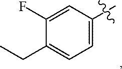 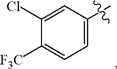 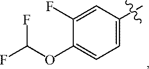 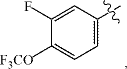 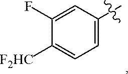 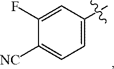 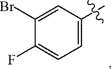 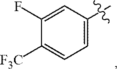 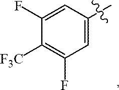 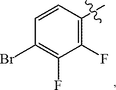 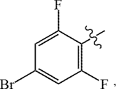 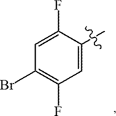 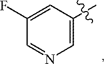 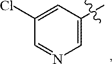 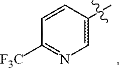 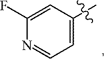 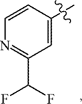 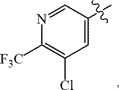 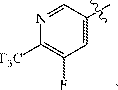 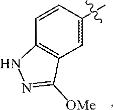 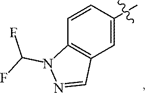 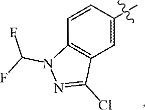 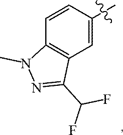 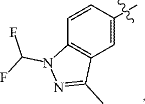 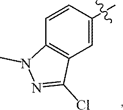 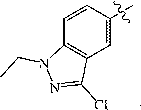 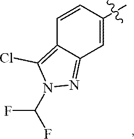 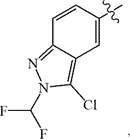 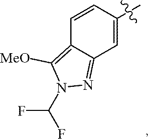 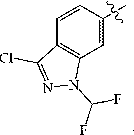 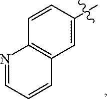 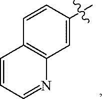 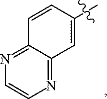 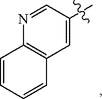 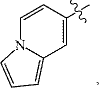 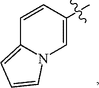 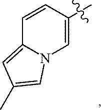 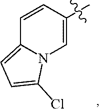 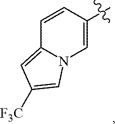 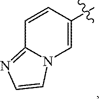 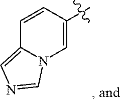 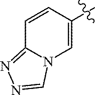
.

16. The compound of claim 1, which is at least one selected from the group consisting of:
3-(3-chloro-4-fluorophenyl)-1-methyl-1-(1-(4-oxo-3,4-dihydrophthalazin-1-yl)ethyl)urea;
3-(3-chloro-4-fluorophenyl)-1-isobutyl-1-(1-(4-oxo-3,4-dihydrophthalazin-1-yl)ethyl)urea;
3-(3-chloro-4-fluorophenyl)-1-isobutyl-1-(1-(3-methyl-4-oxo-3,4-dihydrophthalazin-1-yl)ethyl)urea;
3-(4-fluorophenyl)-1-isobutyl-1-(1-(3-methyl-4-oxo-3,4-dihydrophthalazin-1-yl)ethyl)urea;
3-(3-chloro-4-fluorophenyl)-1-(1-(6-chloro-4-oxo-3,4-dihydrophthalazin-1-yl)ethyl)-1-isobutylurea;
1-(1-(6-chloro-4-oxo-3,4-dihydrophthalazin-1-yl)ethyl)-3-(4-fluorophenyl)-1-isobutylurea;
3-(3-chloro-4-fluorophenyl)-1-(1-(6-chloro-4-oxo-3,4-dihydrophthalazin-1-yl)ethyl)-1-methylurea;
3-(3-chloro-4-fluorophenyl)-1-(1-(6-chloro-4-oxo-3,4-dihydrophthalazin-1-yl)ethyl)-1-(3-hydroxypropyl)urea;
1-(1-(6-chloro-4-oxo-3,4-dihydrophthalazin-1-yl)ethyl)-3-(4-fluorophenyl)-1-(3-hydroxypropyl)urea;
3-(3-chloro-4-fluorophenyl)-1-(1-(6-fluoro-4-oxo-3,4-dihydrophthalazin-1-yl)ethyl)-1-isobutylurea;
1-(1-(6-fluoro-4-oxo-3,4-dihydrophthalazin-1-yl)ethyl)-3-(4-fluorophenyl)-1-isobutylurea;
3-(3-chloro-4-fluorophenyl)-1-(1-(6-fluoro-4-oxo-3,4-dihydrophthalazin-1-yl)ethyl)-1-methylurea;
1-(1-(6-fluoro-4-oxo-3,4-dihydrophthalazin-1-yl)ethyl)-3-(4-fluorophenyl)-1-methylurea;
3-(4-fluorophenyl)-1-isobutyl-1-(1-(4-oxo-3,4-dihydrophthalazin-1-yl)ethyl)urea;
3 -(3 -chloro-4-fluorophenyl)-1 -(1 -(6,7-difluoro-3 -m ethyl-4-oxo-3, 4-dihy drophthalazin- 1 -yl)ethyl)-1-methylurea;
3 -(3 -chloro-4-fluorophenyl)-1 -(1 -(6,7-difluoro-3 -m ethyl-4-oxo-3, 4-dihy drophthal azin- 1 -yl)ethyl)-1-isobutylurea;
1-(1-(6,7-difluoro-3-methyl-4-oxo-3,4-dihydrophthalazin-1-yl)ethyl)-3-(4-fluorophenyl)-1-isobutylurea;
3 -(3 -chloro-4-fluorophenyl)-1 -(1 -(6,7-difluoro-3 -m ethyl-4-oxo-3, 4-dihy drophthal azin- 1 -yl)ethyl)-1-(3-hydroxypropyl)urea;
3-(4-fluorophenyl)- 1 -(1 -(6,7-difluoro-3 -m ethyl-4-oxo-3,4-dihy drophthal azin- 1 -yl)ethyl)- 1 -(3 -hydroxypropyl)urea;
3 -(3 -chloro-4-fluorophenyl)-1 -(1 -(6,7-difluoro-4-oxo-3,4-dihy drophthal azin- 1 -yl)ethyl)-1 -isobutylurea;
1-(1-(6,7-difluoro-4-oxo-3,4-dihydrophthalazin-1-yl)ethyl)-1-isobutyl-3-phenylurea;
3 -(3 -chloro-4-fluorophenyl)-1 -(1 -(6,7-difluoro-4-oxo-3,4-dihy drophthal azin- 1 -yl)ethyl)-1 -methylurea;
2-(3 -(3 -chloro-4-fluorophenyl)-1-(1 -(6,7-difluoro-3 -methyl-4-oxo-3,4-dihydrophthalazin-1-yl)ethyl)ureido)-N-((3-chloro-4-fluorophenyl)carbamoyl)ethane-1-sulfonamide;
3-cyclopropyl-1-(1-(6,7-difluoro-3-methyl-4-oxo-3,4-dihydrophthalazin-1-yl)ethyl)-1-isobutylurea;
1-(1-(6,7-difluoro-3-methyl-4-oxo-3,4-dihydrophthalazin-1-yl)ethyl)-1-isobutyl-3-phenylurea;
3 -cyclopentyl-1 -(1 -(6,7-difluoro-3 -methyl-4-oxo-3, 4-dihy drophthalazin-1 -yl)ethyl)- 1 -isobutylurea;
1-(1-(6,7-difluoro-3-methyl-4-oxo-3,4-dihydrophthalazin-1-yl)ethyl)-3-(4-fluorophenyl)-1-methylurea;
1-(1-(6,7-difluoro-4-oxo-3,4-dihydrophthalazin-1-yl)ethyl)-3-(3,4-difluorophenyl)-1-methylurea;
1-(1-(6,7-difluoro-4-oxo-3,4-dihydrophthalazin-1-yl)ethyl)-1-methyl-3-(3,4,5-trifluorophenyl)urea;
1-(1-(6,7-difluoro-4-oxo-3,4-dihydrophthalazin-1-yl)ethyl)-3 -(4-fluorophenyl)-1-methylurea;
3 -(3 -chloro-4-fluorophenyl)-1-(1 -(5 -fluoro-4-oxo-3,4-dihydrophthalazin-1-yl)ethyl)-1-methylurea;
3 -(3,4-difluorophenyl)-1 -(1 -(5 -fluoro-4-oxo-3,4-dihydrophthalazin-1 -yl)ethyl)-1 -methylurea;
N-(1-(6,7-difluoro-4-oxo-3,4-dihydrophthalazin-1-yl)ethyl)-N-methyl-1H-indole-2-carboxamide;
N-(1-(6,7-difluoro-4-oxo-3,4-dihydrophthalazin-1-yl)ethyl)-N-ethyl-5-fluoro-1H-indole-2-carboxamide;
N-(1-(6,7-difluoro-4-oxo-3,4-dihydrophthalazin-1-yl)ethyl)-N-ethyl-1H-indole-2-carboxamide;
N-(1-(6,7-difluoro-4-oxo-3,4-dihydrophthalazin-1-yl)ethyl)-N-ethyl-4, 5-difluoro-1H-indole-2-carboxamide;
N-(1-(6,7-difluoro-4-oxo-3,4-dihydrophthalazin-1-yl)ethyl)-5,6-difluoro-N-methyl-1H-indole-2-carboxamide;
N-(1-(6,7-difluoro-4-oxo-3,4-dihydrophthalazin-1-yl)ethyl)-5-fluoro-N-methyl-1H-indole-2-carboxamide;
N-(1-(6,7-difluoro-4-oxo-3,4-dihydrophthalazin-1-yl)ethyl)-6-fluoro-N-methyl-1H-indole-2-carboxamide;
N-(1-(6,7-difluoro-4-oxo-3,4-dihydrophthalazin-1-yl)ethyl)-4-fluoro-N-methyl-1H-indole-2-carboxamide;
N-(1-(6,7-difluoro-4-oxo-3,4-dihydrophthalazin-1-yl)ethyl)-4, 5-difluoro-N-methyl-1H-indole-2-carboxamide;
N-(1-(6,7-difluoro-4-oxo-3,4-dihydrophthalazin-1-yl)ethyl)-N,3-dimethyl-1H-indole-2-carboxamide;
N-(1-(6,7-difluoro-4-oxo-3,4-dihydrophthalazin-1-yl)ethyl)-N-isobutyl-1H-indole-2-carboxamide;
N-(1-(6,7-difluoro-4-oxo-3,4-dihydrophthalazin-1-yl)ethyl)-N-methyl-4,5,6,7-tetrahydro-1H-indole-2-carboxamide;
N-(1-(6,7-difluoro-4-oxo-3,4-dihydrophthalazin-1-yl)ethyl)-N-methylindolizine-2-carboxamide;
N-(1-(6,7-difluoro-4-oxo-3,4-dihydrophthalazin-1-yl)ethyl)-4,6-difluoro-N-methyl-1H-indole-2-carboxamide;
N-(1-(6,7-difluoro-4-oxo-3,4-dihydrophthalazin-1-yl)ethyl)-3-fluoro-N-methyl-4-(trifluoromethyl)benzamide;
4-bromo-N-(1-(6,7-difluoro-4-oxo-3,4-dihydrophthalazin-1-yl)ethyl)-3-fluoro-N-methylbenzamide;
4-chloro-N-(1-(6,7-difluoro-4-oxo-3,4-dihydrophthalazin-1-yl)ethyl)-3-fluoro-N-methylbenzamide;
4-bromo-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-N-methyl-4-(trifluoromethyl)benzamide;
N-(1-(6,7-difluoro-4-oxo-3,4-dihydrophthalazin-1-yl)ethyl)-3,4,5-trifluoro-N-methylbenzamide;
3-chloro-N-(1-(6,7-difluoro-4-oxo-3,4-dihydrophthalazin-1-yl)ethyl)-4-fluoro-N-methylbenzamide;
4-chloro-N-(1-(6,7-difluoro-4-oxo-3,4-dihydrophthalazin-1-yl)ethyl)-N-methylbenzamide;
N-(1-(6,7-difluoro-4-oxo-3,4-dihydrophthalazin-1-yl)ethyl)-3,4-difluoro-N-methylbenzamide;
N-(1-(6,7-difluoro-4-oxo-3,4-dihydrophthalazin-1-yl)ethyl)-3-(difluoromethyl)-N-methylbenzamide;
N-(1-(6,7-difluoro-4-oxo-3,4-dihydrophthalazin-1-yl)ethyl)-3-(difluoromethyl)-4-fluoro-N-methylbenzamide;
1-(1 -(6,7-difluoro-4-oxo-3,4-dihydrophthalazin-1 -yl)ethyl)-3 -(3 -(difluoromethyl)-4-fluorophenyl)-1-methylurea;
N-(1-(6,7-difluoro-4-oxo-3,4-dihydrophthalazin-1-yl)ethyl)-N-methylbenzamide;
8-chloro-N-(1-(6,7-difluoro-4-oxo-3,4-dihydrophthalazin-1-yl)ethyl)-N-methylindolizine-2-carboxamide;
3 -(3 -cyano-4-fluorophenyl)-1 -(1 -(6,7-difluoro-4-oxo-3,4-dihydrophthalazin-1 -yl)ethyl)-1 -methylurea;
N-(1-(6,7-difluoro-4-oxo-3,4-dihydrophthalazin-1-yl)ethyl)-8-fluoro-N-methylindolizine-2-carboxamide;
N-(1-(6,7-difluoro-4-oxo-3,4-dihydrophthalazin-1-yl)ethyl)-N-methylindoline-2-carboxamide;
2-chloro-N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-4H-thieno[3,2-b]pyrrole-5-carboxamide;
N-(1-(6,7-difluoro-4-oxo-3,4-dihydrophthalazin-1-yl)ethyl)-N-methyl-2,3-dihydro-1H-indene-5-carboxamide;
N-(1-(6,7-difluoro-4-oxo-3,4-dihydrophthalazin-1-yl)ethyl)-N-methylbenzo[d]thiazole-5-carboxamide;
N-(1-(6,7-difluoro-4-oxo-3,4-dihydrophthalazin-1-yl)ethyl)-N-methylbenzo[d]thiazole-6-carboxamide;
N-(1-(6,7-difluoro-4-oxo-3,4-dihydrophthalazin-1-yl)ethyl)-N-methylbenzo[d]oxazole-5-carboxamide;
N-(1-(6,7-difluoro-4-oxo-3,4-dihydrophthalazin-1-yl)ethyl)-N-methylbenzo[d]oxazole-6-carboxamide;
5-chloro-N-(1-(6,7-difluoro-4-oxo-3,4-dihydrophthalazin-1-yl)ethyl)-N-methyl-6-(trifluoromethyl)nicotinamide;
4-chloro-N-(1-(6,7-difluoro-4-oxo-3,4-dihydrophthalazin-1-yl)ethyl)-3, 5-difluoro-N-methylbenzamide;
N-(1-(6,7-difluoro-4-oxo-3,4-dihydrophthalazin-1-yl)ethyl)-4-(difluoromethyl)-6-fluoro-N-methyl-1H-indole-2-carboxamide;
N-(1-(6,7-difluoro-4-oxo-3,4-dihydrophthalazin-1-yl)ethyl)-8-(difluoromethyl)-N-methylindolizine-2-carboxamide;
4-bromo-N-(1-(6,7-difluoro-4-oxo-3,4-dihydrophthalazin-1-yl)ethyl)-3,5-difluoro-N-methylbenzamide;
N-(1-(6,7-difluoro-4-oxo-3,4-dihydrophthalazin-1-yl)ethyl)-2-fluoro-N-methyl-4H-thieno[3,2-b]pyrrole-5-carboxamide;
N-(1 -(6,7-difluoro-4-oxo-3,4-dihydrophthalazin-1 -yl)ethyl)-N, 1-dimethyl-1H-indole-6-carboxamide;
N-(1-(6,7-difluoro-4-oxo-3,4-dihydrophthalazin-1-yl)ethyl)-N,1-dimethyl-1H-indole-5-carboxamide;
N-(1-(6,7-difluoro-4-oxo-3,4-dihydrophthalazin-1-yl)ethyl)-4-(difluoromethyl)-3,5-difluoro-N-methylbenzamide;
N-(1-(6,7-difluoro-4-oxo-3,4-dihydrophthalazin-1-yl)ethyl)-N-methyl-1H-indazole-5-carboxamide;
N-(1-(6,7-difluoro-4-oxo-3,4-dihydrophthalazin-1-yl)ethyl)-N-methyl-1H-indazole-6-carboxamide; and
N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled derivative, stereoisomer, or tautomer thereof, or any mixtures thereof.

17. The compound of claim 1, which is at least one selected from the group consisting of:
(R)-3-(3-chloro-4-fluorophenyl)-1-methyl-1-(1-(4-oxo-3,4-dihydrophthalazin-1-yl)ethyl)urea;
(S)-3-(3-chloro-4-fluorophenyl)-1-methyl-1-(1-(4-oxo-3,4-dihydrophthalazin-1-yl)ethyl)urea;
(R)-3-(3-chloro-4-fluorophenyl)-1-isobutyl-1-(1-(4-oxo-3,4-dihydrophthalazin-1-yl)ethyl)urea;
(S)-3-(3-chloro-4-fluorophenyl)-1-isobutyl-1-(1-(4-oxo-3,4-dihydrophthalazin-1-yl)ethyl)urea;
(R)-3-(3-chloro-4-fluorophenyl)-1-isobutyl-1-(1-(3-methyl-4-oxo-3,4-dihydrophthalazin-1-yl)ethyl)urea;
(S)-3-(3-chloro-4-fluorophenyl)-1-isobutyl-1-(1-(3-methyl-4-oxo-3,4-dihydrophthalazin-1-yl)ethyl)urea;
(R)-3-(4-fluorophenyl)-1-isobutyl-1-(1-(3-methyl-4-oxo-3,4-dihydrophthalazin-1-yl)ethyl)urea;
(S)-3-(4-fluorophenyl)-1-isobutyl-1-(1-(3-methyl-4-oxo-3,4-dihydrophthalazin-1-yl)ethyl)urea;
(R)-3-(3-chloro-4-fluorophenyl)-1-(1-(6-chloro-4-oxo-3,4-dihydrophthalazin-1-yl)ethyl)-1-isobutylurea;
(S)-3-(3-chloro-4-fluorophenyl)-1-(1-(6-chloro-4-oxo-3,4-dihydrophthalazin-1-yl)ethyl)-1-isobutylurea;
(R)-1-(1-(6-chloro-4-oxo-3,4-dihydrophthalazin-1-yl)ethyl)-3-(4-fluorophenyl)-1-isobutylurea;
(S)-1-(1-(6-chloro-4-oxo-3,4-dihydrophthalazin-1-yl)ethyl)-3-(4-fluorophenyl)-1-isobutylurea;
(R)-3-(3-chloro-4-fluorophenyl)-1-(1-(6-chloro-4-oxo-3,4-dihydrophthalazin-1-yl)ethyl)-1-methylurea;
(S)-3-(3-chloro-4-fluorophenyl)-1-(1-(6-chloro-4-oxo-3,4-dihydrophthalazin-1-yl)ethyl)-1-methylurea;
(R)-3-(3-chloro-4-fluorophenyl)-1-(1-(6-chloro-4-oxo-3,4-dihydrophthalazin-1-yl)ethyl)-1-(3-hydroxypropyl)urea;
(S)-3-(3-chloro-4-fluorophenyl)-1-(1-(6-chloro-4-oxo-3,4-dihydrophthalazin-1-yl)ethyl)-1-(3-hydroxypropyl)urea;
(R)-1-(1-(6-chloro-4-oxo-3,4-dihydrophthalazin-1-yl)ethyl)-3-(4-fluorophenyl)-1-(3-hydroxypropyl)urea;
(S)-1-(1 -(6-chloro-4-oxo-3,4-dihydrophthalazin-1-yl)ethyl)-3 -(4-fluorophenyl)-1-(3 -hydroxypropyl)urea;
(R)-3 -(3 -chloro-4-fluorophenyl)-1-(1 -(6-fluoro-4-oxo-3,4-dihydrophthalazin-1-yl)ethyl)-1-isobutylurea;
(S)-3 -(3 -chloro-4-fluorophenyl)-1-(1 -(6-fluoro-4-oxo-3,4-dihydrophthalazin-1-yl)ethyl)-1-isobutylurea;
(R)-1-(1-(6-fluoro-4-oxo-3,4-dihydrophthalazin-1-yl)ethyl)-3-(4-fluorophenyl)-1-isobutylurea;
(S)- 1 -(1 -(6-fluoro-4-oxo-3,4-dihydrophthalazin-1-yl)ethyl)-3 -(4-fluorophenyl)-1-isobutylurea;
(R)-3 -(3 -chloro-4-fluorophenyl)-1-(1 -(6-fluoro-4-oxo-3,4-dihydrophthalazin-1-yl)ethyl)-1-methylurea;
(S)-3 -(3 -chloro-4-fluorophenyl)-1-(1 -(6-fluoro-4-oxo-3,4-dihydrophthalazin-1-yl)ethyl)-1-methylurea;
(R)-1-(1-(6-fluoro-4-oxo-3,4-dihydrophthalazin-1-yl)ethyl)-3-(4-fluorophenyl)-1-methylurea;
(S)- 1 -(1 -(6-fluoro-4-oxo-3,4-dihydrophthalazin-1-yl)ethyl)-3 -(4-fluorophenyl)-1-methylurea;
(R)-3 -(4-fluorophenyl)-1 -isobutyl-1 -(1-(4-oxo-3,4-dihydrophthal azin- 1 -yl)ethyl)urea;
(S)-3-(4-fluorophenyl)-1-isobutyl-1-(1-(4-oxo-3,4-dihydrophthalazin-1-yl)ethyl)urea;
(R)-3-(3-chloro-4-fluorophenyl)-1-(1-(6,7-difluoro-3-methyl-4-oxo-3,4-dihydrophthalazin-1-yl)ethyl)-1-methylurea;
(S)-3-(3-chloro-4-fluorophenyl)-1-(1-(6,7-difluoro-3-methyl-4-oxo-3,4-dihydrophthalazin-1-yl)ethyl)-1-methylurea;
(R)-3-(3-chloro-4-fluorophenyl)-1-(1-(6,7-difluoro-3-methyl-4-oxo-3,4-dihydrophthalazin-1-yl)ethyl)-1-isobutylurea;
(S)-3-(3-chloro-4-fluorophenyl)-1-(1-(6,7-difluoro-3-m ethyl-4-oxo-3,4-dihydrophthalazin-1-yl)ethyl)-1-isobutylurea;
(R)-1 -(1 -(6,7-difluoro-3 -methyl-4-oxo-3,4-dihydrophthalazin-1-yl)ethyl)-3 -(4-fluorophenyl)-1-isobutylurea;
(S)- 1 -(1 -(6,7-difluoro-3 -methyl-4-oxo-3,4-dihydrophthalazin-1-yl)ethyl)-3 -(4-fluorophenyl)-1-isobutylurea;
(R)-3-(3 -chloro-4-fluorophenyl)-1-(1-(6,7-difluoro-3-methyl-4-oxo-3,4-dihydrophthalazin-1-yl)ethyl)-1-(3-hydroxypropyl)urea;
(S)-3-(3 -chloro-4-fluorophenyl)-1-(1-(6,7-difluoro-3-methyl-4-oxo-3,4-dihydrophthalazin-1-yl)ethyl)-1-(3-hydroxypropyl)urea;
(R)-3 -(4-fluorophenyl)-1 -(1 -(6,7-difluoro-3 -methyl-4-oxo-3,4-dihydrophthalazin-1 -yl)ethyl)-1 -(3 -hydroxypropyl)urea;
(S)-3-(4-fluorophenyl)-1-(1-(6,7-difluoro-3-methyl-4-oxo-3,4-dihydrophthalazin-1-yl)ethyl)-1-(3 -hydroxypropyl)urea;
(R)-3 -(3 -chloro-4-fluorophenyl)-1 -(1 -(6,7-difluoro-4-oxo-3,4-dihydrophthalazin-1 -yl)ethyl)-1 -isobutylurea;
(S)-3 -(3 -chloro-4-fluorophenyl)-1 -(1 -(6,7-difluoro-4-oxo-3,4-dihy drophthal azin- 1 -yl)ethyl)- 1 -isobutylurea;
(R)-1-(1-(6,7-difluoro-4-oxo-3,4-dihydrophthalazin-1-yl)ethyl)-1-isobutyl-3-phenylurea;
(S)-1-(1-(6,7-difluoro-4-oxo-3,4-dihydrophthalazin-1-yl)ethyl)-1-isobutyl-3-phenylurea;
(R)-3 -(3 -chloro-4-fluorophenyl)-1 -(1 -(6,7-difluoro-4-oxo-3,4-dihydrophthalazin-1 -yl)ethyl)-1 -methylurea;
(S)-3 -(3 -chloro-4-fluorophenyl)-1 -(1 -(6,7-difluoro-4-oxo-3,4-dihy drophthal azin- 1 -yl)ethyl)- 1 -methylurea;
(R) -2-(3-(3-chloro-4-fluorophenyl)-I-(I-(6,7-difluoro-3-methyl-4-oxo-3,4-dihydrophthalazin-I-yl)ethyl)ureido)-N-((3-chloro-4-fluorophenyl)carbamoyl)ethane-1-sulfonamide;
(S)-2-(3-(3-chloro-4-fluorophenyl)-1-(1-(6,7-difluoro-3-methyl-4-oxo-3,4-dihydrophthal azin-1-yl)ethyl)ureido)-N-((3-chloro-4-fluorophenyl)carbamoyl)ethane-1-sulfonamide;
(R)-3-cyclopropyl-1-(1-(6,7-difluoro-3-methyl-4-oxo-3,4-dihydrophthalazin-1-yl)ethyl)-1-isobutylurea;
(S)-3-cyclopropyl-1-(1-(6,7-difluoro-3-methyl-4-oxo-3,4-dihydrophthalazin-1-yl)ethyl)-1-isobutylurea;
(R)- 1 -(1 -(6,7-difluoro-3 -m ethyl-4-oxo-3, 4-dihy drophthal azin- 1 -yl)ethyl)- 1 -i sobutyl-3 -phenylurea;
(S)-1-(1-(6,7-difluoro-3-methyl-4-oxo-3,4-dihydrophthalazin-1-yl)ethyl)-1-isobutyl-3-phenylurea;
(R)-3-cyclopentyl-1-(1-(6,7-difluoro-3-methyl-4-oxo-3,4-dihydrophthalazin-1-yl)ethyl)-1-isobutylurea;
(S)-3-cyclopentyl-1-(1-(6,7-difluoro-3-methyl-4-oxo-3,4-dihydrophthalazin-1-yl)ethyl)-1-isobutylurea;
(R)-1 -(1 -(6,7-difluoro-3 -methyl-4-oxo-3,4-dihydrophthalazin-1 -yl)ethyl)-3 -(4-fluorophenyl)-1 -methylurea;
(S)- 1 -(1 -(6,7-difluoro-3 -methyl-4-oxo-3,4-dihydrophthalazin-1 -yl)ethyl)-3 -(4-fluorophenyl)-1 -methylurea;
(R)-1-(1-(6,7-difluoro-4-oxo-3,4-dihydrophthalazin-1-yl)ethyl)-3-(3,4-difluorophenyl)-1 -methylurea;
(S)-1-(1-(6,7-difluoro-4-oxo-3,4-dihydrophthalazin-1-yl)ethyl)-3-(3,4-difluorophenyl)-1-methylurea;
(R)-1-(1-(6,7 -difluoro-4-oxo-3,4-dihydrophthalazin-1-yl)ethyl)-1-methyl-3-(3,4,5-trifluorophenyl)urea;
(S)-1-(1-(6,7-difluoro-4-oxo-3,4-dihydrophthalazin-1-yl)ethyl)-1-methyl-3-(3,4,5-trifluorophenyl)urea;
(R)- 1 -(1 -(6,7-difluoro-4-oxo-3,4-dihydrophthalazin-1 -yl)ethyl)-3 -(4-fluorophenyl)-1 -methylurea;
(S)- 1 -(1 -(6,7-difluoro-4-oxo-3,4-dihydrophthalazin-1 -yl)ethyl)-3 -(4-fluorophenyl)-1 -methylurea;
(R)-3 -(3 -chloro-4-fluorophenyl)-1 -(1 -(5-fluoro-4-oxo-3,4-dihydrophthalazin-1 -yl)ethyl)-1 -methylurea;
(S)-3 -(3 -chloro-4-fluorophenyl)-1 -(1 -(5-fluoro-4-oxo-3,4-dihydrophthalazin-1 -yl)ethyl)-1 -methylurea;
(R)-3-(3,4-difluorophenyl)-1-(1-(5-fluoro-4-oxo-3,4-dihydrophthalazin-1-yl)ethyl)-1-methylurea;
(S)-3-(3,4-difluorophenyl)-1-(1-(5-fluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-N-methyl-1H-indole-2-carboxamide;
(S)-N-(1-(6,7-difluoro-4-oxo-3,4-dihydrophthalazin-1-yl)ethyl)-N-methyl-1H-indole-2-carboxamide;
(R)-N-(1-(6,7-difluoro-4-oxo-3,4-dihydrophthalazin-1-yl)ethyl)-N-ethyl-5-fluoro-1H-indole-2-carboxamide;
(S)-N-(1-(6,7-difluoro-4-oxo-3,4-dihydrophthalazin-1-yl)ethyl)-N-ethyl-5-fluoro-1H-indole-2-carboxamide;
(R)-N-(1-(6,7-difluoro-4-oxo-3,4-dihydrophthalazin-1-yl)ethyl)-N-ethyl-1H-indole-2-carboxamide;
(S)-N-(1-(6,7-difluoro-4-oxo-3,4-dihydrophthalazin-1-yl)ethyl)-N-ethyl-1H-indole-2-carboxamide;
(R)-N-(1-(6,7-difluoro-4-oxo-3,4-dihydrophthalazin-1-yl)ethyl)-N-ethyl-4,5-difluoro-1H-indole-2-carboxamide;
(S)-N-(1-(6,7-difluoro-4-oxo-3,4-dihydrophthalazin-1-yl)ethyl)-N-ethyl-4,5-difluoro-1H-indole-2-carboxamide;
(R)-N-(1-(6,7-difluoro-4-oxo-3,4-dihydrophthalazin-1-yl)ethyl)-5,6-difluoro-N-methyl-1H-indole-2-carboxamide;
(S)-N-(1-(6,7-difluoro-4-oxo-3,4-dihydrophthalazin-1-yl)ethyl)-5,6-difluoro-N-methyl-1H-indole-2-carboxamide;
(R)-N-(1-(6,7-difluoro-4-oxo-3,4-dihydrophthalazin-1-yl)ethyl)-5-fluoro-N-methyl-1H-indole-2-carboxamide;
(S)-N-(1-(6,7-difluoro-4-oxo-3,4-dihydrophthalazin-1-yl)ethyl)-5-fluoro-N-methyl-1H-indole-2-carboxamide;
(R)-N-(1-(6,7-difluoro-4-oxo-3,4-dihydrophthalazin-1-yl)ethyl)-6-fluoro-N-methyl-1H-indole-2-carboxamide;
(S)-N-(1-(6,7-difluoro-4-oxo-3,4-dihydrophthalazin-1-yl)ethyl)-6-fluoro-N-methyl-1H-indole-2-carboxamide;
(R)-N-(1-(6,7-difluoro-4-oxo-3,4-dihydrophthalazin-1-yl)ethyl)-4-fluoro-N-methyl-1H-indole-2-carboxamide;
(S)-N-(1-(6,7-difluoro-4-oxo-3,4-dihydrophthalazin-1-yl)ethyl)-4-fluoro-N-methyl-1H-indole-2-carboxamide;
(R)-N-(1-(6,7-difluoro-4-oxo-3,4-dihydrophthalazin-1-yl)ethyl)-4,5-difluoro-N-methyl-1H-indole-2-carboxamide;
(S)-N-(1-(6,7-difluoro-4-oxo-3,4-dihydrophthalazin-1-yl)ethyl)-4,5-difluoro-N-methyl-1H-indole-2-carboxamide;
(R)-N-(1-(6,7-difluoro-4-oxo-3,4-dihydrophthalazin-1-yl)ethyl)-N,3-dimethyl-1H-indole-2-carboxamide;
(S)-N-(1-(6,7-difluoro-4-oxo-3,4-dihydrophthalazin-1-yl)ethyl)-N,3-dimethyl-1H-indole-2-carboxamide;
(R)-N-(1-(6,7-difluoro-4-oxo-3,4-dihydrophthalazin-1-yl)ethyl)-N-isobutyl-1H-indole-2-carboxamide;
(S)-N-(1-(6,7-difluoro-4-oxo-3,4-dihydrophthalazin-1-yl)ethyl)-N-isobutyl-1H-indole-2-carboxamide;
(R)-N-(1-(6,7-difluoro-4-oxo-3,4-dihydrophthalazin-1-yl)ethyl)-N-methyl-4,5,6,7-tetrahydro-1H-indole-2-carboxamide;
(S)-N-(1-(6,7-difluoro-4-oxo-3,4-dihydrophthalazin-1-yl)ethyl)-N-methyl-4,5,6,7-tetrahydro-1H-indole-2-carboxamide;
(R)-N-(1-(6,7-difluoro-4-oxo-3,4-dihydrophthalazin-1-yl)ethyl)-N-methylindolizine-2-carboxamide;
(S)-N-(1-(6,7-difluoro-4-oxo-3,4-dihydrophthalazin-1-yl)ethyl)-N-methylindolizine-2-carboxamide;
(R)-N-(1-(6,7-difluoro-4-oxo-3,4-dihydrophthalazin-1-yl)ethyl)-4,6-difluoro-N-methyl-1H-indole-2-carboxamide;
(S)-N-(1-(6,7-difluoro-4-oxo-3,4-dihydrophthalazin-1-yl)ethyl)-4,6-difluoro-N-methyl-1H-indole-2-carboxamide;
(R)-N-(1-(6,7-difluoro-4-oxo-3,4-dihydrophthalazin-1-yl)ethyl)-3-fluoro-N-methyl-4-(trifluoromethyl)benzamide;
(S)-N-(1-(6,7-difluoro-4-oxo-3,4-dihydrophthalazin-1-yl)ethyl)-3-fluoro-N-methyl-4-(trifluoromethyl)benzamide;
(R)-4-bromo-N-(1-(6,7-difluoro-4-oxo-3,4-dihydrophthalazin-1-yl)ethyl)-3-fluoro-N-methylbenzamide;
(S)-4-bromo-N-(1-(6,7-difluoro-4-oxo-3,4-dihydrophthalazin-1-yl)ethyl)-3-fluoro-N-methylbenzamide;
(R)-4-chloro-N-(1-(6,7-difluoro-4-oxo-3,4-dihydrophthalazin-1-yl)ethyl)-3-fluoro-N-methylbenzamide;
(S)-4-chloro-N-(1-(6,7-difluoro-4-oxo-3,4-dihydrophthalazin-1-yl)ethyl)-3-fluoro-N-methylbenzamide;
(R)-4-bromo-N-(1-(6,7-difluoro-4-oxo-3,4-dihydrophthalazin-1-yl)ethyl)-N-methylbenzamide;
(S)-4-bromo-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-N-methyl-4-(trifluoromethyl)benzamide;
(S)-N-(1-(6,7-difluoro-4-oxo-3,4-dihydrophthalazin-1-yl)ethyl)-N-methyl-4-(trifluoromethyl)benzamide;
(R)-N-(1-(6,7-difluoro-4-oxo-3,4-dihydrophthalazin-1-yl)ethyl)-3,4,5-trifluoro-N-methylbenzamide;
(S)-N-(1-(6,7-difluoro-4-oxo-3,4-dihydrophthalazin-1-yl)ethyl)-3,4,5-trifluoro-N-methylbenzamide;
(R)-3-chloro-N-(1-(6,7-difluoro-4-oxo-3,4-dihydrophthalazin-1-yl)ethyl)-4-fluoro-N-methylbenzamide;
(S)-3-chloro-N-(1-(6,7-difluoro-4-oxo-3,4-dihydrophthalazin-1-yl)ethyl)-4-fluoro-N-methylbenzamide;
(R)-4-chloro-N-(1-(6,7-difluoro-4-oxo-3,4-dihydrophthalazin-1-yl)ethyl)-N-methylbenzamide;
(S)-4-chloro-N-(1-(6,7-difluoro-4-oxo-3,4-dihydrophthalazin-1-yl)ethyl)-N-methylbenzamide;
(R)-N-(1-(6,7-difluoro-4-oxo-3,4-dihydrophthalazin-1-yl)ethyl)-3,4-difluoro-N-methylbenzamide;
(S)-N-(1-(6,7-difluoro-4-oxo-3,4-dihydrophthalazin-1-yl)ethyl)-3,4-difluoro-N-methylbenzamide;
(R)-N-(1-(6,7-difluoro-4-oxo-3,4-dihydrophthalazin-1-yl)ethyl)-3-(difluoromethyl)-N-methylbenzamide;
(S)-N-(1-(6,7-difluoro-4-oxo-3,4-dihydrophthalazin-1-yl)ethyl)-3-(difluoromethyl)-N-methylbenzamide;
(R)-N-(1-(6,7-difluoro-4-oxo-3,4-dihydrophthalazin-1-yl)ethyl)-3-(difluoromethyl)-4-fluoro-N-methylbenzamide;
(S)-N-(1-(6,7-difluoro-4-oxo-3,4-dihydrophthalazin-1-yl)ethyl)-3-(difluoromethyl)-4-fluoro-N-methylbenzamide;
(R)-1-(1-(6,7-difluoro-4-oxo-3,4-dihydrophthalazin-1-yl)ethyl)-3-(3-(difluoromethyl)-4-fluorophenyl)-1-methylurea;
(S)-1-(1-(6,7-difluoro-4-oxo-3,4-dihydrophthalazin-1-yl)ethyl)-3-(3-(difluoromethyl)-4-fluorophenyl)-1-methylurea;
(R)-N-(1-(6,7-difluoro-4-oxo-3,4-dihydrophthalazin-1-yl)ethyl)-N-methylbenzamide;
(S)-N-(1-(6,7-difluoro-4-oxo-3,4-dihydrophthalazin-1-yl)ethyl)-N-methylbenzamide;
(R)-8-chloro-N-(1-(6,7-difluoro-4-oxo-3,4-dihydrophthalazin-1-yl)ethyl)-N-methylindolizine-2-carboxamide;
(S)-8-chloro-N-(1-(6,7-difluoro-4-oxo-3,4-dihydrophthalazin-1-yl)ethyl)-N-methylindolizine-2-carboxamide;
(R)-3-(3-cyano-4-fluorophenyl)-1-(1-(6,7-difluoro-4-oxo-3,4-dihydrophthalazin-1-yl)ethyl)-1-methylurea;
(S)-3-(3-cyano-4-fluorophenyl)-1-(1-(6,7-difluoro-4-oxo-3,4-dihydrophthalazin-1-yl)ethyl)-1-methylurea;
(R)-N-(1-(6,7-difluoro-4-oxo-3,4-dihydrophthalazin-1-yl)ethyl)-8-fluoro-N-methylindolizine-2-carboxamide;
(S)-N-(1-(6,7-difluoro-4-oxo-3,4-dihydrophthalazin-1-yl)ethyl)-8-fluoro-N-methylindolizine-2-carboxamide;
(2S)-N-(1-(6,7-difluoro-4-oxo-3,4-dihydrophthalazin-1-yl)ethyl)-N-methylindoline-2-carboxamide;
(S)-N-((S)-1-(6,7-difluoro-4-oxo-3,4-dihydrophthalazin-1-yl)ethyl)-N-methylindoline-2-carboxamide;
(S)-N-((R)-1-(6,7-difluoro-4-oxo-3,4-dihydrophthalazin-1-yl)ethyl)-N-methylindoline-2-carboxamide;
(2R)-N-(1-(6,7-difluoro-4-oxo-3,4-dihydrophthalazin-1-yl)ethyl)-N-methylindoline-2-carboxamide;
(R)-N-((R)-1-(6,7-difluoro-4-oxo-3,4-dihydrophthalazin-1-yl)ethyl)-N-methylindoline-2-carboxamide;
(R)-N-((S)-1-(6,7-difluoro-4-oxo-3,4-dihydrophthalazin-1-yl)ethyl)-N-methylindoline-2-carboxamide;
(R)-2-chloro-N-(1-(6,7-difluoro-4-oxo-3,4-dihydrophthalazin-1-yl)ethyl)-N-methyl-4H-thieno[3,2-b]pyrrole-5-carboxamide;
(S)-2-chloro-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-4H-thieno[3,2-b]pyrrole-5-carboxamide;
(S)-N-(1-(6,7-difluoro-4-oxo-3,4-dihydrophthalazin-1-yl)ethyl)-N-methyl-4H-thieno[3,2-b]pyrrole-5-carboxamide;
(R)-N-(1-(6,7-difluoro-4-oxo-3,4-dihydrophthalazin-1-yl)ethyl)-N-methyl-2,3-dihydro-1H-indene-5-carboxamide;
(S)-N-(1-(6,7-difluoro-4-oxo-3,4-dihydrophthalazin-1-yl)ethyl)-N-methyl-2,3-dihydro-1H-indene-5-carboxamide;
(R)-N-(1-(6,7-difluoro-4-oxo-3,4-dihydrophthalazin-1-yl)ethyl)-N-methylbenzo[d]thiazole-5-carboxamide;
(S)-N-(1-(6,7-difluoro-4-oxo-3,4-dihydrophthalazin-1-yl)ethyl)-N-methylbenzo[d]thiazole-5-carboxamide;
(R)-N-(1-(6,7-difluoro-4-oxo-3,4-dihydrophthalazin-1-yl)ethyl)-N-methylbenzo[d]thiazole-6-carboxamide;
(S)-N-(1-(6,7-difluoro-4-oxo-3,4-dihydrophthalazin-1-yl)ethyl)-N-methylbenzo[d]thiazole-6-carboxamide;
(R)-N-(1-(6,7-difluoro-4-oxo-3,4-dihydrophthalazin-1-yl)ethyl)-N-methylbenzo[d]oxazole-5-carboxamide;
(S)-N-(1-(6,7-difluoro-4-oxo-3,4-dihydrophthalazin-1-yl)ethyl)-N-methylbenzo[d]oxazole-5-carboxamide;
(R)-N-(1-(6,7-difluoro-4-oxo-3,4-dihydrophthalazin-1-yl)ethyl)-N-methylbenzo[d]oxazole-6-carboxamide;
(S)-N-(1-(6,7-difluoro-4-oxo-3,4-dihydrophthalazin-1-yl)ethyl)-N-methylbenzo[d]oxazole-6-carboxamide;
(R)-5-chloro-N-(1-(6,7-difluoro-4-oxo-3,4-dihydrophthalazin-1-yl)ethyl)-N-methyl-6-(trifluoromethyl)nicotinamide;
(S)-5-chloro-N-(1-(6,7-difluoro-4-oxo-3,4-dihydrophthalazin-1-yl)ethyl)-N-methyl-6-(trifluoromethyl)nicotinamide;
(R)-4-chloro-N-(1-(6,7-difluoro-4-oxo-3,4-dihydrophthalazin-1-yl)ethyl)-3,5-difluoro-N-methylbenzamide;
(S)-4-chloro-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-4-(difluoromethyl)-6-fluoro-N-methyl-1H-indole-2-carboxamide;
(S)-N-(1-(6,7-difluoro-4-oxo-3,4-dihydrophthalazin-1-yl)ethyl)-4-(difluoromethyl)-6-fluoro-N-methyl-1H-indole-2-carboxamide;
(R)-N-(1-(6,7-difluoro-4-oxo-3,4-dihydrophthalazin-1-yl)ethyl)-8-(difluoromethyl)-N-methylindolizine-2-carboxamide;
(S)-N-(1-(6,7-difluoro-4-oxo-3,4-dihydrophthalazin-1-yl)ethyl)-8-(difluoromethyl)-N-methylindolizine-2-carboxamide;
(R)-4-bromo-N-(1-(6,7-difluoro-4-oxo-3,4-dihydrophthalazin-1-yl)ethyl)-3,5-difluoro-N-methylbenzamide;
(S)-4-bromo-N-(1-(6,7-difluoro-4-oxo-3,4-dihydrophthalazin-1-yl)ethyl)-3,5-difluoro-N-methylbenzamide;
(R)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(S)-N-(1-(6,7-difluoro-4-oxo-3,4-dihydrophthalazin-1-yl)ethyl)-2-fluoro-N-methyl-4H-thieno[3,2-b]pyrrole-5-carboxamide;
(R)-N-(1-(6,7-difluoro-4-oxo-3,4-dihydrophthalazin-1-yl)ethyl)-N,1-dimethyl-1H-indole-6-carboxamide;
(S)-N-(1-(6,7-difluoro-4-oxo-3,4-dihydrophthalazin-1-yl)ethyl)-N,1-dimethyl-1H-indole-6-carboxamide;
(R)-N-(1-(6,7-difluoro-4-oxo-3,4-dihydrophthalazin-1-yl)ethyl)-N,1-dimethyl-1H-indole-5-carboxamide;
(S)-N-(1-(6,7-difluoro-4-oxo-3,4-dihydrophthalazin-1-yl)ethyl)-N,1-dimethyl-1H-indole-5-carboxamide;
(R)-N-(1-(6,7-difluoro-4-oxo-3,4-dihydrophthalazin-1-yl)ethyl)-4-(difluoromethyl)-3,5-difluoro-N-methylbenzamide;
(S)-N-(1-(6,7-difluoro-4-oxo-3,4-dihydrophthalazin-1-yl)ethyl)-4-(difluoromethyl)-3,5-difluoro-N-methylbenzamide;
(R)-N-(1-(6,7-difluoro-4-oxo-3,4-dihydrophthalazin-1-yl)ethyl)-N-methyl-1H-indazole-5-carboxamide;
(S)-N-(1-(6,7-difluoro-4-oxo-3,4-dihydrophthalazin-1-yl)ethyl)-N-methyl-1H-indazole-5-carboxamide;
(R)-N-(1-(6,7-difluoro-4-oxo-3,4-dihydrophthalazin-1-yl)ethyl)-N-methyl-1H-indazole-6-carboxamide;
(S)-N-(1-(6,7-difluoro-4-oxo-3,4-dihydrophthalazin-1-yl)ethyl)-N-methyl-1H-indazole-6-carboxamide;
(R)-N-(1-(6,7-difluoro-4-oxo-3,4-dihydrophthalazin-1-yl)ethyl)-5-fluoro-N-methyl-6-(trifluoromethyl)nicotinamide; and
(S)-N-(1-(6,7-difluoro-4-oxo-3,4-dihydrophthalazin-1-yl)ethyl)-5-fluoro-N-methyl-6-(trifluoromethyl)nicotinamide;
or a salt, solvate, prodrug, isotopically labelled derivative, stereoisomer, or tautomer thereof, or any mixtures thereof.

18. A pharmaceutical composition comprising at least one compound of claim 1 and a pharmaceutically acceptable carrier.

19. The pharmaceutical composition of claim 18, further comprising at least one additional agent useful for treating hepatitis infection.

20. The pharmaceutical composition of claim 19, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

21. The pharmaceutical composition of claim 20, wherein the immunostimulator is a checkpoint inhibitor, optionally wherein the checkpoint inhibitor is a PD-L1 inhibitor.

22. (canceled)

23. A method of treating, ameliorating, or preventing hepatitis B virus (HBV) infection in a subject, the method comprising administering to the subject in need thereof a therapeutically effective amount of at least one compound of claim 1, optionally wherein the compound is administered to the subject in a pharmaceutically acceptable composition.

24. The method of claim 23, wherein the subject is further infected with hepatitis D virus (HDV).

25. (canceled)

26. The method of claim 23, wherein the subject is further administered at least one additional agent useful for treating, ameliorating, or preventing the hepatitis B virus infection.

27. The method of claim 26, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

28. The method of claim 27, wherein the immunostimulator is a checkpoint inhibitor, optionally the checkpoint inhibitor is a PD-L1 inhibitor.

29. (canceled)

30. The method of claim 26, wherein the subject is co-administered the at least one compound and the at least one additional agent; optionally wherein the at least one compound and the at least one additional agent are coformulated.

31. (canceled)

32. A method of inhibiting expression or function of a viral capsid protein directly or indirectly in a hepatitis B virus-infected subject, the method comprising administering to the subject in need thereof a therapeutically effective amount of at least one compound of claim 1, optionally wherein the compound is administered to the subject in a pharmaceutically acceptable composition.

33. The method of claim 32, wherein the subject is further infected with hepatitis D virus (HDV).

34. (canceled)

35. The method of claim 32, wherein the subject is further administered at least one additional agent useful for treating the hepatitis B viral infection.

36. The method of claim 35, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript, and therapeutic vaccine.

37. The method of claim 36, wherein the immunostimulator is a checkpoint inhibitor, optionally wherein the checkpoint inhibitor is a PD-L1 inhibitor.

38. (canceled)

39. The method of claim 32, wherein the subject is co-administered the at least one compound and the at least one additional agent; optionally the at least one compound and the at least one additional agent are coformulated.

40. (canceled)

41. The method of claim 23, wherein the subject is a mammal, optionally wherein the mammal is a human.

42. (canceled)
